(19) United States
(12) Patent Application Publication
(10) Pub. No.: US 2024/0060046 A1
(43) Pub. Date: Feb. 22, 2024

(54) EXPANDED AND STIMULATED NATURAL KILLER CELLS

(71) Applicants: Artiva Biotherapeutics, Inc., San Diego, CA (US); GC Cell Corporation, Yongin-si, Gyeonggi-do (KR)

(72) Inventors: Seungryel Han, Yongin-si, Gyeonggi-do (KR); Bokyung Min, Yongin-si, Gyeonggi-do (KR); Sungyoo Cho, Yongin-si, Gyeonggi-do (KR); Yu Kyeong Hwang, Yongin-si, Gyeonggi-do (KR); Jung Hyun Her, Yongin-si, Gyeonggi-do (KR); Yusun Kim, Yongin-si, Gyeonggi-do (KR); Eunji Kim, Yongin-si, Gyeonggi-do (KR); Hyojin Kim, Yongin-si, Gyeonggi-do (KR); Bitna Yang, Yongin-si, Gyeonggi-do (KR); Peter Flynn, Cardiff by the Sea, CA (US); Jason B. Litten, Del Mar, CA (US); Thomas James Farrell, La Jolla, CA (US); John Kin Chuan Lim, San Diego, CA (US); Mili Mandal, San Diego, CA (US)

(21) Appl. No.: 18/268,167

(22) PCT Filed: Dec. 16, 2021
(86) PCT No.: PCT/US2021/063745

§ 371 Date: Jun. 16, 2023

Related U.S. Application Data
(60) Provisional application No. 63/172,417, filed on Apr. 8, 2021.
(60) Provisional application No. 63/127,098, filed on Dec. 17, 2020.

(51) Int. Cl. C12N 5/0783; A01N 1/02

(52) U.S. Cl. CPC C12N 5/0646; A01N 1/0226; A01N 1/0284; C12N 2501/25; C12N 2502/025

(57) ABSTRACT

Provided here, amongst other things, are populations of expanded and stimulated natural killer cells, pharmaceutical compositions comprising populations of expanded and stimulated natural killer cells, and methods of expanding and stimulating natural killer cells

### CLAIM OF PRIORITY

This application claims the benefit of U.S. Provisional Application Ser. No. 63/127,098, filed on Dec. 17, 2020, and U.S. Provisional Application Ser. No. 63/172,417, filed on Apr. 8, 2021. The entire contents of the foregoing are incorporated herein by reference.

### BACKGROUND

Targeted therapies, including antibody therapy, have revolutionized cancer treatment. One mechanism of action by which antibody therapy induces cytotoxicity is through antibody dependent cell-mediated cytotoxicity (ADCC). Many cancer patients are unable to mount a robust ADCC response. A reduced ADCC response may render any of the indicated monoclonal antibody therapeutics significantly less effective for these patients, which could prevent these patients from responding or lead to relapse. Thus, a reduced ADCC response could negatively impact their clinical outcomes.

Despite recent discoveries and developments of several anti-cancer agents, there is still a need for improved methods and therapeutic agents due to poor prognosis for many types of cancers.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). To initiate ADCC, NK cells engage with antibodies via the CD16 receptor on their surface. NK cells may have an advantage over other immune cells, such as the T cells used in CAR-T cell therapy and other cell therapies. In an exemplary advantage, NK cells can be used as allogeneic therapies, meaning that NK cells from one donor can be safely used in one or many patients without the requirement for HLA matching, gene editing, or other genetic manipulations. Allogeneic NK cells with anti-tumor activity can be administered safely to patients without many of the risks associated with T cell therapies, such as severe cytokine release syndrome (CRS), and neurological toxicities or graft versus host disease (GvHD).

Allogeneic NK cells may provide an important treatment option for cancer patients. In one exemplary advantage, NK cells have been well tolerated without evidence of graft-versus-host disease, neurotoxicity or cytokine release syndrome associated with other cell-based therapies. In another exemplary advantage, NK cells do not require prior antigen exposure or expression of a specific antigen to identify and lyse tumor cells. In another exemplary advantage, NK cells have the inherent ability to bridge between innate immunity and engender a multi-clonal adaptive immune response resulting in long-term anticancer immune memory. All of these features contribute to the potential for NK cell efficacy as cancer treatment options.

For example, NK cells can recruit and activate other components of the immune system. Activated NK cells secrete cytokines and chemokines, such as interferon gamma (IFNγ); tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system.

Additionally, cords with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

The administration of the allogenic NK cells, as described herein, can enhance patients' ADCC responses, e.g., when undergoing monoclonal antibody therapy.

Thus, described herein, are populations of expanded natural killer cells comprising a KIR-B haplotype and homozygous for a CD16 158V polymorphism.

In some embodiments, the expanded natural killer cells are expanded umbilical cord blood natural killer cells.

In some embodiments, the population of expanded natural killer cells comprises at least 60%, e.g., at least 70%, at least 80%, at least 90% at least 95%, at least 99%, or 100% CD16+ cells.

In some embodiments, the population of expanded natural killer cells comprises at least 60%, e.g., at least 70%, at least 80%, at least 90% at least 95%, at least 99%, or 100% NKG2D+ cells.

In some embodiments, the population of expanded natural killer cells comprises at least 60%, e.g., at least 70%, at least 80%, at least 90% at least 95%, at least 99%, or 100% NKp46+ cells.

In some embodiments, the population of expanded natural killer cells comprises at least 60%, e.g., at least 70%, at least 80%, at least 90% at least 95%, at least 99%, or 100% NKp30+ cells.

In some embodiments, the population of expanded natural killer cells comprises at least 60%, e.g., at least 70%, at least 80%, at least 90% at least 95%, at least 99%, or 100% DNAM-1+ cells.

In some embodiments, the population of expanded natural killer cells comprises at least 60%, e.g., at least 70%, at least 80%, at least 90% at least 95%, at least 99%, or 100% NKp44+ cells.

In some embodiments, the population of expanded natural killer cells comprises less than 20%, e.g., 10% or less, 5% or less, 1% or less, 0.5% or less, or 0% CD3+ cells.

In some embodiments, the population of expanded natural killer cells comprises less than 20% or less, e.g., 10% or less, 5% or less, 1% or less, 0.5% or less, or 0% CD14+ cells.

In some embodiments, the population of expanded natural killer cells comprises less than 20% or less, e.g., 10% or less, 5% or less, 1% or less, 0.5% or less, or 0% CD19+ cells.

In some embodiments, the population of expanded natural killer cells comprises less than 20% or less, e.g., 10% or less, 5% or less, 1% or less, 0.5% or less, or 0% CD38+ cells.

In some embodiments, the population of expanded natural killer cells do not comprise a CD16 transgene.

In some embodiments, the population of expanded natural killer cells do not express an exogenous CD16 protein.

In some embodiments, the expanded natural killer cells are not genetically engineered.

In some embodiments, the expanded natural killer cells are derived from the same umbilical cord blood donor.

In some embodiments, the population of expanded natural killer cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the population of expanded natural killer cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce expanded natural killer cells, thereby producing the population of expanded natural killer cells.

In some embodiments, the population of expanded natural killer cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and (d) expanding the master cell bank population of expanded natural killer cells by culturing with a second plurality of Hut78 cells engineered to express a membrane bound IL-21, a mutated TNFα, and a 4-1BBL gene to produce expanded natural killer cells, thereby producing the population of expanded natural killer cells.

In some embodiments, the method further comprises, after step (c), (i) freezing the master cell bank population of expanded natural killer cells in a plurality of containers; and (ii) thawing a container comprising an aliquot of the master cell bank population of expanded natural killer cells, wherein expanding the master cell bank population of expanded natural killer cells in step (d) comprises expanding the aliquot of the master cell bank population of expanded natural killer cells.

In some embodiments, the umbilical cord blood is from a donor with the KIR-B haplotype and homozygous for the CD16 158V polymorphism.

In some embodiments, the method comprises expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold.

In some embodiments, the population of expanded natural killer cells is not enriched or sorted after expansion.

In some embodiments, the percentage of NK cells expressing CD16 in the population of expanded natural killer cells is the same or higher than the percentage of natural killer cells in the seed cells from umbilical cord blood.

In some embodiments, the percentage of NK cells expressing NKG2D in the population of expanded natural killer cells is the same or higher than the percentage of natural killer cells in the seed cells from umbilical cord blood.

In some embodiments, the percentage of NK cells expressing NKp30 in the population of expanded natural killer cells is the same or higher than the percentage of natural killer cells in the seed cells from umbilical cord blood.

In some embodiments, the percentage of NK cells expressing NKp44 in the population of expanded natural killer cells is the same or higher than the percentage of natural killer cells in the seed cells from umbilical cord blood.

In some embodiments, the percentage of NK cells expressing NKp46 in the population of expanded natural killer cells is the same or higher than the percentage of natural killer cells in the seed cells from umbilical cord blood.

In some embodiments, the percentage of NK cells expressing DNAM-1 in the population of expanded natural killer cells is the same or higher than the percentage of natural killer cells in the seed cells from umbilical cord blood.

Also described herein is a vial or cryobag comprising a portion of a population of expanded natural killer cells described herein.

Also described herein is a plurality of vials or cryobags comprising portions of the population of expanded natural killer cells described herein.

In some embodiments, the plurality of vials or cryobags comprises at least 10 vials or cryobags comprising portions of the population of expanded natural killer cells, e.g., 20, 30, 40, 50, 60, 70, 80, 90, 100, 125, 150, 175, 200, 250, 300, 400, 500, 600, 700, 800, 900, 1000, 1100, or 1200 vials or cryobags.

Also described herein is a bioreactor comprising a population of expanded natural killer cells described herein.

Also provided herein are compositions comprising a population of expanded and stimulated natural killer cells described herein; and a cryopreservation solution.

In some embodiments, the cryopreservation solution comprises (a) human albumin; (b) dextran; (c) glucose; (d) DMSO; and (e) a buffer.

In some embodiments, the composition comprises from 30 to 50 mg/mL human albumin.

In some embodiments, the composition comprises 50 mg/mL human albumin.

In some embodiments, the composition comprises 20 to 30 mg/mL dextran.

In some embodiments, the composition comprises 25 mg/mL dextran.

In some embodiments, the dextran is Dextran 40.

In some embodiments, the composition comprises from 12 to 15 mg/mL glucose.

In some embodiments, the composition comprises 12.5 mg/mL glucose.

In some embodiments, the composition comprises less than 27.5 g/L glucose.

In some embodiments, the composition comprises from 50 to 60 ml/mL DMSO.

In some embodiments, the composition comprises 55 mg/mL DMSO.

In some embodiments, the composition comprises 40 to 60% v/v buffer.

In some embodiments, the buffer is phosphate buffered saline.

In some embodiments, the composition comprises (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline.

In some embodiments, the composition further comprises 0.5 mL/mL water.

In some embodiments, the cryopreservation solution is an infusion-ready cryopreservation solution.

In some embodiments, the composition further comprises at least one of genetic material, protein, or cells from a feeder cell line.

In some embodiments, the genetic material from the feeder cell line comprises a nucleic acid encoding a membrane bound IL-21 molecule or a portion thereof.

In some embodiments, the membrane bound IL-21 comprises a CD8 transmembrane domain.

In some embodiments, the genetic material from the feeder cell line that comprises a nucleic acid encoding a membrane bound IL-21 molecule or a portion thereof encodes SEQ ID NO. 11 or a portion thereof.

In some embodiments, the genetic material from the feeder cell line comprises a nucleic acid encoding a mutated TNFα molecule or a portion thereof.

In some embodiments, the genetic material from the feeder cell line that comprises a nucleic acid encoding a mutated TNFα molecule or a portion thereof encodes SEQ ID NO: 12 or a portion thereof.

In some embodiments, the protein from the feeder cell line comprises a membrane bound IL-21 polypeptide or a portion thereof.

In some embodiments, the membrane bound IL-21 comprises a CD8 transmembrane domain.

In some embodiments, the protein from the feeder cell line that comprises a membrane bound IL-21 polypeptide or a portion thereof comprises SEQ ID NO: 11 or a portion thereof.

In some embodiments, the protein from the feeder cell line comprises a mutated TNFα polypeptide or a portion thereof.

In some embodiments, the protein from the feeder cell line that comprises a mutated TNFα polypeptide or a portion thereof comprises SEQ ID NO: 12 or a portion thereof.

In some embodiments, the cells from the feeder cell line are CD4+ T cells.

In some embodiments, the feeder cell line are Hut78 cells.

In some embodiments, the cells from the Hut78 cells are engineered Hut78 (eHut78) cells express 4-1BBL, membrane bound IL-21 and mutant TNFα.

In some embodiments, the cells from the feeder cell line comprise live cells.

In some embodiments, the cells from the feeder cell line comprise dead cells.

In some embodiments, the composition is frozen.

In some embodiments, the pharmaceutical composition has been frozen for at least three months, e.g., at least six months, at least nine months, at least 12 months, at least 15 months, at least 18 months, at least 24 months, or at least 36 months.

In some embodiments, the population of expanded natural killer cells exhibits at least 60%, e.g., at least 70%, at least 80%, at least 90% at least 95%, at least 99%, or 100% viability after it is thawed.

Also described herein are pharmaceutical composition(s) comprising the compositions described herein.

Also described herein are dosage unit(s) comprising the pharmaceutical composition of claim 70.

In some embodiments, the dosage comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

A composition comprising a population of expanded cord blood-derived natural killer cells comprising a KIR-B haplotype and homozygous for a CD16 158V polymorphism and a plurality of engineered HuT78 cells. Provided here, amongst other things, are populations of ex vivo expanded and stimulated natural killer cells, pharmaceutical compositions comprising populations of expanded and stimulated natural killer cells, and methods of expanding and stimulating natural killer cells.

Provided herein is a population of expanded and stimulated natural killer cells comprising at least 80%, e.g., at least 90%, at least 95%, at least 99%, or 100% CD56+CD3-cells.

In some embodiments, the expanded and stimulated natural killer cells comprise at least 60%, e.g., at least 70%, at least 80%, at least 90% at least 95%, at least 99%, or 100% NKG2D+ cells.

In some embodiments, the expanded and stimulated natural killer cells comprise at least 60%, e.g., at least 70%, at least 80%, at least 90% at least 95%, at least 99%, or 100% NKp46+ cells.

In some embodiments, the expanded and stimulated natural killer cells comprise at least 60%, e.g., at least 70%, at least 80%, at least 90% at least 95%, at least 99%, or 100% NKp30+ cells.

In some embodiments, the expanded and stimulated natural killer cells comprise at least 60%, e.g., at least 70%, at least 80%, at least 90% at least 95%, at least 99%, or 100% DNAM-1+ cells.

In some embodiments, the expanded and stimulated natural killer cells comprise at least 60%, e.g., at least 70%, at least 80%, at least 90% at least 95%, at least 99%, or 100% NKp44+ cells.

In some embodiments, the expanded and stimulated natural killer cells comprise 20% or less, e.g., 10% or less, 5% or less, 1% or less, or 0% CD3+ cells.

In some embodiments, the expanded and stimulated natural killer cells comprise 20% or less, e.g., 10% or less, 5% or less, 1% or less, or 0% CD14+ cells.

In some embodiments, the expanded and stimulated natural killer cells comprise 20% or less, e.g., 10% or less, 5% or less, 1% or less, or 0% CD19+ cells.

Also disclosed herein are pharmaceutical compositions comprising these NK cells such as expanded and stimulated NK cells. Some such pharmaceutical compositions any one or more of the populations of expanded and stimulated natural killer cells. Some of such compositions further comprise an infusion-ready cryopreservation solution, which in some cases serves to provide the pharmaceutical compositions with an added functionality of being resistant to cell death upon freeze-thaw cycles, and being capable of direct administration to a patient upon thawing, such that the thawed cells do not need to be further purified away from their cryoprotectant prior to administration to a patient or other user.

Also described herein are methods of expanding and stimulating natural killer cells, comprising: (a) co-culturing a source of natural killer cells and feeder cells to produce a master cell bank (MCB); and (b) co-culturing cells of the MCB with feeder cells to produce expanded and stimulated natural killer cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative and are not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a method for NK cell expansion and stimulation.

FIG. 2 shows that cord blood-derived NK cells (CB-NK) have an approximately ten-fold greater ability to expand in culture than peripheral blood-derived NK cells (PB-NK) in preclinical studies.

FIG. 3 shows that expression of tumor-engaging NK activating immune receptors was higher and more consistent in cord blood-derived drug product compared to that generated from peripheral blood.

FIG. 4 shows phenotypes of expanded and stimulated population of NK cells.

FIG. 5 shows key steps in the manufacture of the AB-101 drug product, which is an example of a cord blood-derived and expanded population of NK cells.

FIG. 6 shows the purity of AB-101 (n=9).

FIG. 7 shows purity of CD3 depleted cells, MCB and DP manufactured in GMP conditions.

FIG. 8 shows expression of NK cell receptors on CD3 depleted cells, MCB and DP manufactured in GMP conditions.

FIG. 9 shows direct cytotoxicity of AB-101 against K562 cells (n=9).

FIG. 10 shows direct cytotoxicity of AB-101 against Ramos cells (n=9).

FIG. 11 shows long-term ADCC of AB-101 in combination with Rituximab against Ramos cells (n=9).

FIG. 12 shows long-term ADCC of AB-101 in combination with Rituximab against Ramos cells (n=9).

FIG. 13 shows long-term ADCC of AB-101 in combination with Rituximab against Raji cells (n=9).

FIG. 14 shows long-term ADCC of AB-101 in combination with Rituximab against Raji cells (n=9).

FIG. 15 shows Cytokine production and CD107a expression of AB-101 against K562 (n=9).

FIG. 16 shows Cytokine production and CD107a expression of AB-101 against Ramos cells (n=9).

FIG. 17 shows Cytokine production and CD107a expression of AB-101 against Raji cells (n=8).

FIG. 18 shows direct cytolytic activity of AB-101, which was assessed by calcein-acetoxymethyl (AM) release assay using target cells K562 (top panels), Ramos (middle panels) and Raji (bottom panels) at an effector-to-target ratios (E:T) of 10:1 to 0.3:1. Data shown is representative of cytolytic activity of seven AB-101 engineering lots (left panels) and two AB-101 GMP lots (right panels).

FIG. 19 shows ADCC of tumor cells by AB-101 assessed by Incucyte S3 live cell-analysis system using target cells Ramos-NucLight (left) and Raji (right) at a 1:1 effector-to-target ratio (E:T). Data shown is representative of cytolytic activity of seven AB-101 engineering lots.

FIG. 20 shows intracellular levels of cytokines (left four panels) and levels of degranulation marker (CD107a) (right two panels) expressed by AB-101, as assessed by flow cytometry following co-incubation with various tumor cells, K562, Ramos, and Raji, or without co-incubation (AB-101 alone). Data are shown as mean percent of AB-101 cells (±s.e.m.) positive for cytokines and CD107a. Data is representative of seven AB-101 engineering lots (top panels and two AB-101 GMP lots (bottom panels).

FIG. 21 shows the dosing schedule for in vivo efficacy of AB-101 in Ramos lymphoma model. SCID mouse transplanted with the Ramos cell line were administered one of the following treatments: vehicle+IgG, rituximab alone, AB-101 alone, or AB-101 plus rituximab. A total of 6 doses of AB-101 and 6 doses of rituximab was given to each mouse.

FIG. 22 shows Kaplan Meier survival curve representative of % survival rate in each group of the Ramos lymphoma model. Data shown is representative of one of three independent experiments; the p-value of difference was calculated with the log-rank test.

FIG. 23 shows Kaplan Meier survival curve representative of % tumor-associated paralysis free mice in each group of the Ramos lymphoma model. Data shown is representative of one of three independent experiments; the p-value of difference was calculated with the log-rank test.

FIG. 24 shows the dosing schedule for in vivo efficacy of AB-101 in Raji lymphoma model. SCID mouse transplanted with the Raji cell line were administered one of the following treatments: vehicle+IgG, rituximab alone, AB-101 alone, or AB-101 plus rituximab. A total of 6 doses of AB-101 and 1 dose of rituximab was given to each mouse.

FIG. 25 shows Kaplan Meier survival curve representative of % survival rate in each group of the Raji lymphoma model. Data shown is representative of one of three independent experiments; the p-value of difference was calculated with the log-rank test.

FIG. 26 shows Kaplan Meier survival curve representative of % tumor-associated paralysis free mice in each group of the Raji lymphoma model. Data shown is representative of one of three independent experiments; the p-value of difference was calculated with the log-rank test.

FIG. 27 shows distribution of AB-101 in several tissues of NSG mouse as determined by calculating amount of AB-101 DNA per μg of mouse blood/tissue DNA. Data are shown as mean concentration (±s.e.m.) of AB-101 DNA in each organ and is representative of 6 mice (3 male, 3 female) per each timepoint.

FIG. 28 shows that CAR-NKs comprising a co-stimulatory domain comprising OX40L exhibited greater cytotoxic potential than those without OX40L.

FIG. 29 depicts a Plate Map of Short-Term Cytotoxicity.

FIG. 30 depicts a Plate map of Long-Term Killing.

FIG. 31 depicts Plate map of in vitro intracellular cytokine staining.

FIG. 32 shows NK purity (CD56+/CD3−) by flow cytometry.

FIG. 33 shows CD38+ expression of expanded NK cells from three different cord blood donors.

FIG. 34 shows CD38+ mean fluorescence intensity of CD38+ NK cells from three different cord blood donors.

FIG. 35 shows differential gene expression patterns between cord blood natural killer cells and AB-101 cells.

FIG. 36 shows differential gene expression patterns between peripheral blood natural killer cells and AB-101 cells.

FIG. 37 shows differential surface protein expression of starting NK cell source compared to AB-101 cells.

FIG. 38 shows differential expression of genes encoding surface proteins between KIR-B/158 v/v selected, CD56+CD3-gated cord blood NK cells (Cord Blood NK D0) and AB-101 cells.

FIG. 39 shows differential expression of genes encoding surface proteins between unselected cord blood NK cells (Cord Blood NK) and AB-101 cells.

FIG. 40 shows differential expression of genes encoding surface proteins between the cord blood NK cells (average of KIR-B/158 v/v selected, CD56+CD3-gated cord blood NK cells and unselected cord blood NK cells and average of AB-101 samples).

FIG. 41 shows FACs sorting of eHuT-78 cells.

FIG. 42 shows FACs sorting of eHuT-78 cells.

FIG. 43 shows FACs sorting of eHuT-78 cells.

FIG. 44 shows portions of eHuT-78 transgenic sequences detected in a qPCR assay.

FIG. 45 shows primer positions for amplifying portions of eHuT-78 transgenic sequences in a qPCR assay.

### DETAILED DESCRIPTION

Provided herein are, amongst other things, Natural Killer (NK) cells, e.g., expanded and stimulated NK cells, methods for producing the NK cells, pharmaceutical compositions comprising the NK cells, and methods of treating patients suffering, e.g., from cancer, with the NK cells.

### I. Expansion and Stimulation of Natural Killer Cells

In some embodiments, natural killer cells are expanded and stimulated, e.g., by culturing and stimulation with feeder cells.

NK cells can be expanded and stimulated as described, for example, in US 2020/0108096 or WO 2020/101361, both of which are incorporated herein by reference in their entirety. Briefly, the source cells can be cultured on modified HuT-78 (ATCC® TIB-161™) cells that have been engineered to express 4-1BBL, membrane bound IL-21, and a mutant TNFα as described in US 2020/0108096.

Suitable NK cells can also be expanded and stimulated as described herein.

In some embodiments, NK cells are expanded and stimulated by a method comprising: (a) providing NK cells, e.g., a composition comprising NK cells, e.g., CD3(+) depleted cells; and (b) culturing in a medium comprising feeder cells and/or stimulation factors, thereby producing a population of expanded and stimulated NK cells.

A. Natural Killer Cell Sources

In some embodiments, the NK cell source is selected from the group consisting of peripheral blood, peripheral blood lymphocytes (PBLs), peripheral blood mononuclear cells (PBMCs), bone marrow, umbilical cord blood (cord blood), isolated NK cells, NK cells derived from induced pluripotent stem cells, NK cells derived from embryonic stem cells, and combinations thereof.

In some embodiments, the NK cell source is a single unit of cord blood.

In some embodiments, the natural killer cell source, e.g., single unit of cord blood, comprises from or from about 1×10⁷ to or to about 1×10⁹ total nucleated cells. In some embodiments, the natural killer cell source, e.g., single unit of cord blood, comprises from or from about 1×10⁸ to or to about 1.5×10⁸ total nucleated cells. In some embodiments, the natural killer cell source, e.g., single unit of cord blood, comprises 1×10⁸ total nucleated cells. In some embodiments, the natural killer cell source, e.g., single unit of cord blood, comprises about 1×10⁸ total nucleated cells. In some embodiments, the natural killer cell source, e.g., single unit of cord blood, comprises 1×10⁹ total nucleated cells. In some embodiments, the natural killer cell source, e.g., single unit of cord blood, comprises about 1×10⁹ total nucleated cells.

In some embodiments, the NK cell source, e.g., the cord blood unit, comprises from about 20% to about 80% CD16+ cells. In some embodiments, the NK cell source, e.g., the cord blood unit, comprises from or from about 20% to or to about 80%, from about 20% to or to about 70%, from about 20% to or to about 60%, from about 20% to or to about 50%, from about 20% to or to about 40%, from about 20% to or to about 30%, from about 30% to or to about 80%, from about 30% to or to about 70%, from about 30% to or to about 60%, from about 30% to or to about 50%, from about 30% to or to about 40%, from about 40% to or to about 80%, from about 40% to or to about 70%, from about 40% to or to about 60%, from about 40% to or to about 50%, from about 50% to or to about 80%, from about 50% to or to about 70%, from about 50% to or to about 60%, from about 60% to or to about 80%, from about 60% to or to about 70%, or from about 70% to or to about 80% CD16+ cells. In some embodiments, the NK cell source, e.g., the cord blood unit, comprises less than or equal to 80% CD16+ cells. Alternately, some NK cell sources may comprise CD16+ cells at a concentration of greater than 80%.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% MLG2A+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2C+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2D+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp46+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp30+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% DNAM-1+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp44+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD25+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD62L+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD69+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CXCR3+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD57+ cells.

In some embodiments, NK cells in the NK cell source comprise a KIR B allele of the KIR receptor family. See, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5:e15115 (2010).

In some embodiments, NK cells in the NK cell source comprise the 158 v/V variant of CD16 (i.e. homozygous CD16 158V polymorphism). See, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).

In some embodiments, NK cells in the cell source comprises both the KIR B allele of the KIR receptor family and the 158 V/V variant of CD16.

In some embodiments, the NK cells in the cell source are not genetically engineered.

In some embodiments, the NK cells in the cell source do not comprise a CD16 transgene.

In some embodiments, the NK cells in the cell source do not express an exogenous CD16 protein.

In some embodiments, the NK cell source is CD3(+) depleted. In some embodiments, the method comprises depleting the NK cell source of CD3(+) cells. In some embodiments, depleting the NK cell source of CD3(+) cells comprises contacting the NK cell source with a CD3 binding antibody or antigen binding fragment thereof. In some embodiments, the CD3 binding antibody or antigen binding fragment thereof is selected from the group consisting of OKT3, UCHT1, and HIT3a, and fragments thereof. In some embodiments, the CD3 binding antibody or antigen binding fragment thereof is OKT3 or an antigen binding fragment thereof. In some embodiments, the antibody or antigen binding fragment thereof is attached to a bead, e.g., a magnetic bead. In some embodiments, the depleting the composition of CD3(+) cells comprises contacting the composition with a CD3 targeting antibody or antigen binding fragment thereof attached to a bead and removing the bead-bound CD3(+) cells from the composition. The composition can be depleted of CD3 cells by immunomagnetic selection, for example, using a CliniMACS T cell depletion set ((LS Depletion set (162-01) Miltenyi Biotec).

In some embodiments, the NK cell source CD56+ enriched, e.g., by gating on CD56 expression.

In some embodiments, the NK cell source is both CD56+ enriched and CD3(+) depleted, e.g., by selecting for cells with CD56+CD3-expression.

In some embodiments, the NK cell source comprises both the KIR B allele of the KIR receptor family and the 158 V/V variant of CD16 and is + enriched and CD3(+) depleted, e.g., by selecting for cells with CD56+CD3-expression.

B. Feeder Cells

Disclosed herein are feeder cells for the expansion of NK cells. These feeder cells advantageously allow NK cells to expand to numbers suitable for the preparation of a pharmaceutical composition as discussed herein. In some cases, the feeder cells allow the expansion of NK cells without the loss of CD16 expression, which often accompanies cell expansion on other types of feeder cells or using other methods. In some cases, the feeder cells make the expanded NK cells more permissive to freezing such that a higher proportion of NK cells remain viable after a freeze/thaw cycle or such that the cells remain viable for longer periods of time while frozen. In some cases, the feeder cells allow the NK cells to retain high levels of cytotoxicity, including ADCC, extend survival, increase persistence, and enhance or retain high levels of CD16. In some cases, the feeder cells allow the NK cells to expand without causing significant levels of exhaustion or senescence.

Feeder cells can be used to stimulate the NK cells and help them to expand more quickly, e.g., by providing substrate, growth factors, and/or cytokines.

NK cells can be stimulated using various types of feeder cells, including, but not limited to peripheral blood mononuclear cells (PBMC), Epstein-Barr virus-transformed B-lymphoblastoid cells (e.g., EBV-LCL), myelogenous leukemia cells (e.g., K562), and CD4(+) T cells (e.g., HuT), and derivatives thereof.

In some embodiments, the feeder cells are inactivated, e.g., by γ-irradiation or mitomycin-c treatment.

Suitable feeder cells for use in the methods described herein are described, for example, in US 2020/0108096, which is hereby incorporated by reference in its entirety.

In some embodiments, the feeder cell(s) are inactivated CD4(+) T cell(s). In some embodiments, the inactivated CD4(+) T cell(s) are HuT-78 cells (ATCC® TIB-161™) or variants or derivatives thereof. In some embodiments, the HuT-78 derivative is H9 (ATCC® HTB-176™).

In some embodiments, the inactivated CD4(+) T cell(s) express OX40L. In some embodiments, the inactivated CD4(+) T cell(s) are HuT-78 cells or variants or derivatives thereof that express OX40L (SEQ ID NO: 13) or a variant thereof.

In some embodiments, the feeder cells are HuT-78 cells engineered to express at least one gene selected from the group consisting of 4-1BBL (UniProtKB P41273, SEQ ID NO: 10), membrane bound IL-21 (SEQ ID NO: 11), and mutant TNFalpha (SEQ ID NO: 12) (“eHut-78 cells”), or variants thereof.

In some embodiments, the inactivated CD4(+) T cell(s) are HuT-78 (ATCC® TIB-161™) cells or variants or derivatives thereof that express an ortholog of OX40L, or variant thereof. In some embodiments, the feeder cells are HuT-78 cells engineered to express at least one gene selected from the group consisting of an 4-1BBL ortholog or variant thereof, a membrane bound IL-21 ortholog or variant thereof, and mutant TNFalpha ortholog, or variant thereof.

In some embodiments, the feeder cells are HuT-78 cell(s) that express OX40L (SEQ ID NO: 13) and are engineered to express 4-1BBL (SEQ ID NO: 10), membrane bound 1L-21 (SEQ ID NO: 11), and mutant TNFalpha (SEQ ID NO: 12) (“eHut-78 cells”) or variants or derivatives thereof.

In some embodiments, the feeder cells are expanded, e.g., from a frozen stock, before culturing with NK cells, e.g., as described in Example 2.

C. Stimulating Factors

NK cells can also be stimulated using one or more stimulation factors other than feeder cells, e.g., signaling factors, in addition to or in place of feeder cells.

In some embodiments, the stimulating factor, e.g., signaling factor, is a component of the culture medium, as described herein. In some embodiments, the stimulating factor, e.g., signaling factor, is a supplement to the culture medium, as described herein.

In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-2.

In some embodiments, the cytokines are a combination of IL-2 and IL-15.

In some embodiments, the cytokines are a combination of IL-2, IL-15, and IL-18.

In some embodiments, the cytokines are a combination of IL-2, IL-18, and IL-21.

D. Culturing

The NK cells can be expanded and stimulated by co-culturing an NK cell source and feeder cells and/or other stimulation factors. Suitable NK cell sources, feeder cells, and stimulation factors are described herein.

In some cases, the resulting population of expanded natural killer cells is enriched and/or sorted after expansion. In some cases, the resulting population of expanded natural killer cells is not enriched and/or sorted after expansion

Also described herein are compositions comprising the various culture compositions described herein, e.g., comprising NK cells. For example, a composition comprising a population of expanded cord blood-derived natural killer cells comprising a KIR-B haplotype and homozygous for a CD16 158V polymorphism and a plurality of engineered HuT78 cells.

Also described herein are vessels, e.g., vials, cryobags, and the like, comprising the resulting populations of expanded natural killer cells. In some cases, a plurality of vessels comprising portions of the resulting populations of expanded natural killer cells, e.g., at least 10, e.g., 20, 30, 40, 50, 60, 70, 80, 90, 100, 125, 150, 175, 200, 250, 300, 400, 500, 600, 700, 800, 900, 1000, 1100, or 1200 vessels.

Also described herein are bioreactors comprising the various culture compositions described herein, e.g., comprising NK cells. For example, a culture comprising natural killer cells from a natural killer cell source, e.g., as described herein, and feeder cells, e.g., as described herein. Also described herein are bioreactors comprising the resulting populations of expanded natural killer cells.

1. Culture Medium

Disclosed herein are culture media for the expansion of NK cells. These culture media advantageously allow NK cells to expand to numbers suitable for the preparation of a pharmaceutical composition as discussed herein. In some cases, the culture media allows NK cells to expand without the loss of CD16 expression that often accompanies cell expansion on other helper cells or in other media.

In some embodiments, the culture medium is a basal culture medium, optionally supplemented with additional components, e.g., as described herein.

In some embodiments, the culture medium, e.g., the basal culture medium, is a serum-free culture medium. In some embodiments, the culture medium, e.g., the basal culture medium, is a serum-free culture medium supplemented with human plasma and/or serum.

Suitable basal culture media include, but are not limited to, DMEM, RPMI 1640, MEM, DMEM/F12, SCGM (CellGenix®, 20802-0500 or 20806-0500), LGM-3™ (Lonza, CC-3211), TexMACS™ (Miltenyi Biotec, 130-097-196), ALyS™ 505NK-AC (Cell Science and Technology Institute, Inc., 01600P02), ALyS™ 505NK-EX (Cell Science and Technology Institute, Inc., 01400P10), CTS™ AIM-VM SFM (ThermoFisher Scientific, A3830801), CTS™ OpTmizer™ (ThermoFisher Scientific, A1048501, ABS-001, StemXxVivoand combinations thereof.

The culture medium may comprise additional components, or be supplemented with additional components, such as growth factors, signaling factors, nutrients, antigen binders, and the like. Supplementation of the culture medium may occur by adding each of the additional component or components to the culture vessel either before, concurrently with, or after the medium is added to the culture vessel. The additional component or components may be added together or separately. When added separately, the additional components need not be added at the same time.

In some embodiments, the culture medium comprises plasma, e.g., human plasma. In some embodiments, the culture medium is supplemented with plasma, e.g., human plasma. In some embodiments, the plasma, e.g., human plasma, comprises an anticoagulant, e.g., trisodium citrate.

In some embodiments, the medium comprises and/or is supplemented with from or from about 0.5% to or to about 10% v/v plasma, e.g., human plasma. In some embodiments, the medium is supplemented with from or from about 0.5% to or to about 9%, from or from about 0.5% to or to about 8%, from or from about 0.5% to or to about 7%, from or from about 0.5% to or to about 6%, from or from about 0.5% to or to about 5%, from or from about 0.5% to or to about 4%, from or from about 0.5% to or to about 3%, from or from about 0.5% to or to about 2%, from or from about 0.5% to or to about 1%, from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 2% to or to about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 4% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v plasma, e.g., human plasma. In some embodiments, the culture medium comprises and/or is supplemented with from 0.8% to 1.2% v/v human plasma. In some embodiments, the culture medium comprises and/or is supplemented with 1.0% v/v human plasma. In some embodiments, the culture medium comprises and/or is supplemented with about 1.0% v/v human plasma.

In some embodiments, the culture medium comprises serum, e.g., human serum. In some embodiments, the culture medium is supplemented with serum, e.g., human serum. In some embodiments, the serum is inactivated, e.g., heat inactivated. In some embodiments, the serum is filtered, e.g., sterile-filtered.

In some embodiments, the culture medium comprises glutamine. In some embodiments, the culture medium is supplemented with glutamine. In some embodiments, the culture medium comprises and/or is supplemented with from or from about 2.0 to or to about 6.0 mM glutamine. In some embodiments, the culture medium comprises and/or is supplemented with from or from about 2.0 to or to about 5.5, from or from about 2.0 to or to about 5.0, from or from about 2.0 to or to about 4.5, from or from about 2.0 to or to about 4.0, from or from about 2.0 to or to about 3.5, from or from about 2.0 to or to about 3.0, from or from about 2.0 to or to about 2.5, from or from about 2.5 to or to about 6.0, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3.0 to or to about 6.0, from or from about 3.0 to or to about 5.5, from or from about 3.0 to or to about 5.0, from or from about 3.0 to or to about 4.5, from or from about 3.0 to or to about 4.0, from or from about 3.0 to or to about 3.5, from or from about 3.5 to or to about 6.0, from or from about 3.5 to or to about 5.5, from or from about 3.5 to or to about 5.0, from or from about 3.5 to or to about 4.5, from or from about 3.5 to or to about 4.0, from or from about 4.0 to or to about 6.0, from or from about 4.0 to or to about 5.5, from or from about 4.0 to or to about 5.0, from or from about 4.0 to or to about 4.5, from or from about 4.5 to or to about 6.0, from or from about 4.5 to or to about 5.5, from or from about 4.5 to or to about 5.0, from or from about 5.0 to or to about 6.0, from or from about 5.0 to or to about 5.5, or from or from about 5.5 to or to about 6.0 mM glutamine. In some embodiments, the culture medium comprises and/or is supplemented with from 3.2 mM glutamine to 4.8 mM glutamine. In some embodiments, the culture medium comprises and/or is supplemented with 4.0 mM glutamine. In some embodiments, the culture medium comprises and/or is supplemented with about 4.0 mM glutamine.

In some embodiments, the culture medium comprises one or more cytokines. In some embodiments, the culture medium is supplemented with one or more cytokines.

In some embodiments, the cytokine is selected from IL-2, IL-12, IL-15, IL-18, and combinations thereof.

In some embodiments, the culture medium comprises and/or is supplemented with IL-2. In some embodiments, the culture medium comprises and/or is supplemented with from or from about 150 to or to about 2,500 IU/mL IL-2. In some embodiments, the culture medium comprises and/or is supplemented with from or from about 200 to or to about 2,250, from or from about 200 to or to about 2,000, from or from about 200 to or to about 1,750, from or from about 200 to or to about 1,500, from or from about 200 to or to about 1,250, from or from 200 to or to about 1,000, from or from about 200 to or to about 750, from or from about 200 to or to about 500, from or from about 200 to or to about 250, from or from about 250 to or to about 2,500, from or from about 250 to or to about 2,250, from or from about 250 to or to about 2,000, from or from about 250 to or to about 1,750, from or from about 250 to or to about 1,500, from or from about 250 to or to about 1,250, from or from about 250 to or to about 1,000, from or from about 250 to or to about 750, from or from about 250 to or to about 500, from or from about 500 to or to about 2,500, from or from about 500 to or to about 2,250, from or from about 500 to or to about 2,000, from or from about 500 to or to about 1,750, from or from about 500 to or to about 1,500, from or from about 500 to or to about 1,250, from or from about 500 to or to about 1,000, from or from about 500 to or to about 750, from or from about 750 to or to about 2,250, from or from about 750 to or to about 2,000, from or from about 750 to or to about 1,750, from or from about 750 to or to about 1,500, from or from about 750 to or to about 1,250, from or from about 750 to or to about 1,000, from or from about 1,000 to or to about 2,500, from or from about 1,000 to or to about 2,250, from or from about 1,000 to or to about 2,000, from or from about 1,000 to or to about 1,750, from or from about 1,000 to or to about 1,500, from or from about 1,000 to or to about 1,250, from or from about 1,250 to or to about 2,500, from or from about 1,250 to or to about 2,250, from or from about 1,250 to or to about 2,000, from or from about 1,250 to or to about 1,750, from or from about 1,250 to or to about 1,500, from or from about 1,500 to or to about 2,500, from or from about 1,500 to or to about 2,250, from or from about 1,500 to or to about 2,000, from or from about 1,500 to or to about 1,750, from or from about 1,750 to or to about 2,500, from or from about 1,750 to or to about 2,250, from or from about 1,750 to or to about 2,000, from or from about 2,000 to or to about 2,500, from or from about 2,000 to or to about 2,250, or from or from about 2,250 to or to about 2,500 IU/mL IL-2.

In some embodiments, the culture medium comprises and/or is supplemented with from 64 μg/L to 96 μg/L IL-2. In some embodiments, the culture medium comprises and/or is supplemented with 80 μg/L IL-2 (approximately 1,333 IU/mL). In some embodiments, the culture medium comprises and/or is supplemented with about 80 μg/L.

In some embodiments, the culture medium comprises and/or is supplemented with a combination of IL-2 and IL-15.

In some embodiments, the culture medium comprises and/or is supplemented with a combination of IL-2, IL-15, and IL-18.

In some embodiments, the culture medium comprises and/or is supplemented with a combination of IL-2, IL-18, and IL-21.

In some embodiments, the culture medium comprises and/or is supplemented with glucose. In some embodiments, the culture medium comprises and/or is supplemented with from or from about 0.5 to or to about 3.5 g/L glucose. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L glucose. In some embodiments, the culture medium comprises and/or is supplemented with from 1.6 to 2.4 g/L glucose. In some embodiments, the culture medium comprises and/or is supplemented with 2.0 g/L glucose. In some embodiments, the culture medium comprises about 2.0 g/L glucose.

In some embodiments, the culture medium comprises and/or is supplemented with sodium pyruvate. In some embodiments, the culture medium comprises and/or is supplemented with from or from about 0.1 to or to about 2.0 mM sodium pyruvate. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 mM sodium pyruvate. In some embodiments, the culture medium comprises from 0.8 to 1.2 mM sodium pyruvate. In some embodiments, the culture medium comprises 1.0 mM sodium pyruvate. In some embodiments, the culture medium comprises about 1.0 mM sodium pyruvate.

In some embodiments, the culture medium comprises and/or is supplemented with sodium hydrogen carbonate. In some embodiments, the culture medium comprises and/or is supplemented with from or from about 0.5 to or to about 3.5 g/L sodium hydrogen carbonate. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L sodium hydrogen carbonate. In some embodiments, the culture medium comprises and/or is supplemented with from 1.6 to 2.4 g/L sodium hydrogen carbonate. In some embodiments, the culture medium comprises and/or is supplemented with 2.0 g/L sodium hydrogen carbonate. In some embodiments, the culture medium comprises about 2.0 g/L sodium hydrogen carbonate.

In some embodiments, the culture medium comprises and/or is supplemented with albumin, e.g., human albumin, e.g., a human albumin solution described herein. In some embodiments, the culture medium comprises and/or is supplemented with from or from about 0.5% to or to about 3.5% v/v of a 20% albumin solution, e.g., a 20% human albumin solution. In some embodiments, the culture medium comprises and/or is supplemented with from or from about 0.5% to or to about 3.0%, from or from about 0.5% to or to about 2.5%, from or from about 0.5% to or to about 2.0%, from or from about 0.5% to or to about 1.5%, from or from about 0.5% to or to about 1.0%, from or from about 1.0% to or to about 3.0%, from or from about 1.0% to or to about 2.5%, from or from about 1.0% to or to about 2.0%, from or from about 1.0% to or to about 1.5%, from or from about 1.5% to or to about 3.0%, from or from about 1.5% to or to about 2.5%, from or from about 1.5% to or to about 2.0%, from or from about 2.0% to or to about 3.0%, from or from about 2.0% to or to about 2.5%, or from or from about 2.5% to or to about 3.0% v/v of a 20% albumin solution, e.g., a 20% human albumin solution. In some embodiments, the culture medium comprises and/or is supplemented with from 1.6% to 2.4% v/v of a 20% albumin solution, e.g., a 20% human albumin solution. In some embodiments, the culture medium comprises and/or is supplemented with 2.0% v/v of a²00/% albumin solution, e.g., a 20% human albumin solution. In some embodiments, the culture medium comprises about 2.0% v/v of a 20% albumin solution, e.g., a 20% human albumin solution.

In some embodiments, the culture medium comprises and/or is supplemented with from or from about 2 to or to about 6 g/L albumin, e.g., human albumin. In some embodiments, the culture medium comprises and/or is supplemented with from or from about 2 to or to about 5.5, from or from about 2 to or to about 5.0, from or from about 2 to or to about 4.5, from or from about 2 to or to about 4, from or from about 2 to or to about 3.5, from or from about 2 to or to about 3, from or from about 2 to or to about 2.5, from or from about 2.5 to or to about 6, from or from about 2.5 to or to about 5.5, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3 to or to about 6, from or from about 3 to or to about 5.5, from or from about 3 to or to about 5, from or from about 3 to or to about 4.5, from or from about 3 to or to about 4, from or from about 3 to or to about 3.5, from or from about 3.5 to or to about 6, from or from about 3.5 to or to about 5.5, from or from about 3.5 to or to about 5, from or from about 3.5 to or to about 4.5, from or from about 3.5 to or to about 4, from or from about 4 to or to about 6, from or from about 4 to or to about 5.5, from or from about 4 to or to about 5, from or from about 4 to or to about 4.5, from or from about 4.5 to or to about 6, from or from about 4.5 to or to about 5.5, from or from about 4.5 to or to about 5, from or from about 5 to or to about 6, from or from about 5 to or to about 5.5, or from or from about 5.5 to or to about 6 g/L albumin, e.g., human albumin. In some embodiments, the culture medium comprises and/or is supplemented with from 3.2 to 4.8 g/L albumin, e.g., human albumin. In some embodiments, the culture medium comprises 4 g/L albumin, e.g., human albumin. In some embodiments, the culture medium comprises about 4 g/L albumin, e.g., human albumin

In some embodiments, the culture medium is supplemented with Poloxamer 188. In some embodiments, the culture medium comprises and/or is supplemented with from or from about 0.1 to or to about 2.0 g/L Poloxamer 188. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 g/L Poloxamer 188. In some embodiments, the culture medium comprises from 0.8 to 1.2 g/L Poloxamer 188. In some embodiments, the culture medium comprises 1.0 g/L Poloxamer 188. In some embodiments, the culture medium comprises about 1.0 g/L Poloxamer 188.

In some embodiments, the culture medium comprises and/or is supplemented with one or more antibiotics.

A first exemplary culture medium is set forth in Table 1.

TABLE 1
Exemplary Culture Medium #1

| | | |
|---|---|---|
| | Exemplary | Exemplary |
| Component | Concentration Range | Concentration |

| | | |
|---|---|---|
| CellgroSCGM liquid | undiluted | undiluted |

| | | | | |
|---|---|---|---|---|
| medium | | | | |
| Human Plasma | 0.8-1.2% | (v/v) | 1.0% | v/v |
| Glutamine | 3.2-4.8 | mM | 4.0 | mM |
| IL-2 | 64-96 | μg/L | 80 | μg/L |

A second exemplary culture medium is set forth in Table 2.

TABLE 2
Exemplary Culture Medium #2

| | Exemplary | | Exemplary | |
|---|---|---|---|---|
| Component | Concentration Range | | Concentration | |
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen Carbonate | 1.6-2.4 | g/L | 2.0 | g/L |
| IL-2 | 64-96 | μg/L | 80 | μg/L |
| Albumin 20% solution | 1.6-2.5% v/v | | 2.0% v/v | |
| | (3.2 to 4.8 g/L) | | (4.0 g/L) | |
| Poloxamer 188 | 0.8-1.2 | g/L | 1.0 | g/L |

2. CD3 Binding Antibodies

In some embodiments, the culture medium comprises and/or is supplemented with a CD3 binding antibody or antigen binding fragment thereof. In some embodiments, the CD3 binding antibody or antigen binding fragment thereof is selected from the group consisting of OKT3, UCHT1, and HIT3a, or variants thereof. In some embodiments, the CD3 binding antibody or antigen binding fragment thereof is OKT3 or an antigen binding fragment thereof.

In some embodiments, the CD3 binding antibody or antigen binding fragment thereof and feeder cells are added to the culture vessel before addition of NK cells and/or culture medium.

In some embodiments, the culture medium comprises and/or is supplemented with from or from about 5 ng/mL to or to about 15 ng/mL OKT3. In some embodiments, the culture medium comprises and/or is supplemented with from or from about 5 to or to about 12.5, from or from about 5 to or to about 10, from or from about 5 to or to about 7.5, from or from about 7.5 to or to about 15, from or from about 7.5 to or to about 12.5, from or from about 7.5 to or to about 10, from or from about 10 to or to about 15, from or from about 10 to or to about 12.5, or from or from about 12.5 to or to about 15 ng/mL OKT3. In some embodiments, the culture medium comprises and/or is supplemented with 10 ng/mL OKT3. In some embodiments, the culture medium comprises and/or is supplemented with about 10 ng/mL OKT3.

3. Culture Vessels

A number of vessels are consistent with the disclosure herein. In some embodiments, the culture vessel is selected from the group consisting of a flask, a bottle, a dish, a multiwall plate, a roller bottle, a bag, and a bioreactor.

In some embodiments, the culture vessel is treated to render it hydrophilic. In some embodiments, the culture vessel is treated to promote attachment and/or proliferation. In some embodiments, the culture vessel surface is coated with serum, collagen, laminin, gelatin, poy-L-lysine, fibronectin, extracellular matrix proteins, and combinations thereof.

In some embodiments, different types of culture vessels are used for different stages of culturing.

In some embodiments, the culture vessel has a volume of from or from about 100 mL to or to about 1,000 L. In some embodiments, the culture vessel has a volume of or about 125 mL, of or about 250 mL, of or about 500 mL, of or about 1 L, of or about 5 L, of about 10 L, or of or about 20 L.

In some embodiments, the culture vessel is a bioreactor.

In some embodiments, the bioreactor is a rocking bed (wave motion) bioreactor. In some embodiments, the bioreactor is a stirred tank bioreactor. In some embodiments, the bioreactor is a rotating wall vessel. In some embodiments, the bioreactor is a perfusion bioreactor. In some embodiments, the bioreactor is an isolation/expansion automated system. In some embodiments, the bioreactor is an automated or semi-automated bioreactor. In some embodiments, the bioreactor is a disposable bag bioreactor.

In some embodiments, the bioreactor has a volume of from about 100 mL to about 1,000 L. In some embodiments, the bioreactor has a volume of from about 10 L to about 1,000 L. In some embodiments, the bioreactor has a volume of from about 100 L to about 900 L. In some embodiments, the bioreactor has a volume of from about 10 L to about 800 L. In some embodiments, the bioreactor has a volume of from about 10 L to about 700 L, about 10 L to about 600 L, about 10 L to about 500 L, about 10 L to about 400 L, about 10 L to about 300 L, about 10 L to about 200 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 1,000 L, about 20 L to about 900 L, about 20 L to about 800 L, about 20 L to about 700 L, about 20 L to about 600 L, about 20 L to about 500 L, about 20 L to about 400 L, about 20 L to about 300 L, about 20 L to about 200 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 1,000 L, about 30 L to about 900 L, about 30 L to about 800 L, about 30 L to about 700 L, about 30 L to about 600 L, about 30 L to about 500 L, about 30 L to about 400 L, about 30 L to about 300 L, about 30 L to about 200 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 1,000 L, about 40 L to about 900 L, about 40 L to about 800 L, about 40 L to about 700 L, about 40 L to about 600 L, about 40 L to about 500 L, about 40 L to about 400 L, about 40 L to about 300 L, about 40 L to about 200 L, about 40 L to about 100 L, about 40 L to about 90 L, about 40 L to about 80 L, about 40 L to about 70 L, about 40 L to about 60 L, about 40 L to about 50 L, about 50 L to about 1,000 L, about 50 L to about 900 L, about 50 L to about 800 L, about 50 L to about 700 L, about 50 L to about 600 L, about 50 L to about 500 L, about 50 L to about 400 L, about 50 L to about 300 L, about 50 L to about 200 L, about 50 L to about 100 L, about 50 L to about 90 L, about 50 L to about 80 L, about 50 L to about 70 L, about 50 L to about 60 L, about 60 L to about 1,000 L, about 60 L to about 900 L, about 60 L to about 800 L, about 60 L to about 700 L, about 60 L to about 600 L, about 60 L to about 500 L, about 60 L to about 400 L, about 60 L to about 300 L, about 60 L to about 200 L, about 60 L to about 100L, about 60 L to about 90 L, about 60 L to about 80 L, about 60 L to about 70 L, about 70 L to about 1,000 L, about 70 L to about 900 L, about 70 L to about 800 L, about 70 L to about 700 L, about 70 L to about 600 L, about 70 L to about 500 L, about 70 L to about 400 L, about 70 L to about 300 L, about 70 L to about 200 L, about 70 L to about 100 L, about 70 L to about 90 L, about 70 L to about 80 L, about 80 L to about 1,000 L, about 80 L to about 900 L, about 80 L to about 800 L, about 80 L to about 700 L, about 80 L to about 600 L, about 80 L to about 500 L, about 80 L to about 400 L, about 80 L to about 300 L, about 80 L to about 200 L, about 80 L to about 100 L, about 80 L to about 90 L, about 90 L to about 1,000 L, about 90 L to about 900 L, about 90 L to about 800 L, about 90 L to about 700 L, about 90 L to about 600 L, about 90 L to about 500 L, about 90 L to about 400 L, about 90 L to about 300 L, about 90 L to about 200 L, about 90 L to about 100 L, about 100 L to about 1,000 L, about 100 L to about 900 L, about 100 L to about 800 L, about 100 L to about 700 L, about 100 L toa bout 600 L, about 100 L to about 500 L, about 100 L to about 400 L, about 100 L to about 300 L, about 100 L to about 200 L, about 200 L to about 1,000 L, about 200 L to about 900 L, about 200 L to about 800 L, about 200 L to about 700 L, about 200 L to about 600 L, about 200 L to about 500 L, about 200 L to about 400 L, about 200 L to about 300 L, about 300 L to about 1,000 L, about 300 L to about 900 L, about 300 L to about 800 L, about 300 L to about 700 L, about 300 L to about 600 L, about 300 L to about 500 L, about 300 L to about 400 L, about 400 L to about 1,000 L, about 400 L to about 900 L, about 400 L to about 800 L, about 400 L to about 700 L, about 400 L to about 600 L, about 400 L to about 500 L, about 500 L to about 1,000 L, about 500 L to about 900 L, about 500 L to about 800 L, about 500 L to about 700 L, about 500 L to about 600 L, about 600 L to about 1,000 L, about 600 L to about 900 L, about 600 L to about 800 L, about 600 L to about 700 L, about 700 L to about 1,000 L, about 700 L to about 900 L, about 700 L to about 800 L, about 800 L to about 1,000 L, about 800 L to about 900 L, or about 900 L to about 1,000 L. In some embodiments, the bioreactor has a volume of about 50 L.

In some embodiments, the bioreactor has a volume of from 100 mL to 1,000 L. In some embodiments, the bioreactor has a volume of from 10 L to 1,000 L. In some embodiments, the bioreactor has a volume of from 100 L to 900 L. In some embodiments, the bioreactor has a volume of from 10 L to 800 L. In some embodiments, the bioreactor has a volume of from 10 L to 700 L, 10 L to 600 L, 10 L to 500 L, 10 L to 400 L, 10 L to 300 L, 10 L to 200 L, 10 L to 100 L, 10 L to 90 L, 10 L to 80 L, 10 L to 70 L, 10 L to 60 L, 10 L to 50 L, 10 L to 40 L, 10 L to 30 L, 10 L to 20 L, 20 L to 1,000 L, 20 L to 900 L, 20 L to 800 L, 20 L to 700 L, 20 L to 600 L, 20 L to 500 L, 20 L to 400 L, 20 L to 300 L, 20 L to 200 L, 20 L to 100 L, 20 L to 90 L, 20 L to 80 L, 20 L to 70 L, 20 L to 60 L, 20 L to 50 L, 20 L to 40 L, 20 L to 30 L, 30 L to 1,000 L, 30 L to 900 L, 30 L to 800 L, 30 L to 700 L, 30 L to 600 L, 30 L to 500 L, 30 L to 400 L, 30 L to 300 L, 30 L to 200 L, 30 L to 100 L, 30 L to 90 L, 30 L to 80 L, 30 L to 70 L, 30 L to 60 L, 30 L to 50 L, 30 L to 40 L, 40 L to 1,000 L, 40 L to 900 L, 40 L to 800 L, 40 L to 700 L, 40 L to 600 L, 40 L to 500 L, 40 L to 400 L, 40 L to 300 L, 40 L to 200 L, 40 L to 100 L, 40 L to 90 L, 40 L to 80 L, 40 L to 70 L, 40 L to 60 L, 40 L to 50 L, 50 L to 1,000 L, 50 L to 900 L, 50 L to 800 L, 50 L to 700 L, 50 L to 600 L, 50 L to 500 L, 50 L to 400 L, 50 L to 300 L, 50 L to 200 L, 50 L to 100 L, 50 L to 90 L, 50 L to 80 L, 50 L to 70 L, 50 L to 60 L, 60 L to 1,000 L, 60 L to 900 L, 60 L to 800 L, 60 L to 700 L, 60 L to 600 L, 60 L to 500 L, 60 L to 400 L, 60 L to 300 L, 60 L to 200 L, 60 L to 100L, 60 L to 90 L, 60 L to 80 L, 60 L to 70 L, 70 L to 1,000 L, 70 L to 900 L, 70 L to 800 L, 70 L to 700 L, 70 L to 600 L, 70 L to 500 L, 70 L to 400 L, 70 L to 300 L, 70 L to 200 L, 70 L to 100 L, 70 L to 90 L, 70 L to 80 L, 80 L to 1,000 L, 80 L to 900 L, 80 L to 800 L, 80 L to 700 L, 80 L to 600 L, 80 L to 500 L, 80 L to 400 L, 80 L to 300 L, 80 L to 200 L, 80 L to 100 L, 80 L to 90 L, 90 L to 1,000 L, 90 L to 900 L, 90 L to 800 L, 90 L to 700 L, 90 L to 600 L, 90 L to 500 L, 90 L to 400 L, 90 L to 300 L, 90 L to 200 L, 90 L to 100 L, 100 L to 1,000 L, 100 L to 900 L, 100 L to 800 L, 100 L to 700 L, 100 L to 600 L, 100 L to 500 L, 100 L to 400 L, 100 L to 300 L, 100 L to 200 L, 200 L to 1,000 L, 200 L to 900 L, 200 L to 800 L, 200 L to 700 L, 200 L to 600 L, 200 L to 500 L, 200 L to 400 L, 200 L to 300 L, 300 L to 1,000 L, 300 L to 900 L, 300 L to 800 L, 300 L to 700 L, 300 L to 600 L, 300 L to 500 L, 300 L to 400 L, 400 L to 1,000 L, 400 L to 900 L, 400 L to 800 L, 400 L to 700 L, 400 L to 600 L, 400 L to 500 L, 500 L to 1,000 L, 500 L to 900 L, 500 L to 800 L, 500 L to 700 L, 500 L to 600 L, 600 L to 1,000 L, 600 L to 900 L, 600 L to 800 L, 600 L to 700 L, 700 L to 1,000 L, 700 L to 900 L, 700 L to 800 L, 800 L to 1,000 L, 800 L to 900 L, or 900 L to 1,000 L. In some embodiments, the bioreactor has a volume of 50 L.

4. Cell Expansion and Stimulation

In some embodiments, the natural killer cell source, e.g., single unit of cord blood, is co-cultured with feeder cells to produce expanded and stimulated NK cells.

In some embodiments, the co-culture is carried out in a culture medium described herein, e.g., exemplary culture medium #1 (Table 1) or exemplary culture medium #2 (Table 2).

In some embodiments, the natural killer cell source, e.g., single unit of cord blood, comprises from or from about 1×10⁷ to or to about 1×10⁹ total nucleated cells prior to expansion. In some embodiments, the natural killer cell source, e.g., single unit of cord blood, comprises from or from about 1×10⁸ to or to about 1.5×10⁸ total nucleated cells prior to expansion. In some embodiments, the natural killer cell source, e.g., single unit of cord blood, comprises 1×10⁸ total nucleated cells prior to expansion. In some embodiments, the natural killer cell source, e.g., single unit of cord blood, comprises about 1×10⁸ total nucleated cells prior to expansion. In some embodiments, the natural killer cell source, e.g., single unit of cord blood, comprises 1×10⁹ total nucleated cells prior to expansion. In some embodiments, the natural killer cell source, e.g., single unit of cord blood, comprises about 1×10⁹ total nucleated cells prior to expansion.

In some embodiments, cells from the co-culture of the natural killer cell source, e.g., single unit of cord blood and feeder cells are harvested and frozen, e.g., in a cryopreservation composition described herein. In some embodiments, the frozen cells from the co-culture are an infusion-ready drug product. In some embodiments, the frozen cells from the co-culture are used as a master cell bank (MCB) from which to produce an infusion-ready drug product, e.g., through one or more additional co-culturing steps, as described herein. Thus, for example, a natural killer cell source can be expanded and stimulated as described herein to produce expanded and stimulated NK cells suitable for use in an infusion-ready drug product without generating any intermediate products. A natural killer cell source can also be expanded and stimulated as described herein to produce an intermediate product, e.g., a first master cell bank (MCB). The first MCB can be used to produce expanded and stimulated NK cells suitable for use in an infusion-ready drug product, or, alternatively, be used to produce another intermediate product, e.g., a second MCB. The second MCB can be used to produce expanded and stimulated NK cells suitable for an infusion-ready drug product, or alternatively, be used to produce another intermediate product, e.g., a third MCB, and so on.

In some embodiments, the ratio of feeder cells to cells of the natural killer cell source or MCB cells inoculated into the co-culture is from or from about 1:1 to or to about 4:1. In some embodiments, the ratio of feeder cells to cells of the natural killer cell source or MCB cells is from or from about 1:1 to or to about 3.5:1, from or from about 1:1 to or to about 3:1, from or from about 1:1 to or to about 2.5:1, from or from about 1.1 to or to about 2:1, from or from about 1:1 to or to about 1.5:1, from or from about 1.5:1 to or to about 4:1, from or from about 1.5:1 to or to about 3.5:1, from or from about 1.5:1 to or to about 3:1, from or from about 1.5:1 to or to about 2.5:1, from or from about 1.5:1 to or to about 2:1, from or from about 2:1 to or to about 4:1, from or from about 2:1 to or to about 3.5:1, from or from about 2:1 to or to about 3:1, from or from about 2:1 to or to about 2.5:1, from or from about 2.5:1 to or to about 4:1, from or from about 2.5:1 to or to about 3.5:1, from or from about 2.5:1 to or to about 3:1, from or from about 3:1 to or to about 4:1, from or from about 3:1 to or to about 3.5:1, or from or from about 3.5:1 to or to about 4:1. In some embodiments, the ratio of feeder cells to cells of the natural killer cell source or MCB inoculated into the co-culture is 2.5:1. In some embodiments, the ratio of feeder cells to cells of the natural killer cell source or MCB inoculated into the co-culture is about 2.5:1.

In some embodiments, the co-culture is carried out in a disposable culture bag, e.g., a 1L disposable culture bag. In some embodiments, the co-culture is carried out in a bioreactor, e.g., a 50L bioreactor. In some embodiments, culture medium is added to the co-culture after the initial inoculation.

In some embodiments, the co-culture is carried out for 1, 2, 3, 4, 5, 6, 7, 8, 9, 10, 11, 12, 13, 14, 15, 16, 17, 18, 19, 20, 21, 22, 23, 24 or more days. In some embodiments, the co-culture is carried out for a maximum of 16 days.

In some embodiments, the co-culture is carried out at 37° C. or about 37° C.

In some embodiments, the co-culture is carried out at pH 7.9 or about pH 7.9.

In some embodiments, the co-culture is carried out at a dissolved oxygen (D0) level of 50% or more.

In some embodiments, exemplary culture medium #1 (Table 1) is used to produce a MCB and exemplary culture medium #2 (Table 2) is used to produce cells suitable for an infusion-ready drug product.

In some embodiments, the co-culture of the natural killer cell source, e.g., single unit of cord blood, with feeder cells yields from or from about 50×10⁸ to or to about 50×10¹² cells, e.g., MCB cells or infusion-ready drug product cells. In some embodiments, the expansion yields from or from about 50×10⁸ to or to about 25×10¹⁰⁰, from or from about 10×10⁸ to or to about 1×10¹⁰, from or from about 50×10⁸ to or to about 75×10⁹, from or from about 50×10⁸ to or to about 50×10⁹, from or from about 50×10⁸ to or to about 25×10⁹, from or from about 50×10⁸ to or to about 1×10⁹, from or from about 50×10⁸ to or to about 75×10⁸, from or from about 75×10⁸ to or to about 50×10¹⁰, from or from about 75×10⁸ to or to about 25×10¹⁰, from or from about 75×10⁸ to or to about 1×10¹⁰, from or from about 75×10⁸ to or to about 75×10⁹, from or from about 75×10⁸ to or to about 50×10⁹, from or from about 75×10⁸ to or to about 25×10⁹, from or from about 75×10⁸ to or to about 1×10⁹, from or from about 1×10⁹ to or to about 50×10¹⁰, from or from about 1×10⁹ to or to about 25×10¹⁰, from or from about 1×10⁹ to or to about 1×10¹⁰, from or from about 1×10⁹ to or to about 75×10⁹, from or from about 1×10⁹ to or to about 50×10⁹, from or from about 1×10⁹ to or to about 25×10⁹, from or from about 25×10⁹ to or to about 50×10¹⁰, from or from about 25×10⁹ to or to about 25×10¹⁰, from or from about 25×10⁹ to or to about 1×10¹⁰, from or from about 25×10⁹ to or to about 75×10⁹, from or from about 25×10⁹ to or to about 50×10⁹, from or from about 50×10⁹ to or to about 50×10¹⁰, from or from about 50×10⁹ to or to about 25×10¹⁰, from or from about 50×10⁹ to or to about 1×10¹⁰, from or from about 50×10⁹ to or to about 75×10⁹, from or from about 75×10⁹ to or to about 50×10¹⁰, from or from about 75×10⁹ to or to about 25×10¹⁰, from or from about 75×10⁹ to or to about 1×10¹⁰, from or from about 1×10¹⁰ to or to about 50×10¹⁰, from or from about 1×10¹⁰ to or to about 25×10¹⁰, or from or from about 25×10¹⁰ to or to about 50×10¹⁰ cells, e.g., e.g., MCB cells or infusion-ready drug product cells.

In some embodiments, the expansion yields from or from about 60 to or to about 100 vials, each comprising from or from about 600 million to or to about 1 billion cells, e.g., MCB cells or infusion-ready drug product cells. In some embodiments, the expansion yields 80 or about 80 vials, each comprising or consisting of 800 million or about 800 million cells, e.g., MCB cells or infusion-ready drug product cells.

In some embodiments, the expansion yields from or from about a 100 to or to about a 500 fold increase in the number of cells, e.g., the number of MCB cells relative to the number of cells, e.g., NK cells, in the natural killer cell source. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of MCB cells relative to the number of cells, e.g., NK cells, in the natural killer cell source.

In some embodiments, the expansion yields from or from about a 100 to or to about a 70,000 fold increase in the number of cells, e.g., the number of MCB cells relative to the number of cells, e.g., NK cells, in the natural killer cell source. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of MCB cells relative to the number of cells, e.g., NK cells, in the natural killer cell source.

In some embodiments, the co-culture of the MCB cells and feeder cells yields from or from about 500 million to or to about 1.5 billion cells, e.g., NK cells suitable for use in an MCB and/or in an infusion-ready drug product. In some embodiments, the co-culture of the MCB cells and feeder cells yields from or from about 500 million to or to about 1.5 billion, from or from about 500 million to or to about 1.25 billion, from or from about 500 million to or to about 1 billion, from or from about 500 million to or to about 750 million, from or from about 750 million to or to about 1.5 billion, from or from about 500 million to or to about 1.25 billion, from or from about 750 million to or to about 1 billion, from or from about 1 billion to or to about 1.5 billion, from or from about 1 billion to or to about 1.25 billion, or from or from about 1.25 billion to or to about 1.5 billion cells, e.g., NK cells suitable for use in an MCB and/or an infusion-ready drug product.

In some embodiments, the co-culture of the MCB cells and feeder cells yields from or from about 50 to or to about 150 vials of cells, e.g., infusion-ready drug product cells, each comprising from or from about 750 million to or to about 1.25 billion cells, e.g., NK cells suitable for use in an MCB and/or an infusion-ready drug product. In some embodiments, the co-culture of the MCB cells and feeder cells yields 100 or about 100 vials, each comprising or consisting of 1 billion or about 1 billion cells, e.g., NK cells suitable for use in an MCB and/or an infusion-ready drug product.

In some embodiments, the expansion yields from or from about a 100 to or to about a 500 fold increase in the number of cells, e.g., the number of NK cells suitable for use in an MCB and/or an infusion-ready drug product relative to the number of starting MCB cells. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of NK cells suitable for use in an MCB and/or an infusion-ready drug product relative to the number of starting MCB cells.

In some embodiments, the expansion yields from or from about a 100 to or to about a 70,000 fold increase in the number of cells, e.g., the number of NK cells suitable for use in an MCB and/or an infusion-ready drug product relative to the number of starting MCB cells. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of NK cells suitable for use in an MCB and/or an infusion-ready drug product relative to the number of starting MCB cells.

In embodiments where the cells are engineered during expansion and stimulation, as described herein, not all of the expanded and stimulated cells will necessarily be engineered successfully, e.g., transduced successfully, e.g., transduced successfully with a vector comprising a heterologous protein, e.g., a heterologous protein comprising a CAR and/or IL-15 as described herein. Thus, the methods described herein can further comprise sorting engineered cells, e.g., engineered cells described herein, away from non-engineered cells.

In some embodiments, the engineered cells, e.g., transduced cells, are sorted from the non-engineered cells, e.g., the non-transduced cells using a reagent specific to an antigen of the engineered cells, e.g., an antibody that targets an antigen of the engineered cells but not the non-engineered cells. In some embodiments, the antigen of the engineered cells is a component of a CAR, e.g., a CAR described herein.

Systems for antigen-based cell separation of cells are available commercially, e.g., the CliniMACS® sorting system (Miltenyi Biotec).

In some embodiments, the engineered cells, e.g., transduced cells, are sorted from the non-engineered cells, e.g., the non-transduced cells using flow cytometry.

In some embodiments, the sorted engineered cells are used as an MCB. In some embodiments, the sorted engineered cells are used as a component in an infusion-ready drug product.

In some embodiments, the engineered cells, e.g., transduced cells, are sorted from the non-engineered cells, e.g., the non-transduced cells using a microfluidic cell sorting method. Microfluidic cell sorting methods are described, for example, in Dalili et al., “A Review of Sorting, Separation and Isolation of Cells and Microbeads for Biomedical Applications: Microfluidic Approaches,” Analyst 144:87 (2019).

In some embodiments, from or from about 1% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein. In some embodiments, from or from about 1% to or to about 90%, from or from about 1% to or to about 80%, from or from about 1% to or to about 70%, from or from about 1% to or to about 60%, from or from about 1% to or to about 50%, from or from about 1% to or to about 40%, from or from about 1% to or to about 30%, from or from about 1% to or to about 20%, from or from about 1% to or to about 10%, from or from about 1% to or to about 5%, from or from about 5% to or to about 99%, from or from about 5% to or to about 90%, from or from about 5% to or to about 80%, from or from about 5% to or to about 70%, from or from about 5% to or to about 60%, from or from about 5% to or to about 50%, from or from about 5% to or to about 40%, from or from about 5% to or to about 30%, from or from about 5% to or to about 20%, from or from about 5% to or to about 10%, from or from about 10% to or to about 99%, from or from about 10% to or to about 90%, from or from about 10% to or to about 80%, from or from about 10% to or to about 70%, from or from about 10% to or to about 60%, from or from about 10% to or to about 50%, from or from about 10% to or to about 40%, from or from about 10% to or to about 30%, from or from about 10% to or to about 20%, from or from about 20% to or to about 99%, from or from about 20% to or to about 90%, from or from about 20% to or to about 80%, from or from about 20% to or to about 70%, from or from about 20% to or to about 60%, from or from about 20% to or to about 50%, from or from about 20% to or to about 40%, from or from about 20% to or to about 30%, from or from about 30% to or to about 99%, from or from about 30% to or to about 90%, from or from about 30% to or to about 80%, from or from about 30% to or to about 70%, from or from about 30% to or to about 60%, from or from about 30% to or to about 50%, from or from about 30% to or to about 40%, from or from about 40% to or to about 99%, from or from about 40% to or to about 90%, from or from about 40% to or to about 80%, from or from about 40% to or to about 70%, from or from about 40% to or to about 70%, from or from about 40% to or to about 60%, from or from about 40% to or to about 50%, from or from about 50% to or to about 99%, from or from about 50% to or to about 90%, from or from about 50% to or to about 80%, from or from about 50% to or to about 70%, from or from about 50% to or to about 60%, from or from about 60% to or to about 99%, from or from about 60% to or to about 90%, from or from about 60% to or to about 80%, from or from about 60% to or to about 70%, from or from about 70% to or to about 99%, from or from about 70% to or to about 90%, from or from about 70% to or to about 80%, from or from about 80% to or to about 99%, from or from about 80% to or to about 90%, or from or from about 90% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein.

In some embodiments, frozen cells of a first or second MCB are thawed and cultured. In some embodiments, a single vial of frozen cells of the first or second MCB e.g., a single vial comprising 800 or about 800 million cells, e.g., first or second MCB cells, are thawed and cultured. In some embodiments, the frozen first or second MCB cells are cultured with additional feeder cells to produce cells suitable for use either as a second or third MCB or in an infusion-ready drug product. In some embodiments, the cells from the co-culture of the first or second MCB are harvested and frozen.

In some embodiments, the cells from the co-culture of the natural killer cell source, a first MCB, or a second MCB are harvested, and frozen in a cryopreservation composition, e.g., a cryopreservation composition described herein. In some embodiments, the cells are washed after harvesting. Thus, provided herein is a pharmaceutical composition comprising activated and stimulated NK cells, e.g., activated and stimulated NK cells produced by the methods described herein, e.g., harvested and washed activated and stimulated NK cells produced by the methods described herein and a cryopreservation composition, e.g., a cryopreservation composition described herein.

In some embodiments, the cells are mixed with a cryopreservation composition, e.g., as described herein, before freezing. In some embodiments, the cells are frozen in cryobags. In some embodiments, the cells are frozen in cryovials.

In some embodiments, the method further comprises isolating NK cells from the population of expanded and stimulated NK cells.

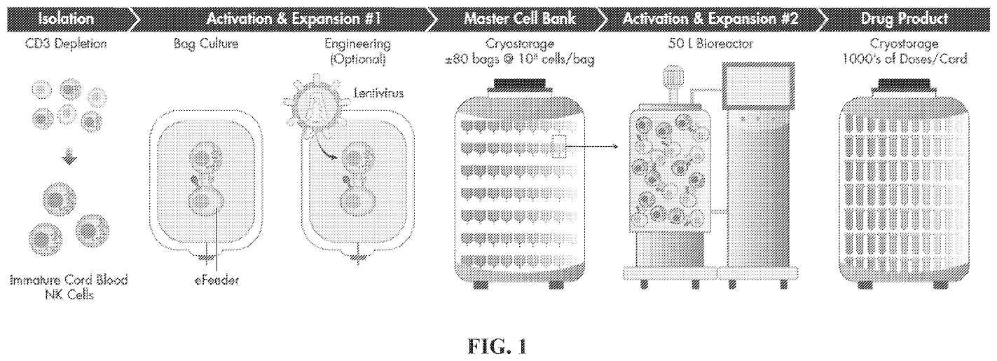

An exemplary process for expanding and stimulating NK cells is shown in FIG. 1.

5. Engineering

In some embodiments, the method further comprises engineering NK cell(s), e.g., to express a heterologous protein, e.g., a heterologous protein described herein, e.g., a heterologous protein comprising a CAR and/or IL-15.

In some embodiments, engineering the NK cell(s) to express a heterologous protein described herein comprises transforming, e.g., stably transforming the NK cells with a vector comprising a polynucleic acid encoding a heterologous protein described herein. Suitable vectors are described herein.

In some embodiments, engineering the NK cell(s) to express a heterologous protein described herein comprises introducing the heterologous protein via gene editing (e.g., zinc finger nuclease (ZFN) gene editing, ARCUS gene editing, CRISPR-Cas9 gene editing, or megaTAL gene editing) combined with adeno-associated virus (AAV) technology.

In some embodiments, the NK cell(s) are engineered to express a heterologous protein described herein, e.g., during or after culturing the composition in a medium comprising feeder cells.

In some embodiments, the method further comprises engineering NK cell(s), e.g., to express, over-express, knock-out, or knock-down gene(s) or gene product(s).

In some embodiments, the natural killer cells are not genetically engineered.

E. Properties of Expanded and Stimulated NK Cells

After having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3− cells.

In some embodiments, the expanded and stimulated NK cells are not genetically engineered.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3(+) depleted, e.g., by gating on CD56+CD3− expression, but no other surface protein expression selection is carried out during expansion and stimulation.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% CD94+(KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+(IL2RB) cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+ NK cells.

In some embodiments, the expanded and stimulated NK cells, e.g., from expansion and stimulation of a single cord blood unit, e.g., as described above, comprises both the KIR B allele of the KIR receptor family and the 158 VN variant of CD16 and comprise 50% or more, e.g., 55%, 60%, 65%, 70%, 75%, 80%, 85%, 90%, or 95% CD16+ NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., Jiao et al., “CD38: Targeted Therapy in Multiple Myeloma and Therapeutic Potential for Solid Cancerrs,” Expert Opinion on Investigational Drugs 29(11):1295-1308 (2020). Yet, when an anti-CD38 antibody is administered with NK cells, because NK cells naturally express CD38, they are at risk for increased fratricide. The NK cells expanded and stimulated by the methods described herein, however, express low levels of CD38 and, therefore, overcome the anticipated fratricide. While other groups have resorted to engineering methods such as genome editing to reduce CD38 expression (see, e.g., Gurney et al., “CD38 Knockout Natural Killer Cells Expressing an Affinity Optimized CD38 Chimeric Antigen Receptor Successfully Target Acute Myeloid Leukemia with Reduced Effector Cell Fratricide,” Haematologica doi:10.3324/haematol.2020.271908 (2020), the NK cells expanded and stimulated by the methods described herein express low levels of CD38 without the need for genetic engineering, which provides a surprising and unexpected benefits, e.g., for treating CD38+ cancers with the NK cells expanded and stimulated as described herein, e.g., in combination with a CD38 antibody.

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+ NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+ NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+(KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+(IL2RB) cells.

In some embodiments, feeder cells do not persist in the expanded and stimulated NK cells, though, residual signature of the feeder cells may be detected, for example, by the presence of residual cells (e.g., by detecting cells with a particular surface protein expression) or residual nucleic acid and/or proteins that are expressed by the feeder cells.

For example, in some cases, the methods described herein include expanding and stimulating natural killer cells using engineered feeder cells, e.g., eHuT-78 feeder cells described above, which are engineered to express sequences that are not expressed by cells in the natural killer cell source, including the natural killer cells. For example, the engineered feeder cells can be engineered to express at least one gene selected from the group consisting of 4-1BBL (UniProtKB P41273, SEQ ID NO: 10), membrane bound IL-21 (SEQ ID NO: 11), and mutant TNFalpha (SEQ ID NO: 12) (“eHut-78 cells”), or variants thereof.

While these feeder cells may not persist in the expanded and stimulated NK cells, the expanded and stimulated NK cells may retain detectable residual amounts of cells, proteins, and/or nucleic acids from the feeder cells. Thus, their residual presence in the expanded and stimulated NK cells may be detected, for example, by detecting the cells themselves (e.g., by flow cytometry), proteins that they express, and/or nucleic acids that they express.

Thus, also described herein is a population of expanded and stimulated NK cells comprising residual feeder cells (live cells or dead cells) or residual feeder cell cellular impurities (e.g., residual feeder cell proteins or portions thereof, and/or genetic material such as a nucleic acid or portion thereof). In some cases, the expanded and stimulated NK cells comprise more than 0% and, but 0.3% or less residual feeder cells, e.g., eHuT-78 feeder cells.

In some cases, the expanded and stimulated NK cells comprise residual feeder cell nucleic acids, e.g., encoding residual 4-1BBL (UniProtKB P41273, SEQ ID NO: 10), membrane bound IL-21 (SEQ ID NO: 11), and/or mutant TNFalpha (SEQ ID NO: 12) or portion(s) thereof. In some cases, the membrane bound IL-21 comprises a CD8 transmembrane domain

In some cases, the expanded and stimulated NK cells comprise a % residual feeder cells of more than 0% and less than or equal to 0.2%, as measured, e.g., by the relative proportion of a feeder cell specific protein or nucleic acid sequence (that is, a protein or nucleic acid sequence not expressed by the natural killer cells) in the sample. For example, by qPCR, e.g., as described herein.

In some embodiments, the residual feeder cells are CD4(+) T cells. In some embodiments, the residual feeder cells are engineered CD4(+) T cells. In some embodiments, the residual feeder cell cells are engineered to express at least one gene selected from the group consisting of 4-1BBL (UniProtKB P41273, SEQ ID NO: 10), membrane bound IL-21 (SEQ ID NO: 11), and mutant TNFalpha (SEQ ID NO: 12) (“eHut-78 cells”), or variants thereof. Thus, in some cases, the feeder cell specific protein is 4-1BBL (UniProtKB P41273, SEQ ID NO: 10), membrane bound IL-21 (SEQ ID NO: 11), and/or mutant TNFalpha (SEQ ID NO: 12). And, therefore, the feeder cell specific nucleic acid is a nucleic acid encoding 4-1BBL (UniProtKB P41273, SEQ ID NO: 10), membrane bound IL-21 (SEQ ID NO: 11), and/or mutant TNFalpha (SEQ ID NO: 12), or portion thereof. In some cases, the membrane bound IL-21 comprises a CD8 transmembrane domain.

In some embodiments, the residual feeder cells are detected by the method described in Example 18.

A wide variety of different methods can be used to analyze and detect the presence of nucleic acids or protein gene products in a biological sample. As used herein, “detecting” can refer to a method used to discover, determine, or confirm the existence or presence of a compound and/or substance (e.g., a cell, a protein and/or a nucleic acid). In some embodiments, a detecting method can be used to detect a protein. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein (Jungblut et al., J Biotechnol. 31; 41(2-3):111-20 (1995); Franco et al., Eur J Morphol. 39(1):3-25 (2001)). In some embodiments, a detecting method can be used to detect a nucleic acid (e.g., DNA and/or RNA). In some embodiments, detecting can include Northern blot analysis, nuclease protection assays (NPA), in situ hybridization, or reverse transcription-polymerase chain reaction (RT-PCR) (Raj et al., Nat. Methods 5, 877-879 (2008); Jin et al., J Clin Lab Anal. 11(1):2-9 (1997); Ahmed, J Environ Sci Health C Environ Carcinog Ecotoxicol Rev. 20(2):77-116 (2002)).

Thus, also described herein, are methods for detecting a population of expanded and stimulated NK cells, e.g., expanded and stimulated using the methods described herein, that have been co-cultured with engineered feeder cells, e.g., eHuT-78 feeder cells described herein.

### II. Natural Killer Cell Engineering

In some embodiments, the natural killer cells are engineered, e.g., to produce CAR-NK(s) and/or IL-15 expressing NK(s).

In some embodiments, the natural killer cells are engineered, e.g., transduced, during expansion and stimulation, e.g., expansion and stimulation described herein. In some embodiments, the natural killer cells are engineered during expansion and stimulation, e.g., during production of a MCB, as described herein. In some embodiments, the natural killer cells are engineered during expansion and stimulation, e.g., during production of NK cells suitable for use in an injection-ready drug product and/or during production of a MCB, as described above. Thus, in some embodiments, the NK cell(s) are host cells and provided herein are NK host cell(s) expressing a heterogeneous protein, e.g., as described herein.

In some embodiments, the natural killer cells are engineered prior to expansion and stimulation. In some embodiments, the natural killer cells are engineered after expansion and stimulation.

In some embodiments, the NK cells are engineered by transducing with a vector. Suitable vectors are described herein, e.g., lentiviral vectors, e.g., a lentiviral vectors comprising a heterologous protein, e.g., as described herein. In some embodiments, the NK cells are transduced during production of a first MCB, as described herein.

In some embodiments, the NK cell(s) are transduced at a multiplicity of infection of from or from about 1 to or to about 40 viral particles per cell. In some embodiments, the NK cell(s) are transduced at a multiplicity of infection of or of about 1, of or of about 5, of or of about 10, of or of about 15, of or of about 20, of or of about 25, of or of about 30, of or of about 35, or of or of about 40 viral particles per cell.

A. Chimeric Antigen Receptors

In some embodiments, the heterologous protein is a fusion protein, e.g., a fusion protein comprising a chimeric antigen receptor (CAR) is introduced into the NK cell, e.g., during the expansion and stimulation process.

In some embodiments, the CAR comprises one or more of: a signal sequence, an extracellular domain, a hinge, a transmembrane domain, and one or more intracellular signaling domain sequences. In some embodiments, the CAR further comprises a spacer sequence.

In some embodiments, the CAR comprises (from N- to C-terminal): a signal sequence, an extracellular domain, a hinge, a spacer, a transmembrane domain, a first signaling domain sequence, a second signaling domain sequence, and a third signaling domain sequence.

In some embodiments, the CAR comprises (from N- to C-terminal): a signal sequence, an extracellular domain, a hinge, a transmembrane domain, a first signaling domain sequence, a second signaling domain sequence, and a third signaling domain sequence.

In some embodiments the extracellular domain comprises an antibody or antigen-binding portion thereof.

In some embodiments, one or more of the intracellular signaling domain sequence(s) is a CD28 intracellular signaling sequence. In some embodiments, the CD28 intracellular signaling sequence comprises or consists of SEQ ID NO: 14.

In some embodiments, one or more of the intracellular signaling domain sequence(s) is an OX40L signaling sequence. In some embodiments, the OX40L signaling sequence comprises or consists of SEQ ID NO: 17.

In some embodiments, one or more of the intracellular signaling sequence(s) is a CD3ζ intracellular signaling domain sequence. In some embodiments, the CD3ζ intracellular signaling sequence comprises of consists of SEQ ID NO: 20.

In some embodiments, the CAR comprises a CD28 intracellular signaling sequence (SEQ ID NO: 14), an OX40L intracellular signaling sequence (SEQ ID NO: 17), and a CD3ζ intracellular signaling sequence (SEQ ID NO: 20).

In some embodiments, the CAR comprises an intracellular signaling domain comprising or consisting of SEQ ID NO: 28.

In some embodiments, the CAR does not comprise an OX40L intracellular signaling domain sequence.

In some embodiments, the CAR comprises a CD28 intracellular signaling sequence (SEQ ID NO: 14), and a CD3ζ intracellular signaling sequence (SEQ ID NO: 20), but not an OX40L intracellular signaling domain sequence.

B. IL-15

In some embodiments, the NK cell is engineered to express IL-15, e.g., human IL-15 (UniProtKB #P40933; NCBI Gene ID #3600), e.g., soluble human IL-15 or an ortholog thereof, or a variant of any of the foregoing. In some embodiments, the IL-15 is expressed as part of a fusion protein further comprising a cleavage site. In some embodiments, the IL-15 is expressed as part of a polyprotein comprising a T2A ribosomal skip sequence site (sometimes referred to as a self-cleaving site).

In some embodiments, the IL-15 comprises or consists of SEQ ID NO: 25.

In some embodiments, the T2A cleavage site comprises or consists of SEQ ID NO: 23.

In some embodiments, the IL-15 is expressed as part of a fusion protein comprising a CAR, e.g., a CAR described herein.

In some embodiments, the fusion protein comprises (oriented from N-terminally to C-terminally): a CAR comprising, a cleavage site, and IL-15.

In some embodiments, the fusion protein comprises SEQ ID NO: 29.

C. Inhibitory Receptors

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRB1, PD-1, IRp60, Siglec-7, LAIR-1, and combinations thereof.

D. Polynucleic Acids, Vectors, and Host Cells

Also provided herein are polynucleic acids encoding the fusion protein(s) or portions thereof, e.g., the polynucleotide sequences encoding the polypeptides described herein, as shown in the Table of sequences provided herein

Also provided herein are vector(s) comprising the polynucleic acids, and cells, e.g., NK cells, comprising the vector(s).

In some embodiments, the vector is a lentivirus vector. See, e.g., Milone et al., “Clinical Use of Lentiviral Vectors,” Leukemia 32:1529-41 (2018). In some embodiments, the vector is a retrovirus vector. In some embodiments, the vector is a gamma retroviral vector. In some embodiments, the vector is a non-viral vector, e.g., a piggyback non-viral vector (PB transposon, see, e.g., Wu et al., “piggyback is a Flexible and Highly Active Transposon as Compared to Sleeping Beauty, Tol2, and Mos1 in Mammalian Cells,” PNAS 103(41):15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52(4):355-380 (2017)), or an mRNA vector.

### III. Cryopreservation

A. Cryopreservation Compositions

Provided herein are cryopreservation compositions, e.g., cryopreservation compositions suitable for intravenous administration, e.g., intravenous administration of NK cells, e.g., the NK cells described herein. In some embodiments, a pharmaceutical composition comprises the cryopreservation composition and cells, e.g., the NK cells described herein.

1. Albumin

In some embodiments, the cryopreservation composition comprises albumin protein, e.g., human albumin protein (UniProtKB Accession P0278, SEQ ID NO: 30) or variant thereof. In some embodiments, the cryopreservation composition comprises an ortholog of an albumin protein, e.g., human albumin protein, or variant thereof. In some embodiments, the cryopreservation composition comprises a biologically active portion of an albumin protein, e.g., human albumin, or variant thereof.

In some embodiments, the albumin, e.g., human albumin, is provided as a solution, also referred to herein as an albumin solution or a human albumin solution. Thus, in some embodiments, the cryopreservation composition is or comprises an albumin solution, e.g., a human albumin solution. In some embodiments, the albumin solution is a serum-free albumin solution.

In some embodiments, the albumin solution is suitable for intravenous use.

In some embodiments, the albumin solution comprises from or from about 40 to or to about 200 g/L albumin. In some embodiments, the albumin solution comprises from or from about 40 to or to about 50 g/L albumin, e.g., human albumin. In some embodiments, the albumin solution comprises about 200 g/L albumin, e.g., human albumin. In some embodiments, the albumin solution comprises 200 g/L albumin, e.g., human albumin.

In some embodiments, the albumin solution comprises a protein composition, of which 95% or more is albumin protein, e.g., human albumin protein. In some embodiments, 96%, 97%, 98%, or 99% or more of the protein is albumin, e.g., human albumin.

In some embodiments, the albumin solution further comprises sodium. In some embodiments, the albumin solution comprises from or from about 100 to or to about 200 mmol sodium. In some embodiments, the albumin solution comprises from or from about 130 to or to about 160 mmol sodium.

In some embodiments, the albumin solution further comprises potassium. In some embodiments, the albumin solution comprises 3 mmol or less potassium. In some embodiments, the albumin solution further comprises 2 mmol or less potassium.

In some embodiments, the albumin solution further comprises one or more stabilizers. In some embodiments, the stabilizer(s) are selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan). In some embodiments, the solution comprises less than 0.1 mmol of each of the one or more stabilizers per gram of protein in the solution. In some embodiments, the solution comprises from or from about 0.05 to or to about 0.1, e.g., from or from about 0.064 to or to about 0.096 mmol of each of the stabilizers per gram of protein in the solution. In some embodiments, the solution comprises less than 0.1 mmol of total stabilizer per gram of protein in the solution. In some embodiments, the solution comprises from or from about 0.05 to or to about 0.1, e.g., from or from about 0.064 to or to about 0.096 mmol of total stabilizer per gram of protein in the solution.

In some embodiments, the albumin solution consists of a protein composition, of which 95% or more is albumin protein, sodium, potassium, and one or more stabilizers selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan) in water.

In some embodiments, the cryopreservation composition comprises from or from about 10% v/v to or to about 50% v/v of an albumin solution, e.g., an albumin solution described herein. In some embodiments, the cryopreservation composition comprises from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of an albumin solution described herein. In some embodiments, the cryopreservation composition comprises about 10%, about 15%, about 20%, about 25%, about 30%, about 35%, about 40%, about 45%, or about 50% v/v of an albumin solution described herein. In some embodiments, the cryopreservation composition comprises 10%, 15%, 20%, 25%, 30%, 35%, 40%, 45%, or 50% v/v of an albumin solution described herein.

In some embodiments, the cryopreservation composition comprises from or from about 20 to or to about 100 g/L albumin, e.g., human albumin. In some embodiments, the cryopreservation composition comprises from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 100, from or from about 80 to or to about 90, or from or from about 90 to or to about 100 g/L albumin, e.g., human albumin.

In some embodiments, the cryopreservation composition comprises 20 g/L albumin, e.g., human albumin. In some embodiments, the cryopreservation composition comprises 40 g/L albumin, e.g., human albumin. In some embodiments, the cryopreservation composition comprises 70 g/L albumin, e.g., human albumin. In some embodiments, the cryopreservation composition comprises 100 g/L albumin, e.g., human albumin.

In some embodiments, the cryopreservation composition comprises about 20 g/L albumin, e.g., human albumin. In some embodiments, the cryopreservation composition comprises about 40 g/L albumin, e.g., human albumin. In some embodiments, the cryopreservation composition comprises about 70 g/L albumin, e.g., human albumin. In some embodiments, the cryopreservation composition comprises about 100 g/L albumin, e.g., human albumin.

In some embodiments, the cryopreservation composition further comprises a stabilizer, e.g., an albumin stabilizer. In some embodiments, the stabilizer(s) are selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan). In some embodiments, the cryopreservation composition comprises less than 0.1 mmol of each of the one or more stabilizers per gram of protein, e.g., per gram of albumin protein, in the composition. In some embodiments, the cryopreservation composition comprises from or from about 0.05 to or to about 0.1, e.g., from or from about 0.064 to or to about 0.096 mmol of each of the stabilizers per gram of protein, e.g., per gram of albumin protein in the composition. In some embodiments, the cryopreservation composition comprises less than 0.1 mmol of total stabilizer per gram of protein, e.g., per gram of albumin protein in the cryopreservation composition. In some embodiments, the cryopreservation composition comprises from or from about 0.05 to or to about 0.1, e.g., from or from about 0.064 to or to about 0.096 mmol of total stabilizer per gram of protein, e.g., per gram of albumin protein, in the cryopreservation composition.

2. Dextran

In some embodiments, the cryopreservation composition comprises Dextran, or a derivative thereof.

Dextran is a polymer of anhydroglucose composed of approximately 95% α-D-(1-6) linkages (designated (C₆H₁₀O₅)ₙ). Dextran fractions are supplied in molecular weights of from about 1,000 Daltons to about 2,000,000 Daltons. They are designated by number (Dextran X), e.g., Dextran 1, Dextran 10, Dextran 40, Dextran 70, and so on, where X corresponds to the mean molecular weight divided by 1,000 Daltons. So, for example, Dextran 40 has an average molecular weight of or about 40,000 Daltons.

In some embodiments, the average molecular weight of the dextran is from or from about 1,000 Daltons to or to about 2,000,000 Daltons. In some embodiments, the average molecular weight of the dextran is or is about 40,000 Daltons. In some embodiments, the average molecular weight of the dextran is or is about 70,000 Daltons.

In some embodiments, the dextran is selected from the group consisting of Dextran 40, Dextran 70, and combinations thereof. In some embodiments, the dextran is Dextran 40.

In some embodiments, the dextran, e.g., Dextran 40, is provided as a solution, also referred to herein as a dextran solution or a Dextran 40 solution. Thus, in some embodiments, the composition comprises a dextran solution, e.g., a Dextran 40 solution.

In some embodiments, the dextran solution is suitable for intravenous use.

In some embodiments, the dextran solution comprises about 5% to about 50% w/w dextran, e.g., Dextran 40. In some embodiments, the dextran solution comprises from or from about 5% to or to about 50%, from or from about 5% to or to about 45%, from or from about 5% to or to about 40%, from or from about 5% to or to about 35%, from or from about 5% to or to about 30%, from or from about 5% to or to about 25%, from or from about 5% to or to about 20%, from or from about 5% to or to about 15%, from or from about 5% to or to about 10%, from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% w/w dextran, e.g., Dextran 40. In some embodiments, the dextran solution comprises 5%, 10%, 15%, 20%, 25%, 30%, 35%, 40%, 45%, or 50% w/w dextran, e.g., Dextran 40. In some embodiments, the dextran solution comprises about 5%, about 10%, about 15%, about 20%, about 25%, about 30%, about 35%, about 40%, about 45%, or about 50% w/w dextran, e.g., Dextran 40.

In some embodiments, the dextran solution comprises from or from about 25 g/L to or to about 200 g/L dextran, e.g., Dextran 40. In some embodiments, the dextran solution comprises from or from about 35 to or to about 200, from or from about 25 to or to about 175, from or from about 25 to or to about 150, from or from about 25 to or to about 125, from or from about 25 to or to about 100, from or from about 25 to or to about 75, from or from about 25 to or to about 50, from or from about 50 to or to about 200, from or from about 50 to or to about 175, from or from about 50 to or to about 150, from or from about 50 to or to about 125, from or from about 50 to or to about 100, from or from about 50 to or to about 75, from or from about 75 to or to about 200, from or from about 75 to or to about 175, from or from about 75 to or to about 150, from or from about 75 to or to about 125, from or from about 75 to or to about 100, from or from about 100 to or to about 200, from or from about 100 to or to about 175, from or from about 100 to or to about 150, from or from about 100 to or to about 125, from or from about 125 to or to about 200, from or from about 125 to or to about 175, from or from about 125 to or to about 150, from or from about 150 to or to about 200, from or from about 150 to or to about 175, or from or from about 175 to or to about 200 g/L dextran e.g., Dextran 40. In some embodiments, the dextran solution comprises 25, 50, 75, 100, 125, 150, 175, or 200 g/L dextran, e.g., Dextran 40. In some embodiments, the dextran solution comprises 100 g/L dextran, e.g., Dextran 40. In some embodiments, the dextran solution comprises about 25, about 50, about 75, about 100, about 125, about 150, about 175, or about 200 g/L dextran, e.g., Dextran 40. In some embodiments, the dextran solution comprises about 100 g/L dextran, e.g., Dextran 40.

In some embodiments, the dextran solution further comprises glucose (also referred to as dextrose). In some embodiments, the dextran solution comprises from or from about 10 g/L to or to about 100 g/L glucose. In some embodiments, the dextran solution comprises from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 90, from or from about 80 to or to about 100, from or from about 80 to or to about 90, or from or from about 90 to or to about 100 g/L glucose. In some embodiments, the dextran solution comprises 10, 20, 30, 40, 50, 60, 70, 80, 90, or 100 g/L glucose. In some embodiments, the dextran solution comprises 50 g/L glucose. In some embodiments, the dextran solution comprises about 10, about 20, about 30, about 40, about 50, about 60, about 70, about 80, about 90, or about 100 g/L glucose. In some embodiments, the dextran solution comprises 50 g/L glucose.

In some embodiments, the dextran solution consists of dextran, e.g., Dextran 40, and glucose in water.

In some embodiments, the cryopreservation composition comprises from or from about 10% v/v to or to about 50% v/v of a dextran solution described herein. In some embodiments, the cryopreservation composition comprises from or from about 10% to 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of a dextran solution, e.g., a dextran solution described herein. In some embodiments, the cryopreservation composition comprises 10%, 15%, 20%, 25%, 30%, 35%, 40%, 45%, or 50% v/v of a dextran solution, e.g., a dextran solution described herein. In some embodiments, the cryopreservation composition comprises about 10%, about 15%, about 20%, about 25%, about 30%, about 35%, about 40%, about 45%, or about 50% v/v of a dextran solution, e.g., a dextran solution described herein.

In some embodiments, the cryopreservation composition comprises from or from about 10 to or to about 50 g/L dextran, e.g., Dextran 40. In some embodiments, the cryopreservation composition comprises from or from about 10 to or to about 50, from or from about 10 to or to about 45, from or from about 10 to or to about 40, from or from about 10 to or to about 35, from or from about 10 to or to about 30, from or from about 10 to or to about 25, from or from about 10 to or to about 20, from or from about 10 to or to about 15, from or from about 15 to or to about 50, from or from about 15 to or to about 45, from or from about 15 to or to about 40, from or from about 15 to or to about 35, from or from about 15 to or to about 30, from or from about 15 to or to about 25, from or from about 15 to or to about 20, from or from about 20 to or to about 50, from or from about 20 to or to about 45, from or from about 20 to or to about 40, from or from about 20 to or to about 30, from or from about 20 to or to about 25, from or from about 25 to or to about 50, from or from about 25 to or to about 45, from or from about 25 to or to about 40, from or from about 25 to or to about 35, from or from about 25 to or to about 30, from or from about 30 to or to about 50, from or from about 30 to or to about 45, from or from about 30 to or to about 40, from or from about 30 to or to about 35, from or from about 35 to or to about 50, from or from about 35 to or to about 45, from or from about 35 to or to about 40, from or from about 40 to or to about 50, from or from about 40 to or to about 45, or from or from about 45 to or to about 50 g/L dextran, e.g., Dextran 40. In some embodiments, the cryopreservation composition comprises 10, 15, 20, 25, 30, 30, 35, 40, 45, or 50 g/L dextran, e.g., Dextran 40. In some embodiments, the cryopreservation composition comprises about 10, about 15, about 20, about 25, about 30, about 30, about 35, about 40, about 45, or about 50 g/L dextran, e.g., Dextran 40.

3. Glucose

In some embodiments, the cryopreservation composition comprises glucose.

In some embodiments, as described above, the cryopreservation composition comprises a Dextran solution comprising glucose.

In some embodiments, the cryopreservation composition comprises a Dextran solution that does not comprise glucose. In some embodiments, e.g., when the Dextran solution does not comprise glucose, glucose is added separately to the cryopreservation composition.

In some embodiments, the cryopreservation composition comprises from or from about 5 to or to about 25 g/L glucose. In some embodiments, the cryopreservation composition comprises from or from about 5 to or to about 25, from or from about 5 to or to about 20, from or from about 5 to or to about 15, from or from about 5 to or to about 10, from or from about 10 to or to about 25, from or from about 10 to or to about 20, from or from about 10 to or to about 15, from or from about 15 to or to about 25, from or from about 15 to or to about 20, or from or from about 20 to or to about 25 g/L glucose. In some embodiments, the cryopreservation composition comprises 5, 7.5, 10, 12.5, 15, 17.5, 20, 22.5, or 25 g/L glucose. In some embodiments, the cryopreservation composition comprises 12.5 g/L glucose. In some embodiments, the cryopreservation composition comprises about 5, about 7.5, about 10, about 12.5, about 15, about 17.5, about 20, about 22.5, or about 25 g/L glucose. In some embodiments, the cryopreservation composition comprises about 12.5 g/L glucose.

In some embodiments, the cryopreservation composition comprises less than 2.75% w/v glucose. In some embodiments, the cryopreservation composition comprises less than 27.5 g/L glucose. In some embodiments, the cryopreservation composition comprises less than 2% w/v glucose. In some embodiments, the cryopreservation composition comprises less than 1.5% w/v glucose. In some embodiments, the cryopreservation composition comprises about 1.25% w/v or less glucose.

4. Dimethyl Sulfoxide

In some embodiments, the cryopreservation composition comprises dimethyl sulfoxide (DMSO, also referred to as methyl sulfoxide and methylsulfinylmethane).

In some embodiments, the DMSO is provided as a solution, also referred to herein as a DMSO solution. Thus, in some embodiments, the cryopreservation composition comprises a DMSO solution.

In some embodiments, the DMSO solution is suitable for intravenous use.

In some embodiments, the DMSO solution comprises 1.1 g/mL DMSO. In some embodiments, the DMSO solution comprises about 1.1 g/mL DMSO.

In some embodiments, the cryopreservation composition comprises from or from about 1% to or to about 10% v/v of the DMSO solution. In some embodiments, the cryopreservation composition comprises from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 90%, from or from about 5% to or to about 8%, from or from about 5% to or to about 7%, from or from about 5% to or to about 6%, from or from about 6% to or to about 10%, from or from about 6% to or to about 90%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v of the DMSO solution. In some embodiments, the cryopreservation composition comprises 1%, 2%, 3%, 4%, 5%, 6%, 7%, 8%, 9%, or 10% v/v of the DMSO solution. In some embodiments, the cryopreservation composition comprises 5% of the DMSO solution. In some embodiments, the cryopreservation composition comprises about 1%, about 2%, about 3%, about 4%, about 5%, about 6%, about 7%, about 8%, about 9%, or about 10% v/v of the DMSO solution. In some embodiments, the cryopreservation composition comprises about 5% of the DMSO solution.

In some embodiments, the cryopreservation composition comprises from or from about 11 to or to about 110 g/L DMSO. In some embodiments, from or from about the cryopreservation composition comprises from or from about 11 to or to about 110, from or from about 11 to or to about 99, from or from about 11 to or to about 88, from or from about 11 to or to about 77, from or from about 11 to or to about 66, from or from about 11 to or to about 55, from or from about 11 to or to about 44, from or from about 11 to or to about 33, from or from about 11 to or to about 22, from or from about 22 to or to about 110, from or from about 22 to or to about 99, from or from about 22 to or to about 88, from or from about 22 to or to about 77, from or from about 22 to or to about 77, from or from about 22 to or to about 66, from or from about 22 to or to about 55, from or from about 22 to or to about 44, from or from about 22 to or to about 33, from or from about 33 to or to about 110, from or from about 33 to or to about 99, from or from about 33 to or to about 88, from or from about 33 to or to about 77, from or from about 33 to or to about 66, from or from about 33 to or to about 55, from or from about 33 to or to about 44, from or from about 44 to or to about 110, from or from about 44 to or to about 99, from or from about 44 to or to about 88, from or from about 44 to or to about 77, from or from about 44 to or to about 66, from or from about 44 to or to about 55, from or from about 55 to or to about 110, from or from about 55 to or to about 99, from or from about 55 to or to about 88, from or from about 55 to or to about 77, from or from about 55 to or to about 66, from or from about 66 to or to about 110, from or from about 66 to or to about 99, from or from about 66 to or to about 88, from or from about 66 to or to about 77, from or from about 77 to or to about 119, from or from about 77 to or to about 88, from or from about 88 to or to about 110, from or from about 88 to or to about 99, or from or from about 99 to or to about 110 g/L DMSO. In some embodiments, the cryopreservation composition comprises 11, 22, 33, 44, 55, 66, 77, 88, 99, or 110 g/L DMSO. In some embodiments, the cryopreservation composition comprises 55 g/L DMSO. In some embodiments, the cryopreservation composition comprises about 11, about 22, about 33, about 44, about 55, about 66, about 77, about 88, about 99, or about 110 g/L DMSO. In some embodiments, the cryopreservation composition comprises about 55 g/L DMSO.

5. Buffers

In some embodiments, the cryopreservation composition comprises a buffer solution, e.g., a buffer solution suitable for intravenous administration.

Buffer solutions include, but are not limited to, phosphate buffered saline (PBS), Ringer's Solution, Tyrode's buffer, Hank's balanced salt solution, Earle's Balanced Salt Solution, saline, and Tris.

In some embodiments, the buffer solution is phosphate buffered saline (PBS).

6. Exemplary Cryopreservation Compositions

In some embodiments, the cryopreservation composition comprises or consists of: 1) albumin, e.g., human albumin, 2) dextran, e.g., Dextran 40, 3) DMSO, and 4) a buffer solution. In some embodiments, the cryopreservation composition further comprises glucose. In some embodiments, the cryopreservation composition consists of 1) albumin, e.g., human albumin, 2) dextran, e.g., Dextran 40, 3) glucose, 4) DMSO, and 5) a buffer solution.

In some embodiments, the cryopreservation composition comprises: 1) an albumin solution described herein, 2) a dextran solution described herein, 3) a DMSO solution described herein, and 4) a buffer solution.

In some embodiments, the cryopreservation composition consists of: 1) an albumin solution described herein, 2) a dextran solution described herein, 3) a DMSO solution described herein, and 4) a buffer solution.

In some embodiments, the cryopreservation composition does not comprise a cell culture medium.

In one embodiment, the cryopreservation composition comprises or comprises about 40 mg/mL human albumin, 25 mg/mL Dextran 40, 12.5 mg/mL glucose, and 55 mg/mL DMSO.

In one embodiment, the cryopreservation composition comprises or comprises about or consists of or consists of about 40 mg/mL human albumin, 25 mg/mL Dextran 40, 12.5 mg/mL glucose, 55 mg/mL DMSO, and 0.5 mL/mL 100% phosphate buffered saline (PBS) in water.

In one embodiment, the cryopreservation composition comprises or comprises about 32 mg/mL human albumin, 25 mg/mL Dextran 40, 12.5 mg/mL glucose, and 55 mg/mL DMSO.

In one embodiment, the cryopreservation composition comprises or comprises about or consists of or consists of about of 32 mg/mL human albumin, 25 mg/mL Dextran 40, 12.5 mg/mL glucose, 55 mg/mL DMSO, and 0.54 mL/mL 100% phosphate buffered saline (PBS) in water.

Exemplary Cryopreservation Compositions are shown in Table 3.

TABLE 3
Exemplary Cryopreservation Compositions

| | | | Exemplary |
|---|---|---|---|
| | Concentration | Exemplary | Range v/v % in |
| Excipient | Range | Solution | Cryopreservation |
| Solution | of Solution | Concentration | Composition |
| Albumin | 40-200 g/L | 200 g/L | 10%-50% |
| Solution | albumin in water | albumin | |
| Dextran 40 | 25-200 g/L | 100 g/L | 10%-50% |
| Solution | Dextran 40; and | Dextran 40; | |
| | 0-100 g/L | 50 g/L | |
| | glucose in water | glucose | |
| DMSO | 11-110 g/L | 1,100 g/L | 1%-10% |
| | DMSO in water | DMSO | |
| Buffer | to volume | to volume | to volume |

TABLE 4
Exemplary Cryopreservation Composition #1

| | | Exemplary | Final |
|---|---|---|---|
| | | v/v % in | Concentration in |
| Excipient | Solution | Cryopreservation | Cryopreservation |
| Solution | Composition | Composition #1 | Composition #1 |
| Albumin | 200 g/L | 20% | 40 mg/mL |
| Solution | albumin in water | | albumin |
| Dextran 40 | 100 g/L | 25% | 25 mg/mL |
| Solution | Dextran 40; and | | Dextran 40; |
| | 50 g/L | | 12.5 mg/mL |
| | glucose in water | | glucose |
| DMSO | 100% DMSO | 5% | 55 mg/mL |
| | (1,100 g/L) | | |
| Buffer | 100% Phosphate | 50% | 0.5 mL/mL |
| | Buffered Saline | | |
| | (PBS) | | |

TABLE 5
Exemplary Cryopreservation Composition #2

| | | Exemplary | Final |
|---|---|---|---|
| | | v/v % in | Concentration in |
| Excipient | Solution | Cryopreservation | Cryopreservation |
| Solution | Composition | Composition #2 | Composition #2 |
| Albumin | 200 g/L | 16% | 32 mg/mL |
| Solution | albumin in water | | albumin |
| Dextran 40 | 100 g/L | 25% | 25 mg/mL |
| Solution | Dextran 40; and | | Dextran 40; |
| | 50 g/L | | 12.5 mg/mL |
| | glucose in water | | glucose |
| DMSO | 100% DMSO | 5% | 55 mg/mL |
| | (1,100 g/L) | | |
| Buffer | 100% Phosphate | 54% | 0.54 mL/mL |
| | Buffered Saline | | |
| | (PBS) | | |

B. Methods of Cryopreserving

The cryopreservation compositions described herein can be used for cryopreserving cell(s), e.g., therapeutic cells, e.g., natural killer (NK) cell(s), e.g., the NK cell(s) described herein.

In some embodiments, the cell(s) are an animal cell(s). In some embodiments, the cell(s) are human cell(s).

In some embodiments, the cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, cryopreserving the cell(s) comprises: mixing the cell(s) with a cryopreservation composition or components thereof described herein to produce a composition, e.g., a pharmaceutical composition; and freezing the mixture.

In some embodiments, cryopreserving the cell(s) comprises: mixing a composition comprising the cell(s) with a cryopreservation composition or components thereof described herein to produce a composition, e.g., a pharmaceutical composition; and freezing the mixture. In some embodiments, the composition comprising the cell(s) comprises: the cell(s) and a buffer. Suitable buffers are described herein.

In some embodiments, cryopreserving the cell(s) comprises: mixing a composition comprising the cell(s) and a buffer, e.g., PBS, with a composition comprising albumin, Dextran, and DMSO, e.g., as described herein; and freezing the mixture.

In some embodiments, cryopreserving the cell(s) comprises: mixing a composition comprising the cell(s) and a buffer, e.g., PBS 1:1 with a composition comprising 40 mg/mL albumin, e.g., human albumin, 25 mg/mL Dextran, e.g., Dextran 40, 12.5 mg/mL glucose and 55 mg/mL DMSO.

In some embodiments, the composition comprising the cell(s) and the buffer, e.g., PBS, comprises from or from about 2×10⁷ to or to about 2×10⁹ cells/mL. In some embodiments, the composition comprising the cell(s) and the buffer, e.g., PBS, comprises 2×10⁸ cells/mL. In some embodiments, the composition comprising the cell(s) and the buffer, e.g., PBS, comprising about 2×10⁸ cells/mL.

In some embodiments, cryopreserving the cell(s) comprising mixing: the cell(s), a buffer, e.g., PBS, albumin, e.g., human albumin, Dextran, e.g., Dextran 40, and DMSO; and freezing the mixture.

In some embodiments, the mixture comprises from or from about 1×10⁷ to or to about 1×10⁹ cells/mL. In some embodiments, the mixture comprises 1×10⁸ cells/mL. In some embodiments, the mixture comprises about 1×10⁸ cells/mL.

Suitable ranges for albumin, Dextran, and DMSO are set forth above.

In some embodiments, the composition is frozen at or below −135° C.

In some embodiments, the composition is frozen at a controlled rate.

### IV. Pharmaceutical Compositions

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### V. Methods of Treatment

The NK cells described herein, find use for treating cancer or other proliferative disorders.

Thus, also provided herein are methods of treating a patient suffering from a disorder, e.g., a disorder associated with a cancer, cancer, comprising administering the NK cells, e.g., the NK cells described herein, and optionally an antibody.

Also provided herein are methods of preventing, reducing and/or inhibiting the recurrence, growth, proliferation, migration and/or metastasis of a cancer cell or population of cancer cells in a subject in need thereof, comprising administering the NK cells, e.g., the NK cells described herein, and optionally an antibody.

Also provided herein are methods of enhancing, improving, and/or increasing the response to an anticancer therapy in a subject in need thereof, comprising administering the NK cells, e.g., the NK cells described herein, and optionally an antibody.

Also provided herein are methods for inducing the immune system in a subject in need thereof comprising administering the NK cells, e.g., the NK cells described herein, and optionally an antibody.

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. Generally, the methods include administering a therapeutically effective amount of a treatment as described herein, to a subject who is in need of, or who has been determined to be in need of, such treatment. In some embodiments, the methods include administering a therapeutically effective amount of a treatment comprising an NK cells, e.g., NK cells described herein, and optionally an antibody.

As used herein, the terms “treatment,” “treat,” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition”, as it relates to cancer and/or cancer cell proliferation, refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

A. Disorders

Methods and manufactured compositions disclosed herein find use in targeting a number of disorders, such as cellular proliferative disorders. A benefit of the approaches herein is that allogenic cells are used in combination with exogenous antibody administration to target specific proliferating cells targeted by the exogenous antibody. Unlike previous therapies, such as chemo or radiotherapy, using the approaches and pharmaceutical compositions herein, one is able to specifically target cells exhibiting detrimental proliferative activity, potentially without administering a systemic drug or toxin that impacts proliferating cells indiscriminately.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the cancer is selected from the group consisting of: acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, and Wilms tumor.

In some embodiments, the cancer is a solid tumor.

In some embodiments, the cancer is metastatic.

B. Patients

Suitable patients for the compositions and methods herein include those who are suffering from, who have been diagnosed with, or who are suspected of having a cellular proliferative and/or differentiative disorder, e.g., a cancer. Patients subjected to technology of the disclosure herein generally respond better to the methods and compositions herein, in part because the pharmaceutical compositions are allogeneic and target cells identified by the antibodies, rather than targeting proliferating cells generally. As a result, there is less off-target impact and the patients are more likely to complete treatment regimens without substantial detrimental off-target effects.

In some embodiments, the methods of treatment provided herein may be used to treat a subject (e.g., human, monkey, dog, cat, mouse) who has been diagnosed with or is suspected of having a cellular proliferative and/or differentiative disorder, e.g., a cancer. In some embodiments, the subject is a mammal. In some embodiments, the subject is a human.

As used herein, a subject refers to a mammal, including, for example, a human.

In some embodiments, the mammal is selected from the group consisting of an armadillo, an ass, a bat, a bear, a beaver, a cat, a chimpanzee, a cow, a coyote, a deer, a dog, a dolphin, an elephant, a fox, a panda, a gibbon, a giraffe, a goat, a gopher, a hedgehog, a hippopotamus, a horse, a humpback whale, a jaguar, a kangaroo, a koala, a leopard, a lion, a llama, a lynx, a mole, a monkey, a mouse, a narwhal, an orangutan, an orca, an otter, an ox, a pig, a polar bear, a porcupine, a puma, a rabbit, a raccoon, a rat, a rhinoceros, a sheep, a squirrel, a tiger, a walrus, a weasel, a wolf, a zebra, a goat, a horse, and combinations thereof.

In some embodiments, the mammal is a human.

The subject, e.g., the human subject, can be a child, e.g., from or from about 0 to or to about 14 years in age. The subject can be a youth, e.g., from or from about 15 to or to about 24 years in age. The subject can be an adult, e.g., from or from about 25 to or to about 64 years in age. The subject can be a senior, e.g., 65+ years in age.

In some embodiments, the subject may be a human who exhibits one or more symptoms associated with a cellular proliferative and/or differentiative disorder, e.g., a cancer, e.g., a tumor. Any of the methods of treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. The methods of treatment described herein can promote reduction or retraction of a tumor, decrease or inhibit tumor growth or cancer cell proliferation, and/or induce, increase or promote tumor cell killing. In some embodiments, the subject is in cancer remission. The methods of treatment described herein can prevent or delay metastasis or recurrence of cancer.

In some embodiments, the subject is at risk, or genetically or otherwise predisposed (e.g., risk factor), to developing a cellular proliferative and/or differentiative disorder, e.g., a cancer, that has or has not been diagnosed.

As used herein, an “at risk” individual is an individual who is at risk of developing a condition to be treated, e.g., a cellular proliferative and/or differentiative disorder, e.g., a cancer. Generally, an “at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

C. Lymphodepletion

In some embodiments, the patient is lymphodepleted before treatment.

Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 250 to about 500 mg/m² of cyclophosphamide, e.g., from or from about 250 to or to about 500, 250, 400, 500, about 250, about 400, or about 500 mg/m² of cyclophosphamide.

In some embodiments, lymphodepletion comprises administration of or of about 20 mg/m²/day to or to about 40 mg/m²/day fludarabine, e.g., 30 or about 30 mg/m²/day.

In some embodiments, lymphodepletion comprises administration of both cyclophosmamide and fludarabine.

In some embodiments, the patient is lymphodepleted by intravenous administration of cyclophosphamide (250 mg/m²/day) and fludarabine (30 mg/m²/day).

In some embodiments, the patient is lymphodepleted by intravenous administration of cyclophosphamide (500 mg/m²/day) and fludarabine (30 mg/m²/day).

In some embodiments, the lymphodepletion occurs no more than 5 days prior to the first dose of NK cells. In some embodiments, the lymphodepletion occurs no more than 7 days prior to the first dose of NK cells.

In some embodiments, lymphodepletion occurs daily for 3 consecutive days, starting 5 days before the first dose of NK cells (i.e., from Day −5 through Day −3).

In some embodiments, the lymphodepletion occurs on day −5, day −4 and day −3.

D. Administration

1. NK Cells

In some embodiments, the NK cells are administered as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells drawn from a common batch or donor.

In some embodiments, the NK cells, e.g., the NK cells described herein are administered at or at about 1×10⁸ to or to about 8×10⁹ NK cells per dose. In some embodiments, the NK cells are administered at or at about 1×10⁸, at or at about 1×10⁹, at or at about 4×10⁹, or at or at about 8×10⁹ NK cells per dose.

In some embodiments, the NK cells are administered weekly. In some embodiments, the NK cells are administered for or for about weeks. In some embodiments, the NK cells are administered weekly for or for about 8 weeks.

In some embodiments, the NK cells are cryopreserved in an infusion-ready media, e.g., a cryopreservation composition suitable for intravenous administration, e.g., as described herein.

In some embodiments, the NK cells are cryopreserved in vials containing from or from about 1×10⁸ to or to about 8×10⁹ cells per vial. In some embodiments, the NK cells are cryopreserved in vials containing a single dose.

In some embodiments, the cells are thawed, e.g., in a 37° C. water bath, prior to administration.

In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity.

In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing.

In some embodiments, the pharmaceutical composition is administered intravenously via syringe.

In some embodiments, 1 mL, 4 mL, or 10 mL of drug product is administered to the patient intravenously via syringe.

2. Antibodies

In some embodiments, the NK cell(s) described herein, e.g., the pharmaceutical compositions comprising NK cell(s) described herein, are administered in combination with an antibody. In some embodiments, an antibody is administered together with the NK cells as part of a pharmaceutical composition. In some embodiments, an antibody is administered separately from the NK cells, e.g., as part of a separate pharmaceutical composition. Antibodies can be administered prior to, subsequent to, or simultaneously with administration of the NK cells.

In some embodiments, the antibody is administered before the NK cells. In some embodiments, the antibody is administered after the NK cells.

In some embodiments, the NK cells are administered at least 30 minutes, 60 minutes, 90 minutes, 120 minutes, 150 minutes, 180 minutes, 210 minutes, or 240 minutes after completing administration of the antibody.

In some embodiments, the NK cells are administered the day after the antibody is administered.

In some embodiments, the NK cells are administered at each administration, while the antibody is administered at a subset of the administrations. For example, in some embodiments, the NK cells are administered once a week and the antibody is administered once a month.

In some embodiments, the antibody is administered weekly for 8 weeks. In some embodiments, the antibody is administered every two weeks for 8 weeks.

In some embodiments, a dose of antibody is given prior to the first dose of cells. In some embodiments, a debulking dose of the antibody is given prior to the first dose of cells.

3. Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition.

In some embodiments, the cytokine is IL-2.

In some embodiments, the IL-2 is administered subcutaneously.

In some embodiments, the IL-2 is administered from between 1 to 4 or about 1 to about 4 hours following the conclusion of NK cell administration. In some embodiments, the IL-2 is administered at least 1 hour following the conclusion of NK cell administration. In some embodiments, the IL-2 is administered no more than 4 hours following the conclusion of NK cell administration. In some embodiments, the IL-2 is administered at least 1 hour after and no more than 4 hours following the conclusion of NK cell administration.

In some embodiments, the IL-2 is administered at up to 10 million IU/M², e.g., up to 1 million, 2 million, 3 million, 4 million, 5 million, 6 million, 7 million, 8 million, 9 million, or 10 million IU/m².

In some embodiments, the IL-2 is administered at or at about 1 million, at or at about 2 million, at or at about 3 million, at or at about 4 million, at or at about 5 million, at or at about 6 million, at or at about 7 million, at or at about 8 million, at or at about 9 million, at or at about 10 million IU/M²

In some embodiments, the IL-2 is administered at or at about 1×10⁶ IU/M². In some embodiments, the IL-2 is administered at or at about 2×10⁶ IU/M².

In some embodiments, less than 1×10⁶IU/M² IL-2 is administered to the patient.

In some embodiments, a flat dose of IL-2 is administered to the patient. In some embodiments, a flat dose of 6 million IU or about 6 million IU is administered to the patient.

In some embodiments, IL-2 is not administered to the patient.

E. Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

F. Combination Therapies

In some embodiments, the method comprises administering the NK cells described herein, e.g., the NK cells described herein, in combination with another therapy, e.g., an antibody, an NK cell engager, an antibody drug conjugate (ADC), a chemotherapy drug, e.g., a small molecule drug, an immune checkpoint inhibitor, and combinations thereof.

1. Antibodies

In some embodiments, the other therapy is an antibody.

In some embodiments, the antibody binds to a target selected from the group consisting of CD20, HER-2, EGFR, CD38, SLAMF7, GD2, ALK1, AMHR2, CCR2, CD137, CD19, CD26, CD32b, CD33, CD37, CD70, CD73, CD74, CD248, CLDN6, Clever-1, c-MET, CSF-1R, CXCR4, DKK1, DR5, Epha3, FGFR2b, FGFR3, FLT3, FOLR1, Globo-H, Glypican3, GM1, Grp78, HER-3, HGF, IGF-IR, IL1RAP, IL-8R, ILT4, Integrin alpha V, M-CSF, Mesothelin, MIF, MUC1, MUC16, MUC5AC, Myostatin, NKG2A, NOTCH, NOTCH2/3, PIGF, PRL3, PSMA, ROR1, SEMA4D, Sialyl Lewis A, Siglec15, TGF-b, TNFR3, TRAIL-R2, VEGF, VEGFR1, VEGFR2, Vimentin, and combinations thereof.

Suitable antibodies include, but are not limited to those shown in Table 6.

TABLE 6
Antibodies for Combination Therapy

| Target | Drug Name | Brand Name | Indication(s) | Reference |
|---|---|---|---|---|
| CD20 | Rituxan | Rituximab | DLBCL/FL, | Du et al., Auto Immun |
| | | | NHL, CLL, RA, | Highlights (2017) 8(1): 12 |
| | | | GPA, MPA | |
| CD20 | Gazyva | Obinutuzumab | CLL, FL | Gagez et al., Curr Opin |
| | | | | Oncol. 2014 September; 26(5): |
| | | | | 484-91 |
| CD20 | Arzerra | Ofatumumab | CLL | Robak, Curr Opin Mol Ther. |
| | | | | 2008 June; 10(3): 294-309 |
| CD20 | Ocrevus | Ocrelizumab | RMS, PPMS | Genovese et al., Arthritis |
| | | | | Rheum. 2008 |
| | | | | September; 58(9): 2652-61 |
| CD20 | Zevalin | Ibritumomab | NHL | Wiseman et al., Eur J Nucl |
| | | | | Med. 2000 July; 27(7): 766-77 |
| CD20 | | Veltuzumab | NHL, CLL | Kalaycio et al. Leuk |
| | | | | Lymphoma. 2016; 57(4): 803-11 |
| CD20 | Bexxar | Tositumomab | NHL | Vose et al., J Clin Oncol. |
| | | and Iodine I 131 | | 2000 March; 18(6): 1316-23 |
| | | tositumomab | | |
| CD20 | | Ublituximab | NHL, CLL, | Sawas et al., Br J Haematol. |
| | | | RMS | 2017 April; 177(2): 243-253 |
| HER-2 | Herceptin | Trastuzumab | Breast, Gastric | Goldenberg, Clin Ther. 1999 |
| | | | | February; 21(2): 309-18 |
| HER-2 | Perjeta | Pertuzumab | Breast | Agus et al., J Clin Oncol. |
| | | | | 2005 Apr. 10; 23(11): 2534-43 |
| HER-2 | Margenza | Margetuximab | Breast | Bang et al., Ann Oncol. 2017 |
| | | | | Apr. 1; 28(4): 855-861 |
| EGFR | Erbitux | Cetuximab | CRC, HNC | Jonker et al., N Engl J Med |
| | | | | 2007; 357: 2040-2048 |
| EGFR | Vectibix | Panitumumab | CRC | Gibson et al., Clin Colorectal |
| | | | | Cancer. 2006 May; 6(1): 29-31 |
| EGFR | Portrazza | Necitumumab | NSCLC | Kuenen et al., Clin Cancer |
| | | | | Res. 2010 Mar. |
| | | | | 15; 16(6): 1915-23 |
| CD38 | Darzalex | Daratumumab | MM | de Weers et al., J Immunol. |
| | | | | 2011 Feb. 1; 186(3): 1840-8 |
| CD38 | Sarclisa | Isatuximab | MM | Martin et al., Blood Cancer J. |
| | | | | 2019 Mar. 29; 9(4): 41 |
| SLAMF7 | Empliciti | Elotuzumab | MM | Lonial et al., N Engl J Med |
| | | | | 2015; 373: 621-631 |
| GD2 | Unituxin | Dinutuximab | NB | Hoy, Target Oncol. 2016 |
| | | | | April; 11(2): 247-53 |
| GD2 | Danyelza | Naxitamab | NB | Markham, Drugs. 2021 |
| | | | | February; 81(2): 291-296 |
| ALK1 | PF-03446962 | Ascrinvacumab | Liver cancer | Simonelli et al., Ann Oncol. |
| | | | | 2016 September; 27(9): 1782-7 |
| AMHR2 | GM-102 | Murlentamab | Ovarian Cancer | Leary et al., J Clin Oncol. |
| | | | | 2019 37: 15_suppl, 2521-2521 |
| CCR2 | TAK-202 | Plozalizumab | Atherosclerosis, | Gilbert et al., Am J Cardiol. |
| | | | Melanoma | 2011 Mar. 15; 107(6): 906-11 |
| CD137 | BMS-663513 | Urelumab | Melanoma, | Segal et al., Clin Cancer Res. |
| | | | Myeloma, | 2017 Apr. 15; 23(8): 1929-1936 |
| | | | NSCLC | |
| CD137 | PF-05082566 | Utomilumab | Ovarian Cancer | Segal et al., Clin Cancer Res. |
| | | | | 2018 Apr. 15; 24(8): 1816-1823 |
| CD19 | AMG103 | Blinatumomab | ALL, NHL | Nadafi et al., Int J Mol Cell |
| | | | | Med (2015) 4(3): 143-151 |
| CD19 | SAR3419 | Coltuximab | ALL, NHL | Nadafi et al. |
| | | Ravtansine | | |
| CD19 | XmAb 5574 | MOR208 | ALL, NHL, CLL | Nadafi et al. |
| CD19 | MEDI-551 | MEDI-551 | B-cell | Nadafi et al. |
| | | | malignancies, | |
| | | | CLL, Multiple | |
| | | | Myeloma, | |
| | | | Scleroderma | |
| CD19 | SGN-19A | Denintuzumab | NHL | Nadafi et al. |
| | | Mafodotin | | |
| CD19 | | DI-B4 | B-cell | Nadafi et al. |
| | | | malignancies | |
| CD19 | Taplitumomabpaptox | Taplitumomabpaptox | B-cell | Nadafi et al. |
| | | | malignancies | |
| CD19 | XmAb 5871 | XmAb 5871 | Autoimmune | Nadafi et al. |
| | | | Diseases | |
| CD19 | MDX-1342 | MDX-1342 | CLL, | Nadafi et al. |
| | | | Rheumatoid | |
| | | | Arthritis | |
| CD19 | AFM11 | AFM11 | NHL | Nadafi et al. |
| CD19 | ADCT-402 | Loncastuximab | ALL, NHL | Yu et al., Journal of |
| | | Tesirine | | Hematology & Oncology |
| | | | | (2019) 12(94) |
| CD19 | Monjuvi | Tafasitamab | NHL (DLBCL) | Hoy, Drugs. 2020 |
| | | | | November; 80(16): 1731-1737 |
| CD26 | Begedina | Begelomab | Graft versus host | Bacigalupo et al., Bone |
| | | | disease | Marrow Transplant. 2020 |
| | | | | August; 55(8): 1580-1587 |
| CD32b | BI-1206 | BI-1206 | BCL, CLL | Trial ID: NCT04219254 |
| CD33 | Mylotarg | Gemtuzumab | AML | Stasi, Expert Opin Biol Ther. |
| | | Ozogamicin | | 2008 April; 8(4): 527-40 |
| CD33 | SGN-33 | Lintuzumab | AML | Trial ID: NCT02998047 |
| CD37 | BI 836826 | BI 836826 | DLBCL, CLL, | Trial ID: NCT02538614 |
| | | | NHL | |
| CD37 | IMGN529 | Naratuximab | DLBCL, NHL | Yu et al., Journal of |
| | | emtansine | | Hematology & Oncology |
| | | | | (2019) 12(94) |
| CD37 | AGS67E | AGS67E | DLBCL, NHL | Yu et al. |
| CD70 | BMS-936561 | MDX-1203 | DLBCL, MCL | Yu et al. |
| CD70 | SGN-75 | Vorsetuzumab | NHL | Yu et al. |
| | | mafodotin | | |
| CD73 | MEDI9447 | Oleclumab | Pancreatic | Geoghegan et al., MAbs. |
| | | | cancer | 2016; 8(3): 454-67 |
| CD73 | AK119 | AK119 | Covid-19, Solid | Trial ID: NCT04516564 |
| | | | Tumors | |
| CD74 | hLL1-DOX | Milatuzumab | MM | Yu et al. |
| | | doxorubicin | | |
| CD74 | STRO-001 | STRO-001 | MM, NHL | Trial ID: NCT03424603 |
| CD248 | Ontecizumab | Ontuxizumab | MM, Soft tissue | D'Angelo et al., Invest New |
| | | | sarcoma | Drugs. 2018 February: 36(1): |
| | | | | 103-113 |
| CLDN6 | IMAB027 | ASP1650 | Testicular cancer | Trial ID: NCT03760081 |
| Clever-1 | Clevegen | Bexmarilimab | Solid tumors | Trial ID: NCT03733990 |
| c-MET | MetMAb | Onartuzumab | NSCLC | Hughes et al., Trends Cancer |
| | | | | (2018) 4(2): 94-97 |
| c-MET | AMG-102 | Rilotumumab | Gastric cancer | Waddell et al., |
| | | | | Immunotherapy. |
| | | | | 2014; 6(12): 1243-53 |
| CSF-1R | FPA-008 | Cabiralizumab | MM, NSCLC | Trial ID: NCT04050462 |
| CSF-1R | RG-7155 | Emactuzumab | Ovarian cancer | Trial ID: NCT03708224 |
| CSF-1R | IMC CS4 | LY3022855 | MM | Trial ID: NCT03153410 |
| CSF-1R | AMB 051 | AMG 820 | Solid tumors | Trial ID: NCT04731675 |
| CSF-1R | SNDX-6352 | Axatilimab | Graft versus host | Trial ID: NCT04710576 |
| | | | disease | |
| CXCR4 | BMS-936564 | Ulocuplumab | Leukemia | Bobkov et al., Mol |
| | | | | Pharmacol (2019) 96: 753- |
| | | | | 764 |
| CXCR4 | LY2624587 | LY2624587 | Metastatic | Bobkov et al. |
| | | | Cancer | |
| CXCR4 | PF-06747143 | PF-06747143 | AML | Bobkov et al. |
| CXCR4 | F50067 | hz515H7 | MM | Bobkov et al. |
| CXCR4 | MEDI3185 | MEDI3185 | Hematologic | Bobkov et al. |
| | | | malignancies | |
| DKK1 | DKN-01 | DKN-01 | Gastric cancer | Wall et al., Expert Opin |
| | | | | Investig Drugs. 2020 |
| | | | | July; 29(7): 639-644 |
| DKK1 | BHQ880 | BHQ880 | MM | Fulciniti et al., Blood. 2009 |
| | | | | Jul. 9; 114(2): 371-9 |
| DR5 | AD5-10 | Zaptuzumab | Solid tumors | Zhang et al., Theranostics. |
| | | | | 2019 Jul. 13; 9(18): 5412-5423 |
| DR5 | AMG655 | Conatumumab | Colon, | Rosevear et al., Curr Opin |
| | | | pancreatic cancer | Investig Drugs. 2010 |
| | | | | June; 11(6): 688-98 |
| DR5 | PRO955780 | Drozitumab | NHL, NSCLC | Kang et al., Clin Cancer Res. |
| | | | | 2011 May 15; 17(10): 3181-92 |
| DR5 | ETR2-ST01 | Lexatumumab | Solid tumors | Plummer et al., Clin Cancer |
| | | | | Res. 2007 Oct. |
| | | | | 15; 13(20): 6187-94 |
| DR5 | CS-1008 | Tigatuzumab | Solid tumors | Reck et al., Lung Cancer. |
| | | | | 2013 December; 82(3): 441-8 |
| DR5 | | DS-8273a | Solid tumors | Forero et al., Invest New |
| | | | | Drugs. 2017 June; 35(3): 298- |
| | | | | 306 |
| Epha3 | KB004 | KB004 | Glioblastoma | Swords et al., Leuk Res. |
| | | | | 2016 November; 50: 123-131 |
| FGFR2b | FPA-144 | Bemarituzumab | Gastric cancer | Catenacci et al., J Clin |
| | | | | Oncol. 2020 Jul. |
| | | | | 20; 38(21): 2418-2426 |
| FGFR2b | BAY | Aprutumab | Solid tumors | Kim et al., Target Oncol. |
| | 1187982 | ixadotin | | 2019 October; 14(5): 591-601 |
| FGFR2b | BAY- | Aprutumab | Solid tumors | Trial ID: NCT01881217 |
| | 1179470 | | | |
| FGFR3 | LY3076226 | LY3076226 | Solid tumors | Trial ID: NCT02529553 |
| FLT3 | IMC-EB10 | IMC-EB10 | AML | Piloto et al., Cancer Res. |
| | | | | 2006 May 1; 66(9): 4843-51 |
| | AGS 62P1 | ASP1235 | AML | Trial ID: NCT02864290 |
| FOLR1 | MORAb-003 | Farletuzumab | Ovarian cancer | Sato et al., Onco Targets |
| | | | | Ther. 2016 Mar. 7; 9: 1181-8 |
| Globo-H | OBI-833 | OBI-833 | Solid tumors | Trial ID: NCT02310464 |
| Globo-H | OBI-888 | OBI-888 | Solid tumors | Trial ID: NCT03573544 |
| Globo-H | OBI-999 | OBI-999 | Solid tumors | Trial ID: NCT04084366 |
| Glypican3 | GC33 | Codrituzumab | Liver cancer | Abou-Alfa et al., J Hepatol. |
| | | | | 2016 August; 65(2): 289-95 |
| Glypican3 | | ERY974 | Solid tumors | Ishiguro et al., Sci Transl |
| | | | | Med. 2017 Oct. 4; 9(410) |
| GM1 | BMS986012 | BMS-986012 | Lung cancer | Ponath et al., Clin Cancer |
| | | | | Res. 2018 Oct. |
| | | | | 15; 24(20): 5178-5189 |
| Grp78 | PAT-SM6 | PAT-SM6 | Multiple | Hensel et al., Melanoma Res. |
| | | | myeloma | 2013 August; 23(4): 264-75 |
| HER-3 | U3-1402 | Patritumab | NSCLC, Solid | Hashimoto et al., Clin Cancer |
| | | deruxtecan | tumors | Res. 2019 Dec. |
| | | | | 1; 25(23): 7151-7161 |
| HGF | AMG-102 | Rilotumumab | Solid tumors | Waddell et al., |
| | | | | Immunotherapy. |
| | | | | 2014; 6(12): 1243-53 |
| HGF | AV-299 | Ficlatuzumab | AML, NSCLC | Bauman et al., Cancers |
| | | | | (Basel). 2020 Jun. |
| | | | | 11; 12(6): 1537 |
| HGF | L2G7 | TAK-701 | Solid tumors | Okamoto et al., Mol Cancer |
| | | | | Ther. 2010 October; 9(10): |
| | | | | 2785-92 |
| IGF-1R | IMC-A12 | Cixutumumab | EWS, HCC | Chen et al., Chin J Cancer |
| | | | | (2013) 32(5): 242-252 |
| IGF-1R | CP-751 | Figitumumab | EWS, ACC | Chen et al. |
| IGF-1R | MK-0646 | Dalotuzumab | Colorectal | Chen et al. |
| | | | cancer | |
| IGF-1R | AMG 479 | Ganitumab | EWS, DRCT | Chen et al. |
| IGF-1R | | R1507 | EWS | Chen et al. |
| IGF-1R | AVE-1642 | VRDN 001 | MM, Breast | Trial ID: NCT01233895 |
| | | | cancer | |
| IL1RAP | CAN04 | Nidanilimab | NSCLC | Awada et al., J Clin Oncol. |
| | | | | 2019 May; 37: 2504-2504 |
| IL-8R | BMS-986253 | HuMax-IL8 | Covid-19, | Bilusic et al., J Immunother |
| | | | NSCLC | Cancer. 2019 Sep. 5; 7(1): 240 |
| ILT4 | JTX-8064 | JTX-8064 | Solid tumors | Trial ID: NCT04669899 |
| Integrin | IMGN388 | IMGN388 | Solid tumors | Trial ID: NCT00721669 |
| alpha V | | | | |
| Integrin | CNTO-95 | Intetumumab | MM | O'Day et al., Br J Cancer. |
| alpha V | | | | 2011 Jul. 26; 105(3): 346-52 |
| Integrin | EMD525797 | Abituzumab | Colorectal | Jiang et al., Mol Cancer Res. |
| alpha V | | | cancer | 2017 July; 15(7): 875-883 |
| Integrin | MEDI-522 | Etaracizumab | MM, Colorectal | Hersey et al., Cancer. 2010 |
| alpha V | | | cancer | Mar. 15; 116(6): 1526-34 |
| Integrin | VPI-2690B | VPI-2690B | Diabetic | Trial ID: NCT02251067 |
| alpha V | | | nephropathies | |
| M-CSF | MCS-110 | Lacnotuzumab | Breast cancer, | Pognan et al., J Pharmacol |
| | | | Gastric cancer | Exp Ther. 2019 |
| | | | | June; 369(3): 428-442 |
| Mesothelin | MORAb-009 | amatuximab | Mesothelioma | Baldo et al., Onco Targets |
| | | | | Ther. 2017 Nov. 8; 10: 5337- |
| | | | | 5353 |
| Mesothelin | SSI(dsFv)- | SS1P | Neoplasms | Hassan et al., J Clin Oncol. |
| | PE38 | | | 2016 December; 34(34): 4171- |
| | | | | 4179 |
| Mesothelin | BAY 94- | Anetumab | Mesothelioma | Hassan et al., J Clin Oncol. |
| | 9343 | ravtansine | | 2020 Jun. 1; 38(16): 1824- |
| | | | | 1835 |
| Mesothelin | RG7600 | DMOT4039A | Pancreatic | Hassan et al., J Clin Oncol. |
| | | | cancer, ovarian | 2016 December; 34(34): 4171- |
| | | | cancer | 4179 |
| Mesothelin | BMS-986148 | BMS-986148 | Solid Tumors | Hassan et al., J Clin Oncol. |
| | | | | 2016 December; 34(34): 4171- |
| | | | | 4179 |
| MIF | BAX69 | Imalumab | Colorectal | Mahalingham et al., Br J Clin |
| | | | cancer | Pharmacol. 2020 |
| | | | | September; 86(9): 1836-1848 |
| MUC1 | huC242- | Cantuzumab | Pancreatic | Tolcher et al., J Clin Oncol. |
| | DM1 | mertansine | cancer | 2003 Jan. 15; 21(2): 211-22 |
| MUC1 | hPAM4 | Clivatuzumab | Pancreatic | Liu et al., Oncotarget. 2015 |
| | | | cancer | Feb. 28; 6(6): 4274-85 |
| MUC1 | GT-MAB | Gatipotuzumab | Ovarian cancer | Heublin et al., Int J Mol Sci. |
| | 2.5-GEX ™ | | | 2019 Jan. 12; 20(2): 295 |
| MUC1 | mAb-AR20.5 | AR20.5 | Pancreatic | de Bono et al., Ann Oncol. |
| | | | cancer | 2004 December; 15(12): 1825-33 |
| MUC16 | ACA 125 | Abagovomab | Ovarian cancer | Sabbatini et al., J Clin Oncol. |
| | | | | 2013 Apr. 20; 31(12): 1554-61 |
| MUC16 | DMUC5754A | Sofituzumab | Ovarian cancer | Liu et al., Ann Oncol. 2016 |
| | | vedotin | | November; 27(11): 2124-2130 |
| MUC16 | DMUC4064A | THIOMAB ™ | Ovarian cancer | Trial ID: NCT02146313 |
| MUC5AC | PAM4 | Clivatuzumab | PDAC | Gold et al., Molecular Cancer |
| | | | | (2013) 12: 143 |
| MUC5AC | NPC-1C | Ensituximab | Pancreatic | Kim et al., Clin Cancer Res. |
| | | | cancer | 2020 Jul. 15; 26(14): 3557- |
| | | | | 3564 |
| Myostatin | MYO-029 | Stamulumab | Muscular | Trial ID: NCT00563810 |
| | | | atrophy, | |
| | | | Muscular | |
| | | | dystrophies | |
| Myostatin | PF-06252616 | Domagrozumab | Duchenne | Wagner et al., Neuromuscul |
| | | | muscular | Disord. 2020 June; 30(6): 492- |
| | | | dystrophy | 502 |
| Myostatin | LY-2495655 | Landogrozumab | Muscular | Golan et al., J Cachexia |
| | | | atrophy, | Sarcopenia Muscle. 2018 |
| | | | Pancreatic | October; 9(5): 871-879 |
| | | | cancer | |
| Myostatin | REGN-1033 | Trevogrumab | Muscular | Trial ID: NCT01720576 |
| | | | atrophy | |
| Myostatin | SRK-015 | Apitegromab | Spinal muscular | Trial ID: NCT03921528 |
| | | | atrophy | |
| NKG2A | IPH2201 | Monalizumab | Breast cancer; | Andre et al., Cell. 2018 Dec. |
| | | | NSCLC | 13; 175(7): 1731-1743 |
| NOTCH | OMP-21M18 | Demcizumab | NSCLC | Takebe et al., Pharmacol |
| | | | | Ther (2014) 141(2): 140-149 |
| NOTCH | REGN421/ | Enoticumab | NSCLC, Ovarian | Takebe et al. |
| | SAR153192 | | cancer | |
| NOTCH | OPM-52M51 | Brontictuzumab | Solid tumors | Takebe et al. |
| NOTCH2/3 | OMP-59R5 | Tarextumab | Sarcomas, Rectal | Takebe et al. |
| | | | cancer | |
| PIGF | RO5323441 | TB-403 | Solid tumors | Martinsson-Niskanen et al., |
| | | | | Clin Ther. 2011 |
| | | | | September; 33(9): 1142-9 |
| PRL3 | PRL3- | PRL3-zumab | Solid tumors | Trial ID: NCT04452955 |
| | ZUMAB | | | |
| PSMA | Capromab | Capromab | Prostate cancer | Trial ID: NCT00992745 |
| | | pendetide | | |
| PSMA | MT112 | Pasotuxizumab | Prostate cancer | Hummel et al., |
| | | | | Immunotherapy. 2021 |
| | | | | February; 13(2): 125-141 |
| PSMA | | MDX1201-A488 | Prostate cancer | Trial ID: NCT02048150 |
| PSMA | APVO 414 | MOR209/ES414 | Prostate cancer | Hernandez-Hoyos et al., Mol |
| | | | | Cancer Ther 2016 |
| | | | | September; 15(9): 2155-65 |
| PSMA | ARX-517 | ARX517 | Prostate cancer | Trial ID: NCT04662580 |
| PSMA | ADCT 401 | MEDI3726 | Prostate cancer | Cho et al., Mol Cancer Ther. |
| | | | | 2018 October; 17(10): 2176-2186 |
| PSMA | | JNJ-63898081 | Prostate cancer | Trial ID: NCT03926013 |
| PSMA | PSMA TTC | BAY 2315497 | Prostate cancer | Hammer et al., Clin Cancer |
| | | | | Res. 2020 Apr. |
| | | | | 15; 26(8): 1985-1996 |
| PSMA | | TLX592 | Prostate cancer | Trial ID: NCT04726033 |
| PSMA | DOTA-HUJ- | Rosopatamab | Prostate cancer | Vallabhajosula et al., Curr |
| | 591 | tetraxetan | | Radiopharm. 2016; 9(1): 44-53 |
| PSMA | | PSMA ADC | Prostate cancer | Petrylak et al., Prostate. 2020 |
| | | | | Janruary; 80(1): 99-108 |
| ROR1 | UC-961 | Cirmtuzumab | CLL, MCL | Choi et al., Cell Stem Cell. |
| | | | | 2018 Jun. 1; 22(6): 951-959 |
| SEMA4D | VX15/2503 | Pepinemab | NSCLC, MM | |
| Sialyl Lewis | MVT-5873 | MVT-5873 | Colorectal | Gupta et al., J Gastrointest |
| A | | | cancer | Oncol. 2020 April; 11(2): |
| | | | | 231-235 |
| Sialyl Lewis | AbGn-7 | AbGn-7 | Gastric cancer | Trial ID: NCT01466569 |
| A | | | | |
| Siglec15 | NC318 | NC318 | Solid tumors | Trial ID: NCT03665285 |
| TGF-b | | SRK-181 | Solid tumors | Trial ID: NCT04291079 |
| TGF-b | M-7824 | Bintrafusp alfa | NSCLC, Solid | Yoo et al., J Immunother |
| | | | tumors | Cancer. 2020 |
| | | | | May; 8(1): e000564 |
| TGF-b | GC-1008 | Fresolimumab | MM | Rice et al., J Clin Invest. |
| | | | | 2015 Jul. 1; 125(7): 2795-807 |
| TGF-b | | LY2382770 | Diabetic | Trial ID: NCT01113801 |
| | | | nephropathies | |
| TGF-b | NIS-793 | NIS793 | Pancreatic | Trial ID: NCT04390763: |
| | | | cancer | |
| TGF-b | | SAR439459 | Solid tumors | Trial ID: NCT03192345 |
| TGF-b | | Metelimumab | Cancer, | Lord et al., MAbs. 2018 |
| | | | Scleroderma | April, 10(3): 444-452 |
| TGF-b | IMC TR1 | LY3022859 | Solid tumors | Tolcher et al., Cancer |
| | | | | Chemother Pharmacol. 2017 |
| | | | | April, 79(4): 673-680 |
| TNFR3 | Baminercept | BG9924 | Rheumatoid | Trial ID: NCT00664716 |
| | | | arthritis | |
| TRAIL-R2 | CS-1008 | Tigatuzumab | Breast cancer, | Cheng et al., J Hepatol. 2015 |
| | | | NSCLC | October; 63(4): 896-904 |
| TRAIL-R2 | AMG-655 | Conatumumab | Solid tumors | Bajaj et al., Expert Opin Biol |
| | | | | Ther. 2011 November; 11(11): |
| | | | | 1519-24 |
| TRAIL-R2 | PRO-95780 | Drozitumab | NHL, NSCLC | Lima et al., Cancer Invest. |
| | | | | 2012 December; 30(10): 727-31 |
| TRAIL-R2 | HGS-ETR2 | Lexatumumab | Solid tumors | Plummer et al., Clin Cancer |
| | | | | Res. 2007 Oct. |
| | | | | 15; 13(20): 6187-94 |
| TRAIL-R2 | TAS-266 | TAS266 | Solid tumors | Trial ID: NCT01529307 |
| TRAIL-R2 | GEN1029 | Benufutamab | Solid tumors | Overdijk et al., Mol Cancer |
| | | | | Ther. 2020 October; 19(10): |
| | | | | 2126-2138 |
| TRAIL-R2 | RO-6874813 | RG7386 | Solid tumors | Brunker et al., Mol Cancer |
| | | | | Ther. 2016 May; 15(5): 946-57 |
| TRAIL-R2 | JCT-205 | INBRX-109 | Solid tumors | Trial ID: NCT03715933 |
| VEGF | Avastin | Bevacizumab | NSCLC, MM | Garcia et al., Cancer Treat |
| | | | | Rev. 2020 June; 86: 102017 |
| VEGF | Lucentis | Ranibizumab | Macular | Gross et al., JAMA |
| | | | degeneration | Ophthalmol. 2018 Oct. |
| | | | | 1; 136(10): 1138-1148 |
| VEGFR1 | IMC-18F1 | Icrucumab | Breast cancer | LoRusso et al., Invest New |
| | | | | Drugs. 2014 April; 32(2): |
| | | | | 303-11 |
| VEGFR2 | Cyramza | Ramucirumab | NSCLC, | Khan et al., Expert Opin Biol |
| | | | Colorectal | Ther. 2019 November; 19(11): |
| | | | cancer | 1135-1141 |
| VEGFR2 | Tanibirumab | Olinvacimab | Glioblastoma | Lee et al., Drug Des Devel |
| | | | | Ther. 2018 Mar. 8; 12: 495- |
| | | | | 504 |
| VEGFR2 | | Gentuximab | Solid tumors | Chamie et al., JAMA Oncol. |
| | | | | 2017 Jul. 1; 3(7): 913-920 |
| VEGFR2 | CDP-791 | Alacizumab | NSCLC | Trial ID: NCT00152477 |
| | | pegol | | |
| VEGFR2 | HLX-06 | Vulinacimab | Solid tumors | Trial ID: NCT03494231 |
| VEGFR2 | | MSB0254 | Solid tumors | Trial ID: NCT04381325 |
| VEGFR2 | | AK109 | Solid tumors | Trial ID: NCT04547205 |
| Vimentin | CLNH11 | Pritumumab | Glioma | Babic et al., Hum Antibodies. |
| | | | | 2018 Feb. 5; 26(2): 95-101 |
| Vimentin | | 86C | Glioblastoma | Stoubalova et al., Cancers |
| | | | | (2020) 12(1): 184 |

2. Small Molecule/Chemotherapy Drugs

In some embodiments, the additional therapy is a small molecule drug. In some embodiments, the additional therapy is a chemotherapy drug. In some embodiments, the additional therapy is a small molecule chemotherapy drug. Such small molecule drugs can include existing standard-of-care treatment regimens to which adoptive NK cell therapy is added. In some cases, the use of the NK cells described herein can enhance the effects of small molecule drugs, including by enhancing the efficacy, reducing the amount of small molecule drug necessary to achieve a desired effect, or reducing the toxicity of the small molecule drug.

In some embodiments, the drug is selected from the group consisting of

In some embodiments, the drug is [(1S,2S,3R,4S,7R,9S,10S,12R,15S)-4-acetyloxy-1,9,12-tri hydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.0^{3,10}.0^{4,7}]heptadec-13-en-2-yl]benzoate (docetaxel) or a pharmaceutically acceptable salt thereof.

In some embodiments, the drug is [(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0^{4,7}]heptadec-13-en-2-yl]benzoate (paclitaxel) or a pharmaceutically acceptable salt thereof.

In some embodiments, the drug is 6-N-(4,4-dimethyl-5H-1,3-oxazol-2-yl)-4-N-[3-methyl-4-([1,2,4]triazolo[1,5-a]pyridin-7-yloxy)phenyl]quinazoline-4,6-diamine (tucatinib) or a pharmaceutically acceptable salt thereof.

In some embodiments, the drug is pentyl N-[1-[(2R,3R,4S,5R)-3,4-dihydroxy-5-methyloxolan-2-yl]-5-fluoro-2-oxopyrimidin-4-yl]carbamate (capecitabine) or a pharmaceutically acceptable salt thereof.

In some embodiments, the drug is azanide; cyclobutane-1,1-dicarboxylic acid; platinum (2+) (carboplatin) or a pharmaceutically acceptable salt thereof.

In some embodiments, the drug is methyl (1R,9R,10S,11R,12R,19R)-11-acetyloxy-12-ethyl-4-[(12S,14R)-16-ethyl-12-methoxycarbonyl-1,10-diazatetracyclo[12.3.1.0^{3,11}.0^{4,9}]octadeca-3(11),4,6,8,15-pentaen-12-yl]-10-hydroxy-5-methoxy-8-methyl-8,16-diazapentacyclo[10.6.1.0.^{1,9}.0^{2,7}.0^{16,19}]nonadeca-2,4,6,13-tetraene-10-carboxylate (vinorelbine) or a pharmaceutically acceptable salt thereof.

In some embodiments, the drug is N-[3-chloro-4-[(3-fluorophenyl)methoxy]phenyl]-6-[5-[(2-methylsulfonylethylamino)methyl]furan-2-yl]quinazolin-4-amine (lapatinib) or a pharmaceutically acceptable salt thereof.

In some embodiments, the drug is (E)-N-[4-[3-chloro-4-(pyridin-2-ylmethoxy)anilino]-3-cyano-7-ethoxyquinolin-6-yl]-4-(dimethylamino)but-2-enamide (neratinib) or a pharmaceutically acceptable salt thereof.

In some embodiments, the drug is 6-acetyl-8-cyclopentyl-5-methyl-2-[(5-piperazin-1-ylpyridin-2-yl)amino]pyrido[2,3-d]pyrimidin-7-one (palbociclib) or a pharmaceutically acceptable salt thereof.

In some embodiments, the drug is 7-cyclopentyl-N,N-dimethyl-2-[(5-piperazin-1-ylpyridin-2-yl)amino]pyrrolo[2,3-d]pyrimidine-6-carboxamide (ribociclib) or a pharmaceutically acceptable salt thereof.

In some embodiments, the drug is N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (abemaciclib) or a pharmaceutically acceptable salt thereof.

In some embodiments, the drug is (1R,9S,12S,15R,16E,18R,19R,21R,23E,24E,261E28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatiaconta-16,24,26,28-tetraene-2,3,10,14,20-pentone (everolimus) or a pharmaceutically acceptable salt thereof.

In some embodiments, the drug is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib) or a pharmaceutically acceptable salt thereof.

In some embodiments, the drug is 4-[[3-[4-(cyclopropanecartonyl)piperazine-1-carbonyl]-4-fluorophenyl]methyl]-2H-phthalazin-1-one (olaparib) or a pharmaceutically acceptable salt thereof.

In some embodiments, the drug is (I 1S,12R)-7-fluoro-11-(4-fluorophenyl)-12-(2-methyl-1,2,4-triazol-3-yl)-2,3,10-triazatricyclo[7.3.1.0^{5,13}]trideca-1,5(13),6,8-tetraen-4-one (talazoparib) or a pharmaceutically acceptable salt thereof.

In some embodiments, the drug is N-[2-[2-(dimethylamino)ethyl-methylamino]-4-methoxy-5-[[4-(1-methylindol-3-yl)pyrimidin-2-yl]amino]phenyl]prop-2-enamid (osimertinib) or a pharmaceutically acceptable salt thereof.

In some embodiments, the drug is N-(3-chloro-4-fluorophenyl)-7-methoxy-6-(3-morpholin-4-ylpropoxy)quinazolin-4-amine (gefitinib) or a pharmaceutically acceptable salt thereof.

In some embodiments, the drug is N-(3-ethynylphenyl)-6,7-bis(2-methoxyethoxy)quinazolin-4-amine (erlotinib) or a pharmaceutically acceptable salt thereof.

In some embodiments, the drug is (E)-N-[4-(3-chloro-4-fluoroanilino)-7-[(3S)-oxolan-3-yl]oxyquinazolin-6-yl]-4-(dimethylamino)but-2-enamide (afatinib) or a pharmaceutically acceptable salt thereof.

In some embodiments, the drug is azane; dichloroplatinum (cisplatin, platinol) or a pharmaceutically acceptable salt thereof.

In some embodiments, the drug is azanide; cyclobutane-1,1-dicarboxylic acid; platinum(2+) (carboplatin) or a pharmaceutically acceptable salt thereof.

In some embodiments, the drug is 4-amino-1-[(2R,4R,5R)-3,3-difluoro-4-hydroxy-5-(hydroxymethyl)oxolan-2-yl]pyrimidin-2-one (gemcitabine) or a pharmaceutically acceptable salt thereof.

In some embodiments, the drug is (2S)-2-[[4-[2-(2-amino-4-oxo-3,7-dihydropyrrolo[2,3-d]pyrimidin-5-yl)ethyl]benzoyl]amino]pentanedioic acid (pemetrexed) or a pharmaceutically acceptable salt thereof.

In some embodiments, the drug is N,N-bis(2-chloroethyl)-2-oxo-1,3,2λ⁵-oxazaphosphinan-2-amine (cyclophosphamide) or a pharmaceutically acceptable salt thereof.

In some embodiments, the drug is (2R,3S,4S,5R)-2-(6-amino-2-fluoropurin-9-yl)-5-(hydroxymethyl)oxolane-3,4-diol (fludarabine) or a pharmaceutically acceptable salt thereof.

In some embodiments, the drug is (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7-tetracene-5,12-dione (doxorubicin) or a pharmaceutically acceptable salt thereof.

In some embodiments, the drug is methyl (1R,9R,10S,11R,12R,19R)-11-acetyloxy-12-ethyl-4-[(13S,15S,17S)-17-ethyl-17-hydroxy-13-methoxycarbonyl-1,11-diazatetracyclo[13.3.1.0^{4,12}.0^{5,10}]nonadeca-4(12),5,7,9-tetraen-13-yl]-8-formyl-10-hydroxy-5-methoxy-8,16-diazapentacyclo[10.6.1.0^{1,9}.0^{2,7}.0^{16,19}]nonadeca-2,4,6,13-tetraene-10-carboxylate (vincristine) or a pharmaceutically acceptable salt thereof.

In some embodiments, the drug is (8S,9S,10R,13S,14S,17R)-17-hydroxy-17-(2-hydroxyacetyl)-10,13-dimethyl-6,7,8,9,12,14,15,16-octahydrocyclopenta[a]phenanthrene-3,11-dione (prednisone) or a pharmaceutically acceptable salt thereof.

In some embodiments, the drug is N,3-bis(2-chloroethyl)-2-oxo-1,3,2λ⁵-oxazaphosphinan-2-amine (ifosfamide) or a pharmaceutically acceptable salt thereof.

In some embodiments, the drug is (5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one (etopside) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (dexamethasone) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (cytarabine) or a pharmaceutically acceptable salt thereof.

3. NK Cell Engagers

In some embodiments, the additional therapy is an NK cell engager, e.g., a bispecific or trispecific antibody.

In some embodiments, the NK cell engager is a bispecific antibody against CD16 and a disease-associated antigen, e.g., cancer-associated antigen, e.g., an antigen of cancers described herein. In some embodiments, the NK cell engager is a trispecific antibody against CD16 and two disease-associated antigens, e.g., cancer-associated antigens, e.g., antigens of cancers described herein.

4. Checkpoint Inhibitors

In some embodiments, the additional therapy is an immune checkpoint inhibitor.

In some embodiments, the immune checkpoint inhibitor is selected from the group consisting of a PD-1 inhibitor, a PD-L1 inhibitor, a CTLA-4 inhibitor, and combinations thereof.

In some embodiments, the immune checkpoint inhibitor is selected from the group consisting of a PD-1 inhibitor, a PD-L1 inhibitor, a CTLA-4 inhibitor, a VISTA inhibitor, a BTLA inhibitor, a TIM-3 inhibitor, a KIR inhibitor, a LAG-3 inhibitor, a TIGIT inhibitor, a CD-96 inhibitor, a SIRPα inhibitor, and combinations thereof.

In some embodiments, the immune checkpoint inhibitor is selected from the group consisting of a PD-1 inhibitor, a PD-L1 inhibitor, a CTLA-4 inhibitor, a LAG-3 (CD223) inhibitor, a TIM-3 inhibitor, a 7-H3 inhibitor, a B7-H4 inhibitor, an A2aR inhibitor, a CD73 inhibitor, a NKG2A inhibitor, a PVRIG/PVRL2 inhibitor, a CEACAM1 inhibitor, a CEACAM 5 inhibitor, a CEACAM 6 inhibitor, a FAK inhibitor, a CCL2 inhibitor, a CCR2 inhibitor, a LIF inhibitor, a CD47 inhibitor, a SIRPα inhibitor, a CSF-1 inhibitor, an M-CSF inhibitor, a CSF-R inhibitor, an IL-1 inhibitor, an IL-1R3 inhibitor, an IL-RAP inhibitor, an IL-8 inhibitor, a SEMA4D inhibitor, an Ang-2 inhibitor, a CEL3ER-1 inhibitor, an Ax inhibitor, a phosphatidylserine inhibitor, and combinations thereof.

In some embodiments, the immune checkpoint inhibitor is selected from those shown in Table 7, or combinations thereof.

TABLE 7
Exemplary Immune Checkpoint Inhibitors

| Target | Inhibitor |
|---|---|
| LAG-3 (CD223) | LAG525 (IMP701), REGN3767 (R3767), BI |
| | 754,091, tebotelimab (MGD013), eftilagimod |
| | alpha (IMP321), FS118 |
| TIM-3 | MBG453, Sym023, TSR-022 |
| B7-H3, B7-H4 | MGC018, FPA150 |
| A2aR | EOS100850, AB928 |
| CD73 | CPI-006 |
| NKG2A | Monalizumab |
| PVRIG/PVRL2 | COM701 |
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRPα | Hu5F9-G4 (5F9), ALX148, TTI-662, RRx-001 |
| CSF-1 | Lacnotuzumab (MCS110), LY302285S, |
| (M-CSF)/CSF-1R | SNDX-6352, emactuzumab (RG7155), |
| | pexidartinib (PLX3397) |
| IL-1 and IL-1R3 | CAN04, Canakinumab (ACZ885) |
| (IL-1RAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phosphatidylserine | Bavituximab |

In some embodiments, the immune checkpoint inhibitor is an antibody.

In some embodiments, the PD-1 inhibitor is selected from the group consisting of pembrolizumab, nivolumab, toripalimab, cemiplimab-rwlc, sintilimab, and combinations thereof.

In some embodiments, the PD-L1 inhibitor is selected from the group consisting of atezolizumab, durvalumab, avelumab, and combinations thereof.

In some embodiments, the CTLA-4 inhibitor is ipilimumab.

In some embodiments, the PD-1 inhibitor is selected from the group of inhibitors shown in Table 8.

TABLE 8
Exemplary PD-1 Inhibitor Antibodies

| Name | Internal Name | Antigen | Company |
|---|---|---|---|
| nivolumab | Opdivo, ONO-4538, | PD-1 | BMS, Medarex, Ono |
| | MDX-1106, BMS- | | |
| | 936558, 5C4 | | |
| pembrolizumab | Keytruda, MK-3475, | PD-1 | Merck (MSD), Schering- |
| | SCH 900475, | | Plough |
| | lambrolizumab | | |
| toripalimab | JS001, JS-001, | PD-1 | Junmeng |
| | TAB001, Triprizumab | | Biosciences, Shanghai |
| | | | Junshi, TopAlliance Bio |
| cemiplimab-rwlc | Libtayo, cemiplimab, | PD-1 | Regeneron, Sanofi |
| | REGN2810 | | |
| sintilimab | Tyvyt, IBI308 | PD-1 | Adimab, Innovent, Lilly |
| MEDI0680 | AMP-514 | PD-1 | Amplimmune, Medimmune |
| LZM009 | | PD-1 | Livzon |
| vudalimab | XmAb20717 | CTLA4, PD-1 | Xencor |
| SI-B003 | | CTLA4, PD-1 | Sichuan Baili |
| | | | Pharma, Systimmune |
| Sym021 | Symphogen patent anti- | PD-1 | Symphogen |
| | PD-1 | | |
| LVGN3616 | | PD-1 | Lyvgen Biopharma |
| MGD019 | | CTLA4, PD-1 | MacroGenics |
| MEDI5752 | | CTLA4, PD-1 | Medimmune |
| CS1003 | | PD-1 | CStone Pharma |
| IBI319 | IBI-319 | PD-1, | Innovent, Lilly |
| | | Undisclosed | |
| IBI315 | IBI-315 | HER2/neu, | Beijing Hanmi, Innovent |
| | | PD-1 | |
| budigalimab | ABBV-181, PR- | PD-1 | Abbvie |
| | 1648817 | | |
| Sunshine Guojian | 609A | PD-1 | Sunshine Guojian Pharma |
| patent anti-PD-1 | | | |
| F520 | | PD-1 | Shandong New Time |
| | | | Pharma |
| RO7247669 | | LAG-3, PD-1 | Roche |
| izuralimab | XmAb23104 | ICOS, PD-1 | Xencor |
| LY3434172 | | PD-1, PD-L1 | Lilly, Zymeworks |
| SG001 | | PD-1 | CSPC Pharma |
| QL1706 | PSB205 | CTLA4, PD-1 | Sound Biologics |
| AMG 404 | AMG404 | PD-1 | Amgen |
| MW11 | | PD-1 | Mabwell |
| GNR-051 | | PD-1 | IBC Generium |
| Ningbo Cancer | HerinCAR-PD1 | PD-1 | Ningbo Cancer Hosp. |
| Hosp. anti-PD-1 | | | |
| CAR | | | |
| Chinese PLA | | PD-1 | Chinese PLA Gen. Hosp. |
| Gen. Hosp. anti- | | | |
| PD-1 | | | |
| cetrelimab | JNJ-63723283 | PD-1 | Janssen Biotech |
| TY101 | | PD-1 | Tayu Huaxia |
| AK112 | | PD-1, VEGF | Akeso |
| EMB-02 | | LAG-3, PD-1 | EpimAb |
| pidilizumab | CT-011, hBat-1, | PD-1 | CureTech, Medivation, Teva |
| | MDV9300 | | |
| sasanlimab | PF-06801591, RN-888 | PD-1 | Pfizer |
| balstilimab | AGEN2034, AGEN- | PD-1 | Agenus, Ludwig |
| | 2034 | | Inst., Sloan-Kettering |
| geptanolimab | CBT-501, GB226, | PD-1 | CBT Pharma, Genor |
| | GB226, Genolimzumab, | | |
| | Genormab | | |
| RO7121661 | | PD-1, TIM-3 | Roche |
| AK104 | | CTLA4, PD-1 | Akeso |
| pimivalimab | JTX-4014 | PD-1 | Jounce |
| IBI318 | IBI-318 | PD-1, PD-L1 | Innovent, Lilly |
| BAT1306 | | PD-1 | Bio-Thera Solutions |
| ezabenlimab | BI754091, BI 754091 | PD-1 | Boehringer |
| Henan Cancer | Teripalimab | PD-1 | Henan Cancer Hospital |
| Hospital anti-PD-1 | | | |
| tebotelimab | | LAG-3, PD-1 | MacroGenics |
| sindelizumab | | PD-1 | Nanjing Medical U. |
| dostarlimab | ANB011, TSR-042, | PD-1 | AnaptysBio, Tesaro |
| | ABT1 | | |
| tislelizumab | BGB-A317 | PD-1 | BeiGene, Celgene |
| spartalizumab | PDR001, BAP049 | PD-1 | Dana-Farber, Novartis |
| retifanlimab | MGA012, | PD-1 | Incyte, MacroGenics |
| | INCMGA00012 | | |
| camrelizumab | SHR-1210 | PD-1 | Incyte, Jiangsu |
| | | | Hengrui, Shanghai Hengrui |
| zimberelimab | WBP3055, GLS-010, | PD-1 | Arcus, Guangzhou Gloria |
| | AB122 | | Bio, Harbin Gloria |
| | | | Pharma, WuXi Biologics |
| penpulimab | AK105 | PD-1 | Akeso, HanX Bio, Taizhou |
| | | | Hanzhong Bio |
| prolgolimab | BCD-100 | PD-1 | Biocad |
| HX008 | | PD-1 | Taizhou Hanzhong |
| | | | Bio, Taizhou HoudeAoke |
| | | | Bio |
| SCT-I10A | | PD-1 | Sinocelltech |
| serplulimab | HLX10 | PD-1 | Henlix |

In some embodiments, the PD-L1 inhibitor is selected from the group of inhibitors shown in Table 9.

TABLE 9
Exemplary PD-L1 Inhibitor Antibodies

| Name | Internal Name | Antigen | Company |
|---|---|---|---|
| durvalumab | Imfinzi, MEDI-4736, | PD-L1 | AstraZeneca, Celgene, |
| | MEDI4736 | | Medimmune |
| atezolizumab | Tecentriq, | PD-L1 | Genentech |
| | MPDL3280A, RG7446, | | |
| | YW243.55.S70, | | |
| | RO5541267 | | |
| avelumab | Bavencio, | PD-L1 | Merck Serono, Pfizer |
| | MSB0010718C, A09- | | |
| | 246-2 | | |
| AMP-224 | | PD-L1 | Amplimmune, GSK, |
| | | | Medimmune |
| cosibelimab | CK-301, TG-1501 | PD-L1 | Checkpoint |
| | | | Therapeutics, Dana- |
| | | | Farber, Novartis, TG |
| | | | Therapeutics |
| lodapolimab | LY3300054 | PD-L1 | Lilly |
| MCLA-145 | | 4-1BB, PD-L1 | Merus |
| FS118 | | LAG-3, PD-L1 | f-star, Merck Serono |
| INBRX-105 | ES101 | 4-1BB, PD-L1 | Elpiscience, Inhibrx |
| Suzhou Nanomab | | PD-L1 | Suzhou Nanomab |
| patent anti-PD-L1 | | | |
| MSB2311 | | PD-L1 | Mabspace |
| BCD-13 | | PD-L1 | Biocad |
| opucolimab | HLX20, HLX09 | PD-L1 | Henlix |
| IBI322 | IBI-322 | CD47, PD-L1 | Innovent |
| LY3415244 | | PD-L1, TIM-3 | Lilly, Zymeworks |
| GR1405 | | PD-L1 | Genrix Biopharma |
| LY3434172 | | PD-1, PD-L1 | Lilly, Zymeworks |
| CDX-527 | | CD27, PD-L1 | Celldex |
| FS222 | | 4-1BB, PD-L1 | f-star |
| LDP | | PD-L1 | Dragonboat Biopharma |
| ABL503 | | 4-1BB, PD-L1 | ABL Bio |
| HB0025 | | PD-L1, VEGF | Huabo Biopharm |
| MDX-1105 | BMS-936559, 12A4 | PD-L1 | Medarex |
| garivulimab | BGB-A333 | PD-L1 | BeiGene |
| GEN1046 | | 4-1BB, PD-L1 | BioNTech, Genmab |
| NM21-1480 | | 4-1BB, PD- | Numab |
| | | L1, Serum | |
| | | Albumin | |
| bintrafusp alfa | M7824, MSB0011359C | PD- | Merck Serono, NCI |
| | | L1, TGFβRII | |
| pacmilimab | CX-072 | PD-L1 | CytomX |
| A167 | KL-A167 | PD-L1 | Harbour Biomed |
| | | | Ltd., Sichuan Kelun Pharma |
| IBI318 | IBI-318 | PD-1, PD-L1 | Innovent, Lilly |
| KN046 | | CTLA4, PD- | Alphamab |
| | | L1 | |
| STI-3031 | IMC-001 | PD-L1 | Sorrento |
| SHR-1701 | | PD-L1 | Jiangsu Hengrui |
| LP002 | | PD-L1 | Taizhou HoudeAoke Bio |
| STI-1014 | ZKAB001 | PD-L1 | Lee's Pharm, Sorrento |
| envafolimab | KN035 | PD-L1 | Alphamab |
| adebrelimab | SHR-1316 | PD-L1 | Jiangsu Hengrui, Shanghai |
| | | | Hengrui |
| CS1001 | | PD-L1 | CStone Pharma |
| TQB2450 | CBT-502 | PD-L1 | CBT Pharma, Chia Tai |
| | | | Tianqing Pharma |

In some embodiments, the CTLA-4 inhibitor is selected from the group of inhibitors shown in

TABLE 10
Exemplary CTLA4 Inhibitor Antibodies

| Name | Internal Name | Antigen | Company |
|---|---|---|---|
| ipilimumab | Yervoy, MDX-010, | CTLA4 | Medarex |
| | MDX101, 10D1, BMS- | | |
| | 734016 | | |
| ATOR-1015 | ADC-1015 | CTLA4, OX40 | Alligator |
| vudalimab | XmAb20717 | CTLA4, PD-1 | Xencor |
| SI-B003 | | CTLA4, PD-1 | Sichuan Baili |
| | | | Pharma, Systimmune |
| MGD019 | | CTLA4, PD-1 | MacroGenics |
| MEDI5752 | | CTLA4, PD-1 | Medimmune |
| ADU-1604 | | CTLA4 | Aduro |
| BCD-145 | Q3W | CTLA4 | Biocad |
| CS1002 | | CTLA4 | CStone Pharma |
| REGN4659 | | CTLA4 | Regeneron |
| pavunalimab | XmAb22841 | CTLA4, LAG-3 | Xencor |
| AGEN1181 | | CTLA4 | Agenus |
| QL1706 | PSB205 | CTLA4, PD-1 | Sound Biologics |
| ADG126 | | CTLA4 | Adagene |
| KN044 | | CTLA4 | Changchun Intelli-Crown |
| ONC-392 | | CTLA4 | OncoImmune, Pfizer |
| BMS-986218 | | CTLA4 | BMS |
| BMS-986249 | | CTLA4 | BMS |
| BT-001 | TG6030 | CTLA4 | BioInvent |
| quavonlimab | MK-1308 | CTLA4 | Merck (MSD) |
| zalifrelimab | AGEN1884 | CTLA4 | Agenus, Ludwig |
| | | | Inst., Sloan-Kettering |
| AK104 | | CTLA4, PD-1 | Akeso |
| IBI310 | IBI-310 | CTLA4 | Innovent |
| KN046 | | CTLA4, PD-L1 | Alphamab |
| tremelimumab | ticilimumab, CP-675206, | CTLA4 | Amgen, Medimmune, |
| | clone 11.2.1 | | Pfizer |

In some embodiments, the immune checkpoint inhibitor is a small molecule drug. Small molecule checkpoint inhibitors are described, e.g., in WO2015/034820A1, WO2015/160641A2, WO2018/009505 A1, WO2017/066227 A1, WO2018/044963 A1, WO2018/026971 A1, WO2018/045142 A1, WO2018/005374 A1, WO2017/202275 A1, WO2017/202273 A1, WO2017/202276 A1, WO2018/006795 A1, WO2016/142852 A1, WO2016/142894 A1, WO2015/033301 A1, WO2015/033299 A1, WO2016/142886 A2, WO2016/142833 A1, WO2018/051255 A1, WO2018/051254 A1, WO2017/205464 A1, US2017/0107216 A1, WO2017/070089A1, WO2017/106634A1, US2017/0174679 A1, US2018/0057486 A1, WO2018/013789 A1, US2017/0362253 A1, WO2017/192961 A1, WO2017/118762 A1, US2014/199334 A1, WO2015/036927 A1, US2014/0294898 A1, US2016/0340391 A1, WO2016/039749 A1, WO2017/176608 A1, WO2016/077518 A1, WO2016/100608 A1, US2017/0252432 A1, WO2016/126646 A1, WO2015/044900 A1, US2015/0125491 A1, WO2015/033303 A1, WO2016/142835 A1, WO2019/008154 A1, WO2019/008152 A1, and WO2019023575A1.

In some embodiments, the PD-1 inhibitor is 2-[[4-amino-1-[5-(1-amino-2-hydroxypropyl)-1,3,4-oxadiazol-2-yl]-4-oxobutyl]carbamoylamino]-3-hydroxypropanoic acid (CA-170).

In some embodiments, the immune checkpoint inhibitor is (S)-1-(3-Bromo-4-((2-bromo-[1,1′-biphenyl]-3-yl)methoxy)benzyl)piperidine-2-carboxylic Acid.

In some embodiments, the immune checkpoint inhibitor is a peptide. See, e.g., Sasikumar et al., “Peptide and Peptide-Inspired Checkpoint Inhibitors: Protein Fragments to Cancer Immunotherapy,” Medicine in Drug Discovery 8:100073 (2020).

### VI. Treatment of Cancer with NK Cells and a CD20 Targeted Antibody

NHLs are a heterogeneous group of lymphoproliferative malignancies that usually originate in lymphoid tissues and can spread to other organs. Prognosis for NHL patients depends on histologic type, stage, and response to treatment. NHL can be divided into 2 prognostic groups: the indolent lymphomas and the aggressive lymphomas. Indolent NHLs offer a relatively good prognosis with a median survival of up to 20 years and are generally responsive to immunotherapy, radiation therapy, and chemotherapy. However, a continuous rate of relapse is seen in advanced stages of indolent NHLs. In contrast, aggressive NHLs present acutely and are more commonly resistant or refractory to frontline therapy.

In general, patients with newly diagnosed NHL are treated with chemotherapy combined with rituximab that confers long-term remissions in most patients. NHL patients who are refractory to front-line treatment or those who relapse soon after completing front-line therapies, have poor outcomes. These patients are typically treated with a second line of chemotherapy (ICE or DHAP), often combined with an approved therapeutic monoclonal antibody (mAb). Depending on their response to this therapy and the patient's physical condition, autologous stem cell transplant (ASCT) or an approved chimeric antigen receptor T-cell therapy (CAR-T) may be offered. For patients who are ineligible for ASCT, treatment options are limited, and median overall survival is 3.3 months. For patients who have experienced disease progression after ASCT or CAR-T, treatment options and survival are poor (Van Den Neste 2016 Bone Marrow Transplantation 51:51-57). Relapsed and refractory NHL of B-cell origin is, therefore, an area of unmet medical need.

Described herein are methods for treating a patient suffering from a CD20+ cancer, the methods include: administering allogenic natural killer cells (NK cells) and an antibody targeted to human CD20, wherein the NK cells are allogenic to the patient, are KIR-B haplotype and express CD16 having the VN polymorphism at F158.

In various embodiments: the cancer is non-Hodgkins lymphoma (NHL) (e.g., indolent NHL or aggressive NHL); the patient has relapsed after treatment with an anti-CD20 antibody; patient has the experienced disease progression after treatment with autologous stem cell transplant or chimeric antigen receptor T-cell therapy (CAR-T); the patient is administered 1×10⁸ to 1×10¹⁰ NK cells, the patient is administered 1×10⁹ to 8×10⁹ NK cells; the patient is administered 4×10⁸, 1×10⁹, 4×10⁹, or 8×10⁹ NK cells; 100 to 500 mg/m² of the antibody targeted to human CD20; each administration of NK cells is administration of 1×10⁹ to 5×10⁹ NK cells; each administration of NK cells is administration of 1×10⁹ to 5×10⁹ NK cells; the patient is administered 375 mg/m² of the antibody targeted to human CD20; the antibody targeted to human CD20 is rituximab; the patient is subjected to lymphodepleting chemotherapy (e.g., non-myeloablative chemotherapy by administering at least one of or both of cyclophosphamide and fludarabine) prior to treatment with the NK cells. The lymphodepleting chemotherapy can include, in various embodiments: treatment with cyclophosphamide and fludarabine, administration of cyclophosphamide at between 100 and 500 mg/m²/day; administration of cyclophosphamide at 250 mg/m²/day; administration of fludarabine at between 10 and 50 mg/m²/day or at 30 mg/m²/day.

In various embodiments: the method further comprising administering IL-2 (e.g., a dose of 1×10⁶ IU/m² of IL-2). In some embodiments, administration of IL-2 occurs within 1-4 hrs of administration of the NK cells.

In various embodiments: the administration of the NK cells and the antibody targeted to human CD20 occurs weekly; the NK cells and the antibody targeted to human CD20 are administered weekly for 4 to 8 weeks; the NK cells are not genetically modified; at least 70% of the NK cells are CD56+ and CD16+; at least 85% of the NK cells are CD56+ and CD3−; 1% or less of the NK cells are CD3+, 1% or less of the NK cells are CD19+ and 1% or less of the NK cells are CD14+.

In various embodiments: the indolent NHL is selected from the group consisting of Follicular lymphoma, Lymphoplasmacytic lymphoma/Waldenstrom macroglobulinemia, Gastric MALT, Non-gastric MALT, Nodal marginal zone lymphoma, Splenic marginal zone lymphoma, Small-cell lymphocytic lymphoma (SLL), and Chronic lymphocytic lymphoma (CLL); the Small-cell lymphocytic lymphoma (SLL) or Chronic lymphocytic lymphoma (CLL) comprises nodal or splenic involvement; the aggressive NHL is selected from the group consisting of Diffuse large B-cell lymphoma, Mantle cell lymphoma, Transformed follicular lymphoma, Follicular lymphoma (Grade IIIB), Transformed mucosa-associated lymphoid tissue (MALT) lymphoma, Primary mediastinal B-cell lymphoma, Lymphoblastic lymphoma, High-grade B-cell lymphomas with translocations of MYC and BCL2; the high-grade B-cell lymphomas with translocations of MYC and BLC2 further comprises a translocation of BCL6.

Suitable NK cells for use in treatment of NHL can be prepared as described in US 2020/0108096 or WO 2020/101361, both of which are incorporated herein by reference. Briefly, the source cells are cultured on modified HuT-78 (ATCC® TIB-161™) cells that have been engineered to express 4-1BBL, membrane bound IL-21 and a mutant TNFalpha as described in US 2020/0108096.

As one example, suitable NK cells can be prepared as follows using HuT-78 cells transduced to express 4-1BBL, membrane bound IL-21 and mutant TNFalpha (“eHut-78P cells”) as feeder cells. The feeder cells are suspended in 1% (v/v) CellGro medium at 2.5×10⁶ cells/ml and are irradiated with 20,000 cGy in a gamma-ray irradiator. Seed cells (e.g., CD3-depleted PBMC or CD3-depleted cord blood cells) are grown on the feeder cells in CellGro medium containing 1% (v/v) human plasma, glutamine, 500 IU of IL-2, 10 ng/ml of OKT-3 at a ratio of 1:2.5 (seed cells:feeder cells) in in static culture at 37° C. The cells are split every 2-4 days. The total culture time can be 19 days. The NK cells are harvested by centrifugation and cryopreserved. Thawed NK are administration to patients in infusion medium consisting of Phosphate Buffered Saline (50% v/v) with albumin (human) 20% (20% v/v), Dextran 40 in Dextrose (25% v/v) and dimethyl sulfoxide (DMSO) (5% v/v).

In some case, the seed cells are CD3-depleted cord blood cells. Preferably, the cord blood seed cells are selected to express CD16 having the V/V polymorphism at F158 (Fc gamma RIIIa-158 V/V genotype) (Musolino et al. 2008 J Clin Oncol 26:1789). Preferably, the cord blood seed cells are KIR-B haplotype. A cell fraction can be depleted of CD3 cells by immunomagnetic selection, for example, using a CliniMACS T cell depletion set ((LS Depletion set (162-01) Miltenyi Biotec).

Rituximab (e.g., Rituxan®) is a preferred IL-20 targeted antibody. Rituximab is preferably administered at 375 mg/m², preferably at least 1 hour prior to each administration of NK cells.

IL-2 is preferably administered at 1×10⁶ IU/m², will be administered subcutaneously, at least 1 hour and no more than 4 hours following the conclusion of each administration.

The methods described herein can be used to treat patients suffering from a CD20+ cancer, for example, indolent or aggressive non-Hodgkin's lymphoma (NHL), particularly relapsed or refractory indolent or aggressive NHL of B-cell origin. Among the aggressive and indolent subtypes are those in Table 11.

TABLE 11
Exemplary Aggressive and Indolent NHL

| Aggressive Subtype | Indolent Subtype |
|---|---|
| Diffuse large B-cell lymphoma | Follicular lymphoma (Grades I, II, and IIIA) |
| Mantle cell lymphoma | Lymphoplasmacytic lymphoma/Waldenström |
| | macroglobulinemia |
| Transformed follicular lymphoma | Gastric MALT (MZL) |
| Follicular lymphoma (Grade IIIB) | Non-gastric MALT (MZL) |
| Transformed mucosa-associated lymphoid | Nodal marginal zone lymphoma (MZL) |
| tissue (MALT) lymphoma | |
| Primary mediastinal B-cell lymphoma | Splenic marginal zone lymphoma (MZL) |
| Lymphoblastic lymphoma | Small-cell lymphocytic lymphoma |
| | (SLL)/Chronic lymphocytic lymphoma (CLL) |
| | with nodal or splenic involvement |
| High-grade B-cell lymphomas with | |
| translocations of MYC and BCL2 and/or | |
| BCL6 (double/triple hit lymphoma) | |

Prior to treatment, the patient is preferably lymphodepleted by intravenous administration of cyclophosphamide (250 mg/m²/day) and fludarabine (30 mg/m²/day) daily for 3 consecutive days, starting 5 days before the first dose of NK cells (i.e., from Day −5 through Day −3).

The NK cells (for example AB-101, Artiva Biotherapeutics, Inc.) are preferably administered weekly with each administration of 1×10⁹ or 4×10⁹ NK cells. The cells are preferably cryopreserved NK cells suspended in infusion-ready media (50% PBS, 25% Dextran 40, 20% albumin (human), 5% DMSO) in vials containing approximately 1×10⁹ cells. The cells are thawed in a 37° C. water bath prior to administration. The thawed vial(s) of NK cells are aseptically transferred to a single administration bag using a vial adapter and a sterile syringe. The NK cells are administered to the patient from the bag through a Y-type blood/solution set with filter as an IV infusion, by gravity. The NK cells are preferably should be administered as soon as practical, preferably within 30 minutes and no longer than 90 minutes after thawing.

IL-2, dosed at 1×10⁶ IU/m², is administered subcutaneously, at least 1 hour and no more than 4 hours following the conclusion of each dose of NK cells. Rituximab is preferably administered at 375 mg/m², preferably at least 1 hour prior to each administration of NK cells.

Administration of the NK cells preferably occurs weekly for 8 weeks.

Thus, described herein are methods for treating a patient suffering from a CD20+ cancer, the method comprising administering allogenic natural killer cells (NK cells) and an antibody targeted to human CD20, wherein the NK cells are allogenic to the patient, are KIR-B haplotype and express CD16 having the VN polymorphism at F158.

In some embodiments, the cancer is non-Hodgkins lymphoma (NHL).

In some embodiments, the NHL is indolent NHL.

In some embodiments, the NHL is aggressive NHL.

In some embodiments, the patient has relapsed after treatment with an anti-CD20 antibody.

In some embodiments, the patient has experienced disease progression after treatment with autologous stem cell transplant or chimeric antigen receptor T-cell therapy (CAR-T).

In some embodiments, the patient is administered 1×10⁸ to 1×10¹⁰ NK cells.

In some embodiments, the patient is administered 1×10⁹ to 8×10⁹ NK cells.

In some embodiments, the patient is administered 4×10⁸, 1×10⁹, 4×10⁹, or 8×10⁹ NK cells.

In some embodiments, the patient is administered 100 to 500 mg/m² of the antibody.

In some embodiments, the patient is administered 375 mg/m² of the antibody.

In some embodiments, the antibody is rituximab.

In some embodiments, the patient is subjected to lymphodepleting chemotherapy prior to treatment.

In some embodiments, the lymphodepleting chemotherapy is non-myeloablative chemotherapy.

In some embodiments, the lymphodepleting chemotherapy comprises treatment with at least one of cyclophosphamide and fludarabine.

In some embodiments, the lymphodepleting chemotherapy comprises treatment with cyclophosphamide and fludarabine.

In some embodiments, the cyclophosphamide is administered between 100 and 500 mg/m²/day.

In some embodiments, the cyclophosphamide is administered 250 mg/m²/day.

In some embodiments, the fludarabine is administered between 10 and 50 mg/m²/day.

In some embodiments, the fludarabine is administered 30 mg/m²/day.

In some embodiments, the method further comprises administering IL-2.

In some embodiments, the patient is administered 1×10⁶ IU/m² of IL-2.

In some embodiments, administration of IL-2 occurs within 1-4 hrs of administration of the NK cells.

In some embodiments, the administration of the NK cells and the antibody targeted to human CD20 occurs weekly.

In some embodiments, the NK cells and the antibody targeted to human CD20 are administered weekly for 4 to 8 weeks.

In some embodiments, the NK cells are not genetically modified.

In some embodiments, at least 70% of the NK cells are CD56+ and CD16+.

In some embodiments, at least 85% of the NK cells are CD56+ and CD3−.

In some embodiments, 1% or less of the NK cells are CD3+, 1% or less of the NK cells are CD19+ and 1% or less of the NK cells are CD14+.

In some embodiments, the indolent NHL is selected from the group consisting of Follicular lymphoma, Lymphoplasmacytic lymphoma/Waldenstrom macroglobulinemia, Gastric MALT, Non-gastric MALT, Nodal marginal zone lymphoma, Splenic marginal zone lymphoma, Small-cell lymphocytic lymphoma (SLL), and Chronic lymphocytic lymphoma (CLL).

In some embodiments, the Small-cell lymphocytic lymphoma (SLL) or Chronic lymphocytic lymphoma (CLL) comprises nodal or splenic involvement.

In some embodiments, the aggressive NHL is selected from the group consisting of Diffuse large B-cell lymphoma, Mantle cell lymphoma, Transformed follicular lymphoma, Follicular lymphoma (Grade IIIB), Transformed mucosa-associated lymphoid tissue (MALT) lymphoma, Primary mediastinal B-cell lymphoma, Lymphoblastic lymphoma, High-grade B-cell lymphomas with translocations of MYC and BCL2.

In some embodiments, the high-grade B-cell lymphomas with translocations of MYC and BLC2 further comprises a translocation of BCL6.

In some embodiments, each administration of NK cells is administration of 1×10⁹ to 5×10⁹ NK cells.

In some embodiments, each administration of NK cells is administration of 1×10⁹ to 5×10⁹ NK cells.

### VII. Variants

In some embodiments, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### VIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa.

As used herein, the term “cell culture medium” refers to a mixture for growth and proliferation of cells in vitro, which contains essential elements for growth and proliferation of cells such as sugars, amino acids, various nutrients, inorganic substances, etc.

A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### IX. Examples

The following examples are included for illustrative purposes only and are not intended to limit the scope of the invention.

### Example 1: Off-the-Shelf NK Cell Therapy Platform

One example of a method by which NK cells were expanded and stimulated is shown FIG. 1.

A single unit of FDA-licensed, frozen cord blood that has a high affinity variant of the receptor CD16 (the 158 VN variant, see, e.g., Koene et al., “FcγRIIIa-158V/F Polymorphism Influences the Binding of IgG by Natural Killer Cell FcgammaRIIa, Independently of the FcgammaRIIIa-48L/R/H Phenotype,” Blood 90:1109-14 (1997).) and the KIR-B genotype (KIR B allele of the KIR receptor family, see, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5:e15115 (2010)) was selected as the source of NK cells.

The cord blood unit was thawed and the freezing medium was removed via centrifugation. The cell preparation was then depleted of T cells using the QuadroMACS Cell Selection System (Miltenyi) and CD3 (T cell) MicroBeads. A population of 6×10⁸ total nucleated cells (TNC) were labelled with the MicroBeads and separated using the QuadroMACS device and buffer. Following depletion of T cells, the remaining cells, which were predominantly monocytes and NK cells, were washed and collected in antibiotic-free medium (CellgroSCGM). The cell preparation was then evaluated for total nucleated cell count, viability, and % CD3+ cells. As shown in FIG. 1, the cord blood NK cells were CD3 depleted.

The CD3− cell preparation was inoculated into a gas permeable cell expansion bag containing growth medium. As FIG. 1, the cells were co-cultured with replication incompetent engineered HuT-78 (eHUT-78) feeder cells to enhance expansion for master cell bank (MCB) production. The CellgroSCGM growth media was initially supplemented with anti-CD3 antibody (OKT3), human plasma, glutamine, and IL-2.

As shown in FIG. 1, the NK cells are optionally engineered, e.g., to introduce CARs into the NK cells, e.g., with a lentiviral vector, during one of the co-culturing steps.

The cells were incubated as a static culture for 12-16 days at 37° C. in a 5% CO₂ balanced air environment, with additional exchanges of media occurring every 2 to 4 days. After the culture expanded more than 100-fold, the cultured cells were harvested and then suspended in freezing medium and filled into cryobags. In this example, 80 bags or vials at 10⁸ cells per bag or vial were produced during the co-culture. The cryobags were frozen using a controlled rate freezer and stored in vapor phase liquid nitrogen (LN₂) tanks below −150° C. These cryopreserved NK cells derived from the FDA-licensed cord blood unit served as the master cell bank (MCB).

To produce the drug product, a bag of frozen cells from the MCB was thawed and the freezing medium was removed. The thawed cells were inoculated into a disposable culture bag and co-cultured with feeder cells, e.g., eHUT78 feeder cells to produce the drug product. In this example, the cells are cultured in a 50 L bioreactor to produce thousands of lots of the drug product per unit of cord blood (e.g., 4,000-8,000 cryovials at 10⁹ cells/vial), which are mixed with a cryopreservation composition and frozen in a plurality of storage vessels such as cryovials. The drug product is an off-the-shelf infusion ready product that can be used for direct infusion. Each lot of the drug product can be used to infuse hundreds to thousands of patients (e.g., 100-1,000 patients, e.g. with a target dose of 4×10⁹ cells).

### Example 2: Feeder Cell Expansion

As one example, suitable feeder cells, e.g., eHut-78 cells, were thawed from a frozen stock and expanded and cultured in a 125 mL flask in growth medium comprising RPMI1640 (Life Technologies) 89% v/v, inactivated fetal bovine serum (FBS) (Life Technologies) (10% v/v), and glutamine (hyclone) (2 mM) at or at about 37° C. and at or at about 3-7% CO₂ for or for about 18-24 days. The cells were split every 2-3 days into 125 mL-2L flasks. The cells were harvested by centrifugation and gamma irradiated. The harvested and irradiated cells were mixed with a cryopreservation medium (Cryostor CS10) in 2 mL cryovials and frozen in a controlled rate freezer, with a decrease in temperature of about 15° C. every 5 minutes to a final temperature of or of about −90° C., after which they were transferred to a liquid nitrogen tank or freezer to a final temperature of or of about −150° C.

After freezing, cell viability was greater than or equal to 70% of the original number of cells (here, at least 1.0×10⁸ viable cells/mL), and 85% or more of the cells expressed mTNF-α, 85% or more of the cells expressed mbIL-21+, and 85% or more of the cells expressed 4-1BBL.

### Example 3: NK Cell Expansion and Stimulation

As one example, suitable NK cells can be prepared as follows using HuT-78 cells transduced to express 4-1BBL, membrane bound IL-21 and mutant TNFalpha (“eHut-78P cells”) as feeder cells. The feeder cells are suspended in 1% (v/v) CellGro medium and are irradiated with 20,000 cGy in a gamma-ray irradiator. Seed cells (e.g., CD3-depleted PBMC or CD3-depleted cord blood cells) are grown on the feeder cells in CellGro medium containing human plasma, glutamine, IL-2, and OKT-3 in static culture at 37° C. The cells are split every 2-4 days. The total culture time was 19 days. The NK cells are harvested by centrifugation and cryopreserved. Thawed NK are administered to patients in infusion medium consisting of: Phosphate Buffered Saline (PBS 1×, FujiFilm Irvine) (50% v/v), albumin (human) (20% v/v of OctaPharma albumin solution containing: 200 g/L protein, of which >96% is human albumin, 130-160 mmol sodium; ≤2 mmol potassium, 0.064-0.096 mmol/g protein N-acetyl-DL-tryptophan, 0.064-0.096 mmol/g protein, caprylic acid, ad. 1000 ml water), Dextran 40 in Dextrose (25% v/v of Hospira Dextran 40 in Dextrose Injection, USP containing: 10 g/100 mL Dextran 40 and 5 g/100 mL dextrose hydrous in water) and dimethyl sulfoxide (DMSO) (5% v/v of Avantor DMSL solution with a density of 1.101 g/cm³ at 20° C.).

In some case, the seed cells are CD3-depleted cord blood cells. A cell fraction can be depleted of CD3 cells by immunomagnetic selection, for example, using a CliniMACS T cell depletion set ((LS Depletion set (162-01) Miltenyi Biotec).

Preferably, the cord blood seed cells are selected to express CD16 having the V/V polymorphism at F158 (Fc gamma RIIIa-158 V/V genotype) (Musolino et al. 2008 J Clin Oncol 26:1789). Preferably, the cord blood seed cells are KIR-B haplotype.

### Example 4: Cord Blood as an NK Cell Source

NK cells make up five to 15% of peripheral blood lymphocytes. Traditionally, peripheral blood has been used as the source for NK cells for therapeutic use. However, as shown herein, NK cells derived from cord blood have a nearly ten-fold greater potential for expansion in the culture systems described herein than those derived from peripheral blood, without premature exhaustion or senescence of the cells. The expression of receptors of interest on the surface of NK cells, such as those involved in the activation of NK cells on engagement of tumor cells, was seen to be more consistent donor-to-donor for cord blood NKs than peripheral-blood NK cells. The use of the manufacturing process described herein consistently activated the NK cells in cord blood in a donor-independent manner, resulting in a highly scaled, active and consistent NK cell product.

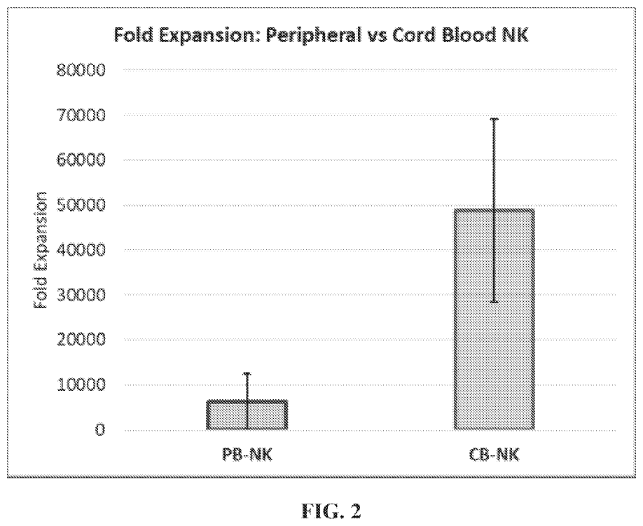

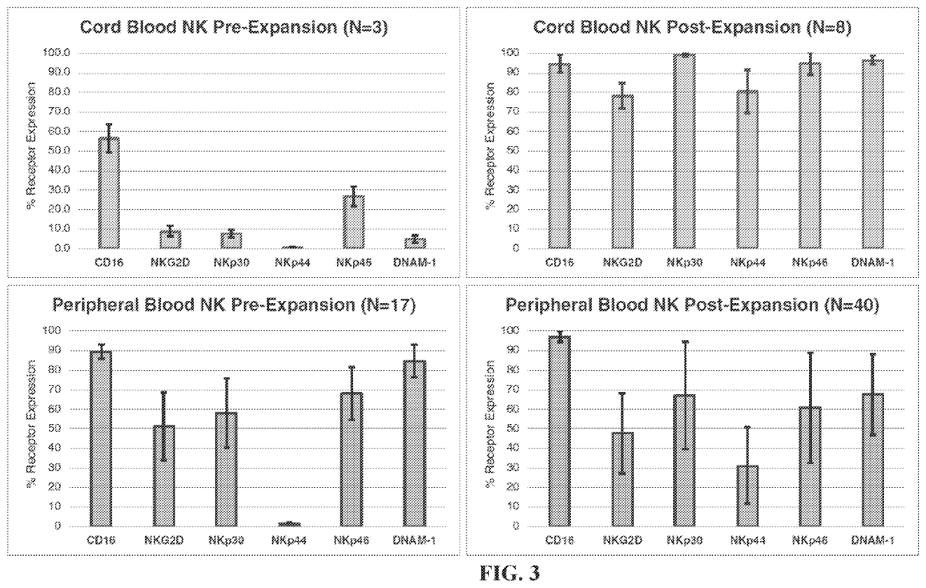

As shown in FIG. 2, cord blood-derived NK cells (CB-NK) have an approximately ten-fold greater ability to expand in culture than peripheral blood-derived NK cells (PB-NK) in preclinical studies. As shown in FIG. 3, expression of tumor-engaging NK activating immune receptors was higher and more consistent in cord blood-derived drug product compared to that generated from peripheral blood.

### Example 5: Expanded and Stimulated NK-Cell Phenotype

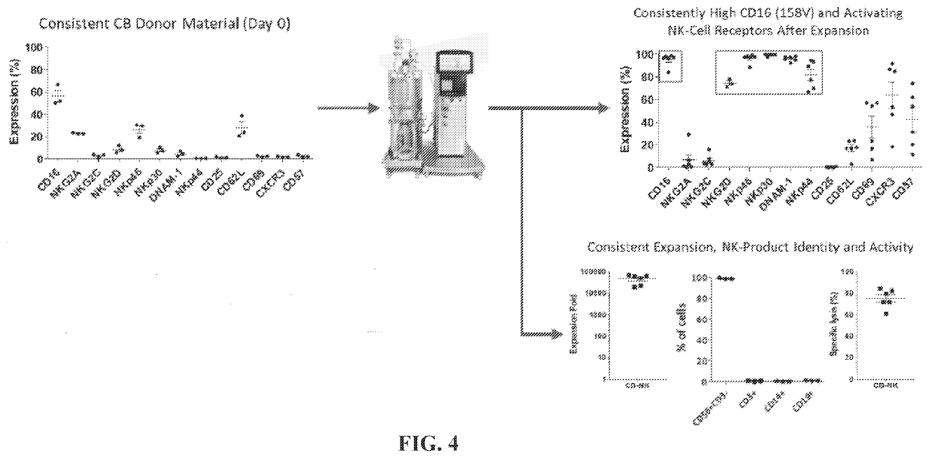

In one example, NK cells from a cord blood unit are expanded and stimulated with eHut-78 cells, according to the expansion and stimulation process described in Example 1. As shown in FIG. 4, the resulting expanded and stimulated population of NK cells have consistently high CD16 (158V) and activating NK-cell receptor expression.

### Example 6: AB-101

AB-101 is a universal, off-the-shelf, cryopreserved allogeneic cord blood derived NK cell therapy product comprising ex vivo expanded and activated effector cells designed to enhance ADCC anti-tumor responses in patients, e.g., patients treated with monoclonal antibodies or NK cell engagers. AB-101 is comprised of cord blood derived mononuclear cells (CBMCs) enriched for NK cells by depletion of T lymphocytes, and co-cultured with an engineered, replication incompetent T cell feeder line supplemented with IL-2 and anti-CD3 antibody (OKT3).

AB-101 is an allogeneic NK-cell product derived from FDA licensed cord blood, specifically designed to treat hematological and solid tumors in combination with therapeutic monoclonal antibodies (mAbs). The AB-101 manufacturing process leads to an NK cell product with the following attributes:
Consistent NK cell profile. High surface receptor expression of antibody engaging CD16 and tumor antigen-engaging/activating receptors such as NKG2D, NKp46, Nkp30 and NKp44.
KIR-B-haplotype. KIR-B haplotype has been associated with improved clinical outcomes in the haploidentical transplant setting and greater therapeutic potential in the allogeneic setting
CD16 F158V polymorphism. The higher-affinity CD16 F158V variant binding to mAb Fc-domain is seen to facilitate enhanced antibody dependent cellular cytotoxicity (ADCC).
Unmodified NK cells. No genetic enhancement or gene editing is required for, or is a part of, the AB-101 drug product.

The components and composition of AB-10 are listed in Table 12. AB-01 is comprised of NK cells (CD16⁺, CD56⁺) expressing the natural cytotoxicity receptors NKp3 and NKp46 indicative of mature NK cells. AB-10 contains negligible T cells, B cells and macrophages (≤0.2% CD3⁺, ≤1.0% CD19⁺, ≤1.0% CD14⁺). Residual eHuT-78P feeder cells used in the culturing of AB-10 are ≤0.2% of the drug product.

TABLE 12
Components and Compositions of AB-101

| Component | Solution | | | Quantity per Unit |
|---|---|---|---|---|
| Solution | Composition | Conc | Conc | (11 mL fill) |
| AB-101 drug | Approximately | 50% v/v | 0.5 mL/mL | 5.5 mL |
| substance (ex vivo- | 1.1 × 10⁹ | | | (0.9 × 10⁹-1.3 × 10⁹ |
| expanded allogeneic | viable cells | | | viable cells per |
| natural killer cells) | | | | vial in 5.27-6.23 |
| PBS | 100% Phosphate | | | mL of PBS) |
| | Buffered Saline | | | |
| | (PBS) | | | |
| Albumin Solution | 200 g/L | 20% v/v | 40 mg/mL | 2.2 mL |
| | albumin in water | | albumin | (1.98-2.42 mL) |
| Dextran 40 Solution | 100 g/L | 25% v/v | 25 mg/mL | 2.75 mL |
| | Dextran 40; and | | Dextran 40; | (2.475-3.025 mL) |
| | 50 g/L | | 12.5 mg/mL | |
| | glucose in water | | glucose | |
| DMSO | 100% DMSO | 5% v/v | 55 mg/mL | 0.55 mL |
| | (1,100 g/L) | | | (0.495-0.605 mL) |

Initial stability studies indicate that AB-101 is stable for up to six months in the vapor phase of liquid nitrogen. Long-term stability studies to assess product stability beyond six months are ongoing, and the most current stability information will be captured on the certificate of analysis.

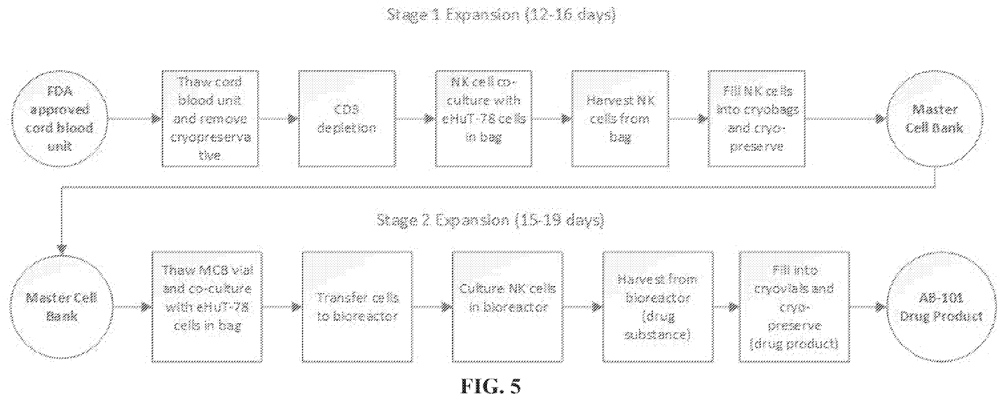

The manufacture of the AB-101 drug product is comprised of the following key steps (FIG. 5):
Thaw of the FDA licensed cord blood unit (Hemacord, BLA 125937).
Removal of cyro-preservation medium from the cord blood unit (CBU)
CD3 depletion using FDA cleared Vario MACS Cell Selection System (Miltenyi)
Expansion and co-culture in bags with an engineered feeder cell line (eHuT-78 cells)
Testing and cryopreservation of the AB-101 master cell bank (approximately 200 bags)
Thaw (single bag), expand and co-culture with engineered HuT-78 cells
Further expansion in bioreactor
Harvest and fill (1×10⁹ NK cells per vial)
Cryopreservation of the AB-0 drug product (approximately 150 vials)
Extensive characterization to determine consistency, purity, potency and safety.

As shown in Table 13, this manufacturing process reproducibly generates very large quantities of highly pure and active AB-101 drug product NK cells. Data points represent products generated from three independent cord blood units.

TABLE 13
AB-101 Product Characterization

| | | | Engineering Batches | | | Clinical Batches | | | |
|---|---|---|---|---|---|---|---|---|---|
| Test Attribute | | Acceptance Criterion | 1 | 2 | 3 | 1 | 2 | 3 | 4 |
| Cell Count | | 0.9-1.3 × 10⁹ | 1.3 × 10⁹ | 1.1 × 10⁹ | 1.0 × 10⁹ | 1.3 × 10⁹ | 1.2 × 10⁹ | 1.2 × 10⁹ | 1.0 × 10⁹ |
| (cells/vial) | | | | | | | | | |
| Cell Viability | | ≥70% | 96% | 95% | 94% | 93% | 94% | 94% | 94% |
| Endotoxin (EU/mL) | | ≤5 | ≤1 | ≤1 | ≤1 | ≤1 | <1 | ≤1 | <1 |
| Identity | CD3−, | ≥85% | 99.16% | 99.79% | 99.43% | 99.53% | 98.40% | 97.87% | 98.54% |
| | CD56+ | | | | | | | | |
| | % | | | | | | | | |
| | CD56+, | ≥70% | 94.42% | 94.20% | 99.04% | 93.24% | 91.72% | 95.22% | 90.21% |
| | CD16+ | | | | | | | | |
| | % | | | | | | | | |
| Purity | CD3+ | (CD3+) ≤ 0.20% | ≤0.00% | 0.00% | 0.00% | 0.06% | 0.00% | 0.00% | 0.02% |
| | % | | | | | | | | |
| | CD14+ | (CD14+) ≤ 1.00% | ≤0.02% | 0.00% | 0.00% | 0.02% | 0.03% | 0.01% | 0.10% |
| | % | | | | | | | | |
| | CD19+ | (CD19+) ≤ 1.00% | ≤0.01% | 0.01% | 0.00% | 0.00% | 0.00% | 0.05% | 0.05% |
| | % | | | | | | | | |
| Potency | | ≥50% killing at 4 | 69.00% | 60.20% | 64.10% | 64.50% | 67.10% | 54.80% | 67.40% |
| | | hours | | | | | | | |

### Appearance, Suspension

Appearance is performed through visual observation of AB-101 Drug Product vials assessing clarity, color and presence or absence of particulates.

### Cell Count

Cell count is performed using an ADAM Cell Counting System. This ADAM system uses two types of staining solutions: (1) Propidium iodide (PI) and lysis solution for counting total cells and (2) Propidium iodide (PI) and PBS for counting nonviable cells. AB-101 Drug Product sample is stained with Propidium iodide and loaded into Accuchip 4×. The Accuchip is loaded into ADAM Cell Counting System and cell count, cell concentration and cell viability are determined.

### Cell Viability

Viability of AB-101 Drug Product is performed using ADAM Cell Counting System as described above.

### Mycoplasma (USP <63>)

Mycoplasma testing is performed by the agar and broth media procedure proposed in USP <63>, An aliquot of AB-101 Drug Product is added to agar and broth media, respectively. The medium is then cultured under aerobic (5% CO₂) conditions for 14 days, and anaerobic (5% CO₂ in N₂) conditions for 28 days as the “Broth Medium Test”. If the drug substance is contaminated with mycoplasma, the agar media will demonstrate colonies and the broth media show color changes.

### Sterility (USP <71>)

Sterility testing performed according to “Direct Inoculation” method described in USP <71>, “Sterility Test”. An aliquot of the test sample is directly transferred into growth-promoted culture media that have the ability to grow microorganisms. Incubation occurs at a suitable temperature for the recommended duration proposed in USP. After incubation, the growth of microorganisms is determined visually.

### Endotoxin (USP <85)

Endotoxin testing is performed according to the “Kinetic Turbidimetric” method described in USP <85>. Bacterial endotoxins are a component of the cell wall of Gram-negative bacteria. The bacterial endotoxin test is an assay used to detect or quantify endotoxins from Gram-negative bacteria. The endotoxin content of the test article is determined by reading the results for the diluted test article samples against the standard curve based on the rate of turbidity of the lysate reagent reaching specific absorbance in the presence of endotoxin and adjusting for the dilution factor.

### Karyology (G-Band)

G-banded karyotyping for AB-101 Drug Product is performed. The assay has a maximum resolution of 5-10 megabase pairs. The method detects balanced and unbalanced translocations.

### Cytogenetic CNV Analysis (High Density SNP Arrays)

Copy Number Variation (CNV) assessment of AB-101 Drug Product is performed using cytogenetic analysis with high density SNP arrays to detect copy number variants, duplications/deletions, unbalanced translocations and aneuploidies. For measurement of CNV, genomic DNA is isolated, quantified, amplified, fragmented and hybridized to the bead chip for analysis. Fluorescence type and intensity of each probe is analyzed by software.

### Identity (CD3−, CD56+)

The frequency of CD3−, CD56+ cells are used to assess the identity of AB-101 Drug Product. A sample of AB-101 Drug Product is thawed and resuspended in a staining buffer. The resuspended sample is added to fluorochrome-labeled antibodies that bind to CD3+ and CD56+ surface antigens. Flow cytometry is used to determine percent populations of CD3−, CD56+ as a measure of product identity.

### Identity (CD56+, CD16+)

The frequency of CD56+, CD16+ cells are used to assess the identity of AB-101 Drug Product. A sample of AB-101 Drug Product is thawed and resuspended in a staining buffer. The resuspended sample is added to fluorochrome-labeled antibodies that bind to CD56+ and CD16+ surface antigens. Flow cytometry is used to determine percent populations of CD56+, CD16+ as a measure of product identity.

### Purity (CD3+)

Measurement of CD3+ expressing cells are used to assess the purity of AB-101 Drug Product. Flow cytometry method is used to determine the purity of the drug product for CD3+ expressing cells. The percent population of CD3+ cells is used as a measure of product purity.

### Purity (CD14+)

Measurement of CD14+ expressing cells are used to assess the purity of AB-101 Drug Product. Flow cytometry method is used to determine the purity of the drug product for CD14+ expressing cells. The percent population of CD14+ cells is used as a measure of product purity.

### Purity (CD19+)

Measurement of CD19+ expressing cells are used to assess the purity of AB-101 Drug Product. Flow cytometry method is used to determine the purity of the drug product for CD19+ expressing cells. The percent population of CD19+ cells is used as a measure of product purity.

Purity: Residual eHuT-78P (Residual eHuT-78P Cells)

Residual eHuT-78P cells in AB-101 drug product are measured by flow cytometry (FACS). FACS is used detect residual eHuT-78 in AB-101 DP by quantifying the live CD3+4-1BBLhigh+ eHuT-78P. The FACS gating strategy (See FIG. 1), which sequentially gates, singlet, 7-AAD and CD3+4-1 BBL+, was used because eHuT-78 is derived from a HuT-78 cell line that expresses CD3 as cutaneous T lymphocyte. The HuT-78 cell line was transduced by 4-1BB ligand (4-1BBL), membrane tumor necrosis factor-a (mTNF-α) and membrane bound IL-21 (mbIL-21). An eHuT-78 single cell that highly expresses the three genes was selected, and research, master and working cell banks were successively established. Among the three genes, 4-1 BBL was utilized for the FACS gating strategy because it showed the highest expression in AB-101 cell bank and final drug product.

### Potency (Cytotoxicity at 10:1 AB-101 DP Cells to K562 Cells)

Potency of AB-101 Drug Product is determined by evaluating capacity for cellular cytotoxicity against K562 tumor cells. Cytotoxicity of the drug product will be assessed by fluorometric assay. K562 tumor cells are stained with 30 μM calcein-AM (Molecular probe) for 1 hour at 37° C. A sample of the drug product and the labeled tumor cells are co-cultured in a 96-well plate in triplicate at 37° C. and 5% CO2 for 4 hours with light protection. RPMI1640 medium containing 10% FBS or 2% triton-X100 was added to the targets to provide spontaneous and maximum release. RPMI1640 medium containing 10% FBS or 2% triton-X100 is added to each well to determine background fluorescence. The measurement of fluorescence is conducted at excitation of 485 nm and emission 535 nm with a florescent reader. The percent specific cytotoxicity is calculated by the following formula.

$$\% Specific cytotoxicity = 100 \times \frac{\% specific death - \% spontaneous death}{100 - \% spontaneous death}$$

### Potency (Cytotoxicity at 10:1 AB-101 DP Cells to Ramos Cells)

Potency of AB-101 Drug Product is also determined by evaluating the capacity for cellular cytotoxicity against Ramos tumor cells using the same method and calculation described above. The specification for this testing is being determined.

### Example 7: AB-101 Phenotypic Characterization

The purity as well as expression of antibody-engaging CD16 and activating, inhibitory and chemokine receptors of multiple batches of AB-101 were measured via flow cytometry.

AB-101 purity was measured using cell surface markers: AB-101 batches were seen to comprise >99% CD3-CD56+ NK cells and <0.1% CD3+, CD14+ and CD19+ cells. CD16 expression of AB-101 was measured. 95.11±2.51% of AB-101 cells were CD16+ with mean and median MFI of CD16 15311±6186 and 13097±5592 respectively. NK cells are known to express various NK specific activating and inhibitory receptors. For the various AB-101 batches that were tested, >80% of cells expressed CD16, NKG2A, NKG2D, CD94, NKp30, 2B4, Tim-3, CD44, 40˜70% of cells expressed NKp44, NKp46, DNAM-1, approximately 30% of cells expressed CD161 and CD96, 15% of cells expressed CXCR3, and less than 5% of cells expressed other activating inhibitory receptors.

Two GMP batches of AB-101 were included in the study to assess the phenotypic characteristics of NK cells at three different stages of the manufacturing process: Cord blood cells post CD3+ cell depletion; master cell bank (MCB) as intermediate, and AB-101 final drug product (DP). The CD3 depleted cells, MCB and DP, each were measured for purity and NK cell receptors. Based on the results, it was seen that NK cells initially derived from CB showed immature NK phenotypes. The NK phenotype matured during the manufacturing process. At the MCB stage, more than 90% of cells already expressed the phenotypic characteristic seen in matured NK cells, and markers of other cell types were <0.1%. The expression level for most of the NK cell-specific receptors increased throughout the manufacturing process from CD3 depleted cells, to MCB and finally DP

List of Abbreviations: NK: Natural killer; mAb: Monoclonal antibody; TNF-α: Tumor necrosis factor alpha; CXCR: CXC chemokine receptors; DNAM-1: DNAX Accessory Molecule-1; CRACC: CD2-like receptor-activating cytotoxic cell; ILT2: Ig-like transcript 2; Tim-3: T-cell immunoglobulin mucin-3; 7AAD: 7-amino-actinomycin D; ULBP: UL16-binding protein; MICA/B: MHC class I chain-related protein A and B; RAE1: Ribonucleic Acid Export 1; H60: NKG2D interacts with two cell surface ligands related to class I MHC molecules; MULT1: mouse UL16-binding protein-like transcript 1; MHC: Major histocompatibility complex; HLA: Human Leukocyte Antigen.

Phenotype and purity staining protocol: 1. Adjust NK cell concentration at 2.0×106 cells/mL in cold FACS buffer. 2. Refer to the table below, make an antibody mixture. 3. Add and mix antibody mixture with 100 μL diluted cells in a 5 mL round bottom tube. 4. Stain the cells for 30 minutes under blocking light and 4° C. conditions. 5. After staining, add 2 mL of FACS and then centrifuge for 3-minutes under 2000 rpm and 4° C. conditions. 6. Discard supernatant and vortex the cell pellet. Then add 200 μL of FACS buffer. 7. Analyze cells on the flow cytometer (LSR Fortessa) 8. Analyze the expression level of each marker by using Flow-Jo software. 9. Gate phenotype as follow gating option. a. Gate singlet in FSC-A/FSC-H panel b. Gate live cell in 7-AAD/SSC-A panel c. Gate lymphocyte in FSC-A/SSC-A panel d. Gate NK cell (CD3-CD56+) in CD3/CD56 e. Draw quadrant according to isotype control and then analyze CD3/CD56, CD16/CD56, and CD14/CD19. f. Based on Fluorescence Minus One (FMO) in NK cells gating, each PE fluorescent expression of the markers (no. 1 and 3-30 in the table 1, % of expression) is counted. In case of CD16, mean ratio and median is counted.

A list of antibody combinations for NK cell phenotype staining is shown in Table 14.

TABLE 14
List of antibody combinations for NK cell phenotype staining

| | FITC | | PE-Cy7 | PerCP-Cy5.5 |
|---|---|---|---|---|
| | (Fluorescein | PE | (Phycoerythrin- | (Peridinin-chlorophyll- |
| No. | isothiocyanate) | (phycoerythrin) | Cyanine7) | protein Complex: CY5.5 |
| 1 | CD3 | CD16 | CD56 | 7-AAD |
| 2 | CD14 | CD19 | CD3 | |
| 3 | CD3 | NKG2A | CD56 | |
| 4 | | NKG2C | | |
| 5 | | NKG2D | | |
| 6 | | NKp30 | | |
| 7 | | NKp44 | | |
| 8 | | NKp46 | | |
| 9 | | NKp80 | | |
| 10 | | CXCR3 | | |
| 11 | | CXCR4 | | |
| 12 | | CXCR5 | | |
| 13 | | CXCR6 | | |
| 14 | | CD195 | | |
| 15 | | CD244 | | |
| 16 | | DNAM-1 | | |
| 17 | | CD44 | | |
| 18 | | CD57 | | |
| 19 | | CD62L | | |
| 20 | | CD69 | | |
| 21 | | CD94 | | |
| 22 | | CD96 | | |
| 23 | | CD161 | | |
| 24 | | CRACC | | |
| 25 | | ILT-2 | | |
| 26 | | OX40L | | |
| 27 | | Tim-3 | | |
| 28 (FMO) | | mIgG1 | | |
| 29 (Iso- | mIgG1 | mIgG1 | mIgG1 | |
| type) | | | | |

Purity of AB-101 (n=9)

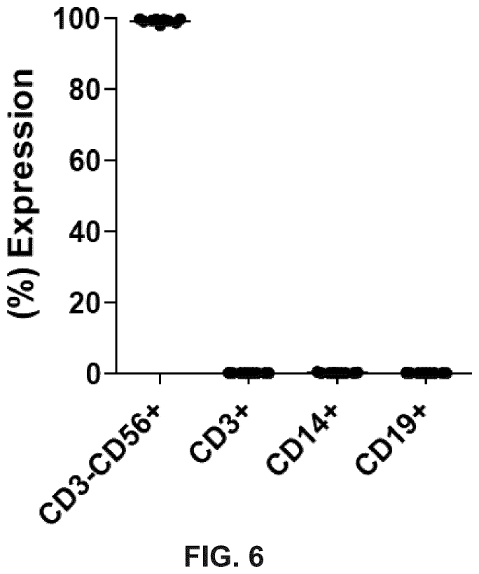

The purity of AB-101 is represented as CD3-CD56+ cells for NK cells, CD3+ cells for T-cells, CD14+ cells for monocytes and CD19+ cells for B-cells. Total 9 batches of AB-101 were measured for the purity. The results showed 99.27±0.59% (mean±SD) for CD3-CD56+ cells, 0.02±0.03% for CD3+ cells, 0.10±0.12% for CD14+ cells, and 0.02±0.04% for CD19+ cells (FIG. 6). Therefore, it was confirmed that AB-101 is composed of high-purity of NK cells, and the other types of cells as impurities were rarely present.

### Comparison of Purity of CD3 Depleted Cells, MCB. And DP Manufactured in GMP Conditions

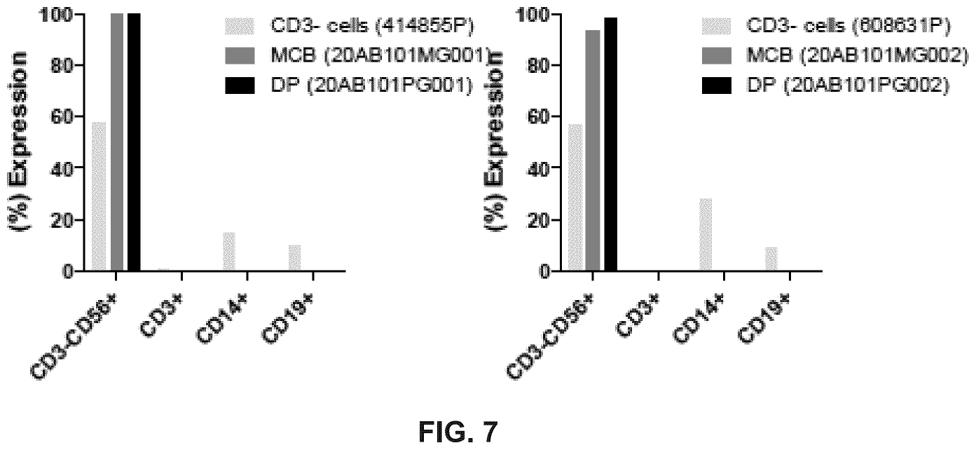

Two GMP batches of AB-101 were utilized to assess the purity of AB-101 starting material (CD3 depleted cells), intermediate (master cell bank, MCB), and final drug product (DP). 50˜60% of cells in CD3 depleted cell fraction were NK cells, and these percentages increased to more than 90% in MCB and DP. CD14+ cells and CD19+ cells were representative of 20˜30% of CD3 depleted cell fraction, and these cell percentages decreased to less than 0.1% in MCB and DP indicative of purity of AB-101 MCB and AB-101 final drug products (FIG. 7, Table 15).

TABLE 15
Cell Purity

| | GMP batch #1 | | | GMP batch #2 | | |
|---|---|---|---|---|---|---|
| | CD3- cells | MCB (20AB101 | DP (20AB101 | CD3- cells | MCB (20AB101 | DP (20AB101 |
| Marker | (414855P) | MG001) | PG001) | (608631P) | MG002) | PG002) |
| CD3-CD56+ (%) | 58.0 | 99.43 | 99.80 | 56.70 | 93.14 | 97.98 |
| CD3+ (%) | 0.79 | 0.05 | 0.01 | 0.21 | 0.03 | 0.02 |
| CD14+ (%) | 15.01 | 0.02 | 0.01 | 28.00 | 0.03 | 0.02 |
| CD19+ (%) | 9.83 | 0.01 | 0.00 | 9.17 | 0.00 | 0.00 |

### Comparison of NK Cell Receptors of CD3 Depleted Cells, MCB, and DP Manufactured in GMP Conditions

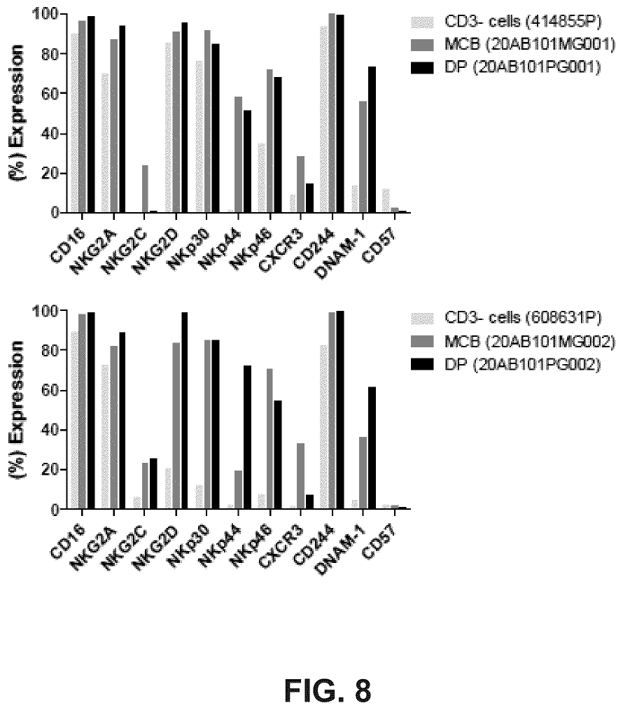

Two GMP batches of AB-101 were also utilized to assess the expression of various NK cell receptors on AB-20 starting material (CD3 depleted cells), intermediate (master cell bank, MCB), and final drug product (DP). It was observed that several NK cell and activating receptors such as CD16, NKG2D, NKG2C, NKp30, NKp44, NKp46 and DNAM-1 were expressed in higher levels by MCB, final drug product when compared to AB-101 starting material (CD3 depleted cells). The CD57 expression was lower in MCB and final drug product when compared to AB-KG2 starting material (CD3 depleted cells) (FIG. 8, Table 16). Overall, data shows an increase in expression of NK cell activating receptors in MCB and DP indicative of AB-101 being effective against tumors.

TABLE 16
Cell Receptor Expression

| | GMP batch #1 | | | GMP batch #2 | | |
|---|---|---|---|---|---|---|
| | CD3- cells | MCB (20AB101 | DP (20AB101 | CD3- cells | MCB (20AB101 | DP (20AB101 |
| Marker | (414855P | MG001) | PG001) | (608631P) | MG002) | PG002) |
| Cd16 | 90.27 | 96.45 | 98.50 | 89.27 | 97.70 | 98.30 |
| NKG2A | 69.99 | 87.05 | 93.70 | 72.94 | 81.92 | 88.43 |
| NKG2C | 0.26 | 23.87 | 1.11 | 6.32 | 22.91 | 25.04 |
| NKG2D | 85.52 | 91.13 | 95.17 | 20.70 | 83.16 | 98.77 |
| NKp30 | 76.29 | 91.55 | 94.64 | 12.61 | 85.19 | 85.22 |
| NKp44 | 1.29 | 58.27 | 51.14 | 2.48 | 19.15 | 72.03 |
| NKp46 | 35.12 | 71.83 | 67.77 | 7.64 | 70.54 | 54.46 |
| CXCR3 | 9.10 | 28.39 | 14.40 | 1.79 | 33.13 | 7.01 |
| 2B4 | 93.66 | 99.75 | 99.20 | 82.63 | 98.29 | 99.46 |
| DNAM-1 | 13.94 | 55.64 | 73.07 | 5.12 | 36.24 | 61.13 |
| CD57 | 12.24 | 1.92 | 0.65 | 2.63 | 1.63 | 0.74 |

### Conclusion

The use of surface marker analysis supported the identity and purity and batch-to-batch consistency of the AB-101 product. Further, extensive assessment of NK-specific activating and inhibitory cell surface markers established the consistent profile of the AB-101 product post manufacturing expansion process. It is known that CB derived NK cells have immature phenotype such as high expression of NKG2A and low expression of NKG2C, CD62L, CD57, IL-2R, CD16, DNAM-1 comparing to peripheral blood (PB) derived NK cells, and it is also known that CB derived NK cells with the immature phenotypes exhibit low cytotoxicity against tumor cells. Data from this report shows that AB-101, an allogeneic cord blood (CB) derived NK cell product, expresses high levels of major activating receptors indicative of potential higher cytotoxicity against tumor cells.

### Example 8: AB-101 Non Clinical Studies

Natural killer (NK) cells play a crucial role in the host immune system and form a first line of defense against viral infections and cancer. In comparison to other lymphocytes, NK cells are unique in their capability to elicit rapid tumoricidal responses without the need for antigen presentation or prior sensitization (Miller J S. Therapeutic applications: natural killer cells in the clinic. Hematology Am Soc HematolEauc Program. 2013; 2013:247-53; Malmberg K J, Carlsten M, Bjorklund A et al., Natural killer cell-mediated immunosurveillance of human cancer. Semin Immunol. 2017 June; 31:20-29). Nonclinical studies of AB-101 characterized the expected functional characteristics, mechanism of action, cellular kinetics, and toxicology of the product to inform its clinical use.

Non-clinical studies described in the following examples include: 1) Data characterizing the cellular components and phenotype of the cells present in the AB-101 drug product; 2) Data demonstrating cytotoxicity against human leukemia and lymphoma cell lines (Ramos and Raji), 3) Data illustrating specificity for cancer cell targets and showing production of pro-inflammatory cytokines upon tumor cell stimulation, 4) Data illustrating enhanced in vitro effector functions and in vivo anti-tumor activity of AB-101 in combination with rituximab, and 5) Data from the GLP in vivo toxicity study and an in vivo biodistribution and persistence study demonstrating that AB-101 was well tolerated, had a tissue distribution consistent with the intravenous route of administration and lacked long-term persistence. Major findings of in vitro and in vivo preclinical efficacy studies of AB-101 are summarized in Table 17.

TABLE 17
Summary of Nonclinical Studies

| Studies | Assay | Major Findings |
|---|---|---|
| In vitro | Fluorometric-based | AB-101 demonstrated cytotoxic activity |
| cytotoxicity of | (calcein- | against tumor cell lines. |
| AB-101 (K562, | acetoxymethyl | AB-101 showed improved expression of |
| Raji, Ramos) | release) cytotoxicity | intracellular effector cytokines and |
| | assay | degranulation markers following co-culture |
| | Flowcytometry | with various tumor cell lines. |
| | analysis of | |
| | intracellular | |
| | cytokines and | |
| | degranulation marker | |
| In vivo cytotoxicity | Survival and | AB-101 in combination with rituximab |
| of AB-101 (Raji and | monitoring of | demonstrated enhanced anti-tumor activity |
| Ramos tumor | hindlimb paraplegia | on comparison with both AB-101 and |
| models) | in SCID Xenograft | rituximab monotherapies. |
| | models | |
| Pharmacokinetics | In vivo | Biodistribution of AB-101 cells in vivo is |
| | biodistribution and | consistent with the intravenous route of |
| | persistence of AB- | administration of cellular products. The |
| | 101 by qPCR | cells lack long-term persistence potential |
| | following repeat | and were cleared after 7 days post- |
| | intravenous injection | administration with no evidence of |
| | at escalating doses in | permanent engraftment. |
| | immunodeficient | |
| | NSG mice | |
| Dose Range | In vivo assessment of | Three doses and two schedules of AB-101 |
| Finding Study | safe dose range of | were tested. 2.5 × 10⁷ cells/dose delivered |
| | AB-101 cells in NSG | intravenously once weekly for 8 weeks to |
| | mice following | NSG mice was determined as the |
| | repeat intravenous | Maximum Tolerated Dose (MTD). |
| | injections | |
| GLP Toxicity | In vivo assessment of | Once weekly intravenous administration of |
| Study | potential toxicity of | AB-101 at dose levels of 0.5 × 10⁷ and 2 × |
| | AB-101 in NSG | 10⁷ viable cells, in mice, resulted in no test |
| | mice | article related mortalities, changes in body |
| | | weight, ophthalmology, clinical pathology, |
| | | or anatomic pathology endpoints. Based on |
| | | a lack of adverse findings, the No- |
| | | Observed-Effect-Level (NOEL) was 2 × 10⁷ |
| | | viable cells. |

The nonclinical data summarized below and in Example 9, Example 10, Example 11, and Example 12 indicate that the administration of AB-101 is safe and exhibits anti-tumor activity alone or in combination with rituximab. Secretion of cytokines and chemokines and ability to safely and effectively deliver multiple doses in the preclinical model supports clinical use of AB-101.

The preclinical studies indicate that AB-101 displays a phenotype and a range of inhibitory and activating receptors consistent with and characteristic of normal NK cell phenotype. Moreover, the described studies show AB-101 displays directed cytotoxicity, in vitro. The tumor derived cell lines used in the study include representatives of disease settings where antibodies, e.g., rituximab, have been applied and, in some cases, shown to encounter resistance. Furthermore, AB-101 demonstrated the capacity to produce IFNγ and TNFα in response to tumor cell engagement. Secretion of these cytokines is expected to facilitate recruitment and activation of endogenous T cells and bridge the innate and adaptive immune response.

In xenograft models of human lymphoma cancer, AB-101 displayed significant reduction of tumor burden when administered in a multi-dose schedule, supporting the clinical schema and dosing strategy. Notably, AB-101 showed consistent specificity to the tumor target cells. Collectively, these data demonstrate that AB-101 exhibits the primary characteristics of NK cells including specific induction of cytotoxicity and cytokine production in response to engagement with malignant cells and maintenance of appropriate tolerance to normal, non-cancerous cells.

Repeat dosing in NSG mice, reflective of the proposed clinical schema, demonstrated that AB-101 distributed predominantly to highly perfused tissues, as expected, following intravenous administration and lacked long-term persistence or engraftment. There was no evidence of toxicity (acute and delayed) related to the administration of AB-101.

Based on the preclinical studies described above, AB-101 is expected to be a safe and functional NK cell product with potential clinical utility, e.g., for lymphoma patients, as a monotherapy or when combined with antibodie(s), e.g., rituximab.

### Objective

The purpose of this study was to evaluate in vitro anti-tumor efficacy of cord blood derived NK cells (CB-NK), AB-101. Assessments included, direct cellular cytotoxicity, antibody dependent cellular cytotoxicity (ADCC) and the intracellular cytokine production and the degranulation marker (CD107a) expression of AB-101 against tumor cell lines.

List of Abbreviations: K562: A human erythroleukemic cell line; Ramos: CD20+ human Burkitt's lymphoma cell line; Raji: CD20+ human B-lymphocytes of Burkitt's lymphoma cell; line; CB-NK: Cord blood derived NK cells; ADCC: Antibody dependent cellular cytotoxicity; Rituximab: (RTX) Rituxan or Mabthera. A monoclonal antibody to target CD20; MM well: The well containing medium (RPMI1640 and 10% FBS, afterwards “R-10” medium) only for analysis and for correcting the fluorescence value of media itself; MT well. The well containing an equal amount of R-10 media and 2% Triton-X100 (final 1% Triton-X100) and for correcting the fluorescence value of media itself; Spon. Well: The well for measuring the fluorescence dye spontaneously emitted in the medium when the Calcein-AM stained tumor cell line is suspended in R-10. Max. well: The well for measuring the fluorescence value emitted when the Calcein-AM stained tumor cell line is dissolved 100% with 1% Triton-X 100. IFN-γ: Interferon gamma; TNF-α: Tumor necrosis factor-α; FACS: Fluorescence-activated cell sorting; Ramos-NucLight: For an imaging assay, the Ramos cell line was transfected by lentiviral vector expressing red fluorescent; Raji-NucLight For an imaging assay, the Raji cell line was transfected by lentiviral vector expressing red fluorescent; PLO (Poly-LOrthinine) Synthetic amino acid polymer to adhere the cells on the surface of well; E:T ratio A ratio of effector cells to target cells

### Summary

AB-101 is allogeneic cord blood derived natural killer cells, which is currently developed as an anti-tumor immune cell therapy targeting lymphoma. It is known that NK cells can directly kill tumors without recognition of specific antigens, or indirectly eliminate them with recognition of tumor specific antibodies, and also indirectly kill them by stimulating the acquired immune systems via secreting a variety of cytokines. In this study, the direct cytotoxicity, long-term ADCC and intracellular cytokine staining (ICS) were performed to evaluate in vitro anti-tumor efficacy of AB-101.

1. To evaluate the anti-cancer efficacy of AB-101, cytotoxicity against hematopoietic cancer derived tumor cell lines was determined using short-term cytotoxicity assay. AB-101 showed effector cell to target cell ratio (E:T ratio)-dependent cytotoxicity upon coculture with tumor cell lines for a duration of 4 hours. At an E:T ratio of 10:1, the mean cytotoxicity activity across 9 batches of AB-101 against K562, Ramos and Raji cells was 73.9±4.6%, 57.1±8% and 77.0±2.8% respectively. The deviation among the batches was less than 10%. These results demonstrate direct cytotoxicity of AB-101 against K562, Ramos and Raji tumor cells and the consistency of cytotoxic activity between batches of AB-101 product.

2. To evaluate the efficacy of combining of AB-101 and Rituximab (RTX, a CD20 targeted antibody), long-term ADCC was evaluated against CD20 positive lymphoma Ramos and Raji cell lines. AB-101 consistently showed cytotoxicity against Ramos and Raji cell lines over a 72 hour period, and the cytotoxicity was enhanced when it is combined with RTX. At the 72 hour timepoint, the percent of live Ramos cells (compared to Ramos cells alone) were 37.6±15.4% for AB-101 alone, 42.5±15.9% for AB-101+hIgG, and 19.0±11.9% for AB-101+RTX culture conditions respectively. The percent of live Raji cells were 20.5±12.2% for AB-101 alone, 20.5±12.2% for AB-101+hIgG, and 10.1±4.6% for AB-101+RTX culture conditions respectively. The deviation among the batches of AB-101 in this long-term ADCC culture condition was less than 15% for Ramos cells and 5% Raji cells. Thus, AB-101+RTX combination demonstrated a significantly increased long-term cytotoxicity i.e. lysis of ˜80-90% of tumor cells when compared to AB-101 alone or AB-101+hIgG.

In conclusion, results obtained from these in vitro assays confirmed that a) AB-101 had a direct cytotoxic activity against the tested tumor cell lines, b) cytotoxicity of AB-101 against lymphoma cell lines expressing CD20 antigen could be significantly increased by combining it with rituximab and this increase in cytotoxicity could be attributed to ADCC and, c) AB-101 could significantly express immune modulating cytokines and marker of degranulation (CD107a) in response to target cells stimulation when compared to unstimulated condition.

### Introduction

NK cells have an innate ability to kill tumor cells or virus-infected cells either by direct or indirect mechanisms without the restriction of major histocompatibility complex (MHC) or preimmunization. Cytolytic activity of NK cells against tumors is dependent on the balance of inhibitory and activating receptors. NK cell mediated killing of tumor cells can be categorized into three different mechanisms a) by the release cytoplasmic granules including perforin and granzymes that induce apoptosis of tumor cells through caspase-dependent or independent path [1, 2], b) by inducing apoptosis of tumor cells which is mediated by signals of death-receptors such as Fas-FasL, TRAIL-TRAILR and TNF-a-TNFR [3-8] and, c) by recognizing the tumor specific antibodies using cell surface CD16 and killing the tumor cells by ADCC [9]. In addition to direct and indirect killing mechanisms, NK cells demonstrate anti-tumor efficacy by secreting various effector molecules including IFN-γ which suppress angiogenesis of tumors or stimulate adaptive immune system [10-15]. The effector functions of AB-101 i.e., their capacity to express effector cytokines and marker of degranulation upon malignant cell engagement and to elicit cytotoxicity i.e., direct and ADCC against malignant cells was assessed in a series of studies.

TABLE 18
Test Article Information/Identification:

| | Product Name | | | |
|---|---|---|---|---|
| | AB-101 | | | |
| | Product Description | | | |
| | Human cord blood (CB)-derived Natural Killer cell | | | |
| | Batch Number | Batch Type | Start and End of production | Purpose of production |
| Product | 19AB101PN001 | Engineering | 2019 Sep. 18 to 2019 Oct. 1 | DRF Tox study/ |
| Information | | Lots | | Stability (~6M) |
| | 19AB101PN004 | | 2019 Oct. 29 to 2019 Dec. 27 | GLP Tox study |
| | 19AB101PN005 | | 2019 Dec. 11 to 2019 Dec. 27 | GLP Tox study |
| | 20AB101PN001 | | 2020 Jan. 2 to 2020 Jan. 16 | Stability for IND |
| | 20AB101PN002 | | 2020 Feb. 5 to 2020 Feb. 19 | Equipment PQ |
| | 20AB101PN003 | | 2020 Mar. 4 to 2020 Mar. 20 | Stability for IND/ |
| | | | | Equipment PQ |
| | 20AB101PN004 | | 2020 Mar. 18 to 2020 Apr. 2 | Equipment PQ |
| | | | | (Br, KS, AF) |
| | 20AB101PG001 | GMP lots | 2020 May 30 to 2020 Jun. 12 | Stability for IND |
| | 20AB101PG002 | | 2020 Jun. 10 to 2020 Jun. 22 | Stability for IND |

| | |
|---|---|
| Storage | <−135 in the vapor phase of liquid nitrogen in a liquid nitrogen freezer |
| Condition | |
| Supplier | GC LabCell |

TABLE 19
Target Cell Line Information/Identification:

| Product Name | K562 | |
|---|---|---|
| Product Description | A human erythroleukemic cell line | |
| Product Information | ATCC/Cat No. CCL-243 | |
| Storage Condition | <−135° C. in the vapor phase of liquid nitrogen in a liquid nitrogen | |
| | tank | |
| Supplier | GC LabCell | |
| Product Name | Ramos | |
| Product Description | A human Burkitt's lymphoma cell line | |
| Product Information | ATCC/Cat No. CRL-1596/Lot No. 70016960 | |
| Storage Condition | <−135° C. in the vapor phase of liquid nitrogen in a liquid nitrogen | |
| | tank | |
| Supplier | ATCC | |
| Product Name | Raji | |
| Product Description | A human B-lymphocytes of Burkitt's lymphoma cell line | |
| Product Information | ATCC/Cat No. CCL-86 | |
| Storage Condition | <−135° C. in the vapor phase of liquid nitrogen in a liquid nitrogen | |
| | tank | |
| Supplier | ATCC | |
| Product Name | Ramos-NucLight cell line (Self-manufactured by GC LabCell) | |
| Product Description | The Ramos cell line made in-house to emit red fluorescence in the | |
| | nucleus of cells using NucLight red lentivirus reagent for an imaging | |
| | assay | |
| Product Information | NucLight red lentivirus reagent | Cat No: 4625 |
| | (Sartorius) | Lot No: LDA062918.02-022219 |
| Storage Condition | <−135° C. in the vapor phase of liquid nitrogen in a liquid nitrogen | |
| | tank | |
| Supplier | GC LabCell | |
| Product Name | Raji-NucLight cell line (Self-manufactured by GC LabCell) | |
| Product Description | The Raji cell line made in-house to emit red fluorescence in the | |
| | nucleus of cells using NucLight red lentivirus reagent for an imaging | |
| | assay | |
| Product Information | NucLight red lentivirus reagent | Cat No: 4625 |
| | (Sartorius) | Lot No: LDA062918.02-022219 |

| | |
|---|---|
| Storage Condition | <−135° C. in the vapor phase of liquid nitrogen in a liquid nitrogen |
| | tank |
| Supplier | GC LabCell |

TABLE 20
Therapeutic Antibody Information:

| | |
|---|---|
| Product Name | Rituximab (Mabthera or Rituxan) |
| Product Description | Anti CD20 monoclonal antibody, IDEC-C2B |
| Product Information | N7297B43 |
| Storage Condition | 2-8° C. |
| Supplier | Roche Pharma (Schweiz) Ltd. |
| Product Name | Human IgG (hIgG) |
| Product Description | Immunoglobulin G obtained from human serum |
| Product Information | Cat No.: 14506/Lot No.: SLBR0560V |
| Storage Condition | 2-8° C. |
| Supplier | Sigma-Aldrich |

### In Vitro Direct Cell Cytotoxicity Protocol:

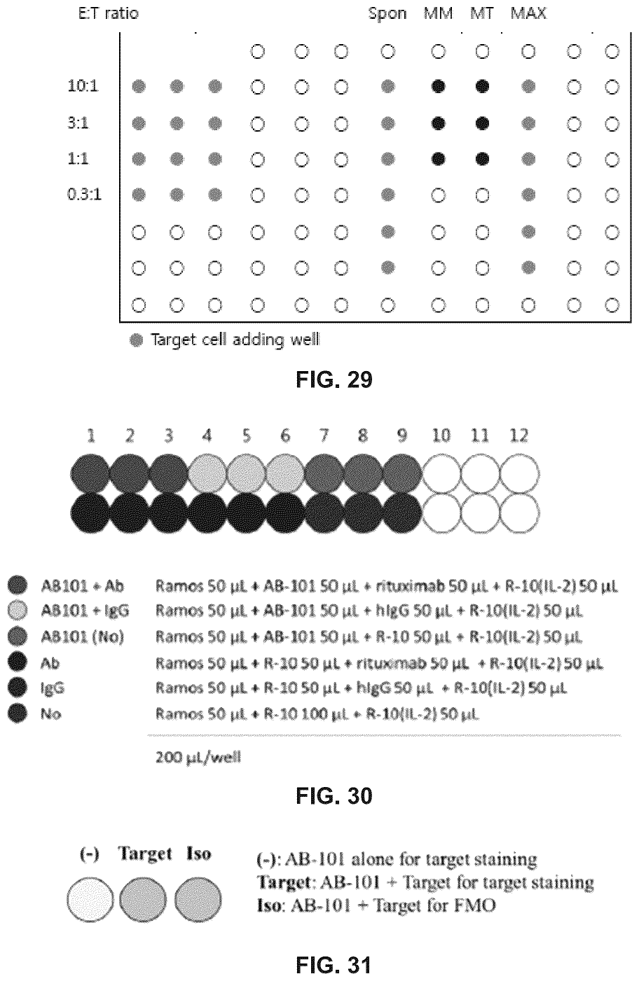

1. Resuspend the target cell line in RPMI1640-10%/FBS (R-10) medium to prepare 1×106 cells/mL. 2. Add 30 μL of 1 mM calcein-AM to 1 mL of the target cell line and vortex the tube. Stainthe cells for 1 hour in a CO2 incubator at 37° C. 3. Approximately 1 hour later, add 10 mL of the R-10 medium and remove the supernatantvia centrifugation (1200 rpm, 5 min, 4° C.). Repeat this step one more time. 4. Add 10 mL of the R-10 medium and resuspend at 1×105 cells/mL, and transfer 100 μL of the target cell line into a 96 well round bottom plate. 5. Dilute the effector cells (AB-101 cells) according to the following E:T ratios such as, 10:1, 3:1, 1:1, 0.3:1 and add 100 μL of each into the wells containing the target cell line. Perform this in triplicate. 6. Add 100 μL of the target cell line into both “Spon well” and “MAX well”, and add 100 μL of the R-10 medium into “Spon well” and 100 μL of the 2% Triton-X100 solution into “MAX well” each. 7. Add 200 μL of the R-10 medium into “MM well” and add 100 μL of the R-10 medium and 100 μL of the 2% Triton-X100 solution into “MT” well”. 8. Wrap the 96 well plate with aluminum foil to prevent from light and incubate the plate in a CO2 incubator at 37° C. for 4 hours. (FIG. 29) 9. After 4 hours, take out the 96-well plate and centrifuge it (2000 rpm, 3 min, 4° C.). 10. Transfer 100 μL of the supernatant to a 96 well black plate and measure the fluorescence at Excitation (485 nm)/Emission (535 nm) using a fluorimeter. 11. Convert the cytotoxicity as follows:

$$Calculation Method 1$$

$$A ( A corrects the default fluorescence of medium ) = Mean fluorescence of MM well - Mean fluoresence of MT well$$

$$Specific lysis ( \% ) = Mean fluorescence of Sample well - Mean fluorescence of Spon well \div \{ ( Mean fluorescence of Max well + A ) - mean fluorescence of Spon well )$$

### In Vitro Long-Term ADCC Protocol

1. Add 50 μL/well of PLO (Poly-L-omithine) into a 96-well flat-bottom plate to attach the target cell line that floats and grows suspended in the culture medium. Leave the plate at room temperature for an hour and then remove the solution. Dry the plate for 30 minutes.

2. Resuspend the target cell line expressing fluorescence (Ramos-NucLight and Raji-NucLight) in the R-10 medium at 2×10⁵ cells/mL and transfer 50 μL/well.

3. Resuspend the effector cells (AB-101) in the R-10 medium at 2×10⁵ cells/mL and transfer 50 μL/well.

4. Prepare Rituximab and hIgG antibody in the R-10 medium at 40 μg/mL and transfer 50 μL/well (Final-concentration: 10 μg/mL).

5. Add 500 IU/mL of rhIL-2 into the R-10 medium and transfer 50 μL/well (Final cell density: 125 IU/mL). (FIG. 30)

6. Insert the plate in the live-cell analyzer (Incucyte) and scan images for 72 hours.

7. After scanning, analyze the plate using IncuCyte Software (v2019B).

8. When the analysis of images is completed, the images can be presented as “Total red objective counter per image (live cell number/image)”. They are quantified as follows:

$$Calculation Method 2$$

$$Normalized live cell ( \% ) = \frac{Live cell number of ramos with AB - 101 and / or Antibody}{Live cell number of Ramos alone} \times 100 \%$$

### In Vitro Intracellular Cytokine Staining Protocol

1. Resuspend the AB-101 cells in the R-10 medium at 5×10⁶ cells/mL.

2. Resuspend the target cell line in the R-10 medium at 5×10⁶ cells/mL.

3. Prepare a 96 well U-bottom plate. Add APC anti-human CD107a antibody (1 μL) into the (−) well and target well and add APC mouse IgG1,κ isotype control (5 μL) into the isotype control well.

4. Mix AB-101 with Golgisto and Golgiplug to prevent intracellular cytokines from being released. Transfer 100 μL of the R-10 and 100 μL of the AB-101 cells into the (−) well instead of the target cell line, and add 100 μL of the AB-101 cells and 100 μL of the target cell line into the target and iso wells of the 96 well u-bottom plate containing the antibody.

5. Wrap the 96 well plate with aluminum foil to prevent from light and incubate the plate in a CO₂ incubator at 37° C. for 4 hours. (FIG. 31)

6. After 4 hours, take out the plate and remove the supernatant via centrifugation (2000 rpm, 3 minutes, 4° C.).

7. Add 200μ of FACS buffer and mix, and then remove the supernatant via centrifugation (2000 rpm, 3 minutes, 4° C.).

8. Add 100 L of FACS buffer into each well. Add 1 uL of anti-CD3-PerCP-Cy5.5, 1 uL of anti-CD56-APC-e780 and 4 μL of 7-AAD for staining the cell surface, and then incubate at 4° C. for 30 minutes.

9. After adding 100 μL of FACS buffer, remove the supernatant via centrifugation (2000 rpm, 3 minutes, 4° C.). After adding 200 μL of FACS buffer, remove the supernatant via centrifugation (2000 rpm, 3 minutes, 4° C.).

10. Add 150 μL of Fixation/Permeabilization solution for staining the intracellular antibody staining, and then incubate at 4° C. for 30 minutes.

11. After centrifugation (2000 rpm, 3 minutes, 4° C.), add 200 μL of 1× Perm wash buffer and centrifuge again (2000 rpm, 3 minutes, 4° C.).

12. Add 100 μL of 1× Perm wash buffer into each well and add antibody as below for intracellular staining, and then incubate at 4° C. for 30 minutes.

| (—), Target | | Iso well | |
|---|---|---|---|
| FITC | PE-Cy7 | FITC | PE-Cy7 |
| IFN-γ (1 μL) | TNF-α (1 μL) | Mouse IgG₁, κ | Mouse IgG₁, κ |
| | | Isotype control (5 μL) | Isotype control (1 μL) |

13. Add 100 μL of 1× Perm wash buffer and remove the supernatant via centrifugation (2000 rpm, 3 minutes, 4° C.). A dd 200 μL of 1× Perm wash buffer and centrifuge again (2000 rpm, 3 minutes, 4° C.).

14. Remove the supernatant, add 200 μL of Fixation buffer, and release the cell pellet by pipetting.

15. Measure the fluorescence using LSR Fortessa (FACS equipment).

16. After the measurement, analyze the results using FlowJo program.

17. Analyze the expression of CD107a, IFN-γ and TNF-α as below gating strategies:
1) FSC-A/FSC-H gating (Singlet)
2) FSC-A/SSC-A gating (Lymphocyte)
3) 7-AAD−, CD3−/CD56+ gating (Live NK cell)
4) Obtain each % of expression by gating the positive population of CD107a/CD56,
IFN-γ/CD56, and TNF-α/CD56 dot plot.

### Statistical Analysis:

All statistical analyses were performed by the unpaired t-test using GraphPad Prism software (GraphPad Software Inc.). A calculated P value of <0.05 was considered statistically significant.

### Data Analysis and Results

### 1. Direct Cell Cytotoxicity of AB-101

### A. Cytotoxicity of AB-101 Against K562 Cells

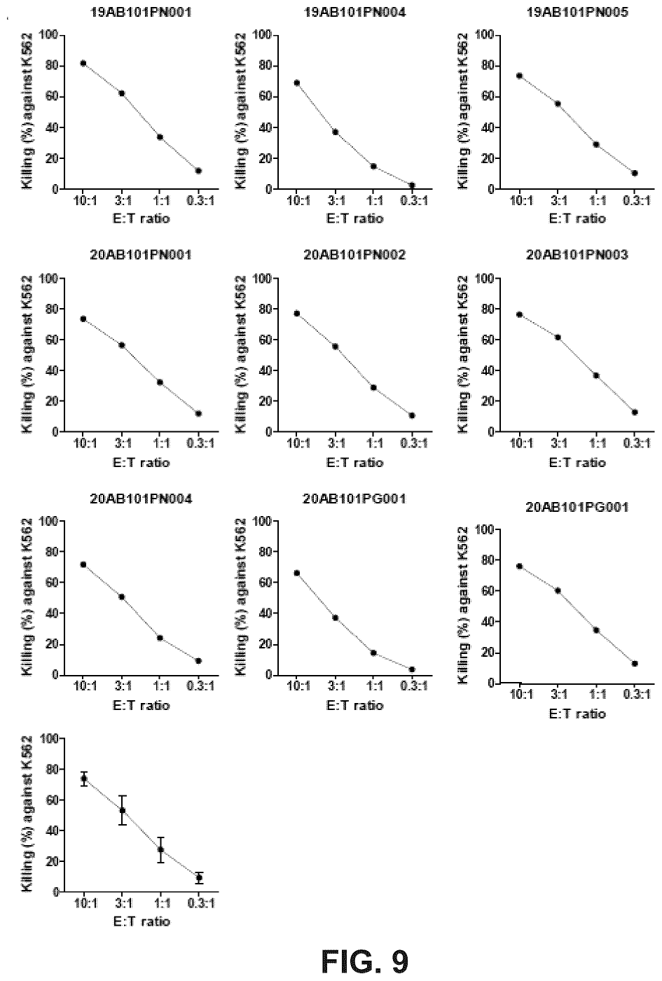

The direct cell cytotoxicity of AB-101 was measured at different E:T ratios from 10:1 to 0.3:1 against K562, an erythroleukemic cell line (FIG. 9, Table 21 and Table 22). K562 cell line is known as a NK-sensitive target due to lack of MHC class I antigens [16]. The direct cell cytotoxicity of AB-101 against K562 was E:T ratio-dependent. The results from testing 9 batches (7 Eng. and 2 GMP batches) showed that the cytotoxicity of AB-101 against K562 was 73.9±4.6% (Mean±SD) at E:T ratio of 10:1, 53.0±9.7% at E:T ratio of 3:1, 27.6±8.3% at E:T ratio of 1:1 and 9.5±3.9% at E:T ratio of 0.3:1. At 10:1 E:T ratio, the cytotoxicity of 9 batches was in the range of 66.3% (min) to 81.7% (max) (Table 22). The deviation among the batches (at all E:T ratios) was from 3.9% to 9.7% (Table 21, Table 22).

TABLE 21
Summary of direct cytotoxicity of AB-101 against tumor cells

| Specific | K562 cells | | Ramos cells | | Raji cells | |
|---|---|---|---|---|---|---|
| lysis (%) | Mean | SD | Mean | SD | Mean | SD |
| E:T = 10:1 | 73.9 | 4.6 | 57.1 | 8.0 | 77.0 | 2.8 |
| E:T = 3:1 | 53.0 | 9.7 | 41.1 | 6.5 | 67.3 | 5.9 |
| E:T = 1:1 | 27.6 | 8.3 | 22.4 | 7.7 | 45.1 | 7.4 |
| E:T = 0.3:1 | 9.5 | 3.9 | 7.1 | 6.3 | 15.0 | 4.9 |

TABLE 22
In vitro cytotoxicity results (Raw data): Target K562

| | | 19A | 19A | 19A | 20A | 20A | 20A | 20A | 20A | 20A | | |
|---|---|---|---|---|---|---|---|---|---|---|---|---|
| | | B10 | B10 | B10 | B10 | B10 | B10 | B10 | B10 | B10 | | |
| | E:T | 1PN | 1PN | 1PN | 1PN | 1PN | 1PN | 1PN | 1PG | 1PG | | |
| Target | ratio | 001 | 004 | 005 | 001 | 002 | 003 | 004 | 001 | 002 | AVE | SD |
| K562 | E10 | 81.7 | 69.0 | 73.6 | 73.5 | 77.0 | 76.3 | 71.8 | 66.3 | 76.1 | 73.9 | 4.6 |
| | E3 | 62.2 | 36.9 | 55.4 | 56.5 | 55.6 | 61.6 | 50.8 | 37.3 | 60.3 | 53.0 | 9.7 |
| | E1 | 33.6 | 14.7 | 28.9 | 32.2 | 28.8 | 36.8 | 24.1 | 14.3 | 34.7 | 27.6 | 8.3 |
| | E0.3 | 11.8 | 2.5 | 10.3 | 11.9 | 10.7 | 12.8 | 9.2 | 3.6 | 12.8 | 9.5 | 3.9 |

### B. Cytotoxicity of AB-101 Against Ramos

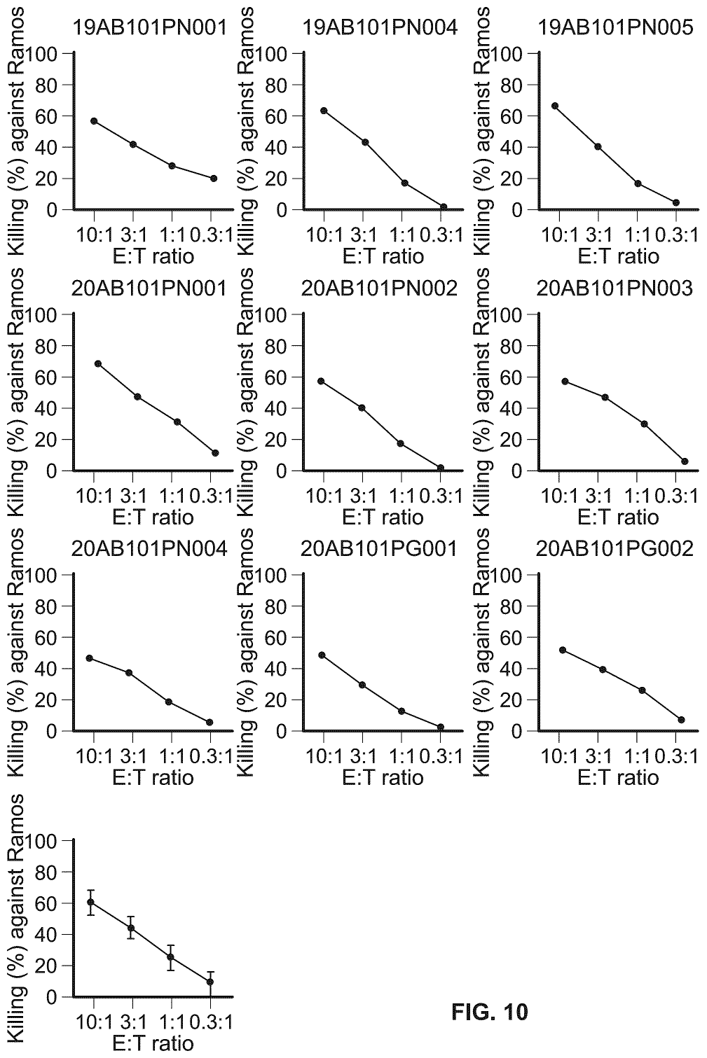

The direct cell cytotoxicity of AB-101 was measured at different E:T ratios from 10:1 to 0.3:1 against Ramos, Burkitt's lymphoma derived B lymphocyte cell line (FIG. 10, Table 21 and Table 23). The direct cell cytotoxicity of AB-101 against Ramos cells was E:T ratiodependent. The results from testing 9 batches (7 Eng. and 2 GMP batches) showed that the cytotoxicity of AB-101 against Ramos was 57.1±8.0 (Mean±SD) % at E:T ratio of 10:1, 41.1±6.5% at E:T ratio of 3:1, 22.4±7.7% at E:T ratio of 1:1 and 7.1±6.3% at E:T ratio of 0.3:1 (FIG. 10, Table 21 and Table 23). At 10:1 E:T ratio, the cytotoxicity was 46.1% (min) to 68.0% (max) (Table 23). The deviation among the batches (at all E:T ratios) was from 6.3% to 8.0% (Table 21, Table 23).

TABLE 23
In vitro cytotoxicity Results (Raw data): Target Ramos

| | | 19A | 19A | 19A | 20A | 20A | 20A | 20A | 20A | 20A | | |
|---|---|---|---|---|---|---|---|---|---|---|---|---|
| | | B10 | B10 | B10 | B10 | B10 | B10 | B10 | B10 | B10 | | |
| | E:T | 1PN | 1PN | 1PN | 1PN | 1PN | 1PN | 1PN | 1PG | 1PG | | |
| Target | ratio | 001 | 004 | 005 | 001 | 002 | 003 | 004 | 001 | 002 | AVE | SD |
| Ramos | E10 | 56.5 | 63.1 | 65.9 | 68.0 | 55.0 | 61.6 | 46.1 | 47.4 | 50.5 | 57.1 | 8.0 |
| | E3 | 41.5 | 43.6 | 42.9 | 47.5 | 37.9 | 51.2 | 37.1 | 28.7 | 39.4 | 41.1 | 6.5 |
| | E1 | 27.9 | 17.5 | 18.7 | 31.3 | 15.1 | 34.3 | 18.8 | 12.0 | 26.1 | 22.4 | 7.7 |
| | E0.3 | 20.0 | 1.8 | 5.5 | 11.6 | 0.0 | 10.9 | 4.9 | 1.6 | 7.2 | 7.1 | 6.3 |

### C. Cytotoxicity of AB-101 Against Raji

The direct cell cytotoxicity of AB-101 was measured at different E:T ratios from 10:1 to 0.3:1 against Raji, Burkitt's lymphoma derived B lymphocyte cell line (FIG. 6, Table 1 and Appendix 3). The direct cell cytotoxicity of AB-101 against Raji cells was E:T ratio-dependent. The results from testing 9 batches (7 Eng. and 2 GMP batches) showed that the cytotoxicity of AB-101 against Raji cells was 77.0+2.8 (Mean +SD)% at E:T ratio of 10:1, 67.3+5.9% at E:T ratio of 3:1, 45.4+7.4% at E:T ratio of 1:1 and 15.0+4.9% at E:T ratio of 0.3:1. Table 21 and Table 24). At 10:1 E:T ratio, the cytotoxicity was 73.4% (min) to 83.2% (max) (Table 24). The deviation among the batches (at all E:T ratios) was from 2.8% to 7.4% (Table 21, Table 24).

TABLE 24
In vitro cytotoxicity results (Raw data): Target Raji

| | | 19A | 19A | 19A | 20A | 20A | 20A | 20A | 20A | 20A | | |
|---|---|---|---|---|---|---|---|---|---|---|---|---|
| | | B10 | B10 | B10 | B10 | B10 | B10 | B10 | B10 | B10 | | |
| | E:T | 1PN | 1PN | 1PN | 1PN | 1PN | 1PN | 1PN | 1PG | 1PG | | |
| Target | ratio | 001 | 004 | 005 | 001 | 002 | 003 | 004 | 001 | 002 | AVE | SD |
| Raji | E10 | 75.9 | 78.7 | 83.2 | 78.2 | 76.4 | 75.7 | 75.9 | 73.4 | 75.5 | 77.0 | 2.8 |
| | E3 | 68.0 | 70.4 | 74.0 | 70.1 | 64.5 | 68.0 | 62.6 | 55 | 72.9 | 67.3 | 5.9 |
| | E1 | 45.4 | 47.1 | 50.6 | 52.1 | 41.3 | 43.8 | 37.6 | 32.1 | 55.8 | 45.1 | 7.4 |
| | E0.3 | 17.7 | 14.4 | 17.4 | 18.3 | 10.7 | 16.5 | 11.5 | 6.1 | 22.5 | 15.0 | 4.9 |

### 2. Antibody Dependent Cellular Cytotoxicity (ADCC) of AB-101

### A. Long-Term ADCC of AB-101 and Rituximab Combination Against Ramos Cells

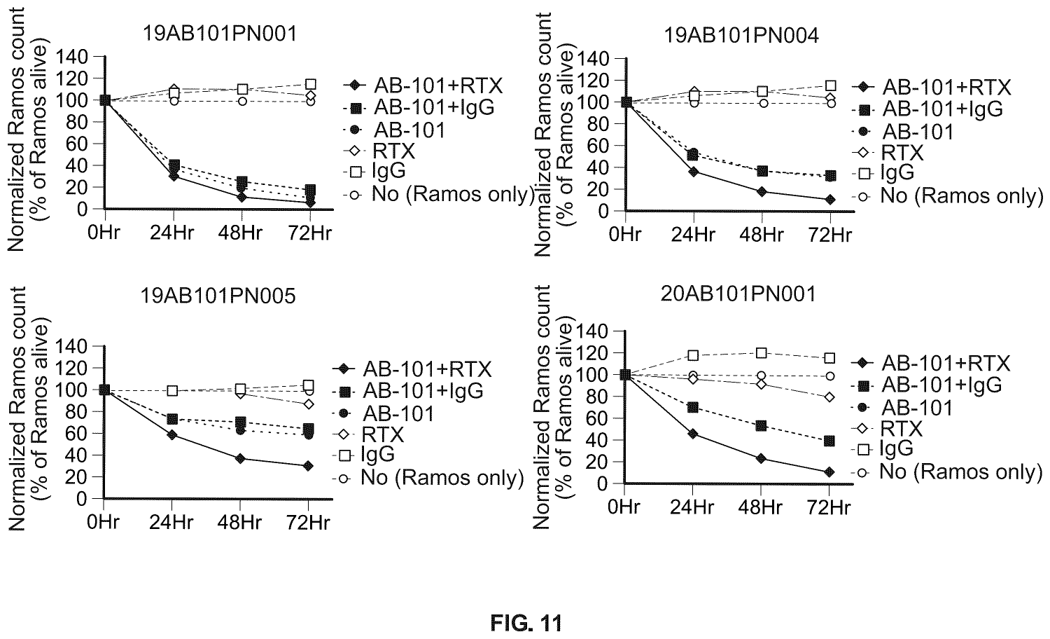

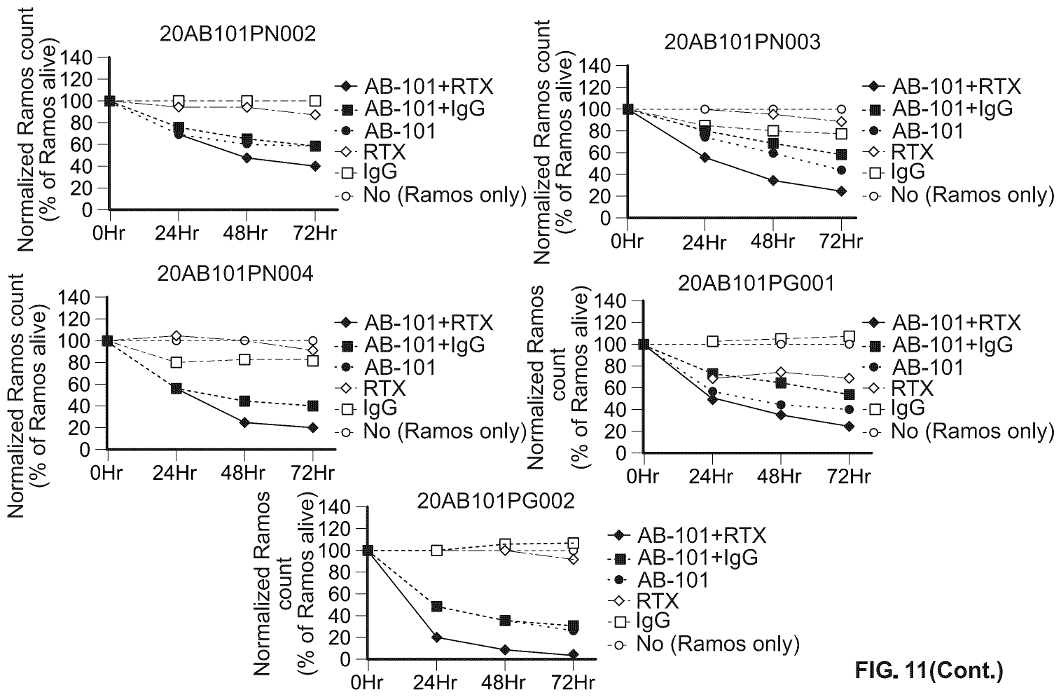

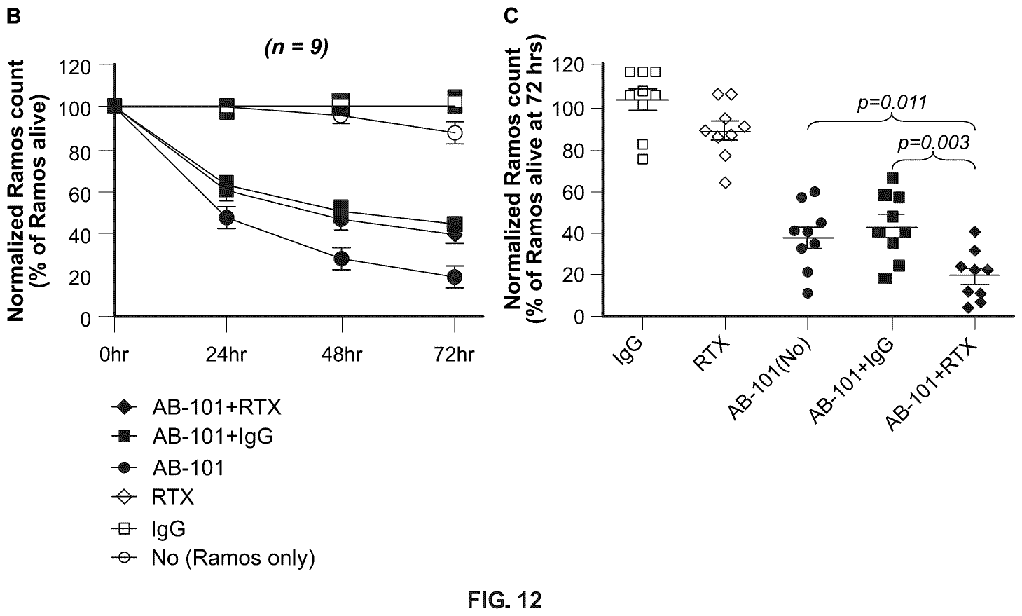

The ADCC of AB-101 in combination with rituximab was tested against Ramos tumor cell line using IncuCyte. Real-time images of tumor cells were obtained for 72 hrs during their co-culture with AB-101 in the presence or absence of RTX. As described in materials and methods, longterm ADCC of AB-101 in the presence or absence of RTX was determined by calculating % of live Ramos cells in the culture at any given time during culture period. To determine long-term ADCC of AB-101, total 6 conditions were tested 1) Ramos only, 2) Human IgG (hIgG), 3) Rituximab (RTX), 4) AB-101 alone, 5) AB-101+IgG, and 6) AB-101+Rituximab (RTX). In the AB-101 alone and AB-101+RTX culture conditions, the results showed that the % of live Ramos cells in the culture continuously decreased over time, and the lysis of target cell was observed up to 72 hours (FIG. 11, FIG. 12, left).

At 24 hours culture period, the % live Ramos cells in the AB-101+RTX condition was 47.9±15.5%, which is suggestive of lysis of more than 50% of target tumor cells that went into culture at 0 hr timepoint. On the other hand, the % live Ramos cells in the AB-101 alone and AB-101+hIgG culture conditions was more than 60%. The % live Ramos cells (%) at 72 hours was 37.6±15.4%, 42.5±15.9% and 19.0±11.9% (mean±SD) for AB-101 alone, AB-101+hIgG and AB-101+RTX culture conditions respectively (FIG. 12 right, Table 25). At 72 hours, the % live Ramos cells in culture conditions AB-101 alone, AB-101+IgG and AB-101+RTX was in the range of 11%-58.9%, 18.3%-65.9% and 4.1%-40.3% respectively.

The deviation among different batches for different culture conditions was in the range of 12.5%-16.3% (Table 25, Table 26). This data shows that AB-101 in combination with rituximab demonstrates significant increase in ADCC against Ramos cells at 72 hrs when compared to AB-101 alone (p=0.011) and AB-101+hIgG (p=0.003) (FIG. 12 right).

TABLE 25
Summary of long-term ADCC of AB-101 in combination
with rituximab against Ramos cells

| Viable | | | | | | |
|---|---|---|---|---|---|---|
| Ramos | AB-101 | | AB-101 + hIgG | | AB-101 + RTX | |
| cells (%) | Mean | SD | Mean | SD | Mean | SD |
| 0 hr | 100.0 | 0.0 | 100.0 | 0.0 | 100.0 | 0.0 |
| 24 hrs | 60.0 | 12.5 | 62.5 | 14.1 | 47.6 | 15.5 |
| 48 hrs | 45.8 | 14.7 | 50.5 | 16.3 | 28.1 | 14.3 |
| 72 hrs | 37.6 | 15.4 | 42.5 | 15.9 | 19.0 | 11.96 |

TABLE 26
In vitro long-term ADCC results (Raw data): Target Ramos, % of Ramos alive

| | | 19A | 19A | 19A | 20A | 20A | 20A | 20A | 20A | 20A | | |
|---|---|---|---|---|---|---|---|---|---|---|---|---|
| | | B101 | B101 | B101 | B101 | B101 | B101 | B101 | B101 | B101 | | |
| | | PN0 | PN0 | PN0 | PN0 | PN0 | PN0 | PN0 | PG0 | PG0 | | |
| Treatment | Time | 01 | 04 | 05 | 01 | 02 | 03 | 04 | 01 | 02 | AVE | SD |
| AB-101 + | 0 h | 100.0 | 100.0 | 100.0 | 100.0 | 100.0 | 100.0 | 100.0 | 100.0 | 100.0 | 100.0 | 0.0 |
| RTX | 24 h | 29.5 | 37.1 | 59.7 | 46.2 | 69.7 | 57.7 | 54.8 | 54.1 | 22.1 | 47.9 | 15.5 |
| | 48 h | 12.1 | 18.6 | 38.1 | 22.8 | 53.0 | 34.4 | 29.2 | 37.4 | 7.7 | 28.1 | 14.3 |
| | 72 h | 6.6 | 11.4 | 30.9 | 11.0 | 40.3 | 21.8 | 21.3 | 23.5 | 4.1 | 19.0 | 11.9 |
| AB-101 + | 0 h | 100.0 | 100.0 | 100.0 | 100.0 | 100.0 | 100.0 | 100.0 | 100.0 | 100.0 | 100.0 | 0.0 |
| hIgG | 24 h | 40.9 | 51.1 | 74.0 | 68.9 | 74.6 | 77.8 | 54.9 | 73.9 | 46.6 | 62.5 | 14.1 |
| | 48 h | 26.1 | 37.8 | 70.8 | 53.3 | 66.6 | 64.4 | 44.0 | 59.8 | 31.3 | 50.5 | 16.3 |
| | 72 h | 18.3 | 33.7 | 65.9 | 39.8 | 57.7 | 56.0 | 39.5 | 47.7 | 23.8 | 42.5 | 15.9 |
| AB-101 | 0 h | 100.0 | 100.0 | 100.0 | 100.0 | 100.0 | 100.0 | 100.0 | 100.0 | 100.0 | 100.0 | 0.0 |
| | 24 h | 36.4 | 54.8 | 74.5 | 71.5 | 68.6 | 70.9 | 55.2 | 58.9 | 49.5 | 60.0 | 12.5 |
| | 48 h | 19.8 | 36.5 | 63.5 | 53.4 | 62.4 | 55.4 | 44.4 | 45.8 | 30.9 | 45.8 | 14.7 |
| | 72 h | 11.0 | 31.9 | 58.9 | 40.5 | 56.5 | 44.1 | 40.2 | 34.2 | 21.1 | 37.6 | 15.4 |
| Rituximab | 0 h | 100.0 | 100.0 | 100.0 | 100.0 | 100.0 | 100.0 | 100.0 | 100.0 | 100.0 | 100.0 | 0.0 |
| (RTX) | 24 h | 110.7 | 110.7 | 98.5 | 97.3 | 97.4 | 100.1 | 104.1 | 71.1 | 100.7 | 98.9 | 11.7 |
| | 48 h | 109.6 | 109.6 | 97.3 | 90.3 | 95.7 | 93.0 | 99.2 | 69.6 | 98.8 | 95.9 | 12.0 |
| | 72 h | 105.3 | 105.3 | 88.2 | 76.5 | 85.0 | 86.2 | 90.1 | 63.5 | 93.6 | 88.2 | 13.1 |
| Human | 0 h | 100.0 | 100.0 | 100.0 | 100.0 | 100.0 | 100.0 | 100.0 | 100.0 | 100.0 | 100.0 | 0.0 |
| IgG | 24 h | 106.5 | 106.5 | 100.1 | 118.2 | 99.6 | 81.5 | 90.4 | 101.8 | 100.6 | 99.5 | 12.0 |
| (hIgG) | 48 h | 111.0 | 111.1 | 102.5 | 120.8 | 100.9 | 77.6 | 81.5 | 103.0 | 105.0 | 101.5 | 13.9 |
| | 72 h | 116.6 | 116.6 | 105.0 | 115.9 | 101.1 | 74.4 | 81.9 | 104.7 | 107.4 | 102.6 | 15.1 |
| No | 0 h | 100.0 | 100.0 | 100.0 | 100.0 | 100.0 | 100.0 | 100.0 | 100.0 | 100.0 | 100.0 | 0.0 |
| (Ramos | 24 h | 100.0 | 100.0 | 100.0 | 100.0 | 100.0 | 100.0 | 100.0 | 100.0 | 100.0 | 100.0 | 0.0 |
| only) | 48 h | 100.0 | 100.0 | 100.0 | 100.0 | 100.0 | 100.0 | 100.0 | 100.0 | 100.0 | 100.0 | 0.0 |
| | 72 h | 100.0 | 100.0 | 100.0 | 100.0 | 100.0 | 100.0 | 100.0 | 100.0 | 100.0 | 100.0 | 0.0 |

### B. Long-Term ADCC of AB-101 and Rituximab Combination Against Raji

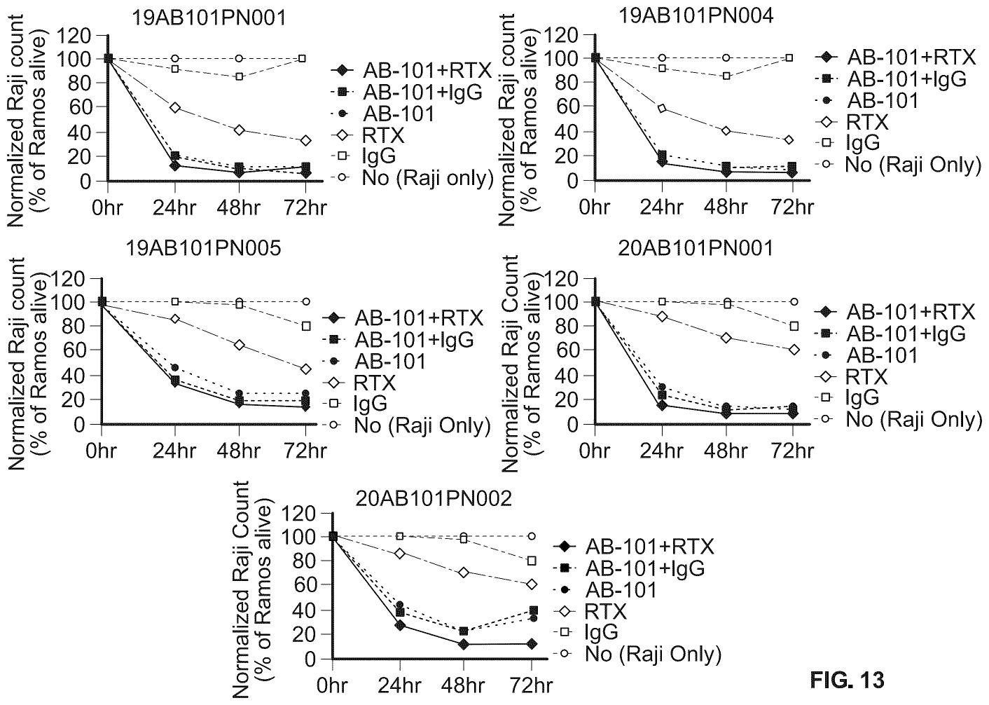

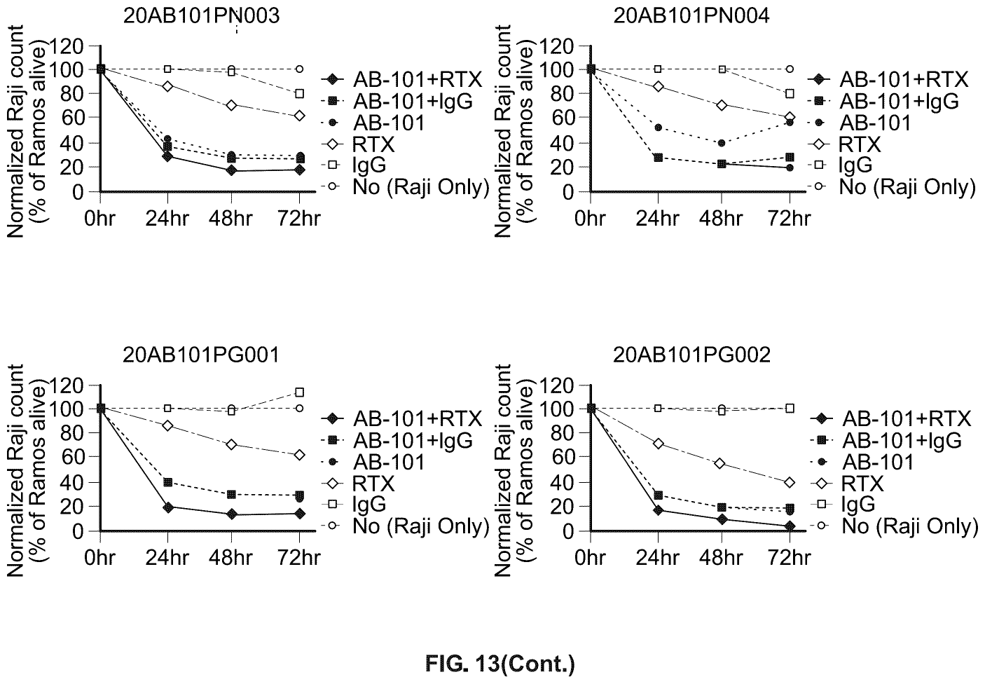

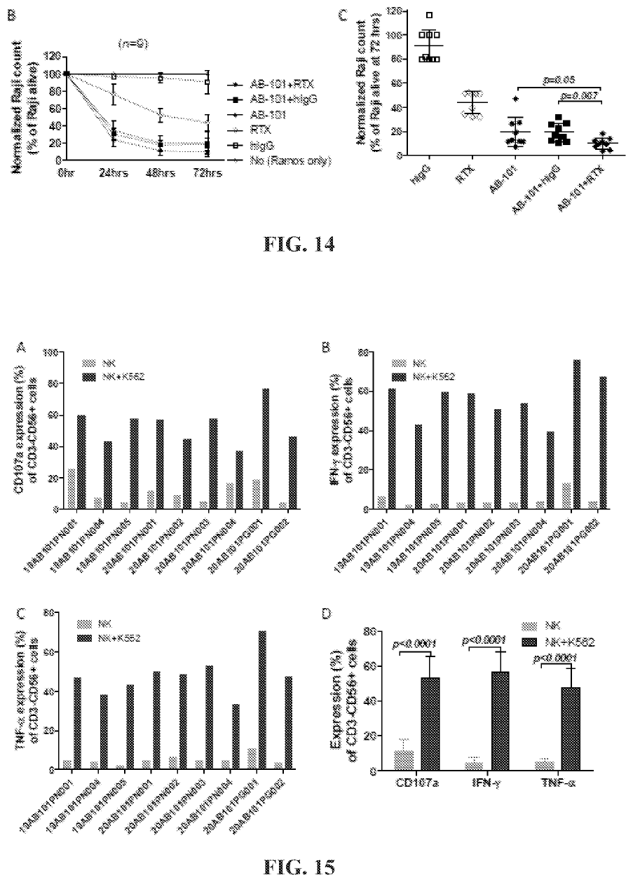

The ADCC of AB-101 in combination with rituximab was tested against Raji tumor cell line using IncuCyte. The test methods and conditions were identical to the long-term ADCC assay of Ramos described above. To determine long-term ADCC of AB-101 against Raji cells, total 6 conditions were tested 1) Raji only, 2) Human IgG (hIgG), 3) Rituximab (RTX), 4) AB-101 alone, 5) AB-101+IgG, and 6) AB-101+Rituximab (RTX). In the AB-101 alone and AB-101+RTX, the results showed that the % of live Raji cells in the culture continuously decreased over time, and the lysis of target cell was observed up to 72 hours (FIG. 13). The % live Raji cells indicative of the long-term ADCC at 72 hours in culture conditions AB-101 alone, AB-101+hIgG and AB-101+RTX was 20.5±12.2%, 19.2±7.6% and 10.1±4.6% (mean±SD) respectively (FIG. 14 left, Table 27). At 72 hours, the % live Raji cells in culture conditions AB-101 alone, AB-101+IgG and AB-101+RTX were in the range of 7%-47%, 10.5%-31.8% and 3.6%-18.3% respectively. The deviation among different batches for different culture conditions was in the range of 4.6%-12.2% (Table 27, Table 28). This data shows that AB-101 in combination with rituximab demonstrates significant increase in ADCC against Raji cells at 72 hrs when compared to AB-101 alone (p=0.05) and AB-101+hIgG (p=0.007) (FIG. 14 right).

TABLE 27
Summary of long-term ADCC of AB-101 in combination
with rituximab against Raji cells

| Viable | | | | | | |
|---|---|---|---|---|---|---|
| Raji cells | AB-101 | | AB-101 + hIgG | | AB-101 + RTX | |
| (%) | Mean | SD | Mean | SD | Mean | SD |
| 0 hr | 100.0 | 0.0 | 100.0 | 0.0 | 100.0 | 0.0 |
| 24 hrs | 35.2 | 10.6 | 30.9 | 7.0 | 23.9 | 7.9 |
| 48 hrs | 20.1 | 9.1 | 18.0 | 5.5 | 11.7 | 4.7 |
| 72 hrs | 20.5 | 12.2 | 19.2 | 7.6 | 10.1 | 4.6 |

TABLE 28
In vitro long-term ADCC results (Raw data): Target Raji, % of Raji alive

| | | 19A | 19A | 19A | 20A | 20A | 20A | 20A | 20A | 20A | | |
|---|---|---|---|---|---|---|---|---|---|---|---|---|
| | | B101 | B101 | B101 | B101 | B101 | B101 | B101 | B101 | B101 | | |
| | | PN0 | PN0 | PN0 | PN0 | PN0 | PN0 | PN0 | PG0 | PG0 | | |
| Treatment | Time | 01 | 04 | 05 | 01 | 02 | 03 | 04 | 01 | 02 | AVE | SD |
| AB-101 + | 0 h | 100.0 | 100.0 | 100.0 | 100.0 | 100.0 | 100.0 | 100.0 | 100.0 | 100.0 | 100.0 | 0.0 |
| RTX | 24 h | 13.8 | 21.1 | 34.1 | 16.8 | 26.6 | 28.4 | 34.3 | 25.8 | 14.3 | 23.9 | 7.9 |
| | 48 h | 6.6 | 8.9 | 18.6 | 9.2 | 12.5 | 13.3 | 18.6 | 11.7 | 5.5 | 11.7 | 4.7 |
| | 72 h | 4.5 | 9.4 | 11.5 | 7.5 | 13.9 | 12.3 | 18.3 | 9.6 | 3.6 | 10.1 | 4.6 |
| AB-101 + | 0 h | 100.0 | 100.0 | 100.0 | 100.0 | 100.0 | 100.0 | 100.0 | 100.0 | 100.0 | 100.0 | 0.0 |
| hIgG | 24 h | 21.2 | 27.4 | 36.9 | 23.1 | 36.9 | 35.2 | 34.4 | 39.5 | 23.6 | 30.9 | 7.0 |
| | 48 h | 12.0 | 12.7 | 21.3 | 11.1 | 23.1 | 23.3 | 21.8 | 23.6 | 13.4 | 18.0 | 5.5 |
| | 72 h | 11.9 | 16.1 | 15.4 | 10.5 | 31.8 | 25.7 | 26.5 | 22.4 | 12.3 | 19.2 | 7.6 |
| AB-101 | 0 h | 100.0 | 100.0 | 100.0 | 100.0 | 100.0 | 100.0 | 100.0 | 100.0 | 100.0 | 100.0 | 0.0 |
| | 24 h | 19.6 | 28.5 | 45.6 | 29.6 | 42.3 | 40.7 | 49.7 | 38.5 | 22.0 | 35.2 | 10.6 |
| | 48 h | 9.1 | 12.2 | 25.5 | 12.9 | 22.0 | 27.1 | 37.1 | 22.5 | 12.4 | 20.1 | 9.1 |
| | 72 h | 7.0 | 11.6 | 21.0 | 13.0 | 26.5 | 27.3 | 47.0 | 19.3 | 11.8 | 20.5 | 12.2 |
| Rituximab | 0 h | 100.0 | 100.0 | 100.0 | 100.0 | 100.0 | 100.0 | 100.0 | 100.0 | 100.0 | 100.0 | 0.0 |
| (RTX) | 24 h | 57.2 | 57.2 | 86.2 | 83.1 | 83.1 | 83.1 | 83.1 | 84.6 | 69.5 | 76.3 | 11.9 |
| | 48 h | 39.3 | 39.3 | 53.6 | 57.0 | 57.0 | 57.0 | 57.0 | 59.3 | 54.5 | 52.6 | 7.8 |
| | 72 h | 31.9 | 31.9 | 39.6 | 51.3 | 51.3 | 51.3 | 51.3 | 52.6 | 34.6 | 44.0 | 9.3 |
| Human | 0 h | 100.0 | 100.0 | 100.0 | 100.0 | 100.0 | 100.0 | 100.0 | 100.0 | 100.0 | 100.0 | 0.0 |
| IgG | 24 h | 90.9 | 90.9 | 99.8 | 98.1 | 98.1 | 98.1 | 98.1 | 98.8 | 98.9 | 96.8 | 3.4 |
| (hIgG) | 48 h | 85.6 | 856 | 96.4 | 98.3 | 98.3 | 98.3 | 98.3 | 97.0 | 98.1 | 95.1 | 5.5 |
| | 72 h | 99.8 | 99.8 | 82.2 | 798 | 79.8 | 79.8 | 79.8 | 116.4 | 100.1 | 90.8 | 13.5 |
| No | 0 h | 100.0 | 100.0 | 100.0 | 100.0 | 100.0 | 100.0 | 100.0 | 100.0 | 100.0 | 100.0 | 0.0 |
| (Raji only) | 24 h | 100.0 | 100.0 | 100.0 | 100.0 | 100.0 | 100.0 | 100.0 | 100.0 | 100.0 | 100.0 | 0.0 |
| | 48 h | 100.0 | 100.0 | 100.0 | 100.0 | 100.0 | 100.0 | 100.0 | 100.0 | 100.0 | 100.0 | 0.0 |
| | 72 h | 100.0 | 100.0 | 100.0 | 100.0 | 100.0 | 100.0 | 100.0 | 100.0 | 100.0 | 100.0 | 0.0 |

### 3. Cytokine Production and Degranulation Marker (CD107a) Expression of AB-101 Against Tumor Cells

### A. Intracellular Cytokine Staining (ICS) of AB-101 Against K562

After co-culture of AB-101 and K562 cells at E:T=1:1 for 4 hours, the effector cytokines TNF-α and IFN-γ) produced from the NK cells and the expression of degranulation marker (CD107a) were measured by flow cytometer. The results from testing 9 batches (7 Eng. And 2 GMP batches) showed that the percent CD107a+, IFN-γ+ and TNFα+AB-101 cells were 11.1±7.3% (Mean±SD), 4.6±3.4% and 4.9±2.4% respectively in AB-101 alone culture condition. On the other hand, the percent CD107a+, IFN-γ+ and TNFα+AB-101 cells were 53.0±12.0%, 56.5±11.5% and 47.8±10.4% in AB-101 plus K562 co-culture condition (FIG. 15, Table 29). The range of percent CD107a+, IFN-γ+ and TNFα+AB-101 cells in AB-101 alone culture condition was 4%-25%, 1.7%-13;% and 2.3%-0.7% respectively and the range of percent CD107a+, IFN-γ+ and TNFα+ AB-101 cells in AB-101 plus K562 coculture condition was 36.7%-76.7%, 39.19 475.93 and 33.2%-70.4% respectively (Table 30, Table 31, Table 32). The deviation between the batches was <10% and <15% in AB-101 alone and AB-101 plus K562 culture conditions respectively (Table 29, Table 30, Table 31, Table 32). This data shows that co-culturing of AB-101 with K562 resulted in significant increase in the production of effector cytokines such as IFN-γ (p<0.0001), TNF-α (p<0.0001) and expression of marker of degranulation CD107a (p<0.0001) when compared to the control, AB-101 culture alone (FIG. 15). These results confirm the activity of AB-101 against tumor cells.

TABLE 29
Summary of ICS data of AB-101 against tumor cells

| | AB-101 | | K562 | | Ramos | | Raji | |
|---|---|---|---|---|---|---|---|---|
| Expression | (No target) | | cells | | cells | | cells | |
| (%) | Mean | SD | Mean | SD | Mean | SD | Mean | SD |
| CD107a | 11.1 | 7.3 | 53.0 | 12.0 | 40.7 | 154 | 60.9 | 17.4 |
| IFN-γ | 4.6 | 3.4 | 56.5 | 11.5 | 35.7 | 9.0 | 57.3 | 10.7 |
| TNF-α | 4.9 | 2.4 | 47.8 | 10.4 | 30.1 | 8.4 | 50.7 | 14.4 |

TABLE 30
CD107a (%) of CD56+: Raw data

| | 19AB | 19AB | 19AB | 20AB | 20AB | 20AB | 20AB | 20AB | 20AB | | |
|---|---|---|---|---|---|---|---|---|---|---|---|
| | 101P | 101P | 101P | 101P | 101P | 101P | 101P | 101P | 101P | | |
| Group | N001 | N004 | N005 | N001 | N002 | N003 | N004 | G001 | G002 | AVE | SD |
| AB-101 | 25.0 | 6.9 | 4.3 | 11.4 | 8.8 | 5.1 | 16.1 | 18.2 | 4.0 | 11.1 | 7.3 |
| only | | | | | | | | | | | |
| K562 | 59.7 | 42.8 | 57.4 | 56.3 | 44.6 | 57.2 | 36.7 | 76.7 | 45.6 | 53.0 | 12.0 |
| Ramos | 56.2 | 48.4 | 39.2 | 67.5 | 34.0 | 32.7 | 33.4 | 68.9 | 156 | 40.7 | 15.4 |
| Raji | 69.2 | 62.4 | N.A. | 66.3 | 73.0 | 62.8 | 55.3 | 76.9 | 21.0 | 60.9 | 17.4 |

TABLE 31
IFN-γ (%) of CD56+: Raw data

| | 19AB | 19AB | 19AB | 20AB | 20AB | 20AB | 20AB | 20AB | 20AB | | |
|---|---|---|---|---|---|---|---|---|---|---|---|
| | 101P | 101P | 101P | 101P | 101P | 101P | 101P | 101P | 101P | | |
| Group | N001 | N004 | N005 | N001 | N002 | N003 | N004 | G001 | G002 | AVE | SD |
| AB-101 | 6.2 | 1.7 | 2.6 | 3.3 | 3.1 | 3.2 | 3.8 | 13.0 | 4.1 | 4.6 | 3.4 |
| only | | | | | | | | | | | |
| K562 | 61.4 | 42.7 | 59.4 | 58.5 | 50.9 | 53.7 | 39.1 | 75.9 | 67.3 | 56.5 | 11.5 |
| Ramos | 43.3 | 33.7 | 32.2 | 32.6 | 31.4 | 27.9 | 27.2 | 55.9 | 37.0 | 35.7 | 9.0 |
| Raji | 62.5 | 46.5 | N.A. | 60.3 | 63.8 | 63.8 | 62.2 | 63.9 | 35.0 | 57.3 | 10.7 |

TABLE 32
TNF-α (%) of CD56+: Raw data

| | 19AB | 19AB | 19AB | 20AB | 20AB | 20AB | 20AB | 20AB | 20AB | | |
|---|---|---|---|---|---|---|---|---|---|---|---|
| | 101P | 101P | 101P | 101P | 101P | 101P | 101P | 101P | 101P | | |
| Group | N001 | N004 | N005 | N001 | N002 | N003 | N004 | G001 | G002 | AVE | SD |
| AB-101 | 4.3 | 4.2 | 2.3 | 4.4 | 6.1 | 4.5 | 4.6 | 10.7 | 3.2 | 4.9 | 2.4 |
| only | | | | | | | | | | | |
| K562 | 46.7 | 38.2 | 43.2 | 49.9 | 48.4 | 53.0 | 33.2 | 70.1 | 47.3 | 47.8 | 10.4 |
| Ramos | 31.2 | 29.3 | 22.0 | 30.9 | 36.2 | 25.1 | 23.7 | 49.0 | 23.8 | 30.1 | 8.4 |
| Raji | 55.1 | 37.5 | N.A. | 52.7 | 67.2 | 58.9 | 53.2 | 59.0 | 21.9 | 50.7 | 14.4 |

### B. Intracellular Cytokine Staining (CCS) of AB-101 Against Ramos

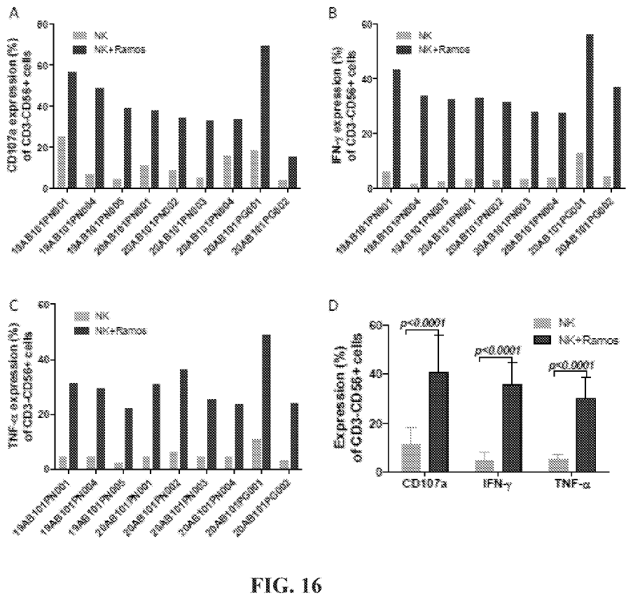

After co-culture of AB-101 and Ramos cells at E:T=1:1 for 4 hours, the effector cytokines (TNF-α and IFN-γ) produced from the NK cells and the expression of degranulation marker (CD107a) were measured by flow cytometer. The results from testing 9 batches (7 Eng. And 2 GMP batches) showed that the percent CD107a+, IFN-γ+ and TNFα+ AB-101 cells were 40.7±15.4%, 35.7±9.0% and 30.1±8.4% in AB-101 plus Ramos cells co-culture condition (FIG. 16, Table 29). The range of percent CD107a+, IFN-γ+ and TNFα+ AB-101 cells in in AB-101 plus Ramos cells co-culture condition was 15.6%-68.99%, 27.2%-55.9% and 22%-49% respectively (Table 30, Table 31, Table 32). The deviation between the batches was <20% in AB-101 plus Ramos cells co-culture condition (Table 29, Table 30, Table 31, Table 32). This data shows that co-culturing of AB-101 with Ramos resulted in significant increase in the production of effector cytokines such as IFN-γ (p<0.0001), TNF-α (p<0.0001) and expression of marker of degranulation CD107a (p<0.0001) when compared to the control, AB-101 culture alone (FIG. 16). These results confirm the activity of AB-101 against tumor cells.

### C. Intracellular Cytokine Staining (ICS) of AB-101 Against Raji

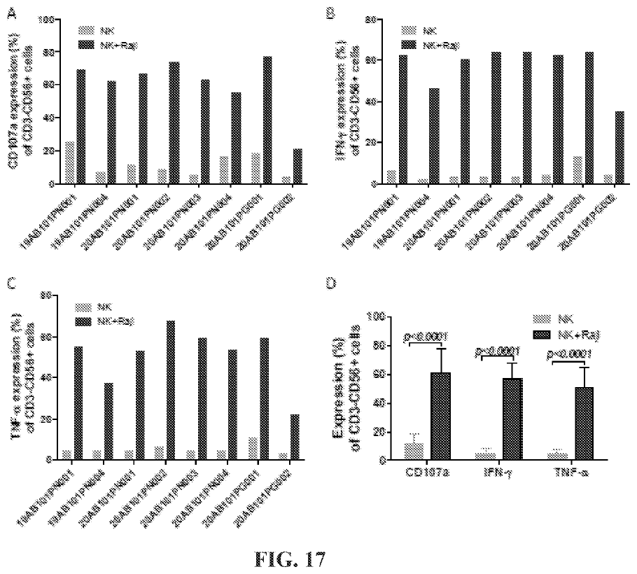

After co-culture of AB-101 and Raji cells at E:T=1:1 for 4 hours, the effector cytokines (TNF-α and IFN-γ) produced from the NK cells and the expression of degranulation marker (CD107a) were measured by flow cytometer. The results from testing 8 batches (6 Eng. And 2 GMP batches) showed that the percent CD107a+, IFN-γ+ and TNFα+AB-101 cells were 60.9±17.4% (Mean±SD), 57.3±10.7% and 50.7±14.4% in AB-101 plus Raji cells coculture condition (FIG. 17, Table 29). The range of percent CD107a+, IFN-γ+ and TNFα+AB-101 cells in in AB-101 plus Raji cells co-culture condition was 21.0%-76.9%, 35.0%-63.9% and 21.9%-67.2% respectively (Table 30, Table 31, Table 32). The deviation between the batches was <20% in AB-101 plus Raji cells co-culture condition (Table 29, Table 30, Table 31, Table 32). This data shows that co-culturing of AB-101 with Raji cells resulted in significant increase in the production of effector cytokines such as IFN-γ (p<0.0001), TNF-α (p<0.0001) and expression of marker of degranulation CD107a (p<0.0001) when compared to the control, AB-101 culture alone (FIG. 17). These results confirm the activity of AB-101 against tumor cells.

### Conclusions

Data demonstrated in this report supports effector functions of AB-101 alone and in combination with rituximab. Direct cytotoxicity of AB-101 on tumor cells was evaluated using short-term (4 hr) effector and target cell co-culture assays. Data obtained from these studies showed that AB-101 can efficiently kill multiple tumor cell lines such as K562, Ramos, Raji and tumor-specific lytic activity of AB-101 increased with an increase in E:T ratio. At an E:T ratio of 1:10, as much as 50%-70% of lysis of target tumor cells was noted. ADCC of AB-101 against tumor cells in combination with rituximab was evaluated using long-term (72 hrs) co-culture assays. In these assays, it was demonstrated that AB-101 when used in combination with rituximab could result in the lysis of 80% to 90% of Ramos and Raji tumor cells. The cytolytic activity of AB-101 against tumor cells observed in combination with rituximab was approximately 2 times higher than the activity observed with AB-101 alone and in combination with hIgG. This data clearly suggests that rituximab enhanced antitumor activity of AB-101 by ADCC mechanism and supports the hypothesis that AB-101 in combination with rituximab can be an effective treatment strategy for CD20+ lymphoma patients. The ability of AB-101 cells to mediate anti-tumor immunity by cytokine secretion and expression of markers of degranulation was evaluated using intracellular cytokine staining assays. Data obtained from these studies suggest that AB-101 in response to tumor cell stimulation expresses ˜4 to 6 times higher CD107a, ˜7 to 10 higher IFN-γ and −6 to 10 times higher TNF-α when compared to unstimulated AB-101 cells suggestive of tumor antigen dependent effector functions of AB-101.

In conclusion, results of these in vitro pharmacology studies performed using nine AB-101 batches demonstrated that AB-101 could specifically kill tumor cells and effectively suppress the proliferation of them by direct cytotoxicity, antibody mediated cytotoxicity and by secretion of the effector cytokines.

### REFERENCES

4. Trapani J A, Davis J, Sutton V R, Smyth M J. Proapoptotic functions of cytotoxic lymphocyte granule constituents in vitro and in vivo. Current opinion in immunology. 2000; 12(3):323-9.
5. Kägi D, Ledermann B, Bürki K, Seiler P, Odermatt B, Olsen K J, et al. Cytotoxicity mediated by T cells and natural killer cells is greatly impaired in perforin-deficient mice. Nature. 1994; 369(6475):31.
6. Sutlu T, Alici E. Natural killer cell-based immunotherapy in cancer: current insights and future prospects. Journal of internal medicine. 2009; 266(2):154-81.
7. Cretney E, Takeda K, Yagita H, Glaccum M, Peschon J J, Smyth M J. Increased susceptibility to tumor initiation and metastasis in TNF-related apoptosis-inducing ligand-deficient mice. The Journal of Immunology. 2002; 168(3):1356-61.
8. Takeda K, Hayakawa Y, Smyth M J, Kayagaki N, Yamaguchi N, Kakuta S, et al. Involvement of tumor necrosis factor-related apoptosis-inducing ligand in surveillance of tumor metastasis by liver natural killer cells. Nature medicine. 2001; 7(1):94.
9. Kayagaki N, Yamaguchi N, Nakayama M, Takeda K, Akiba H, Tsutsui H, et al. Expression and function of TNF-related apoptosis-inducing ligand on murine activated NK cells. The Journal of Immunology. 1999; 163(4):1906-13.
10. Screpanti V, Wallin R P, Ljunggren H-G, Grandien A. A central role for death receptor mediated apoptosis in the rejection of tumors by NK cells. The Journal of Immunology. 2001; 167(4):2068-73.
11. Bradley M, Zeytun A, Rafl-Janajreh A, Nagarkatti P S, Nagarkatti M. Role of spontaneous and interleukin-2-induced natural killer cell activity in the cytotoxicity and rejection of Fas+

### Example 9: AB-101 In Vitro Pharmacology

The anti-tumor function of NK cells can be broadly categorized into three primary effector mechanisms: 1) Direct recognition and killing of tumor cells, 2) Killing of tumor cells by antibody-dependent cell-mediated cytotoxicity (ADCC), and 3) Regulation of immune responses through production of immunostimulatory cytokines and chemokines. The specific mechanism(s) of the effector function of AB-101 was assessed in a series of studies.

Direct cytotoxicity of AB-101 against tumor cell lines was assessed by fluorometric assay. Cytotoxicity of NK cells were quantitatively measured and assessed at a range of NK cell (effector) to tumor cell (target) ratios. Target cells included K562, an immortalized myelogenous leukemia cell line that is widely used in NK cell cytotoxicity assessments, and Ramos and Raji which are CD20+ lymphoma cell lines of B-cell origin.

Cytotoxicity of AB-101 against tumor cell lines was assessed by fluorometric assay. Cytotoxicity of NK cells can be quantitatively measured and assessed at a range of NK cell (effector) to tumor cell (target) ratios. Target cells included a) K562; an immortalized myelogenous leukemia cell line that is widely used in NK cell cytotoxicity assessments, and b) Raji and Ramos cells; CD20+ Lymphoma cell lines of B-cell origin.

Target cells were stained with 30 μM calcein-AM (Molecular probe, USA) for 1 h at 37° C. NK cells and labeled tumor target cells were co-cultured in 96-well plate in triplicate at 37° C. and 5% CO2 for 4 h with light-protection. RPMI1640 medium containing 10% FBS or 2% triton-X100 was added to the targets to provide spontaneous and maximum release. RPMI1640 medium containing 10% FBS or 2% triton-X100 was added to each well to determine background fluorescence. The measurement was conducted at excitation 485 nm and emission 535 nm with the fluorometer. The percentage of specific calcein AM release was calculated according to the formula: % specific release=[(mean experimental release−mean spontaneous release)/(mean maximal release−mean spontaneous release)]×100.

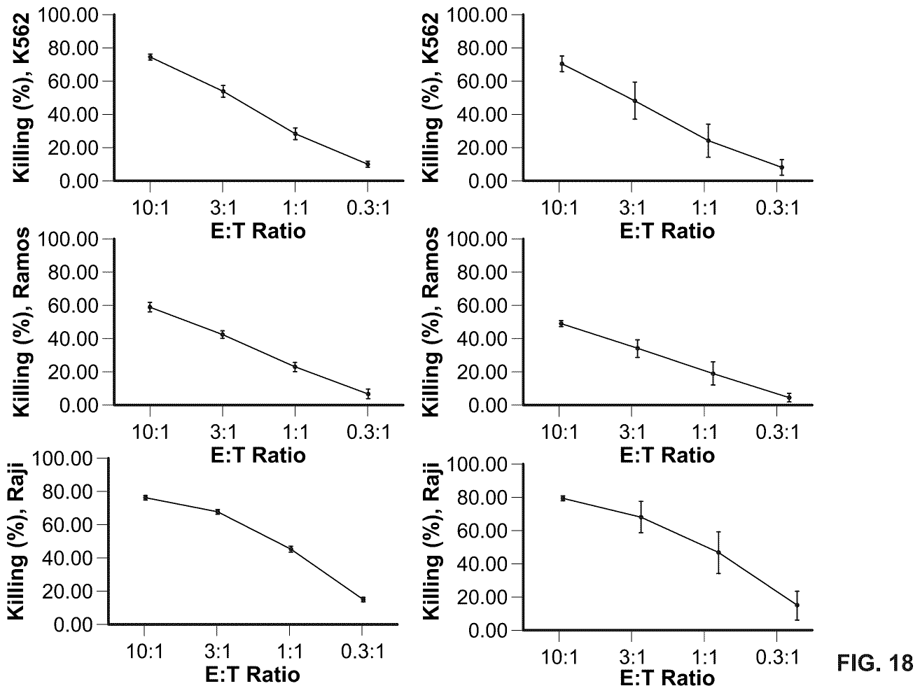

AB-101 demonstrated dose-dependent cytotoxic activity against K562, Ramos and Raji tumor cell lines (FIG. 18). Approximately 60% to 80% of lysis of target cells was observed at highest Effector: Target (E:T) cell ratio. These results indicate consistent cytotoxic activity for AB-101 and its potent cytocidal effect against cancer cells.

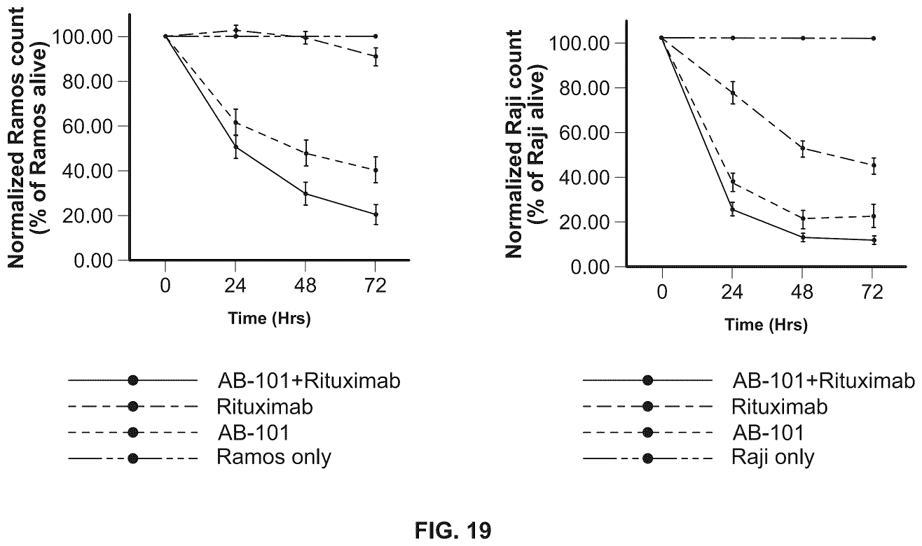

To determine whether AB-101 effects its anti-tumor activity through an ADCC mechanism, target cells were treated with AB-101 in the presence or absence of rituximab, an anti-CD20 antibody drug. ADCC of tumor cells by AB-101 was assessed using a live-cell analysis system where cytotoxicity was quantitatively measured and assessed up to 72 hrs at 1:1 NK cell (effector) to tumor cell (target) ratio. AB-101 demonstrated enhanced cytotoxicity over time against target cell lines Ramos and Raji in the presence of rituximab when compared to AB-101 alone (FIG. 19). In Ramos tumor model, when AB-101 was combined with rituximab, approximately 80% of lysis of target cells was observed at the end of 72 hrs co-culture which was higher than lysis of target cells (approximately 60%) observed in the presence of AB-101 alone (FIG. 19). In Raji tumor model, when AB-101 was combined with rituximab, approximately 90% of lysis of target cells was observed at the end of the 72 hour co-culture and was higher than lysis of target cells (approximately 79%) observed in the presence of AB-101 alone (FIG. 19).

The tumor specific effector functions of AB-101 were determined by measuring intracellular cytokines and markers of degranulation. AB-101 cells were co-cultured with a target tumor cell line (K562, Ramos or Raji) at a ratio of 1:1 for 4 hrs. Golgi-plug™ and Golgi-stop™ were used to prevent extracellular secretion of cytokines and CD107a. Production of intracellular cytokines and expression of degranulation markers by AB-101 in response to stimulation with tumor cells was measured by flow cytometry.

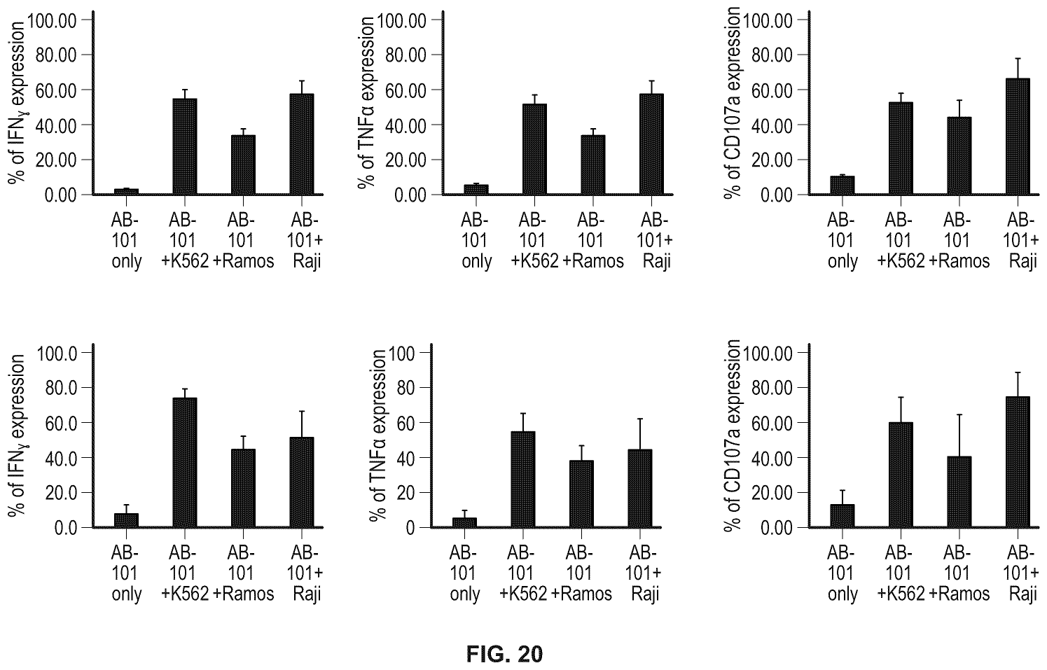

Consistent with the cytotoxic activity as demonstrated in FIG. 18, co-culturing of AB-101 with a cancer cell lines (K562, Ramos or Raji) resulted in increase in production of effector cytokines (IFN-γ, TNFα) and expression of marker of degranulation (CD107a) when compared to the control, AB-101 culture alone. (FIG. 20). These results confirm AB-101 activity in response to tumor cells.

### Example 10: AB-101 In Vivo Pharmacology

The ability of AB-101 to directly kill malignant target cells in vivo was evaluated in SCID mouse xenograft models using the Raji and Ramos CD20+B-cell lymphoma cell lines.

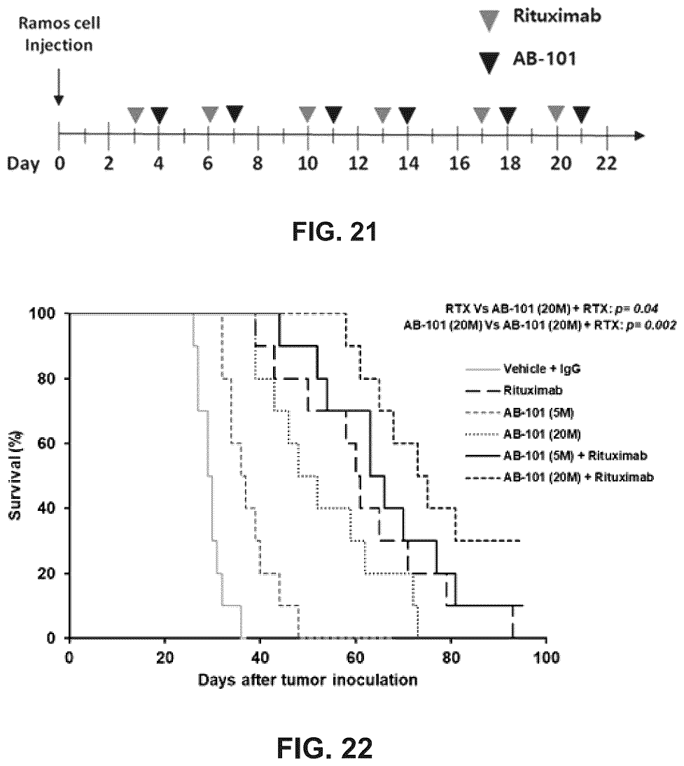

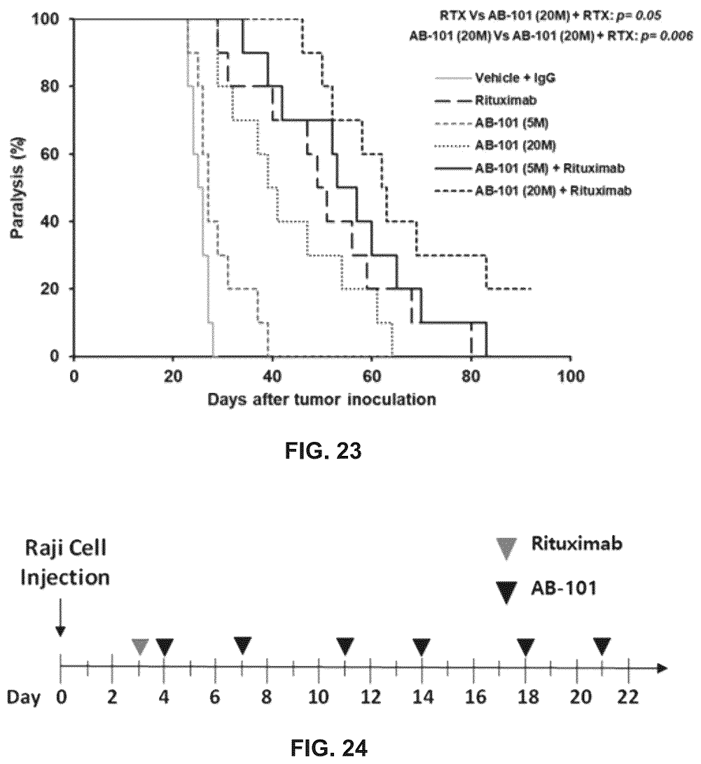

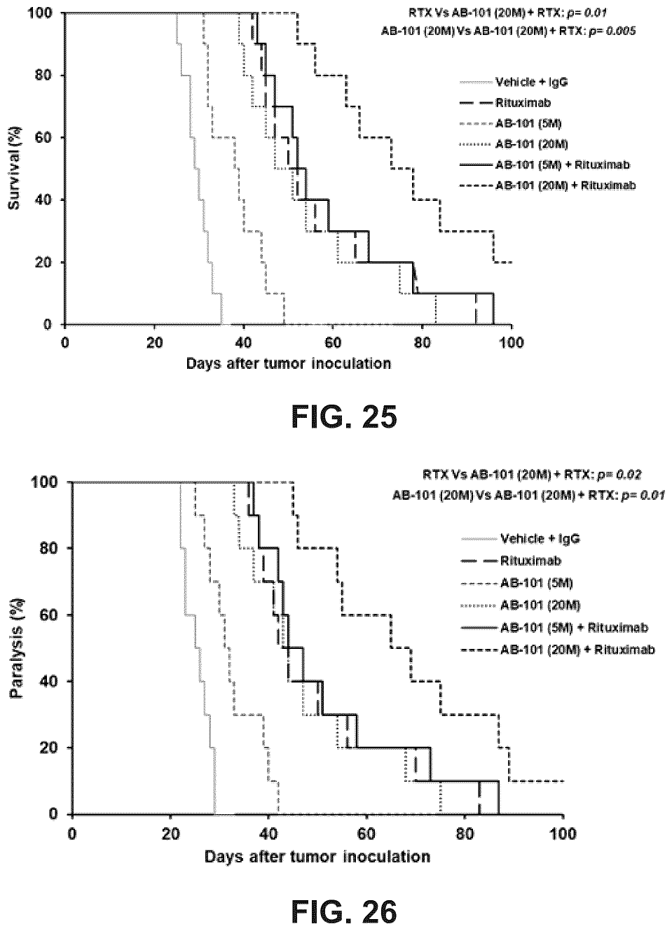

Two doses of AB-101 (0.5×10⁷ cells/dose and 2×10⁷ cells/dose) were tested in in vivo efficacy studies. Both doses levels were administered six times to lymphoma-bearing SCID mice. The dosing schedule and regimen used for Ramos and Raji models is displayed in FIG. 21, FIG. 22, FIG. 23, Table 33, FIG. 24, FIG. 25, FIG. 26, and Table 34.

TABLE 33
AB-101 in vivo Dosing

| | | Median | Median |
|---|---|---|---|
| Ramos cells | | Paralysis- | survival |
| (i.v.) | Group (10 each) | free (days) | (days) |
| 1 × 10⁶ | Vehicle + IgG (0.3 μg) | 25.0 | 30.5 |
| cells/mouse | Rituximab (0.3 μg) | 54.0 | 61.5 |
| | AB-101 (0.5 × 10^{7c} | 31.0 | 37.5 |
| | AB-101 (2 × 10⁷) | 44.0 | 51.0 |
| | Rituximab + AB-101 (0.5 × 10⁷) | 58.0 | 64.5 |
| | Rituximab + AB-101 (2 × 10⁷) | 65.5 | 74.0 |

TABLE 34
AB-101 in vivo Dosing

| | | Median | Median |
|---|---|---|---|
| Raji cells | Group (10 each) | Paralysis- | survival |
| (i.v.) | Dose/mouse | free (days) | (days) |
| 1 × 10⁵ | Vehicle + IgG (0.01 μg) | 26.5 | 31.0 |
| cells/mouse | Rituximab (0.01 μg) | 43.0 | 51.0 |
| | AB-101 (0.5 × 10⁷ cells) | 31.5 | 38.5 |
| | AB-101 (2 × 10⁷ cells) | 43.0 | 46.0 |
| | Rituximab + AB-101 (0.5 × 10⁷ cells) | 45.5 | 53.0 |
| | Rituximab + AB-101 (2 × 10⁷ cells) | 67.0 | 75.5 |

Efficacy of AB-101 and AB-101 in combination with rituximab was assessed by calculating median survival of each group through monitoring mortality after transplantation of tumor cells. Median time to tumor-associated paraplegia of the hind limb was therefore calculated for each treatment group in the following studies as additional evidence of efficacy.

In the Ramos xenograft tumor model experiments, death of animals was observed from day 27 to day 100 (FIG. 21, FIG. 22, FIG. 23, and Table 3). Median survival was 30.5 days in the control group compared to 37.5 days with AB-101 alone (5×10⁶ cells/dose), or 51 days with AB-101 alone (20×10⁶ cells/dose), or 61.5 days with rituximab alone, or 64.5 days with AB-101 (5×10⁶ cells/dose) plus rituximab, 74 days with AB-101 (20×10⁶ cells/dose) plus rituximab.

In the Raji xenograft tumor model experiments, death of animals was observed from day 25 to day 100 (FIG. 24, FIG. 25, FIG. 26, and Table 34). Median survival was 31 days in the control group compared to 38.5 days with AB-101 alone (5×10⁶ cells/dose), or 46 days with AB-101 alone (20×10⁶ cells/dose), or 51 days with rituximab alone, or 53 days with AB-101 (5×10⁶ cells/dose) plus rituximab, 75.5 days with AB-101 (20×10⁶ cells/dose) plus rituximab.

In conclusion, data obtained from three independent experiments in the Ramos model and two independent experiments in the Raji model illustrated that concurrent administration of AB-101 and rituximab increased the median survival of tumor-bearing mice by an average of 19.6 days (range 8.5-38 days) and 25.75 days (range 24.5-27 days) respectively, compared to rituximab alone. These results demonstrate the therapeutic potential of combining AB-101 with a monoclonal antibody to potentiate ADCC response and, more specifically, the therapeutic potential for the combination of AB-101 with rituximab in B-cell lymphomas such as NHL.

### Example 11: AB-101 Pharmacokinetics and Biodistribution

The NOD scid gamma (NSG) mouse model was used to determine the biodistribution and pharmacokinetics (PK) of AB-101. Vehicle (PBS, Dextran, Albumin (human) DMSO) and AB-101 cells (0.5×10⁷ cells/mouse, 2×10⁷ cells/mouse) were administered intravenously (0.25 mL/mouse) for a total of 8 doses. Animals in vehicle and AB-101 groups were sacrificed at timepoints 4 hr, 1, 3, 7, 14 and 78 days (n=3 male mice, n=3 female mice per timepoint) post last dose infusion.

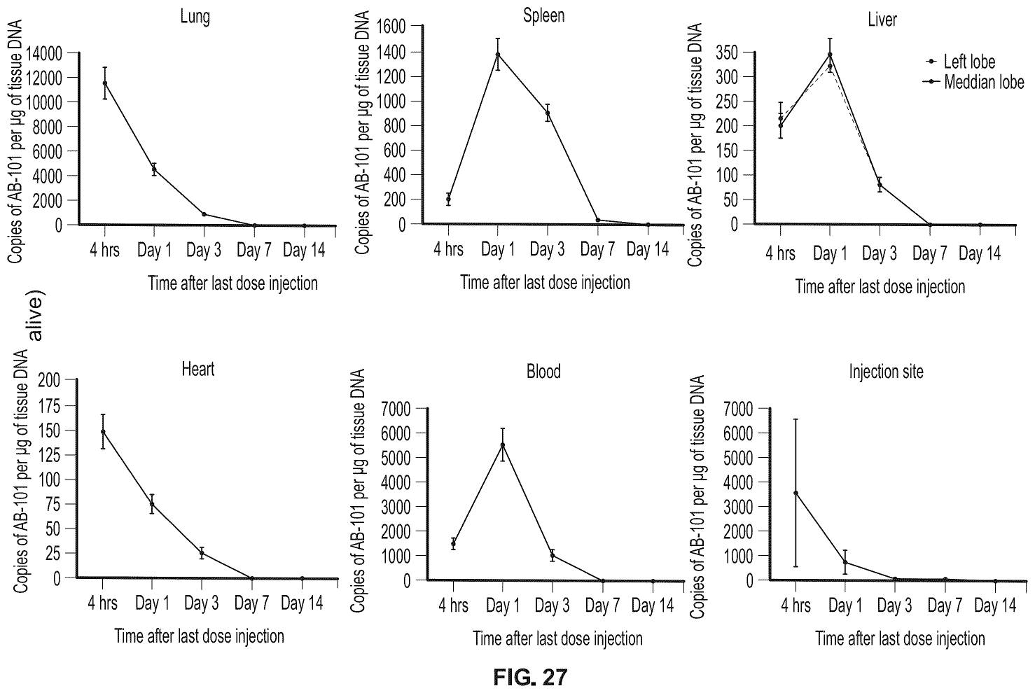

AB-101 was detected predominantly in highly perfused tissues (lungs, spleen, heart and liver) and at the site of injection starting at 4 hrs after administration, until 3 days after administration of final dose of AB-101 (day 53) (FIG. 27). At 7 days after administration of final dose (day 57) AB-101 was detected in lung (3 out of 6 samples), spleen (5 out of 6 samples) and injection site (5 out of 6 samples). At 14 days and 28 days after administration of final dose (day 64 and day 78 respectively), AB-101 was detected in two and one injection site samples, respectively. The sporadic incidence and low concentrations observed from the injection site samples at day 64 and day 78 would not be indicative of systemic persistence of the AB-101 test article.

The results from the biodistribution studies indicate that the distribution of AB-101 in vivo is consistent with the intravenous route of administration and that the cells lack long-term persistence potential with tissue clearance after 7 days post-administration and no evidence of permanent engraftment.

### Example 12: AB-101 Toxicology

Nonclinical toxicity of AB-101 was assessed in a GLP study of NSG mice. The study was designed to evaluate the acute and delayed toxicity profile of AB-101. Two dose levels of AB-101, 0.5×10⁷ and 2×10⁷ cells/animal, were tested in the study. The proposed test dose range was designed to deliver a greater exposure of the product than the planned highest equivalent human dose to be given in a first-in-human study (4×10⁹ cells per dose). Based on allometric scaling (Nair 2016), 0.5×10⁷ cells/mouse corresponded to 14×10⁹ cells/human, and 2×10⁷ cells/mouse corresponded to 56×10⁹ cells/human, assuming a patient weighing 70 kg. AB-101 was administered intravenously once weekly for 8 weeks via the tail vein. Acute toxicity of AB-101 was evaluated 3 days after the eighth dose (i.e., last dose). Delayed toxicity was evaluated at the end of the 28 days recovery period after the eighth dose. Viability, body weight, clinical observations and palpations were recorded for each animal during the in-life portion of the study. Gross necropsy and sample collection for hematology, clinical chemistry and histopathology analysis were performed at the time of euthanasia for all animals.

Each group contained 20 animals in total, with 10 of each gender, to evaluate findings in both sexes and for powered statistical analysis. A vehicle treated control group was included for comparison to the AB-101 treated groups. To minimize treatment bias, animals were assigned to dose groups based on computer-generated (weight-ordered) randomization procedures, with male and females randomized separately. The study adhered to GLP guidelines, including those for data reporting.

No mortality and no adverse clinical observations were recorded related to administration of AB-101 at any of the evaluated dose levels. All minor clinical observations that were noted are common findings in mice and were not considered related to AB-101 administration. Body and organ weight changes were comparable among dose groups and different days of post-treatment assessment (Day 53 for acute toxicity groups and Day 78 for delayed toxicity groups). There were no AB-101-related changes in hematology and clinical chemistry parameters or gross necropsy findings noted in animals at euthanasia in either the acute or delayed toxicity groups. All fluctuations among individual and mean clinical chemistry values, regardless of statistical significance, were considered sporadic, consistent with biologic and procedure-related variation, and/or negligible in magnitude, and therefore deemed not related to AB-101 administration. There were no AB-101-related microscopic findings. In conclusion, results from the GLP toxicity study indicate that AB-101 is well tolerated in NSG mice with repeated dosing of up to 2×10⁷ cells/dose/animal.

### Example 13: Cryopreservation of NK Cells

AB-101 cells were prepared by the process shown in FIG. 5. At the end of the culture period the cells were harvested through the use of a Sartorius kSep® 400 Single-Use Automated Centrifugation System at Relative Centrifugal Field (RCF): 800-1200 g with a flow rate at 60 to 120 mL/min, and washed two times with Phosphate Buffer Solution (PBS). After washing, the AB-101 cells were formulated with: (1) Albumin (human); (2) Dextran 40; (3) DMSO and (4) PBS to a target concentration of 1×10⁸ cells/mL (exemplary cryopreservation composition #1, Table 4). The formulated suspension was then filled at a target volume of 11 mL into 10 mL AT-Closed vial®. Filled vials were inspected, labeled and cryopreserved in a controlled rate freezer at ≤−135° C.

Stability studies were carried out with time=0 as the initial release testing data. The stability storage freezer is a validated vapor phase LN₂ storage freezer which is set to maintain a temperature of ≤−135° C. For sterility timepoints, 10% of the batch size or 4 vials, whichever is greater, was tested. Test articles were thawed at 37° C. to mimic clinical thawing conditions.

As shown in Table 35, viability and activity of cryopreserved AB-101 was shown to be preserved through at least nine months.

TABLE 35
Long Term Viability and Activity of Cryopreserved AB-101

| | | | Cryopreserved (≤135° C.), | | | | | |
|---|---|---|---|---|---|---|---|---|
| | | | Sample times (months) | | | | | |
| | | Acceptance | 0 | 3 | 6 | 9 | 12 | 18 |
| Test Attribute | | Criterion | months | months | months | months | months | months |
| Cell Count | | 0.9-1.3 × 10⁹ | 1.3 × 10⁹ | 1.3 × 10⁹ | 1.4 × 10⁹ | 1.4 × 10⁹ | 1.3 × 10⁹ | 1.4 × 10⁹ |
| (cells/vial) | | | | | | | cells/vial | cells/vial |
| Cell Viability | | ≥70% | 96% | 93% | 94% | 93% | 90% | 87% |
| Endotoxin | | <5 | ≤1 | <1 | <1 | ≤1 | <1.0 | <1.0 |
| (EU/kg/hr) | | | | | | | | |
| Identity | CD3−, | ≥85% | 99.16% | 99.39% | 99.49% | 99.41% | 99.54% | 99.36% |
| | CD56+ | | | | | | | |
| | % | | | | | | | |
| | CD56+, | ≥70% | 94.42% | 94.60% | 94.44% | 93.71% | 94.85% | 90.27% |
| | CD16+ | | | | | | | |
| | % | | | | | | | |
| Purity | CD3+ | ≤0.20% | 0.00% | 0.00% | 0.00% | 0.04% | 0.06% | 0.00% |
| | % | | | | | | | |
| | CD14+ | ≤1.00% | 0.02% | 0.00% | 0.00% | 0.02% | 0.01% | 0.00% |
| | % | | | | | | | |
| | CD19+ | ≤1.00% | 0.01% | 0.00% | 0.01% | 0.02% | 0.00% | 0.00% |
| | % | | | | | | | |

| | | | | | | | |
|---|---|---|---|---|---|---|---|
| Potency (killing | ≥50% | 69.00% | 66.90% | 67.40% | 61.80% | 67.1 | 68.3 |
| at 4 hours) | | | | | | | |

To understand the stability characteristics of AB-101 during handling just prior to administration, a “bedside” short-term stability study was performed. Samples were thawed, transferred to 10 mL syringes, filtered, and the contents stored in Falcon tubes, and kept at that temperature for defined time periods as shown. The collected product was then tested. Short-Term Stability Data for two lots of AB-101 is shown in Table 36.

TABLE 36
Short Term Stability Data for AB-101

| Average data | | Lot | 0 | 5 | 15 | 30 | 60 | 90 | 120 | |
|---|---|---|---|---|---|---|---|---|---|---|
| of 4 vials | | release | min | min | min | min | min | min | min | Flush |
| PG001 | Cell count | 1.18 | 1.10 | 1.11 | 1.11 | 1.10 | 1.12 | 1.07 | 1.03 | 0.07 |
| | (0.8-1.2 × 10⁸ | | | | | | | | | |
| | cells/mL) | | | | | | | | | |
| | Viability (%) | 93 | 94 | 94 | 94.75 | 94 | 93.5 | 93.5 | 93.5 | 93.25 |
| | CD3-56+ | 99.53 | 99.53 | NT | NT | NT | 99.53 | NT | 97.58 | NT |
| | (%) | | | | | | | | | |
| | CD16+CD56 | 93.24 | 97.74 | NT | NT | NT | 97.74 | NT | 97.43 | NT |
| | (%) | | | | | | | | | |
| PG002 | Cell count | 1.09 | 1.13 | 1.08 | 1.14 | 1.14 | 1.08 | 1.11 | 1.05 | 0.08 |
| | (0.8-1.2 × 10⁸ | | | | | | | | | |
| | cells/mL) | | | | | | | | | |
| | Viability (%) | 94 | 93.75 | 94.25 | 94.75 | 95.25 | 94.25 | 94.5 | 94 | 92.75 |
| | CD3-56+ | 98.40 | 99.30 | NT | NT | NT | 99.27 | NT | 99.53 | NT |
| | (%) | | | | | | | | | |
| | CD16+CD56 | 91.72 | 98.88 | NT | NT | NT | 99.55 | NT | 98.40 | NT |
| | (%) | | | | | | | | | |

### Example 14: CAR Costimulatory Structure Comprising OX40L

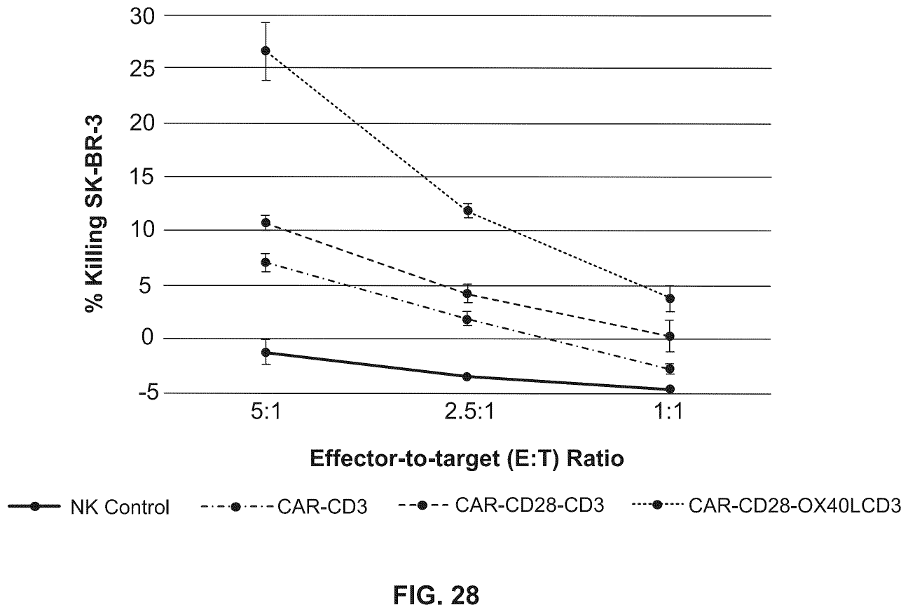

In some embodiments, the NK cells are CAR-NK cells. As shown in FIG. 28, CAR-NKs comprising a co-stimulatory domain comprising OX40L exhibited greater cytotoxic potential than those without OX40L. In this example, the CAR-NK cells comprise an anti-HER2 scFv as described in US20200399397A1, which is hereby incorporated by reference in its entirety.

### Example 15: Cord Blood NK Cells Selected for KIR-B and CD16 158 v/v Exhibit Low CD38 Expression after Expansion

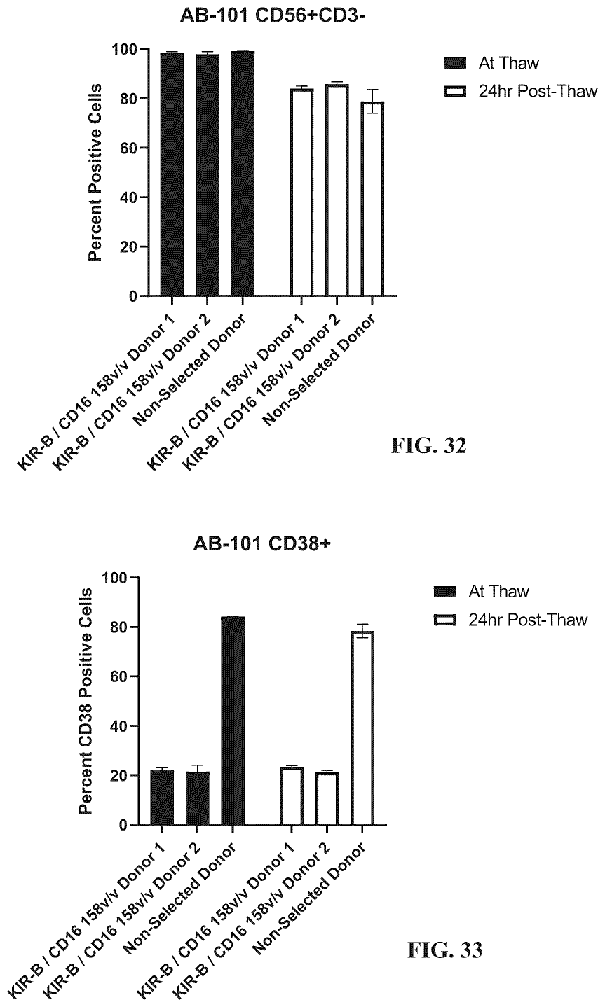

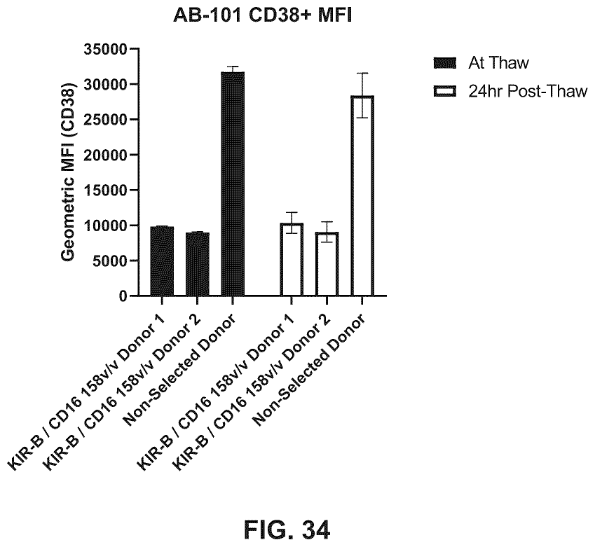

NK cells were expanded, as described in Example 6, using two different cord blood donors selected for KIR-B and CD16 158v/v to generate AB-101 cells, and from one non-selected donor (control). The purity of the resulting cells (percent CD56+CD3−) as measured by flow cytometry, is show in FIG. 32. As shown in FIG. 33 and FIG. 34, CD38 expression is lower in KIR-B/158 v/v NK cells as a population (percent positive, FIG. 33) and individually (mean fluorescence intensity of the positive cells, FIG. 34) compared to non-selected NK cells.

### Example 16: Surface Protein Expression of AB-101

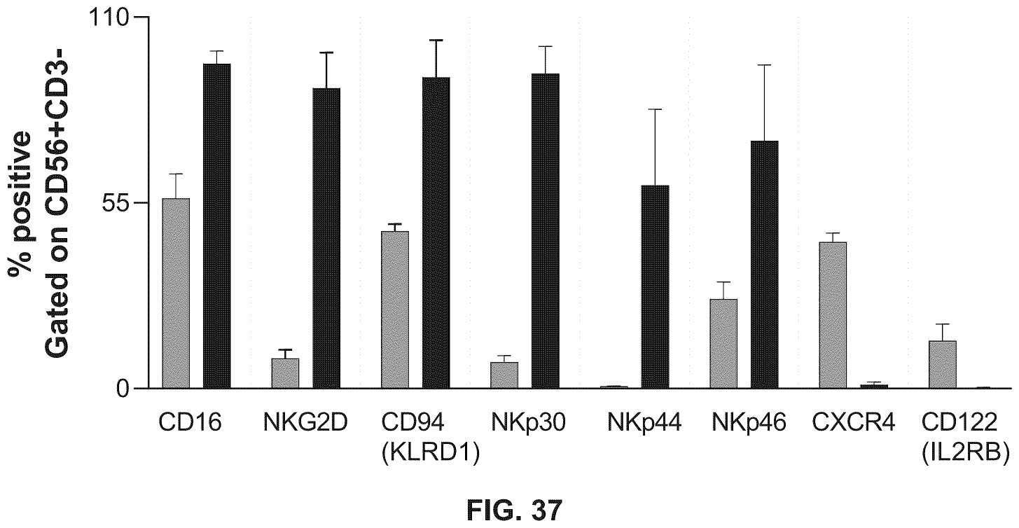

NK cells were expanded, as described in Example 6. Surface protein expression of the starting NK cell source (cord blood gated on CD56+/CD3− expression, n=3) was compared to the resulting expanded NK cells (n=16). As shown in FIG. 37, CD16 expression was high in the resulting cells, increased relative to the starting cells. Expression of NKG2D, CD94, NKp30, NKp44, and NKp46 was also increased, whereas expression of CXCR4 and CD122 was decreased.

### Example 17: Gene Expression of AB-101

NK cells were expanded, as described in Example 6, to generate AB-101 cells. Gene expression was measured for 770 genes and compared to gene expression profiles for cord blood natural killer cells and peripheral blood natural killer cells

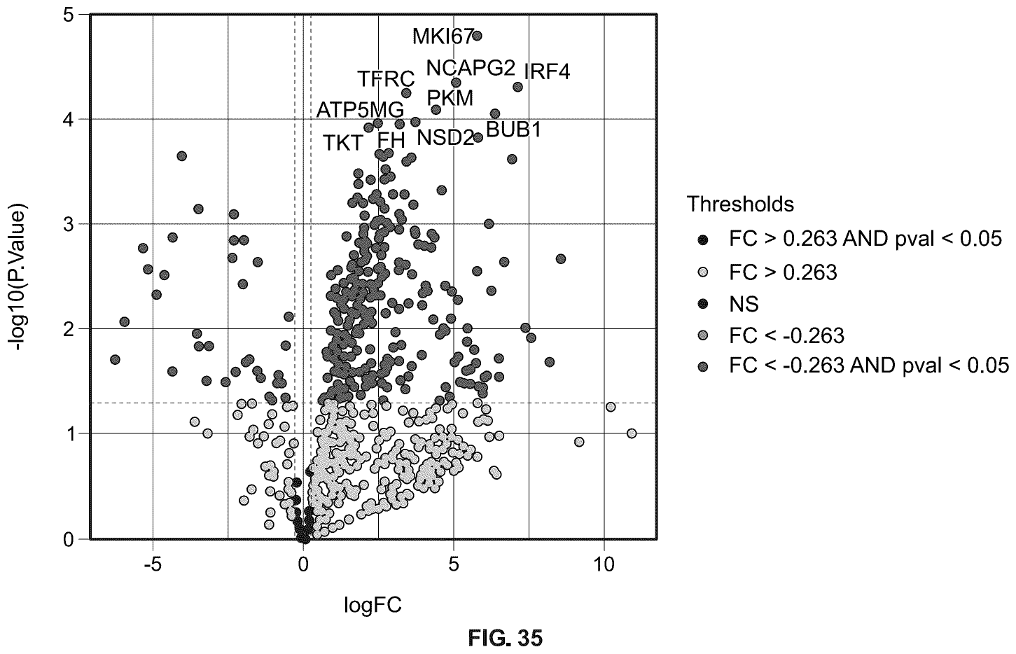

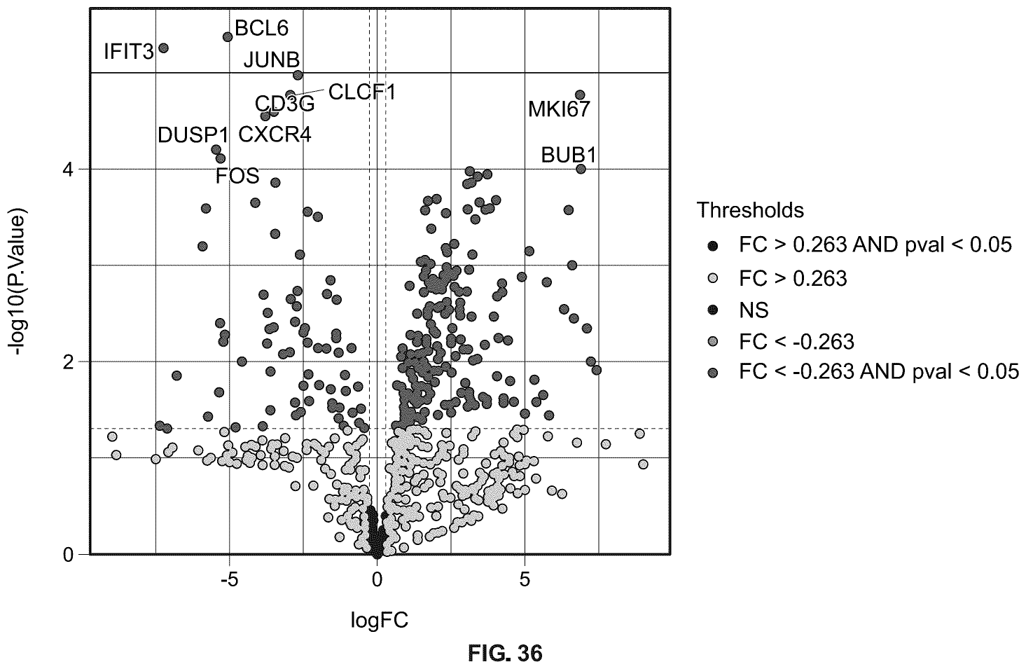

As show in FIG. 35, AB-101 cells differed in their overall expression pattern from cord blood natural killer cells, with 204 of the 770 genes having statistically significant differences expression. Of those 204, 13 were down-regulated and 191 up-regulated in AB-101 compared to cord blood natural killer cells. As shown in FIG. 36, AB-101 cells differed in their overall expression pattern from peripheral blood natural killer cells, with 167 of the 770 genes having statistically significant differences in expression. Of those 167, 44 were down-regulated and 123 up-regulated in AB-101 compared to peripheral blood natural killer cells. 114 differentially expressed genes were common between both groups. Of those 114, 6 genes were down-regulated (Table 37), while 107 genes were upregulated (Table 38) in AB-101 as compared to both peripheral blood and cord blood natural killer cells.

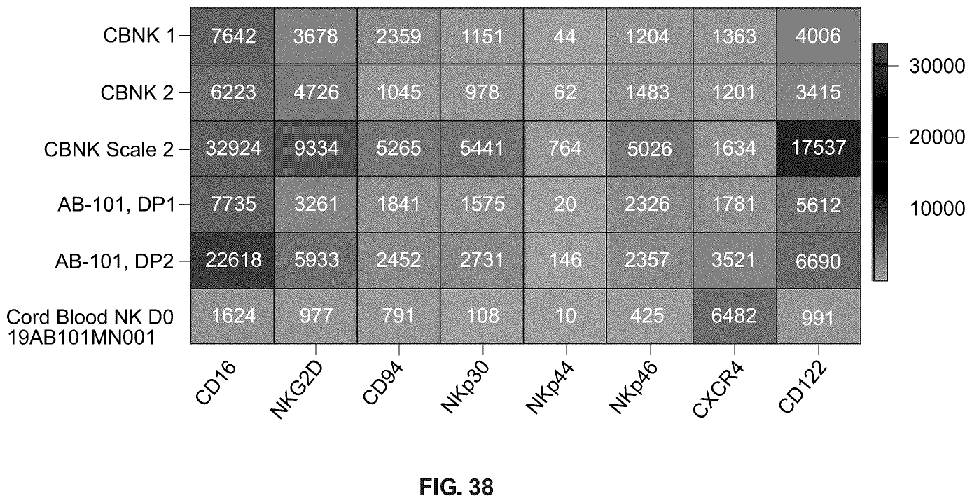

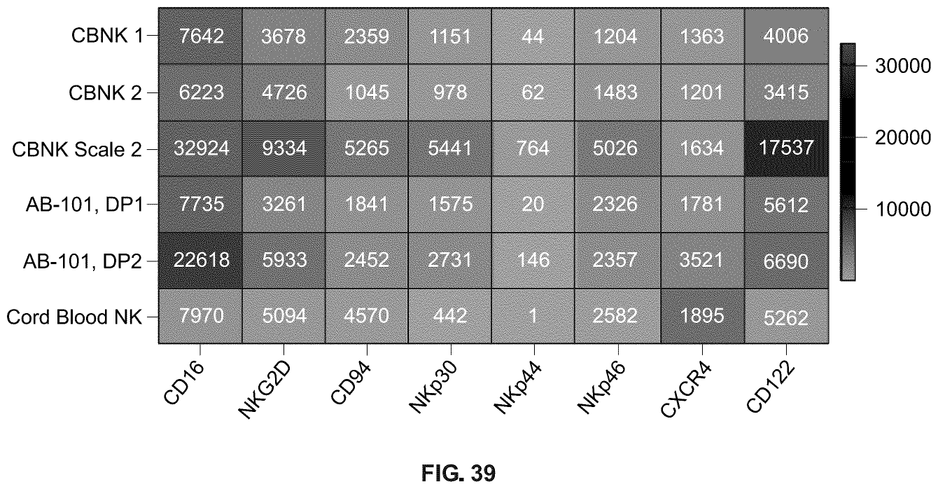

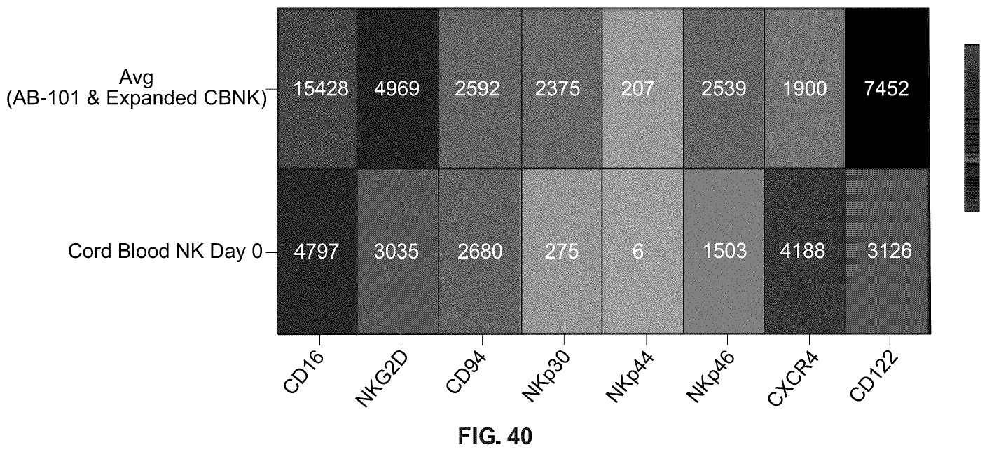

Gene expression signatures for surface expressed proteins (CD16, NKG2D, CD94, NKp30, NKp44, NKp46, CXCR4, and CD122) also differed between AB-101 (selected for KIR-B/158 v/v expression) and cord blood natural killer cells (Cord Blood NK Day 0 (D0); not selected for KIR-B/158 v/v expression. Expanded cord blood cells (CBNK 1, CBNK2, CBNK Scale 2; not selected for KIR-B/158 v/v expression; showed similar gene expression patterns to AB-101 (FIG. 38 and FIG. 39). FIG. 40 shows an average of gene expression of expanded cord blood NK samples (both AB-101 and expanded cord blood NK samples) and non-expanded cord blood NK cells.

TABLE 37
Genes downregulated in AB-101 compared to cord
blood and peripheral blood natural killer cells

| Gene Name | Related pathways |
|---|---|
| BCL6 | Signaling events mediated by HDAC Class II and Innate Immune System |
| VAV3 | Coregulation of Androgen receptor activity and Cytoskeletal Signaling |
| GZMM | Granzyme pathway and creation of C4 and C2 activators |
| MX1 | Innate Immune System and Interferon gamma signaling |
| CD160 | Innate Lymphoid Cells Differentiation and Innate Immune System |
| KLRG1 | Innate Immune System and Immunoregulatory interactions between a |
| | Lymphoid and a non-Lymphoid cell |

TABLE 38
Genes upregulated in AB-101 compared to cord blood
and peripheral blood natural killer cells

| Gene Name | Related pathways |
|---|---|
| GPI | Glucose metabolism |
| PFKP | |
| ALDOA | Glucose metabolism and HIF-1-alpha transcription factor network |
| PKM | |
| PFKL | |
| PGK1 | |
| CS | Glucose metabolism and Pyruvate metabolism and Citric Acid (TCA) cycle |
| MDH2 | |
| FH | |
| GOT1 | CDK-mediated phosphorylation and removal of Cdc6 and Glucose |
| | metabolism |
| PGAM1 | Glucose metabolism and Cori Cycle |
| ENTPDI | Purine metabolism and ATP/ITP metabolism |
| ATP5MG | Purine nucleotides de novo biosynthesis and Respiratory electron transport, |
| ATP5MF | ATP synthesis by chemiosmotic coupling, and heat production by |
| | uncoupling proteins |
| COX7C | Respiratory electron transport, ATP synthesis by chemiosmotic coupling, |
| COX7A2 | and heat production by uncoupling proteins |
| COX6B1 | |
| NDUFA2 | |
| NDUFA6 | |
| NDUFB9 | |
| UQCR10 | |
| UQCRQ | |
| COX5B | TP53 Regulates Metabolic Genes and Respiratory electron transport, ATP |
| NDUFA4 | synthesis by chemiosmotic coupling, and heat production by uncoupling |
| | proteins |
| SDHB | Pyruvate metabolism and Citric Acid (TCA) cycle and Respiratory electron |
| | transport, ATP synthesis by chemiosmotic coupling, and heat production by |
| | uncoupling proteins |
| BUB1 | Cell Cycle, Mitotic and Mitotic Metaphase and Anaphase |
| SGO2 | |
| NCAPD2 | Cell Cycle, Mitotic and Cell cycle, Chromosome condensation in |
| NCAPG2 | prometaphase |
| NCAPH | |
| SMC2 | |
| NEK2 | cell cycle, mitotic and CDK-mediated phosphorylation |
| PSMA6 | |
| PSMB10 | |
| PSMA2 | |
| PSMA3 | |
| NSD2 | Cell Cycle, Mitotic and Homology Directed Repair |
| TFDP1 | Cell cycle, mitotic and pre-NOTCH expression and processing |
| RBX1 | Cell cycle, mitotic and signaling by NOTCH1 |
| AURKA | Cell cycle, mitotic and SUMOylation |
| UBE2I | Cell Cycle, Mitotic and Coregulation of Androgen receptor activity |
| HDAC8 | Cell Cycle, Mitotic and CREB Pathway |
| CKAP5 | Cell Cycle, Mitotic and Cytoskeletal Signaling |
| AKT1 | PI3K/AKT activation and cell cycle |
| KIR3DL1/2 | Innate Immune System and Immunoregulatory interactions between a |
| | Lymphoid and a non-Lymphoid cell |
| SH2D1A | Innate Immune System and Tyrosine Kinases/Adaptors |
| LIF | Innate Immune System and Interleukin-6 family signaling |
| MIF | Cell cycle Role of SCF complex in cell cycle regulation and Innate Immune |
| | System |
| SOCS2 | TGF-Beta Pathway and Innate Immune System |
| TRIM26 | Interferon gamma signaling and Innate Immune System |
| TRBC1/2 | Innate Immune System and CD28 co-stimulation |
| UBA5 | Innate Immune System and protein ubiquitylation |
| IRF4 | Interferon gamma signaling and IL-4 Signaling and its Primary Biological |
| | Effects in Different Immune Cell Types |
| NME1 | Granzyme Pathway and Mesodermal Commitment Pathway |
| PRF1 | IL12 signaling mediated by STAT4 and Granzyme Pathway |
| IL4R | IL-4 Signaling |
| CISH | TGF-Beta Pathway and Development Thrombopoetin signaling via JAK- |
| | STAT pathway |
| BCL2 | TNFR1 Pathway and CNTF Signaling |
| GZMB | Th17 Differentiation and Granzyme Pathway |
| IL26 | TGF-Beta Pathway and PEDF Induced Signaling |
| BCL2L1 | TNFR1 Pathway and Development Thrombopoetin signaling via JAK- |
| | STAT pathway |
| CD276 | NF-kappaB signaling |
| MAP3K7 | TLR4 signalling and MAP Kinase Signaling |
| CXCR3 | innate lymphoid cells differentiation |
| LPAR6 | RET signaling and Signaling by GPCR |
| VAV1 | PI3K/AKT activation and RET signaling |
| IL2RA | p70S6K Signaling and RET signaling |
| OPA1 | Apoptosis and Autophagy and CDK-mediated phosphorylation and removal |
| | of Cdc6 |
| CASP3 | Apoptosis, TNFR1 pathway and ERK signaling |
| DAP3 | Mitochondrial translation and all-trans-Retinoic Acid Mediated Apoptosis |
| MTHFD1 | Metabolism of water-soluble vitamins and cofactors and Trans-sulfuration |
| SHMT1 | and one carbon metabolism |
| SHMT2 | |
| MTHFD2 | |
| MKI67 | Proliferation |
| PARP1 | Differentiation, proliferation |
| TFRC | Cytoskeletal Signaling and HIF-1-alpha transcription factor network |
| MAP2K2 | VEGF Signaling Pathway and CNTF Signaling |
| LTB | CDK-mediated phosphorylation and removal of Cdc6 and Innate Lymphoid |
| | Cells Differentiation |
| NDUFAB1 | palmitate biosynthesis and acyl protien metabolism |
| HSD11B1 | Bupropion Pathway, Pharmacokinetics and Metabolism of steroid hormones |
| G6PD | Cori Cycle and TP53 Regulates Metabolic Genes |
| FASN | palmitate biosynthesis and angiopoietin like protien 8 regulatory pathway |
| PTCD1 | Regulation of translation |
| TBC1D10B | vesicle-mediated transport |
| RPTOR | mTOR signaling and MAPK signaling |
| PRICKLE3 | assembly, stability, and function of mitochondrial membrane ATP synthase |
| GART | Trans-sulfuration and one carbon metabolism and Methotrexate Pathway |
| CCNC | Signaling by NOTCH1 and Regulation of lipid metabolism by Peroxisome |
| | proliferator-activated receptor alpha |
| PPAT | Methotrexate Pathway (Cancer Cell) and Purine metabolism |
| FKBP1A | Transcriptional activity of SMAD2/SMAD3-SMAD4 |
| | heterotrimer and DNA Damage/Telomere Stress Induced Senescence |
| NME2 | Synthesis and interconversion of nucleotide di- and |
| | triphosphates and superpathway of pyrimidine deoxyribonucleotides de |
| | novo biosynthesis |
| HMGCR | Regulation of lipid metabolism by Peroxisome proliferator-activated |
| | receptor alpha (PPARalpha) and Integrated Breast Cancer Pathway |
| COX16 | TP53 Regulates Metabolic Genes |
| AFDN | Cytoskeleton remodeling Regulation of actin cytoskeleton by Rho |
| | GTPases and Cytoskeletal Signaling |
| CCR8 | Chemokine Superfamily: Human/Mouse Ligand-Receptor |
| | Interactions and Akt Signaling |
| NMT1 | HIV Life Cycle and Metabolism of fat-soluble vitamins |
| SRR | serine and glycine biosynthesis |
| TIMM23 | Mitochondrial protein import and Metabolism of proteins |
| GNG10 | Aquaporin-mediated transport and Inwardly rectifying K+ channels |
| CD9 | differentiation, adhesion, and signal transduction, and expression of this |
| | gene plays a critical role in the suppression of cancer cell motility and |
| | metastasis |
| ACACA | Mesodermal Commitment Pathway and Fatty Acid Biosynthesis |
| PYCR3 | Amino acid synthesis and interconversion (transamination) and Peptide |
| | chain elongation |
| CD99 | Cell surface interactions at the vascular wall and Integrin Pathway |
| DECR1 | Fatty Acid Biosynthesis and Mitochondrial Fatty Acid Beta-Oxidation |
| SCD | Angiopoietin Like Protein 8 Regulatory Pathway and Fatty Acid |
| | Biosynthesis |
| CPT1A | Regulation of lipid metabolism by Peroxisome proliferator-activated |
| | receptor alpha (PPARalpha) and Import of palmitoyl-CoA into the |
| | mitochondrial matrix |

### Example 18: Detection of Residual eHuT-78 Cells, Proteins, and DNA

The manufacturing process of AB-101 includes co-culturing with eHuT-78 feeder cells, which are engineered to express mTNF-α (SEQ ID NO. 12), MbIL-21 (SEQ ID NO: 11), and 4-1BBL (SEQ ID NO: 10). Described in this Example are methods for detecting residual eHuT-78 cells, proteins, and DNA, which can be used, for example, to measure the purity of the AB-101 cells, but also to identify cells that have been expanded and stimulated with eHuT-78 cells, as described, for example, in Example 6.

(A) Residual eHuT-78P (Cells)

In one example, residual eHuT-78P cells in AB-101 drug product are measured by flow cytometry (FACS). FACS is used to detect residual eHuT-78 in AB-101 DP by quantifying the live and dead CD3⁺4-1BBL^{high+} eHuT-78P. The FACS gating strategy, which sequentially gates: singlet, 7-AAD- and CD3⁺4-1BBL⁺, was used because eHuT-78 is derived from a HuT-78 cell line that expresses CD3 as cutaneous T lymphocyte. The HuT-78 cell line was transduced by 4-1BB ligand (4-1BBL), mutated tumor necrosis factor-α (mTNF-α) and membrane bound IL-21 (mbIL-21). Therefore, this assay is specific to eHuT-78 cells (as opposed, for example, to HuT-78 cells).

### Preparation of the Specimen

After the AB-101 drug product was thawed, the assay was performed within 30 minutes. 1 mL of cells were placed in a new 50 mL tube and 10 mL of BD FACSFlow Sheath Fluid (hereafter, sheath fluid) was slowly added using a pipette-aid. Cells mixed with the sheath fluid were centrifuged at 1200 rpm for 10 minutes, and when centrifugation was complete, the supernatant was removed. The bottom of the tube was tapped about 10 times to release the cell pellet so as not to clump, 15 mL of sheath fluid was then added into the tube, and the cell suspension was prepared to 3×10⁶ cells/mL.

### Cell Staining

The cells were stained by adding the antibody according to Table 39 below.

TABLE 39
Antibodies for Cell Staining

| | | | | | | PerCP-Cy5.5 | |
|---|---|---|---|---|---|---|---|
| | | FITC | | APC | | (7-AAD) | |
| Tube | | Antibody | usage | Antibody | usage | Antibody | usage |
| 1 | Un | MsIgG | 5 μL | MsIgG | 5 μL | MsIgG | 1 μL |
| | | | | (BD) | | | |
| 2 | FITC | CD56 | 1 μL | MsIgG | 5 μL | MsIgG | 1 μL |
| | | | | (BD) | | | |
| 3 | APC | MsIgG | 5 μL | CD56 | 5 μL | MsIgG | 1 μL |
| 4 | PerCP- | MsIgG | 5 μL | MsIgG | 1 μL | CD56 | 1 μL |
| | Cy5.5 | | | (BD) | | | |
| 5 | FMO | CD3 | 5 μL | MsIgG | 1 μL | 7-AAD | 4 μL |
| | | | | (Invitrogen) | | | |
| 6 | Sample | CD3 | 5 μL | 4-1BBL | 1 μL | 7-AAD | 4 μL |

100 μL of the prepared cell suspension was then added to each tube. The entire tube was vortexed so that cells and antibodies are well mixed. The tube was covered with foil so that it was not exposed to light and incubated in a refrigerator at 2-8° C. for 30 minutes.

After the reaction was complete, 2 mL of sheath fluid was added to the tube and centrifuged at 2000 rpm for 3 minutes. After centrifugation, the supernatant was discarded, 150 μL of BD cytofix was added to resuspend, and the cells were incubated in a refrigerator at 2-8° C. for at least 15 minutes. After the reaction has been completed, the cells were wrapped in foil and stored in the refrigerator, and measured within 72 hours.

### Flow Cytometry

After loading Tube 1 of the Compensation tube first, the voltage was adjusted to set the position of each isotype control uniformly. The compensation was adjusted after loading the remaining tubes 2-4 of the compensation tube. After completing the cytosetting, the sample tube and FMO tube were loaded to check the eHuT-78P cellular impurity. At this time, 50,000 events were recorded based on 7-AAD negative cells. After the flow cytometry analysis, the residual amount (%) of eHuT-78P cells were analyzed.

Analysis of eHuT-78P Residual Amount

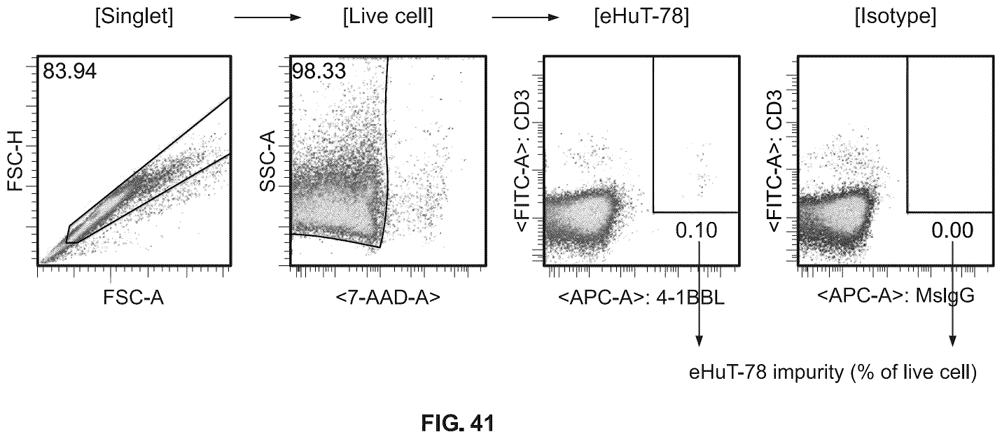

The residual amount (%) of eHuT-78P was analyzed as described herein using FlowJo software for the results obtained using LSR Fortessa equipment. Gating strategy proceeds as shown in FIG. 41.

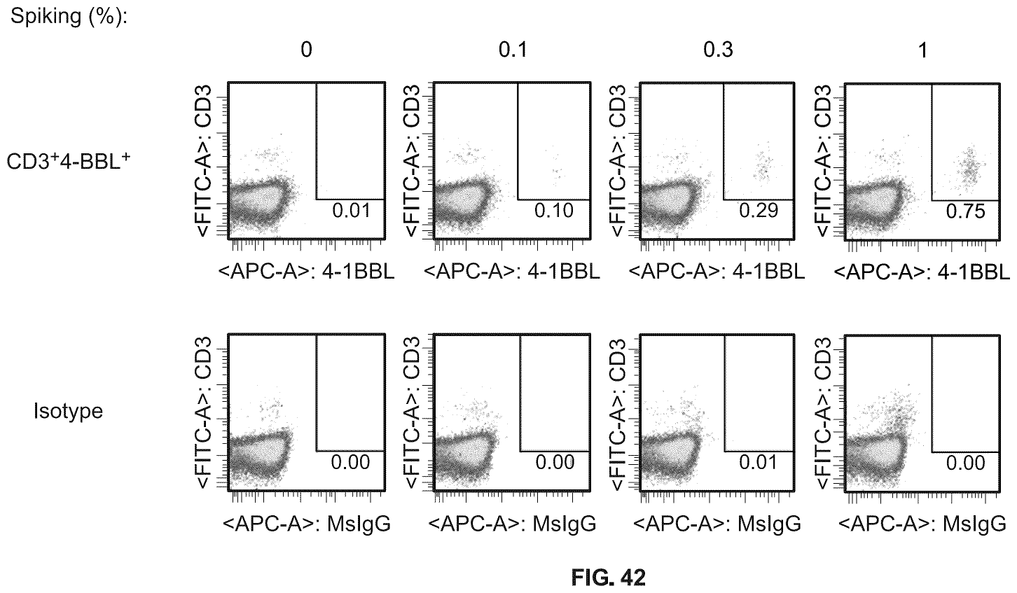

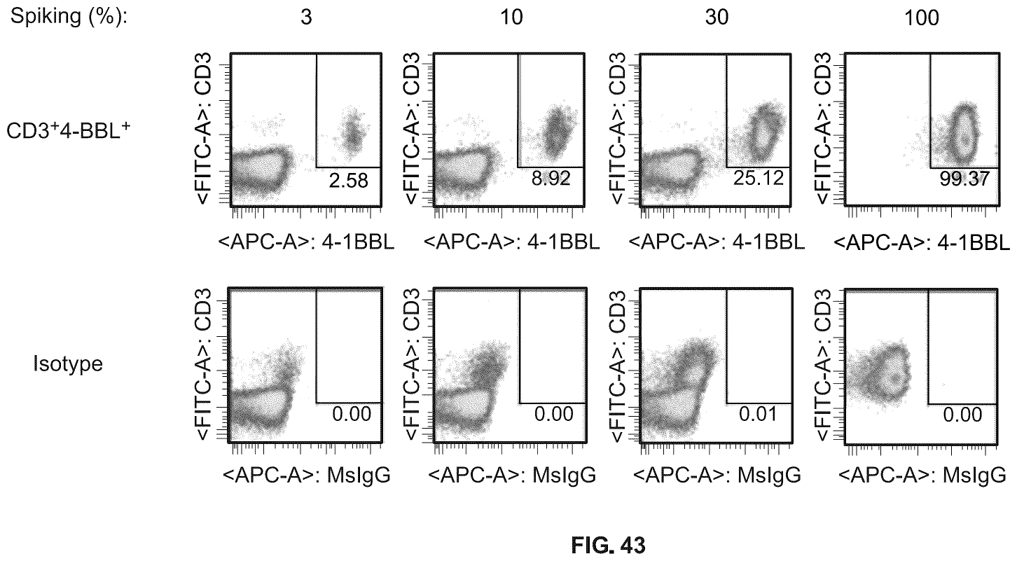

Singlet (FSC-A/FSC-H) gating, Live cell (7-AAD/SSC-A) gating, and 7-AAD(−) gating were performed, wherein eHuT-78P cell residual impurity (CD3+/4-1BBL^{high}) was shown as % of live cells. An eHuT-78 single cell that highly expressed the three genes was selected, wherein among the three genes, 4-1BBL was utilized for the FACS gating strategy because it showed the highest expression in AB-101 cell bank and final drug product (FIG. 42; FIG. 43;).

AB-101 cells were also spiked with varying amounts of eHuT-78 feeder cells to test the assay. The amount of eHuT-78 cells added to each condition and the amount detected by the assay are shown in Table 40, below.

TABLE 40
Specificity and Sensitivity of FACS assay for peripheral blood natural killer cells spiked with eHuT-78P

| Spiking % | 0% | 0.03% | 0.1% | 0.3% | 1% | 3% | 10% | 30% | 100% |
|---|---|---|---|---|---|---|---|---|---|
| PB-NK 1 (%) | 0.01 | 0.07 | 0.10 | 0.29 | 0.75 | 2.58 | 8.92 | 25.12 | 99.37 |
| PB-NK 2 (%) | 0.01 | 0.04 | 0.14 | 0.26 | 1.03 | 2.62 | 8.11 | 23.26 | 99.28 |
| PB-NK 3 (%) | 0.00 | 0.02 | 0.15 | 0.31 | 1.13 | 2.34 | 6.19 | 26.24 | 99.14 |
| PB-NK 4 (%) | 0.00 | 0.05 | 0.12 | 0.34 | 1.40 | 3.63 | 13.62 | 36.41 | 99.08 |
| Mean (%) | 0.01 | 0.05 | 0.13 | 0.31 | 1.08 | 2.79 | 9.21 | 27.76 | 99.22 |
| Cell Recovery | | 150 | 128 | 103 | 108 | 93 | 92 | 93 | 99 |
| (%) | | | | | | | | | |

(B) Residual eHuT-78P (DNA)

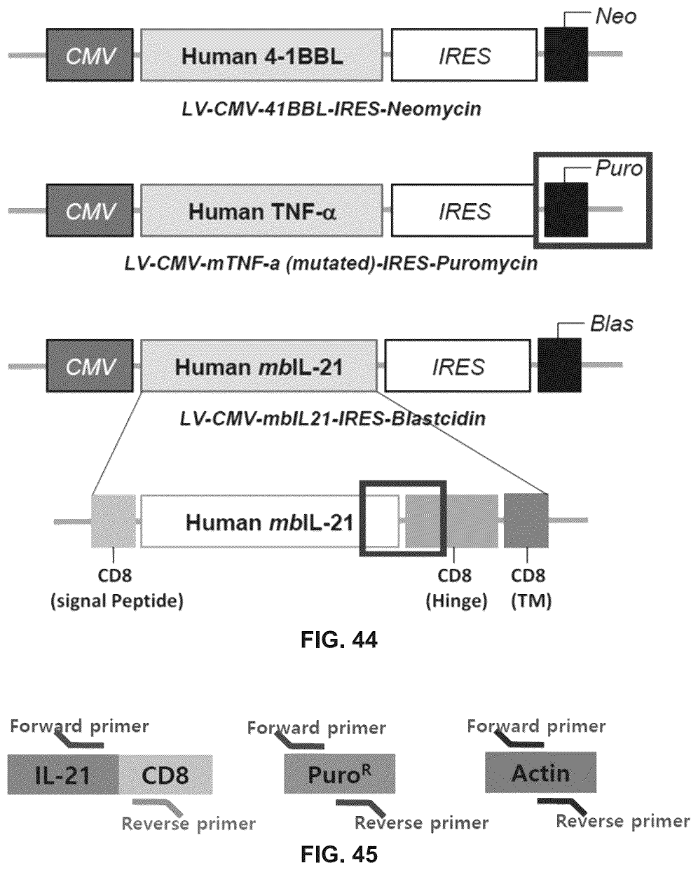

In one example, eHuT-78P cellular impurities in AB-101 drug product were measured by qPCR in cell populations by measuring expression level of genomic fragments derived from eHuT-78P (IL21-CD8 and Puro (SEQ ID NO: 31)) cells (FIG. 44). While these markers may be detected in the final drug product, it is preferable that they not exceed 0.2000% in the final drug product, e.g., with % residual eHuT 78 measured as set forth below.

A standard curve is generated using a series of NK cell samples spiked with different amounts of eHuT-78P cells. To prepare the standards, 2×10⁶ NK cells were combined with 0, 60, 200, 600, 2000, 6000, 20000 eHuT-78P cells and the genomic DNA was extracted as described herein. qPCR was conducted and the data was analyzed to obtain value of relative gene expression (2^{-ΔCT}), with actin expression serving as a control.

### Genomic DNA Extraction

200 μL of buffer T1 was added into a tube containing the cells, and to lyse the cells, 25 μL of proteinase K solution and 200 μL of buffer B3 was then added to the tube and mixed for 10 seconds using a vortex mixer. The tube was centrifuged at 1200 rpm at room temperature for 10 seconds and incubated in Eppendorf Thermo Mixer® C at 70° C., 300 rpm for 10-15 min. 210 μL of 100% Ethanol was added and mixed thoroughly for at least 15 seconds with a vortex mixer. The prepared sample was mounted to the Nucleo Spin® Tissue Column (hereinafter column) in the New Collection tube, and centrifuged in a high-performance centrifuge (4° C., 13000 rpm, 1 min). The solution that has been centrifuged into the collection tube was discarded, and the sample was put back on the column. Lysed proteins and RNA from cells, salt and buffer B5 remaining in the column, were all completely removed and the extracted DNA was collected in a 1.5 mL tube after centrifugation at 13000 rpm at 4° C. for 1 minute.

### QPCR Preparation and Result Analysis

Primers and probes for each gene were prepared (FIG. 45; Table 41).

TABLE 41
Primers and Probes for eHut 78 detection

| Name/SEQ ID NO: | Sequence (5′→3′) |
|---|---|
| SEQ ID NO: 1 | /56-FAM/TCGACATCG/ZEN/GCAAGGTGTGGGT/3IABKFQ/ |
| Puromycin resistance | |
| gene probe | |
| SEQ ID NO: 2 | GTCACCGAGCTGCAAGAA |
| Puromycin resistance | |
| gene primer 1 | |
| SEQ ID NO: 3 | CCGATCTCGGCGAACAC |
| Puromycin resistance | |
| gene primer 2 | |
| SEQ ID NO: 4 | /56-FAM/TCCTCGCTG/ZEN/CCGTGGGTCCG/3IABKFQ/ |
| IL21-CD8 probe | |
| SEQ ID NO: 5 | AATGATCCACCAGCACCTGA |
| IL21-CD8 primer 1 | |
| SEQ ID NO: 6 | ATGCTTCAGGCCTCAGTGAC |
| IL21-CD8 primer 2 | |
| SEQ ID NO: 7 | /56-FAM/ACCAACTGG/ZEN/GACGACATGGAGAAA/3IABKFQ/ |
| Actin probe | |
| SEQ ID NO: 8 | AGGCCCAGAGCAAGAGA |
| Actin primer 1 | |
| SEQ ID NO: 9 | GCTCATTGTAGAAGGTGTGGT |
| Actin primer 2 | |

The synthesized pre-mixed primer was stored at room temperature until use in a state in which exposure to light is blocked. A PCR mixture was prepared for each target gene on a MicroAmp® Optical 96-Well Reaction Plate, wherein a minimum of three repetitions for each sample was performed. The samples were loaded by inserting the MicroAmp® Optical 96-well reaction plate into a splash-free 96-well base in order to prevent foreign substances from sticking to the lower part of the plate, and 16 μL of each triplicate was dispensed with a 20P pipette into each well.

Using the Ct Mean value for Puromycin resistance gene, IL21-CD8, and Actin from the results, the ΔCt value for each target was obtained as shown below:

ΔCt=Ct Mean of target gene−Ct Mean of Actin

The relative expression of each target gene was calculated using the formula below:

Relative expression (Y)=2^{-(ΔCt)}×10⁴

The standard curve was created based on relative gene expression of standards (Table 42). Relative gene expression of AB-101 DP was applied to the standard curve to calculate the number of residual eHuT-78P. Calculated number of eHuT-78P indicates number of residual eHuT-78P per 1×10⁶ of AB-101 DP.

$$\% of residual eHut - 78 P = \frac{\# residual eHut - 78 P cells}{( 1 \times {10}^{6} )} \times 100$$

eHuT-78 free PB-NK showed now amplification of puro^{r} and mbIL21-CD8 sequences.

The number of residual eHuT 78 per 10⁶ cells of two different AB-101 drug product samples detected by this assay was 171.769 and 121.710, respectively, as detected by IL-21-CD8 and 214.221 and 141.040, respectively, as detected by Puro. This translates to a % residual eHuT 78 in the AB-101 samples of 0.01718 and 0.01217, respectively, as measured by IL-21-CD8, and of 0.02142 and 0.01410, respectively, as measured by Puro.

TABLE 42
Residual eHuT-78 qPCR detection assay

| | | | | | | | relative | | relative | |
|---|---|---|---|---|---|---|---|---|---|---|
| | | Ct | | | ACT | | expression | | expression *10⁴ | |
| | | | mbIL21- | | | mbIL20- | | mbIL21- | | mbIL21- |
| Template | | Puro | CD8 | Actin | Paro | CD8 | Puro | CD8 | Puro | CD8 |
| eHUT-78 | | 23.7343 | 24.2317 | 21.1737 | 2.5606 | 3.0581 | 0.1695013 | 0.1200663 | 1695.0134 | 1200.6632 |
| eHuT-78 # | 0 | 0 | 0 | 19.6661 | 0 | 0 | 0 | 0 | 0 | 0 |
| per IM of | 30 | 36.7706 | 36.3399 | 19.6614 | 17.1092 | 16.6785 | 0.0000071 | 0.0000095 | 0.0707 | 0.0953 |
| PB-NK | 100 | 35.180 | 34.6223 | 19.6026 | 15.4154 | 15.0197 | 0.0000229 | 0.0000301 | 0.2288 | 0.3010 |
| | 300 | 33.2721 | 33.5840 | 19.5269 | 13.7452 | 14.0571 | 0.0000728 | 0.0000587 | 0.7283 | 0.5867 |
| | 1K | 32.2611 | 32.4771 | 20.2242 | 12.0368 | 12.2529 | 0.0002380 | 0.0002049 | 2.3798 | 2.0489 |
| | 3K | 29.9459 | 30.2502 | 19.3906 | 10.5553 | 10.5896 | 0.0006646 | 0.0005382 | 6.6458 | 5.3818 |
| | 10K | 28.5420 | 288.9698 | 19.8661 | 8.6759 | 9.1037 | 0.0024451 | 0.0018177 | 24.4512 | 18.1769 |

| | | | | | | | | | |
|---|---|---|---|---|---|---|---|---|---|
| AB101 | 34.0023 | 34.5775 | 20.4972 | 13.5050 | 14.0803 | 0.0000860 | 0.0000577 | 0.8603 | 0.5773 |

SEQUENCES

| SEQ ID NO: and | |
|---|---|
| DESCRIPTION | SEQUENCE |
| SEQ ID NO: 10 | MEYASDASLDPEAPWPPAPRARACRVLPWALVAGLLLLLLLAAACAVE |
| Sequence of 4- | LACPWAVSGARASPGSAASPRLREGPELSPDDPAGLLDLRQGMFAQLV |
| 1BBL that can be | AQNVLLIDGPLSWYSDPGLAGVSLTGGLSYKEDTKELVVAKAGVYYVE |
| expressed by feeder | FQLELRRVVAGEGSGSVSLALHLQPLRSAAGAAALALTVDLPPASSEA |
| cells | RNSAFGFQGRLLHLSAGQRLGVHLHTEARARHAWQLTQGATVLGLFRV |
| | TPEIPAGLPSPRSE |
| SEQ ID NO: 11 | MALPVTALLLPLALLLHAARPQDRHMIRMRQLIDIVDQLKNYVNDLVP |
| Sequence of a | EFLPAPEDVETNCEWSAFSCFQKAQLKSANTGNNERIINVSIKKLKRK |
| membrane bound | PPSTNAGRRQKHRLTCPSCDSYEKKPPKEFLEREKSLLQKMIHQHLSS |
| IL-21(mbIL-21) | RTHGSEDSAKPTTTPAPRPPTPAPTIASQPLSLRPEACRPAAGGAVHT |
| that can be | RGLDFACDIYIWAPLAGTCGVLLLSLVITLY |
| expressed by feeder | |
| cells | |
| SEQ ID NO: 12 | MSTESMIRDVELAEEALPKKTGGPQGSRRCLFLSLFSFLIVAGATTLF |
| Sequence of a | CLLHFGVIGPQREEFPRDLSLISPLAQPVRSSSRTPSDKPVAHVVANP |
| mutated TNF alpha | QAEGQLQWLNRRANALLANGVELRDNQLVVPSEGLYLIYSQVLEKGQG |
| (mTNF-a) that can | CPSTHVLLTHTISRIAVSYQTKVNLLSAIKSPCQRETPEGAEAKPWYE |
| be expressed by | PIYLGGVFQLEKGDRLSAEINRPDYLDFAESGQVYFGIIAL |
| feeder cells | |
| SEQ ID NO: 13 | MERVQPLEENVGNAARPRFERNKLLLVASVIQGLGLLLCFTYICLHES |
| Sequence of | ALQVSHRYPRIQSIKVQFTEYKKEKGFILTSQKEDEIMKVQNNSVIIN |
| OX40L that can be | CDGFYLISLKGYFSQEVNISLHYQKDEEPLFQLKKVRSVNSLMVASLT |
| expressed by feeder | YKDKVYLNVTTDNTSLDDFHVNGGELILIHONPGEFCVL |
| cells | |
| SEQ ID NO: 14 | RSKRSRLLHSDYMNMTPRRPGPTRKHYQPYAPPRDFAAYRS |
| CD28 intracellular | |
| signaling domain | |
| SEQ ID NO: 15 | AGGAGTAAGAGGAGCAGGCTCCTGCACAGTGACTACATGAACATGACT |
| CD28 intracellular | CCCCGCCGCCCCGGGCCCACCCGCAAGCATTACCAGCCCTATGCCCCA |
| signaling domain | CCACGCGACTTCGCAGCCTATCGCTCC |
| SEQ ID NO: 16 | CGGAGCAAGAGGTCCCGCCTGCTGCACAGCGACTATATGAACATGACC |
| Codon Optimized | CCACGGAGACCCGGCCCTACACGGAAACATTACCAGCCCTATGCTCCA |
| CD28 intracellular | CCCCGGGACTTCGCAGCTTACAGAAGT |
| signaling domain | |
| SEQ ID NO: 17 | ERVQPLEENVGNAARPRFERNK |
| OX40L | |
| intracellular | |
| signaling domain | |
| SEQ ID NO: 18 | GAAAGGGTCCAACCCCTGGAAGAGAATGTGGGAAATGCAGCCAGGCCA |
| OX40L | AGATTCGAGAGGAACAAG |
| intracellular | |
| signaling domain | |
| SEQ ID NO: 19 | GAAAGAGTGCAGCCCCTGGAAGAGAATGTCGGGAATGCCGCTCGCCCA |
| Codon optimized | AGATTTGAAAGGAACAAA |
| OX40L | |
| intracellular | |
| signaling domain | |
| SEQ ID NO: 20 | RVKFSRSADAPAYQQGQNQLYNELNLGRREEYDVLDKRRGRDPEMGGK |
| CD3ζ signaling | PRRKNPQEGLYNELQKDKMAEAYSEIGMKGERRRGKGHDGLYQGLSTA |
| domain | TKDTYDALHMQALPPR |
| SEQ ID NO: 21 | AGAGTGAAGTTCAGCAGGAGCGCAGACGCCCCCGCGIACCAGCAGGGC |
| CD3ζ signaling | CAGAACCAGCTCTATAACGAGCTCAATCTAGGACGAAGAGAGGAGTAC |
| domain | GATGTTTTGGACAAGAGACGTGGCCGGGACCCTGAGATGGGGGGAAAG |
| | CCGAGAAGGAAGAACCCTCAGGAAGGCCTGTACAATGAACTGCAGAAA |
| | GATAAGATGGCGGAGGCCTACAGTGAGATTGGGATGAAAGGCGAGCGC |
| | CGGAGGGGCAAGGGGCACGATGGCCTTTACCAGGGTCTCAGTACAGCC |
| | ACCAAGGACACCTACGACGCCCTTCACATGCAGGCCCTGCCCCCTCGC |
| SEQ ID NO: 22 | CGAGTGAAGTTCAGCAGGTCCGCCGACGCTCCTGCATACCAGCAGGGA |
| Codon optimized | CAGAACCAGCTGTATAACGAGCTGAATCTGGGCCGGAGAGAGGAATAC |
| CD3ζ signaling | GACGTGCTGGACAAAAGGCGGGGCCGGGACCCCGAAATGGGAGGGAAG |
| domain | CCACGACGGAAAAACCCCCAGGAGGGCCTGTACAATGAGCTGCAAAAG |
| | GACAAAATGGCCGAGGCTTATTCTGAAATCGGGATGAAGGGAGAGAGA |
| | AGGCGCGGAAAAGGCCACGATGGCCTGTACCAGGGGCTGAGCACCGCT |
| | ACAAAGGACACCTATGATGCACTGCACATGCAGGCCCTGCCCCCTCGG |
| SEQ ID NO: 23 | GSGEGRGSLLTCGDVEENPGP |
| T2A cleavage site | |
| SEQ ID NO: 24 | GGCTCAGGTGAGGGGCGCGGGAGCCTGCTGACTTGTGGGGATGTAGAG |
| T2A cleavage site | GAAAATCCTGGTCCT |
| SEQ ID NO: 25 | MRISKPHLRSISIQCYLCLLLNSHELTEAGIHVFILGCFSAGLPKTEA |
| IL-15 | NWVNVISDLKKIEDLIQSMHIDATLYTESDVHPSCKVTAMKCFLLELQ |
| | VISLESGDASIHDTVENLIILANNSLSSNGNVTESGCKECEELEEKNI |
| | KEFLQSFVHIVQMFINTS- |
| SEQ ID NO: 26 | ATGAGAATCAGCAAACCACACCTCCGGAGCATATCAATCCAGTGTTAC |
| IL-15 | TTGTGCCTTCTTTTGAACTCCCATTTCCTCACCGAGGCAGGCATTCAT |
| | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| SEQ ID NO: 27 | ATGCGCATAAGTAAGCCTCATCTGCGGTCCATTTCTATACAATGTTAT |
| IL-15 | CTGTGCTTGCTTTTGAACTCCCACTTTCTTACGGAAGCAGGCATTCAT |
| | GTGTTCATTCTGGGTTGTTTTTCtGCCGGGCTGCCCAAAACCGAGGCC |
| | AACTGGGTCAACGTGATCAGCGACCTCAAGAAGATCGAGGATTTGATT |
| | CAAAGTATGCATATAGACGCCACACTCTATACTGAGTCCGACGTTCAC |
| | CCGAGTTGTAAAGTTACGGCTATGAAGTGCTTTTTGTTGGAACTCCAG |
| | GTGATTTCCCTTGAATCCGGCGATGCGAGCATCCACGATACGGTAGAG |
| | AATCTTATTATTCTGGCGAATAATTCTCTGTCTTCAAATGGGAATGTA |
| | ACTGAGAGCGGTTGTAAAGAATGCGAAGAACTTGAAGAAAAGAATATC |
| | AAGGAATTTCTTCAGAGTTTCGTGCATATTGTTCAAATGTTCATCAAC |
| | ACATCCTGA |
| SEQ ID NO: 28 | RSKRSRLLHSDYMNMTPRRPGPTRKHYQPYAPPRDFAAYRSERVQPLE |
| CD28/0X40L/CDζ | ENVGNAARPRFERNKRVKFSRSADAPAYQQGQNQLYNELNLGRREEYD |
| | VLDKRRGRDPEMGGKPRRKNPQEGLYNELQKDKMAEAYSEIGMKGERR |
| | RGKGHDGLYQGLSTATKDTYDALHMQALPPR |
| SEQ ID NO: 29 | RSKRSRLLHSDYMNMTPRRPGPTRKHYQPYAPPRDFAAYRSERVQPLE |
| CD28/OX40L/CDζ/ | ENVGNAARPRFERNKRVKFSRSADAPAYQQGQNQLYNELNLGRREEYD |
| T2A/IL1-5 | VLDKRRGRDPEMGGKPRRKNPQEGLYNELQKDKMAEAYSEIGMKGERR |
| | RGKGHDGLYQGLSTATKDTYDALHMQALPPRGSGEGRGSLLTCGDVEE |
| | NPGPMRISKPHLRSISIQCYLCLLLNSHFLTEAGIHVFILGCFSAGLP |
| | KTEANWVNVISDLKKIEDLIQSMHIDATLYTESDVHPSCKVTAMKCEL |
| | LELQVISLESGDASIHDTVENLIILANNSLSSNGNVTESGCKECEELE |
| | EKNIKEFLQSFVHIVQMFINTS- |
| SEQ ID NO: 30 | MKWVTFISLLFLESSAYSRGVERRDAHKSEVAHRFKDLGEENFKALVL |
| Human Albumin | IAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEV |
| | DVMCTAFHDNEETFLKKYLYEIARRHPYFYAPELLFFAKRYKAAFTEC |
| | CQAADKAACLLPKLDELRDEGKASSAKQRLKCASLQKFGERAFKAWAV |
| | ARLSQRFPKAEFAEVSKLVTDLTKVHTECCHGDLLECADDRADLAKYI |
| | CENQDSISSKLKECCEKPLLEKSHCIAEVENDEMPADLPSLAADFVES |
| | KDVCKNYAEAKDVFLGMFLYEYARRHPDYSVVLLLRLAKTYETTLEKC |
| | CAAADPHECYAKVEDEFKPLVEEPQNLIKQNCELFEQLGEYKFQNALL |
| | VRYTKKVPQVSTPTLVEVSRNLGKVGSKCCKHPEAKRMPCAEDYLSVV |
| | LNQLCVLHEKTPVSDRVTKCCTESLVNRRPCFSALEVDETYVPKEFNA |
| | ETFTFHADICTLSEKERQIKKQTALVELVKHKPKATKEQLKAVMDDFA |
| | AFVEKCCKADDKETCFAEEGKKLVAASQAALGL |
| SEQ ID NO: 31 | ATGGCCACCGAGTACAAGCCCACGGTGCGCCTCGCCACCCGCGACGAC |
| Puromycin | GTCCCCCGGGCCGTACGCACCCTCGCCGCCGCGTTCGCCGACTACCCC |
| Resistance Gene | GCCACGCGCCACACCGTCGATCCGGACCGCCACATCGAGCGGGTCACC |
| | GAGCTGCAAGAACTCTTCCTCACGCGCGTCGGGCTCGACATCGGCAAG |
| | GTGTGGGTCGCGGACGACGGCGCCGCGGTGGCGGTCTGGACCACGCCG |
| | GAGAGCGTCGAAGCGGGGGCGGTGTTCGCCGAGATCGGCCCGCGCATG |
| | GCCGAGTTGAGCGGTTCCCGGCTGGCCGCGCAGCAACAGATGGAAGGC |
| | CTCCTGGCGCCGCACCGGCCCAAGGAGCCCGCGTGGTTCCTGGCCACC |
| | GTCGGCGTCTCGCCCGACCACCAGGGCAAGGGTCTGGGCAGCGCCGTC |
| | GTGCTCCCCGGAGTGGAGGCGGCCGAGCGCGCCGGGGTGCCCGCCTTC |
| | CTGGAGACCTCCGCGCCCCGCAACCTCCCCTTCTACGAGCGGCTCGGC |
| | TTCACCGTCACCGCCGACGTCGAGGTGCCCGAAGGACCGCGCACCTGG |
| | TGCATGACCCGCAAGCCCGGTGCCTGA |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A population of expanded natural killer cells comprising a KIR-B haplotype and homozygous for a CD16 158V polymorphism.

2. The population of expanded natural killer cells of claim 1, wherein the expanded natural killer cells are expanded umbilical cord blood natural killer cells.

3. The population of expanded natural killer cells of claim 1 or claim 2, comprising at least 60%, e.g., at least 70%, at least 80%, at least 90% at least 95%, at least 99%, or 100% CD16+ cells.

4. The population of expanded natural killer cells of any one of claims 1-3, comprising at least 60%, e.g., at least 70%, at least 80%, at least 90% at least 95%, at least 99%, or 100% NKG2D+ cells.

5. The population of expanded natural killer cells of any one of claims 1-4, comprising at least 60%, e.g., at least 70%, at least 80%, at least 90% at least 95%, at least 99%, or 100% NKp46+ cells.

6. The population of expanded natural killer cells of any one of claims 1-5, comprising at least 60%, e.g., at least 70%, at least 80%, at least 90% at least 95%, at least 99%, or 100% NKp30+ cells.

7. The population of expanded natural killer cells of any one of claims 1-6, comprising at least 60%, e.g., at least 70%, at least 80%, at least 90% at least 95%, at least 99%, or 100% DNAM-1+ cells.

8. The population of expanded natural killer cells of any one of claims 1-7, comprising at least 60%, e.g., at least 70%, at least 80%, at least 90% at least 95%, at least 99%, or 100% NKp44+ cells.

9. The population of expanded natural killer cells of any one of claims 1-8, comprising less than 20%, e.g., 10% or less, 5% or less, 1% or less, 0.5% or less, or 0% CD3+ cells.

10. The population of expanded natural killer cells of any one of claims 1-9, comprising less than 20% or less, e.g., 10% or less, 5% or less, 1% or less, 0.5% or less, or 0% CD14+ cells.

11. The population of expanded natural killer cells of any one of claims 1-10, comprising less than 20% or less, e.g., 10% or less, 5% or less, 1% or less, 0.5% or less, or 0% CD19+ cells.

12. The population of expanded natural killer cells of any one of claims 1-11, comprising less than 20% or less, e.g., 10% or less, 5% or less, 1% or less, 0.5% or less, or 0% CD38+ cells.

13. The population of expanded natural killer cells of any one of claims 1-12, wherein the expanded natural killer cells do not comprise a CD16 transgene.

14. The population of expanded natural killer cells of any one of claims 1-12, wherein the expanded natural killer cells do not express an exogenous CD16 protein.

15. The population of expanded natural killer cells of any one of claims 1-12, wherein the expanded natural killer cells are not genetically engineered.

16. The population of expanded natural killer cells of any one of claims 1-15, wherein the expanded natural killer cells are derived from the same umbilical cord blood donor.

17. The population of expanded natural killer cells of any one of claims 1-16, wherein the population comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

18. The population of expanded natural killer cells of any one of claims 1-17, wherein the population is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce expanded natural killer cells,
thereby producing the population of expanded natural killer cells.

19. The population of expanded natural killer cells of any one of claims 1-17, wherein the population is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and
(d) expanding the master cell bank population of expanded natural killer cells by culturing with a second plurality of Hut78 cells engineered to express a membrane bound IL-21, a mutated TNFα, and a 4-1BBL gene to produce expanded natural killer cells;
thereby producing the population of expanded natural killer cells.

20. The population of expanded natural killer cells of claim 19, wherein the method further comprises, after step (c),
(i) freezing the master cell bank population of expanded natural killer cells in a plurality of containers; and
(ii) thawing a container comprising an aliquot of the master cell bank population of expanded natural killer cells,
wherein expanding the master cell bank population of expanded natural killer cells in step (d) comprises expanding the aliquot of the master cell bank population of expanded natural killer cells.

21. The population of expanded natural killer cells of any one of claims 18-20, wherein the umbilical cord blood is from a donor with the KIR-B haplotype and homozygous for the CD16 158V polymorphism.

22. The population of expanded natural killer cells of any one of claims 18-21, wherein the method comprises expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold.

23. The population of expanded natural killer cells of any one of claims 18-22, wherein the population of expanded natural killer cells is not enriched or sorted after expansion.

24. The population of expanded natural killer cells of any one of claims 18-23, wherein the percentage of NK cells expressing CD16 in the population of expanded natural killer cells is the same or higher than the percentage of natural killer cells in the seed cells from umbilical cord blood.

25. The population of expanded natural killer cells of any one of claims 18-24, wherein the percentage of NK cells expressing NKG2D in the population of expanded natural killer cells is the same or higher than the percentage of natural killer cells in the seed cells from umbilical cord blood.

26. The population of expanded natural killer cells of any one of claims 18-25, wherein the percentage of NK cells expressing NKp30 in the population of expanded natural killer cells is the same or higher than the percentage of natural killer cells in the seed cells from umbilical cord blood.

27. The population of expanded natural killer cells of any one of claims 18-26, wherein the percentage of NK cells expressing NKp44 in the population of expanded natural killer cells is the same or higher than the percentage of natural killer cells in the seed cells from umbilical cord blood.

28. The population of expanded natural killer cells of any one of claims 18-27, wherein the percentage of NK cells expressing NKp46 in the population of expanded natural killer cells is the same or higher than the percentage of natural killer cells in the seed cells from umbilical cord blood.

29. The population of expanded natural killer cells of any one of claims 18-28, wherein the percentage of NK cells expressing DNAM-1 in the population of expanded natural killer cells is the same or higher than the percentage of natural killer cells in the seed cells from umbilical cord blood.

30. A vial or cryobag comprising a portion of the population of expanded natural killer cells of any one of claims 1-29.

31. A plurality of vials or cryobags comprising portions of the population of expanded natural killer cells of any one of claims 1-29.

32. The plurality of vials or cryobags of claim 31, comprising at least 10 vials or cryobags comprising portions of the population of expanded natural killer cells, e.g., 20, 30, 40, 50, 60, 70, 80, 90, 100, 125, 150, 175, 200, 250, 300, 400, 500, 600, 700, 800, 900, 1000, 1100, or 1200 vials or cryobags.

33. A bioreactor comprising the population of expanded natural killer cells of any one of claims 1-23 or a portion thereof.

34. A composition comprising
the population of expanded and stimulated natural killer cells of any one of claims 1-33; and
a cryopreservation solution.

35. The composition of claim 34, wherein the cryopreservation solution comprises
(a) human albumin;
(b) dextran;
(c) glucose;
(d) DMSO; and
(e) a buffer.

36. The composition of claim 35 comprising from 30 to 50 mg/mL human albumin.

37. The composition of claim 35 comprising 50 mg/mL human albumin.

38. The composition of any one of claims 35-37 comprising 20 to 30 mg/mL dextran.

39. The composition of any one of claims 35-38 comprising 25 mg/mL dextran.

40. The composition of any one of claims 35-39, wherein the dextran is Dextran 40.

41. The composition of any one of claims 35-40 comprising from 12 to 15 mg/mL glucose.

42. The composition of any one of claims 35-41 comprising 12.5 mg/mL glucose.

43. The composition of any one of claims 35-42 comprising less than 27.5 g/L glucose.

44. The composition of any one of claims 35-43 comprising from 50 to 60 ml/mL DMSO.

45. The composition of any one of claims 35-44 comprising 55 mg/mL DMSO.

46. The composition of any one of claims 35-45 comprising 40 to 60% v/v buffer.

47. The composition of any one of claims 35-46, wherein the buffer is phosphate buffered saline.

48. The composition of 35 comprising:
(a) about 40 mg/mL human albumin;
(b) about 25 mg/mL Dextran 40;
(c) about 12.5 mg/mL glucose;
(d) about 55 mg/mL DMSO; and
(e) about 0.5 mL/mL phosphate buffered saline.

49. The composition of any one of claims 34-48, further comprising 0.5 mL/mL water.

50. The composition of any one of claims 34-49, wherein the cryopreservation solution is an infusion-ready cryopreservation solution.

51. The composition of any one of claims 34-49, further comprising at least one of genetic material, protein, or cells from a feeder cell line.

52. The composition of claim 50, wherein the genetic material from the feeder cell line comprises a nucleic acid encoding a membrane bound IL-21 molecule or a portion thereof.

53. The composition of claim 52, wherein the membrane bound IL-21 comprises a CD8 transmembrane domain.

54. The composition of any one of claims 52-53, wherein the genetic material from the feeder cell line that comprises a nucleic acid encoding a membrane bound IL-21 molecule or a portion thereof encodes SEQ ID NO: 11 or a portion thereof.

55. The composition of any one of claims 50-54, wherein the genetic material from the feeder cell line comprises a nucleic acid encoding a mutated TNFα molecule or a portion thereof.

56. The composition of claim 55, wherein the genetic material from the feeder cell line that comprises a nucleic acid encoding a mutated TNFα molecule or a portion thereof encodes SEQ ID NO: 12 or a portion thereof.

57. The composition of any one of claims 50-56, wherein the protein from the feeder cell line comprises a membrane bound IL-21 polypeptide or a portion thereof.

58. The composition of claim 57, wherein the membrane bound IL-21 comprises a CD8 transmembrane domain.

59. The composition of any one of claims 57-58, wherein the protein from the feeder cell line that comprises a membrane bound IL-21 polypeptide or a portion thereof comprises SEQ ID NO: 11 or a portion thereof.

60. The composition of any one of claims 50-59, wherein the protein from the feeder cell line comprises a mutated TNFα polypeptide or a portion thereof.

61. The composition of claim 60, wherein the protein from the feeder cell line that comprises a a mutated TNFα polypeptide or a portion thereof comprises SEQ ID NO: 12 or a portion thereof.

62. The composition of any one of claims 50-61, wherein the cells from the feeder cell line are CD4+ T cells.

63. The composition of claim 62, wherein the cells from the feeder cell line are Hut78 cells.

64. The composition of claim 63, wherein the cells from the Hut78 cells are engineered Hut78 (eHut78) cells express 4-1BBL, membrane bound IL-21 and mutant TNFα.

65. The composition of any one of claims 62-64, wherein the cells from the feeder cell line comprise live cells.

66. The composition of any one of claims 62-65, wherein the cells from the feeder cell line comprise dead cells.

67. The composition of any one of claims 34-66, wherein the composition is frozen.

68. The composition of claim 67, wherein the pharmaceutical composition has been frozen for at least three months, e.g., at least six months, at least nine months, at least 12 months, at least 15 months, at least 18 months, at least 24 months, or at least 36 months.

69. The composition of claim 67 or claim 68, wherein the population of expanded natural killer cells exhibits at least 60%, e.g., at least 70%, at least 80%, at least 90% at least 95%, at least 99%, or 100% viability after it is thawed.

70. A pharmaceutical composition comprising the composition of any one of claims 34-69.

71. A dosage unit comprising the pharmaceutical composition of claim 70.

72. The dosage unit of claim 71 comprising between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

73. A composition comprising a population of expanded cord blood-derived natural killer cells comprising a KIR-B haplotype and homozygous for a CD16 158V polymorphism and a plurality of engineered HuT78 cells.
